(19) United States
(12) Patent Application Publication
(10) Pub. No.: US 2026/0325680 A1
(43) Pub. Date: Oct. 22, 2026

(54) METHODS AND COMPOSITIONS FOR HYDRODYNAMIC GENE DELIVERY

(71) Applicant: HYDROGENE THERAPEUTICS, INC., Houston, TX (US)

(72) Inventor: Robert KRUSE, Houston, TX (US)

(73) Assignee: HYDROGENE THERAPEUTICS, INC., Houston, TX (US)

(21) Appl. No.: 19/107,565

(22) PCT Filed: Aug. 31, 2023
(86) PCT No.: PCT/US2023/031751

§ 371 Date: Feb. 27, 2025

Related U.S. Application Data
(60) Provisional application No. 63/374,058, filed on Aug. 31, 2022.
(60) Provisional application No. 63/374,070, filed on Aug. 31, 2022.
(60) Provisional application No. 63/374,073, filed on Aug. 31, 2022.
(60) Provisional application No. 63/374,216, filed on Aug. 31, 2022.
(60) Provisional application No. 63/374,228, filed on Aug. 31, 2022.
(60) Provisional application No. 63/374,231, filed on Aug. 31, 2022.
(60) Provisional application No. 63/374,234, filed on Aug. 31, 2022.

(51) Int. Cl. A61K 48/00; A61M 5/36

(52) U.S. Cl. CPC A61K 48/0058; A61M 5/36

(57) ABSTRACT

The present disclosure relates to methods of pressure-guided hydrodynamic injection, into tissues such as kidney, pancreas, liver, common bile duct, etc., in certain embodiments for delivery of nucleic acids or viral vectors. This includes optimal pressures that mediate gene delivery and expression into the subject during such hydrodynamic injection. Methods of disentangling individual differences to achieve target pressure during the hydrodynamic procedure are described, while also avoiding tissue injury, ruptures, tears, decreasing the toxicity of hydrodynamic injection, and other considerations relevant to therapeutic delivery.

### CROSS-REFERENCE TO RELATED APPLICATIONS

The present application is related to and claims priority under 35 U.S.C. § 119 (e) to U.S. Provisional Patent Application Nos. 63/374,228, 63/374,231, 63/374,234, 63/374,058, 63/374,070, 63/374,073 and 63/374,216, filed on Aug. 31, 2022. The entire contents of the aforementioned patent applications are incorporated herein by this reference.

### FIELD

The present disclosure relates to compositions and methods for hydrodynamic gene delivery.

### BACKGROUND

The technical problem concerns how to understand what parameters to inject into patients for gene delivery, considering that every patient may have differences in underlying disease status. Gene delivery into specific tissue types holds therapeutic promise.

The kidneys are organs of the body that play an important role in removing toxins from the body, as well as controlling the volume of various body fluids, osmolality, acid-base balance, electrolyte concentrations, etc. Disruption of kidney function leads to a wide variety of rare genetic kidney diseases such as, cystinuria, adult polycystic kidney disease, nephrogenic diabetes insipidus, Gitelman syndrome, Fabry's disease, thin basement membrane disease, Lowe syndrome, hereditary interstitial kidnet disease, tuberous sclerosis, nepthronophthisis, Alport disease. Beyond rare kidney disease, common disorders like chronic kidney disease, end-stage renal disease, and immune-mediated glomerular disorders are also very important. Kidney disease (aka renal disease) generally presents as damage to a kidney. For example, nephritis is an inflammatory kidney disease that can be divided into several categories depending upon the location of the inflammation. Another example is nephrosis, which is a non-inflammatory kidney disease that gives rise to either nephritic or nephrotic syndrome. Kidney disease usually causes a loss of kidney function. Complete loss of kidney function is known as end-stage of kidney disease and can only be treated by dialysis or a kidney transplant. A gene therapy approach could be used in the treatment algorithm of autoimmune, inflammatory, metabolic, toxic, pre-neoplastic, or cancer of the kidney.

One strategy to treat disorders of the kidney is gene therapy, which could deliver genes that are found to be either missing or present at insufficient levels in kidney cells. Additionally, some clinical preventative strategies or disorders may benefit from the addition of genes as a therapeutic approach. However, no gene therapy strategies for treating or preventing genetic disease of the kidney have been successfully implemented to date. Accordingly, there is an urgent need for gene therapy protocols that can be implemented in the kidney to treat and/or prevent genetic, autoimmune, inflammatory, fibrotic, metabolic, toxic, pre-neoplastic, or malignant disease of the kidney.

The pancreas is an important exocrine and endocrine organ of the body that plays a role in food digestion and energy storage. Disruption of pancreatic function leads to a wide variety of disorders such as, for example Cystic fibrosis, Hereditary pancreatitis, Autoimmune pancreatitis, and multiple different types of diabetes, including but not limited to Types I and II, Maturity onset diabetes of the young (MODY), Latent autoimmune diabetes in adults (LADA), Neonatal diabetes, Wolfram syndrome, Alstrom syndrome. For example, the pancreas plays an important role in the pathogenesis of a variety of different diseases, such as Type 1 and Type 2 diabetes, which are caused by a disruption or dysregulation of insulin production. One strategy to treat disorders of the pancreas is gene therapy, which could deliver genes that are found to be deficient in pancreatic cells, or by transforming pancreatic cells. Gene therapy could also be used to deliver proteins that could fight against genetic diseases, early neoplasia, cancer, pancreatic pain, pancreatic inflammation, or autoimmune diseases. Target cells of interest in the pancreas include target cells of interest are including but not limited to: pancreatic acinar cells, ductal cells, islet cells, endothelial cells, neuronal cells.

Various strategies have been attempted for gene therapy of the pancreas. For example, viral strategies have employed systemic or targeted therapy through vascular routes while other studies have shown the feasibility of injecting virus through the pancreatic duct. Disadvantageously, viral vectors such as AAV have significant size limitations (~4.8 kb) with respect to the size of the nucleotide sequence they can package. Non-viral strategies have also been employed by leveraging hydrodynamic injection through the pancreatic artery in rats. Unfortunately, these non-viral strategies have not been translated into large animal models. Accordingly, there is an urgent need for gene therapy protocols that can be implemented in the pancreas to treat and/or prevent this genetic disease of the pancreas.

The biliary system is a promising route for gene delivery into the liver. Hydrodynamic gene delivery can be accomplished through the biliary system with high efficiency achieved from the common hepatic duct. Unfortunately, delivery from other locations in the biliary system have not been successful. Accordingly, there is a need for additional routes of administration for hydrodynamic gene delivery.

Gene therapy is a therapeutic option to treat cancer. Gene therapy could deliver many different types of therapies directly into the tumor to facilitate its removal, but current delivery systems have failed at efficient delivery. Non-viral delivery is particularly limited with efficacy into tumors.

The liver is a major organ that performs many essential biological functions such as detoxification and synthesis of proteins and biochemicals. The liver also plays important roles in metabolism including the regulation of glycogen storage, decomposition of red blood cells, glucose and lipid metabolism, and the production of hormones. Disruption of liver function leads to a wide variety of rare genetic disorders such as Wilson's disease, phenylketonuria, hemophilia A, hemophilia B, progressive familial intrahepatic cholestasis, etc. Disruption of liver function also can lead to a variety of autoimmune disorders such autoimmune hepatitis, primary sclerosing cholangitis, primary biliary cirrhosis, etc. Furthermore, infiltrative diseases can affect the liver such as amyloidosis and malignancy such as hepatocellular carcinoma or metastatic colorectal cancer. Additionally, a variety of metabolic diseases can affect the liver such as non-alcoholic fatty liver disease and non-alcoholic steatohepatitis. There are many liver diseases that could be treated with gene therapy. These include: Hemophilia A, Hemophilia B, Alpha-1 antitrypsin deficiency, Wilson's disease, Hereditary Tyrosinemia, PFIC (progressive familial intrahepatic cholestasis), Heterdiatry hemochromatosis, Crigler Najjar, Familial hypercholesteremia, Acute Hepatic Porphyria, Acute Intemittent Porphyria, von Willenbrand's disease, Primary hyperoxaluria, Atypical HUS, Phenylketonuria, Maple syrup, Methylmalonic acidemia, Proprionic acidemia, NAGS deficiency, CPS I deficiency, OTC deficiency, Aginino-succinic aciduria, Agininemia, Citrullinemia, Fabry Disease, MPS, Pompe Disease, GSD1a, and Cystathionine β-Synthase Deficiency. To describe two examples in more depth, two common inherited liver diseases are hemochromatosis and alpha-1 antitrypsin deficiency.

Hemochromatosis is a disease in which deposits of iron collect in the liver and other organs. The primary form of this disease is one of the most common inherited diseases in the United States, and disease frequency within the U.S. population is estimated to be as high as 1 in every 200 people has the disease. Unfortunately, many people who carry the genetic risk factors for hemochromatosis are unaware that they are carriers.

Alpha-1 antitrypsin is an inherited liver disease that affects an important liver protein known as alpha-1 antitrypsin, which is either missing completely or present at greatly reduced levels in affected individuals. People with alpha-1 antitrypsin deficiency are often able to produce this protein, but the protein fails to enter the bloodstream and instead accumulates within the liver. The alpha-1 antitrypsin protein plays an important role in protecting the lungs from enzymatic damage. Additionally, people with this disease are at risk of developing cirrhosis.

Various strategies have been attempted for gene therapy of the liver. For example, viral strategies have employed systemic or targeted therapy through vascular routes while other studies have shown the feasibility of injecting virus through the bile duct. Disadvantageously, viral vectors such as AAV have significant size limitations (~4.8 kb) with respect to the size of the nucleotide sequence they can package. Non-viral strategies have also been employed by leveraging hydrodynamic injection through the bile duct in rats. Unfortunately, these non-viral strategies have not been translated effectively into large animal models.

One strategy to treat disorders of the liver is gene therapy, which could deliver genes that are found to be either missing or present at insufficient levels in liver cells. However, no gene therapy strategies for treating or preventing genetic disease of the liver have been successfully implemented to date. Accordingly, there is an urgent need for gene therapy protocols that can be implemented in the liver to treat and/or prevent this genetic based liver disease.

### SUMMARY

In certain aspects, the present disclosure provides methods of pressure-guided hydrodynamic injection. Such methods include optimal pressures that mediate gene delivery and expression into the human during biliary hydrodynamic injection. Methods of disentangling individual differences to achieve target pressure during the hydrodynamic procedure are described.

The present disclosure also relates to compositions and methods for treating kidney disease. More particularly, the present disclosure relates to compositions and methods for treating kidney disease by gene therapy. As described in detail below, the present disclosure is based, at least in part, on the surprising discovery that the ureter hydrodynamic injection could mediate rupture of the kidney cortex variety of different flow rates and volumes, such that only limited parameters were observed to be safe.

The present disclosure further relates to compositions and methods for treating diseases of the pancreas. More particularly, the present disclosure relates to compositions and methods for treating diseases of the pancreas by gene therapy. As described in detail below, the present disclosure is based, at least in part, on the surprising discovery that the volume of fluid required for sufficient gene expression is lower than 20 mL, as previously published to be sufficient.

The present disclosure also provides methods of hydrodynamic gene injection proceeding from the common bile duct. The methods herein improve upon previous strategies injecting through the common bile duct by providing optimal flow rates and pressures that mediate gene expression within hepatocytes of the liver.

The present disclosure additionally provides methods of delivery into tumors of the liver and pancreas. These methods encompass the use of hydrodynamic injection through the biliary tract as a method of gene delivery into tumor microenvironment. Methods regarding the procedure injection parameters and efficacious DNA doses into the tumor are disclosed.

The present disclosure further provides methods for mediating efficient gene delivery into the liver of primates. The methods consist of steps of executing hydrodynamic delivery into the biliary system of primate liver. Optimal injection parameters are described that mediate efficient gene delivery into primate liver while reducing side effects. Pressures that mediate gene delivery into the primate liver are also described, as well as dosing paradigms.

The present disclosure also relates to compositions and methods for hydrodynamic gene delivery into the liver through the biliary system. More particularly, the present disclosure relates to compositions and methods for increasing the efficiency of hydrodynamic gene delivery through the biliary system. As described in detail below, the present disclosure is based, at least in part, on the surprising discovery that modifying the DNA vector to substantially reduce the bacterial sequences on the plasmid DNA to less than 500 basepairs, substantially increased the transfected area of hepatocytes observed to over 70% of hepatocytes, an increase from under 50% with a regular plasmid.

In an aspect, the present disclosure provides a method of determining a flow rate for a hydrodynamic injection into an organ, including the steps of:
performing a test injection;
measuring a pressure during the test injection; and
evaluating a difference in stiffness and resistance empirically.

In exemplary embodiments, the organ is selected from a liver, pancreas, or kidney.

In exemplary embodiments, the liver is injected through a biliary system.

In exemplary embodiments, the pancreas is injected through a ductal system.

In exemplary embodiments, the kidney is injected through a ureter system.

In exemplary embodiments, a pressure sensor will be inserted into a catheter through a dedicated lumen.

In exemplary embodiments, the pressure sensor is already present in the catheter.

In exemplary embodiments, the pressure sensor is a transducer attached to the fluid filled lumen for pressure sensing.

In exemplary embodiments, the method further comprising the steps of: reading a baseline of the pressure in the biliary system conducted without the balloon inflated; performing a pressure measurement in the biliary system with balloon inflated; injecting a test solution or a nucleic acid into a duct or a vessel of interest that does not contain the plasmid DNA with the balloon inflated; and monitoring the pressure during the test solution injection

In exemplary embodiments, the test solution has the same osmolarity, osmolality, and viscosity as the DNA injection solution.

In exemplary embodiments, the test solution further does not contain any other active drug substances included in the therapeutic DNA solution.

In exemplary embodiments, the pressure achieved during hydrodynamic injection is at least 50 mmHg, at least 80 mmHg, or at least 120 mmHg.

In exemplary embodiments, the test flow rate is initially at least 2 mL/sec, or at least 3 mL/sec, or at least 4 mL/sec into the liver.

In exemplary embodiments, the total test volume is at most 15 mL, at most 10 mL, at most 7 mL, or at most 5 mL of volume.

In exemplary embodiments, the total test volume is sufficient to measure the column of total fluid resistance in the entire circuit and estimating whether sufficient pressure is achieved.

In exemplary embodiments, the flow rate is increased if the sufficient pressure is not achieved, repeating the test.

In exemplary embodiments, the flow rate is decreased if the pressure above 250 mmHg is achieved, repeating the test.

In exemplary embodiments, the flow rate is increased or decreased by 1 mL/sec or increased or decreased by 0.5 mL/sec for the second test.

In exemplary embodiments, the DNA solution injection proceeds at a programmed flow rate established that will yield the proper pressure.

In exemplary embodiments, a flow rate series is conducted during one single test injection, wherein multiple flow rates are tested and pressure measured throughout the single test injection.

In exemplary embodiments, at least two or more test flow rates are tested within a single test injection.

In exemplary embodiments, the pressure can be correlated to which flow rate yielded the increase.

In exemplary embodiments, the total injection volume is increased to at most 40 mL, 30 mL or 20 mL in total volume, testing all of the injection parameters.

In exemplary embodiments, the minimum pressure for efficient hydrodynamic gene delivery is greater than 50 mmHg, or greater than 80 mmHg, or greater than 100 mmHg.

In exemplary embodiments, the maximum pressure for efficient hydrodynamic gene delivery is less than 200 mmHg or less than 250 mmHg.

In an aspect, the present disclosure provides a method of hydrodynamic retrograde ureter delivery of nucleic acids or viral vectors, including the steps of:
(i) insertion of a cystoscope through the urethra and into the bladder,
(ii) insertion of a balloon catheter through the cystoscope and into the bladder,
(iii) canulation of the ureteral orifice with the balloon catheter,
(iv) inflation of the balloon catheter at the distal ureter near the ureter orifice entrance,
(v) use of power injector to mediate hydrodynamic injection into the kidney,
wherein the method does not require the use of fluoroscopy for catheter placement.

In exemplary embodiments, rupture or tears of the kidney are avoided by using injection parameters equal to or below 20 mL in total volume, and 2 ml/sec

In exemplary embodiments, flow rates equal to or between 0.5 mL/sec1 to 2 mL/sec are optimal for achieving gene delivery without rupture expression.

In exemplary embodiments, optimal volumes for injection are between 10 mL to 20 mL to mediate effective gene delivery without rupture.

In exemplary embodiments, the balloon is positioned in the muscular wall of bladder in the ureter and can be visualized with by the cystoscope camera without the need of fluoroscopy.

a. The method of claim 5, wherein the cystoscope camera can be monitored during the injection for any outflow from the ureter with the complete absence indicating effective for seal during injection.

In exemplary embodiments, the nucleic acids are minimum dosed at 1 mg, 2 mg, 3 mg, 4 mg, or higher in mass for an individual kidney in a subject 30 kg or greater.

In exemplary embodiments, the hydrodynamic parameters are sufficient to achieve protein expression within cells of the kidney after delivery of DNA:
a. The method of claim 7, wherein cell expression is achieved within cells of the glomerulus, tubule, or endothelium inside the kidney.
b. The method of claim 7, wherein differences in cell expression among the glomerulus, tubule, or endothelium correlate to the uses of different promoters in the plasmid DNA.

In an aspect, the disclosure provides a method of hydrodynamic retrograde ureter delivery of nucleic acids or viral vectors, including the steps of:
(i) insertion of a cystoscope through the urethra and into the bladder,
(ii) insertion of a guidewire through the cystoscope and into the bladder,
(iii) canulation of the ureteral orifice with the guidewire,
(iv) advancement of the guidewire toward the kidney,
(v) removal of cystoscope and exchange of balloon catheter over the guidewire,
(vi) inflation of balloon catheter within ureter at proximal location near kidney,
(vii) use of power injector to mediate hydrodynamic injection into the kidney,
wherein the method can utilize balloon catheters that do not fit the working channel of cystoscopes.

In exemplary embodiments, rupture or tears of the kidney are avoided by using injection parameters equal to or below 12 mL and 2 ml/sec

In exemplary embodiments, flow rates equal to or between 1 0.5 to 2 mL/sec are optimal for achieving gene delivery without rupture.

In exemplary embodiments, optimal volumes for injection are between 7 mL to 12 mL mediate effective gene delivery without rupture.

In exemplary embodiments, the balloon is positioned at least 1 cm, 2 cm, or 3 cm away from the renal pelvis into the ureter in order to assure proper seal.

In exemplary embodiments, radiocontrast injection is utilized to confirm positioning of the catheter and seal of the balloon prior to injection.

In exemplary embodiments, the nucleic acids are minimum dosed at 1 mg, 2 mg, 3 mg, 4 mg, or higher in mass for an individual kidney in a subject 30 kg or greater.

In exemplary embodiments, the hydrodynamic parameters are sufficient to achieve protein expression within cells of the kidney after delivery of DNA:
a. The method of claim 15, wherein cell expression is achieved within cells of the glomerulus, tubule, or endothelium inside the kidney.
b. The method of claim 15, wherein differences in cell expression among the glomerulus, tubule, or endothelium correlate to the uses of different promoters in the plasmid DNA.

In exemplary embodiments, the nucleic acids consist of plasmid DNA, minicircle DNA, mRNA, siRNA, or antisense oligonucleotides.

In exemplary embodiments, the viral vector is selected among adenovirus, adeno-associated virus, lentivirus, baculovirus, anellovirus, or Sindbis virus.

In exemplary embodiments, the hydrodynamic injection is used to facilitate better viral vector penetration into tissue, binding to cells, and entry into cells versus viral vector infused at a non-hydrodynamic flow rate.

In exemplary embodiments, the transduction efficiency of kidney cells through retrograde ureter injection is higher with hydrodynamic injection than with a non-hydrodynamic injection (<0.15 mL/sec flow rate).

In exemplary embodiments, the hydrodynamic injection can be further monitored with a pressure sensor to make sure that pressures at a minimum of 50 mmHg, 60 mmHg, 70 mmHg, or 80 mmHg are reached.

In exemplary embodiments, a distal injection into the ureter is sufficient to yield pressure of at least 80 mmHg at flow rates equal to or greater than 0.5 mL/sec.

In exemplary embodiments, a proximal injection into the ureter is sufficient to yield pressure of at least 100 mmHg at flow rates equal to or greater than 1 mL/sec.

In an aspect, the present disclosure provides a method of hydrodynamic gene delivery into the pancreas through the ductal system, including the steps of:
(a) insertion of catheter through endoscopic retrograde cholangio-pancreatography into the pancreatic duct
(b) inflation of balloon to seal and increase pressure during injection
(c) injection at flow rate equal to or between 1 to 2 mL/sec
(d) injection at volume equal to or less than 0.20 mL per gram of pancreas weight
(e) DNA dose injected is at least 10 micrograms per gram of pancreas weight
wherein the method is sufficient to mediate gene expression in all lobes with decreased pancreatic enzyme elevation and tissue necrosis.

In exemplary embodiments, the injection parameters achieve gene expression in ductal cells, islet cells, acinar cells, endothelial cells, and neurons.

In exemplary embodiments, alternatively the total volume injected does not exceed 15 mL for pancreas weighing more than 60 grams.

In exemplary embodiments, if a flow rate of 2 mL/sec is utilized, then a maximum volume of 0.15 mL per gram of pancreas weight is used.

In exemplary embodiments, the DNA dose is preferably greater than 20, or 30 micrograms per gram of pancreas weight.

In exemplary embodiments, amylase or lipase levels are maximally increased 4-fold at day 1 post-injection.

In exemplary embodiments, the catheter can be placed:
(i) through the major duodenal papilla into the main pancreatic duct, distal to the portion of where the pancreatic duct the fuses with the common bile duct, or
(ii) through the minor duodenal papilla into the accessory or dorsal pancreatic duct, with option to advance farther into the main pancreatic duct.

In exemplary embodiments, inflation of a balloon in the catheter near the entrance to the pancreatic duct past the common bile duct to prevent retrograde flow of fluid.

In exemplary embodiments, the maximal balloon size used for sealing the pancreatic duct is 9 mm to avoid injury.

In exemplary embodiments, alternatively two or more flow rate are used during hydrodynamic injection in order to further minimize pancreatic tissue injury while preserving gene delivery.

In exemplary embodiments, wherein the flow rate is initially 1 mL/sec for the first 50% of the injection volume and then increased to 2 mL/sec for the remaining injected volume.

In exemplary embodiments, the flow rate is initially 0.5 mL/sec for the first 50% of the injection volume and then increased to 1.5 mL/sec for the remaining injected volume.

In exemplary embodiments, a side-wall injection catheter is not used to avoid ductal wall injury and prevent pancreatitis.

In one aspect, the disclosure provides a method of hydrodynamic injection into the gallbladder or liver, including the steps of: a) placing a catheter in the common bile duct; b) inflating a balloon in the common bile duct to prevent antegrade flow; c) injecting a DNA solution at high pressure target and/or flow rate into the biliary system, wherein gene expression can be observed in hepatocytes within the liver on immunostaining and cells within the gallbladder.

In exemplary embodiments, the high pressure target is greater than 50 mmHg, greater than 80 mmHg, or greater than 120 mmHg.

In exemplary embodiments, the flow rate is greater than 2 mL/sec, greater than 5 mL/sec, or greater than 10 mL/sec

In exemplary embodiments, the volume injected is greater than 50 mL per kg of liver weigh, or greater than 75 mL per kg of liver weight, or greater than 100 mL per kg of liver weight.

In exemplary embodiments, prior to injection, bile is removed from the biliary system, and normal saline solution is used to flush and prime the biliary system.

In exemplary embodiments, saline solution can be optionally used to fill the gallbladder prior to injection to reduce the pressure differential during injection.

In exemplary embodiments, the preferred DNA dose is at least 20 mg per kg liver weight, or larger.

In exemplary embodiments, the preferred DNA concentration of the injection solution is at least 0.5 mg/mL, or at least 2 mg/mL, or at least 5 mg/mL DNA or more.

In one aspect, the disclosure provides a method of hydrodynamic injection through the biliary system, wherein the injection occurs in the common bile duct with the aid of a biliary stent.

In exemplary embodiments, the biliary stent is placed prior to hydrodynamic injection.

In exemplary embodiments, the biliary stent is placed over the cystic duct in order to prevent fluid from entering the cystic duct.

In exemplary embodiments, the biliary stent is at minimum the greater than the diameter of the bile duct, in order to provide sufficient seal with the duct walls and the stent during the injection.

In exemplary embodiments, the biliary stent is of variable length and can reach from the duodenum to upstream of the cystic duct.

In exemplary embodiments, the balloon catheter is inserted through the stent after placement.

In exemplary embodiments, the balloon catheter can be located anywhere in the stent, including the common bile duct.

In exemplary embodiments, prior to injection, the contrast is injected through the stent to confirm that the cystic duct and gallbladder are not opacified, and that the upstream biliary tree becomes opacified

In exemplary embodiments, DNA solution is injected through at set parameters in order to mediate gene delivery into different cells within the liver.

In exemplary embodiments, a preferred flow rate during the injection is at minimum 1 mL/sec, or at least 2 mL/sec.

In exemplary embodiments, the preferred injection pressure is at least 50 mmHg, or at least 80 mmHg.

In exemplary embodiments, the preferred injection volume is at least 30, 40, 50, or 60 mL per kilogram of liver weight.

In exemplary embodiments, the preferred DNA dose is at least 10 mg per kg liver weight, or larger.

In exemplary embodiments, the preferred DNA concentration of the injection solution is at least 0.2 mg/mL DNA or more.

In exemplary embodiments, the stent is made out of solid continuous and non-fenestrated material, such that fluid cannot go through the walls of the stent.

In an aspect, the disclosure provides a method of delivery of a non-viral DNA vector into a tumor(s) of the liver, including the step of: placing a catheter into abiliary system, preferably in a common hepatic duct; inflating a balloon in the common hepatic duct to prevent antegrade flow, and injecting a DNA solution at hydrodynamic pressure into the biliary system, where an injection achieves expression of the non-viral DNA vector within the tumor cells, regardless of the tumor location within the liver.

In exemplary embodiments, the tumor is in proximity to the biliary system in order to achieve efficient delivery.

In exemplary embodiments, a pressure of at least 50 mmHg, 70 mmHg, or at least 120 mmHg is targeted for an efficient tumor gene delivery.

In exemplary embodiments, a flow rate of at least 2 mL/sec, 4 mL/sec, 7 mL/sec, or at least 10 ml/sec is utilized to achieve the efficient tumor gene delivery.

In exemplary embodiments, a volume of at least 30 mL per kg of liver weight is utilized for the injection.6. The method of claim 1, wherein a non-viral DNA dose of at least 10 mg per kg liver weight, or at least 20 mg per kg liver weight it used for the injection.

In exemplary embodiments, gene expression in tumor cells is highest along the rim of the tumor.

In an aspect, the disclosure provides a method of delivery of a non-viral DNA vector into the tumor(s) of the pancreas, including the steps of: a) placing a catheter into a pancreatic ductal system, upstream of the tumor; b) inflating a balloon in the pancreatic duct to prevent antegrade flow; and c) injecting DNA solution at a hydrodynamic pressure into the pancreatic ductal system; where the injection achieves expression of the non-viral DNA vector within the tumor cells, regardless of the tumor location within the pancreas.

In exemplary embodiments, the tumor is in proximity to the ductal system in order to achieve efficient delivery.

In exemplary embodiments, the pressure of at least 50 mmHg, 70 mmHg, or at least 120 mmHg is controlled for efficient tumor gene delivery.

In exemplary embodiments, the flow rate of at least 1 mL/sec is controlled to achieve efficient tumor delivery.

In exemplary embodiments, the volume of at least 8 mL is injected into the pancreas of an adult human.

In exemplary embodiments, the non-viral DNA dose of at least 1 mg is injected in the pancreas of an adult human.

In exemplary embodiments, the gene expression in tumor cells is highest along the rim of the pancreatic tumor.

In exemplary embodiments, the tumor delivery is most efficient for a pancreatic ductal adenocarcinoma.

In an aspect, the disclosure provides a method of gene delivery into the liver of a primate, including the steps of: inserting a catheter into a common hepatic duct of a primate; inflating a balloon in the common hepatic duct to prevent an antegrade flow; and injecting a DNA solution at a hydrodynamic pressure in a primate liver, where the injection achieves >30% of hepatocytes expressing a gene of interest in the primate liver.

In exemplary embodiments, the common hepatic duct is accessed by an endoscopic retrograde cholangio-pancreatography (ERCP).

In exemplary embodiments, the ampulla of Vater may be cut to increase the size of the opening for the ease of canulation of the common hepatic duct during ERCP.

In exemplary embodiments, a radiocontrast injection is used to localize the catheter that is localized in the common hepatic duct past a cystic duct to avoid injection to a gallbladder.

In exemplary embodiments, the radiocontrast injection verifies that the balloon seals the common hepatic duct during the injection, and that a right and a left hepatic duct is visualized.

In exemplary embodiments, the DNA solution is a normal saline solution with pure recombinant DNA dissolved in the solution.

In exemplary embodiments, a DNA in the DNA solution may be plasmid DNA, minicircle DNA, or linear closed-ended DNA.

In exemplary embodiments, the volume injected is at least 30 milliliters per kilogram of the liver weight, or at least 40/mL/kg or greater.

In exemplary embodiments, the flow rate is at least 1 mL/sec, at least 2 mL/sec, or at 3 mL/sec or greater.

In exemplary embodiments, the pressure parameter is at least 50 mmHg, at least 80 mmHg, or greater than 120 mmHg during a pressure-guided injection.

In exemplary embodiments, the DNA dose will be at least 10, 20, 30, 40, or 50 milligrams per kilogram of a liver weight in certain embodiments.

In exemplary embodiments, the DNA solution is a DNA vector composition encoding a hepatocyte-specific promoter.

In exemplary embodiments, the hepatocyte-specific promoter also contains one or more of a hepatocyte-specific enhancer to drive higher levels of transcription.

In exemplary embodiments, the gene of interest is a codon optimized with codons selected for abundance in the hepatocytes will be selected.

In exemplary embodiments, the DNA vector composition and protocol are provided for the treatment of hemophilia B in primates.

In exemplary embodiments, the DNA vector composition encodes the human factor IX (hFIX) gene.

In exemplary embodiments, the DNA vector composition is a nanoplasmid with a bacterial backbone under 500 basepairs.

In exemplary embodiments, the total DNA vector composition size is less than 3 kb for hFIX.

In exemplary embodiments, a DNA vector composition dose of 20 mg per kg primate liver is sufficient to yield 1000 ng/ml of hFIX in the plasma of primates.

In exemplary embodiments, the DNA vector composition can be redosed in order to secure further gains in expression.

In exemplary embodiments, the procedure may be repeated a second time in the primate, wherein the expression of two different genes is achieved.

In an aspect, the present disclosure provides a method of hydrodynamic gene delivery through the biliary system of a liver of a subject, including the steps of:
(a) insertion of catheter into the common hepatic duct
(b) inflation of balloon to seal the duct and increase pressure during injection
(c) injection at a flow rate at minimum of 2 mL/sec, or alternatively at a minimum pressure of 50 mmHg
(d) delivery of DNA encoding hepatocyte-specific promoter to drive transgene expression
(e) a DNA vector with substantially reduced or absent non-mammalian sequence elements
(f) DNA dose injected is at minimum 10 mg of DNA per kilogram of liver weight
wherein greater than 50% of hepatocytes expressing the gene of interest.

In exemplary embodiments, alternatively, a DNA vector lacks specific modifications, but optimally is injected at a minimum of 20 mg of DNA per kilogram of liver weight to achieve greater than 50% of hepatocytes expressing the gene of interest.

In exemplary embodiments, an optional use of transposon can be utilized to facilitate integration into host chromosomes

In exemplary embodiments, a substantially reduced means total amount of bacterial or phage DNA sequences less than 1000 bp.

In exemplary embodiments, the DNA is a plasmid DNA vector with vector bacterial backbone less than 1 kb in size, or more preferably less than 500 bp in size.

In exemplary embodiments, the plasmid DNA is a nanoplasmid, pFAR, or pCOR vector.

In exemplary embodiments, the DNA is a circular and is a minicircle DNA.

In exemplary embodiments, DNA is a linear DNA from closed-ended DNA, ministring DNA, or doggbone DNA.

In exemplary embodiments, the switch from plasmid backbone including bacterial sequences greater than 1 kb to a DNA vector of claims 5 and 6 increases the observed total transfected area of hepatocytes more than 20%.

In exemplary embodiments, the gene expression among at least 40% of hepatocytes for at least 3 months can be achieved with the use of a transposon system for integration into host genome.

In exemplary embodiments, the duration of expression of non-integrating DNA of claims 5 and 6 is at least 4 months after injection.

In exemplary embodiments, the delivery method expresses for at least 4 months and is able to yield immune tolerance to foreign transgenes in the liver.

In exemplary embodiments, DNA vectors sizes greater than 12 kb, 15 kb, or 20 kb in size can be delivered into multiple cell types through the liver, including hepatocytes, endothelial cells, and bile duct cells yielding protein expression.

In exemplary embodiments, transfection efficiency is maintained with larger plasmid DNA sizes at least 12 kb in size.

In exemplary embodiments, DNA dose can be adjusted according to DNA dose (mg) per liver weight (kg) per kilobases of DNA (kb) in order to adjust for the DNA size in order to maintain equivalent transfection efficiency.

In exemplary embodiments, the formula, 1 mg/kg/kb, can be utilized to project the DNA dose to achieve about 50% of hepatocyte transfection inside the liver.

In exemplary embodiments, the formula 2.5 to 5 mg/kg/kb can be utilized to project the DNA dose to achieve about 70% of hepatocyte transfection inside the liver.

In exemplary embodiments, the procedure can be repeated on a second date with a different DNA expressing the same or a different gene, such that the expression of the first gene is not abolished and expression of both genes is now achieved.

In exemplary embodiments, the second injection achieves similar transfection efficiency to the first injection and can target the same cells.

In exemplary embodiments, the same cells can be observed to express genes after injection.

In exemplary embodiments, the promoter can be altered to achieve expression in a different cell type with the second injection, and that the second injection does not alter expression of the first gene.

In exemplary embodiments, wherein the procedure can be repeated within the same injection procedure with a different DNA expressing the same or a different gene, such that expression of both DNAs is now achieved, and the expression of the first DNA injection is not abolished.

In exemplary embodiments, the second injection achieves similar transfection efficiency to the first and can target the same cells.

In exemplary embodiments, the promoter can be altered to achieve expression in a different cell type with the second injection, and that the second injection does alter expression of the first gene.

In exemplary embodiments, two different DNA molecules can be mixed and delivered during a single injection, such that both DNA molecules enter into the same liver cells.

In exemplary embodiments, DNA doses from 20 mg per kg liver weight to 40 mg per kg liver weight achieve similar transfected area.

In exemplary embodiments, DNA doses up to 40 mg per kg liver weight can be injected without causing significant liver toxicity or physiological distress.

In exemplary embodiments, the flow rate below 1 mL/sec yields no gene expression.

In exemplary embodiments, a flow rate between 1 mL/sec and 2 mL/sec exhibits decreased gene expression compared to greater than 2 mL/sec.

In exemplary embodiments, flow rates above 4 mL/sec yield progressively less efficient hepatocyte delivery.

In exemplary embodiments, flow rates greater than or equal to 7 mL/sec achieve efficient bile duct delivery.

In exemplary embodiments, the preferred injection volumes are between 30 mL/kg to 60 mL/kg per liver tissue.

In exemplary embodiments, injection volumes greater than or equal 70 mL/kg liver tissue are associated with decreased efficiency of gene delivery.

In exemplary embodiments, the gene injection procedure is well-tolerated by subjects of 25 kg or 15 kg or 5 kg in size and yields similar gene delivery efficient to larger mammals.

In exemplary embodiments, the transfected hepatocyte area from biliary hydrodynamic delivery can be further increased by at least 10% of total hepatocytes when incorporating two or more flow rates during the injection.

In exemplary embodiments, the flow rate of 2 mL/sec is used first for 50% to 66% of the total injected volume, followed by 4 mL/sec second for the remaining volume.

In exemplary embodiments, the flow rate is 2 mL/sec is used first for 33% of volume injection, 3 mL/sec is used second 33% of the volume, followed by 4 mL/sec for the remaining injection volume.

In exemplary embodiments, the catheter is inserted into the common hepatic duct through ERCP, EUS, or imaging-guided percutaneous routes.

In exemplary embodiments, the DNA concentration of the injected solution is at minimum 0.30 mg/mL, and more preferably greater than 0.40 mg/mL, 0.50 mg/mL, or 0.60 mg/mL in concentration.

In an aspect, the present disclosure provides a method of achieving expression inside liver sinusoidal endothelial cells (LSECs), including the steps of performing biliary hydrodynamic injection according to claim 1, except using a cell-specific promoter to target expression in LSECs.

In exemplary embodiments, LSECs can be targeted for expression with CD36 promoter or FVIII promoter.

In exemplary embodiments, an injection pressure of 80 mmHg yields more efficient expression than an injection pressure of 50 mmHg.

In exemplary embodiments, a pressure of 150-200 mmHg yields efficient gene expression.

In exemplary embodiments, a pressure above 200 mmHg yields progressively less gene expression.

In a certain aspect, a more efficient balloon seal is obtained by advancing the catheter and inflating the balloon into intrahepatic ducts.

In a certain aspect, placement of the balloon in extrahepatic ducts results in leakage of fluid around the balloon.

In a certain aspect, the balloon size is at least 2 times, at least 3 times, or at least 4 times the duct diameter.

In a certain aspect, the balloon can be inflated to maximal size 3 times the duct diameter, when the balloon is placed in an extrahepatic bile duct.

In a certain aspect, the balloon can be inflated to a minimal size of 4 times the duct diameter, when the balloon is placed in an intrahepatic bile duct.

In a certain aspect, the balloon size does not inflate fully within an intrahepatic duct, but rather additional pressure is generated within the balloon.

In a certain aspect, leakage around the balloon occurs with sizes 8.5 mm or less, whether intrahepatic or extrahepatic, such that those sizes should be avoided.

In a certain aspect, the balloon is inflated less than 15 mm in size in the common hepatic duct in order to avoid rupture.

In a certain aspect, the balloon is placed in the right or left hepatic duct and the injection occurs subsequently in that location, then the injection is repeated again in the opposing duct to ensure both lobes of the liver are injected equally.

In a certain aspect, the balloon seal can be monitored through measuring intraluminal biliary pressure.

In a certain aspect, loss of a plateau wave form, defined by greater than 20 mmHg decrease from the start to the end of the plateau, signifies leakage around the balloon.

In a certain aspect, the balloon seal can be verified by filling the bile ducts above and below the balloon with radiocontrast solution prior to injection.

In a certain aspect, a loss of fluid seal during injection is demonstrated by the clearing of contrast below the balloon, either into the cystic duct and gallbladder, or alternatively into the common bile duct.

In a certain aspect, contrast above the balloon is cleared into the liver to indicate successful injection.

In a certain aspect, the hydrodynamic injection can be repeated multiple times within a single procedure in order to augment DNA delivery.

In a certain aspect, the use of two or more injections is additive toward achieving the final gene expression amount.

In a certain aspect, this strategy allows one to overcome inherent limitations of the angiographic or power injection volume through the use of multiple injections.

In a certain aspect, primate liver tissue is more elastic than porcine tissue, such that different duct properties necessitate changes in balloon sizes and injection parameters to mediate gene delivery in primates.

In a certain aspect, the flow rate must be increased to achieve a given pressure versus injection in porcine models.

In a certain aspect, a flow rate of at least 4 mL/sec is required to achieve a pressure plateau of at least 80 mmHg.

In a certain aspect, primates can tolerate pDNA doses of at least 80 mg without any significant physiological side effects.

In a certain aspect, an injection volume of at least 120 mL volume per 400 gram of liver can be injected into a primate without any significant perturbation in vital signs.

In a certain aspect, an injection speed of up to 12 mL/sec can be tolerated by the primate liver without tissue injury, vital sign changes, or bile duct rupture.

In a certain aspect, flow rates greater than 4 mL/sec, but less than 8 mL/sec, should be used given lack of improved gene delivery at higher flow rates.

In a certain aspect, 30 mL per 400 g of volume can be used, or alternatively up to 150 mL per 400 g of volume can be used.

In a certain aspect, the volume utilized does not impact the efficiency of gene delivery at a given DNA dosage.

In a certain aspect, the vector composition should be dosed by the copies of the transgene expression cassette, such that differing pDNA doses are required if additional extraneous DNA is included in the DNA vector.

In a certain aspect, the use of DNA molecule with reduced backbone allows for a comparatively smaller DNA dose

In a certain aspect, increasing the pDNA dose and/or the vector expression cassette dose per animal leads to quantitatively equivalent increase in expression of a protein therapeutic of interest.

In a certain aspect, a method of hydrodynamic gene delivery through the biliary system of a liver of a subject is provided. In a certain aspect, the method comprises:
(a) insertion of catheter into the bile duct:
(b) inflation of balloon to seal within the bile duct and prevent antegrade flow of solution
   i. The inflated size of the balloon is at least 2×, at least 3×, or at least 4× the diameter of the bile duct to overcome elasticity of primate ducts;
   ii, wherein a method of verifying balloon seal during injection consists of the placement of radiocontrast solution above and below the balloon to detect antegrade motion of fluid by fluoroscopy
(c) injection at a flow rate at minimum of 2 mL/sec, or alternatively injection at a minimum pressure of 50 mmHg
   i. more preferably, a minimum of 4 mL/sec, which can yield minimum of 80 mmHg into primate liver in plateau pressure; wherein the flow rate can be further minimized to less than 12 mL/sec, less than 10 mL/sec, or less than 8 mL/sec without loss in total gene expression;
   ii. volume injected is preferably less than 250 mL/kg liver tissue, less than 150 mL/kg liver tissue, or less than 50 mL/kg liver tissue;
   iii, wherein a plateau pressure during hydrodynamic injection varying less than 10% of mmHg over the course of the injection is obtained and signals sufficient seal; and
   iv, wherein optionally multiple flow rates can be employed within a single injection to alter and vary the pressure obtained; and
(d) delivery a miniaturized DNA vector with substantially reduced or absent non-mammalian sequence elements
   i. wherein the miniaturized DNA vector affords greater persistence of expression lasting at least 4 months;
   ii. wherein the miniaturized DNA vector affords high potency at a given DNA dose in primates, allowing for smaller doses to be used compared to regular plasmid DNA;
   iii. wherein the DNA vector optimally contains a hepatocyte-specific promoter for enhanced expression in the liver.

In a certain aspect, a substantially reduced means total amount of bacterial or phage DNA sequences less than 1000 bp.

In a certain aspect, the DNA is a plasmid DNA vector with vector bacterial backbone or sequence less than 1 kb in size, or more preferably less than 500 bp in size.

In a certain aspect, the plasmid DNA is a nanoplasmid, GenCircle, pFAR, or pCOR vector.

In a certain aspect, the DNA is a circular and is a minicircle DNA or a minivector DNA.

In a certain aspect, DNA is a linear DNA from closed-ended DNA, ministring DNA, or doggbone DNA.

In a certain aspect, the linear DNA only has small foreign sequences at either end, each less than 100 bp's in size, and the mammalian expression sequence of interest is the rest of the vector.

In a certain aspect, a biliary sphincterotomy is performed during the first procedure in a subject to reduce or eliminate the risk of post-ERCP pancreatitis during subsequent ERCP procedures for redosing the genetic medicine.

In a certain aspect, the injection procedure can be repeated twice within one session to increase protein expression.

In a certain aspect, the repeated injection can use the same DNA to boost individual protein expression, or can use two different DNA solutions to yield two different proteins being expressed.

In a certain aspect, the sum of the DNA dose from the repeated injections is similar to or equivalent to the expression obtained from a single DNA injection

In a certain aspect, the injection procedure can repeated again after a single administration, such that the transfection efficiency is the same, and the peak protein expression is equivalent between injections with lack of immunogenicity observed.

In a certain aspect, the injection procedure that is repeated can occur at least one month, at least 3 months, at least 6 months, or at least one year apart.

In a certain aspect, the pressure is monitored using a pressure transducer detecting a fluid-filled column in the pressure catheter, or alternatively is monitored with a pressure sensor that is threaded into the catheter lumen.

In a certain aspect, the pressure sensor readings are recorded in real-time, and the pressure curves interpreted post-injection to ascertain if successful seal and peak expression were achieved.

In a certain aspect, optimal hydrodynamic injections achieve at least a 2× rise in liver enzymes such as ALT and AST levels compared to pre-treatment values by day 1 post-injection

In a certain aspect, the liver enzymes return to normal limits within 7 days of injection In a certain aspect, gene delivery occurs intratumorally and peri-tumorally, in both malignant cells and normal cells, interspersed and surrounding the tumor.

In another aspect, the disclosure provides a method of hydrodynamic injection into the liver through the biliary system, using a partially deployed fully covered metallic or plastic stent wherein: a) the deployment of the stent is distal to proximal (with respect to the stent deployment catheter); b) the location of the released or open component of the stent is in the common hepatic duct and the tip of the stent remains in delivery catheter housing the stent, such that is located in either the common hepatic duct, common bile duct, or in the periampullary duodenum; c) wherein the cystic duct orifice is occluded and/or bypassed by the covered portion of the stent, such that no injected fluid solution is able to enter the cystic duct or gall bladder; d) removing the guidewire from when the stent delivery system; e) injecting a DNA solution at high pressure target and/or flow rate into the biliary system through the guidewire lumen; wherein antegrade flow in the biliary system is prevented by the stent forming a closed system with the catheter due to the partial deployment and continued connection, and/or wherein gene expression can be observed in hepatocytes within the liver on immunostaining.

In an embodiment, the high-pressure target is greater than 50 mmHg, greater than 80 mmHg, or greater than 120 mmHg.

In an embodiment, the flow rate is greater than 2 mL/sec, greater than 5 mL/sec, or greater than 10 mL/sec

In an embodiment, the volume injected is greater than 50 mL per kg of liver weight, or greater then 75 mL per kg of liver weight, or greater than 100 mL per kg of liver weight.

In an embodiment, prior to injection, bile is removed from the biliary system, and normal saline solution is used to flush and wash the biliary system.

In an embodiment, the preferred DNA dose is at least 20 mg per kg liver weight, or larger.

In an embodiment, the preferred DNA concentration of the injection solution is at least 0.5 mg/mL DNA or more.

In an embodiment, the biliary stent is at minimum the diameter of the bile duct, in order to provide sufficient seal with the duct walls and the stent during the injection.

In an embodiment, the biliary stent diameter is at least 150% of the bile duct diameter, or at least 200% of the bile duct diameter.

In an embodiment, the biliary stent is of variable length and can reach from the outside the ampulla to the upstream of the cystic duct.

In an embodiment, the injection occurs from the tip of the “olive” aspect of the stent. The “olive” may be located between the liver end of the stent and the hepatic hilum at the time of the injection.

In an embodiment, the injection occurs at an opening on the catheter at the proximal location where the stent feeds into the catheter, such that the fluid fills the stent as it proceeds retrograde and that the cone of the stent into the catheter prevents any antegrade flow.

In an embodiment, prior to injection, the contrast is injected through the stent to confirm that the cystic duct and gallbladder are not opacified, and that the biliary tree becomes opacified.

In an embodiment, the stent is made out of solid material, such that fluid cannot go through the walls of the stent.

In an embodiment, the liver end of the stent is opened in the left or right main hepatic duct and not in the common hepatic duct.

In an embodiment, once hydrodynamic injection is complete from either the left or right main hepatic duct, the alternative duct is injected using the same technique.

In an embodiment, the stent is partially deployed, meaning only at most 95% of its length, 75% of its length, 50% of its length, or 25% of its length in some embodiments is deployed outside the catheter, with the rest of the length of the stent remaining within or attached to the catheter.

In an embodiment, the partially deployed stent forms a funnel or cone shape at its proximal end where it is attached to the catheter, thereby forming a closed system.

### Definitions

To facilitate an understanding of the present disclosure, a number of terms and phrases are defined below:

Unless specifically stated or obvious from context, as used herein, the term “about” is understood as being within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, polypeptide, or fragments thereof.

By “ameliorate” is meant decrease, suppress, attenuate, diminish, arrest, or stabilize the development or progression of a disease (e.g., a genetic disease of the target tissue (e.g., kidney, pancreas, common bile duct, tumor, liver, etc.) phenotype).

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The phrase “combination therapy” embraces the administration of a gene therapy protocol and one or more additional therapeutic agents (e.g., erythropoietin, corticosteroids, ACE inhibitors) as part of a specific treatment regimen intended to provide a beneficial (additive or synergistic) effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous or overlapping manner. Substantially simultaneous administration can be accomplished, for example, by administering to the subject, for example, one or more copper chelating compounds while administering a gene therapy protocol as disclosed herein. Sequential or substantially simultaneous administration of each therapeutic agent can be affected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection (e.g., a gene therapy protocol) while the other component(s) (e.g., one or more copper chelating compounds) of the combination may be administered orally. The components may be administered in any therapeutically effective sequence.

The phrase “combination” embraces groups of compounds and/or non-drug gene therapies useful as part of a combination therapy as disclosed herein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “disease” is meant any condition or disorder (e.g., genetic disease of the target tissue (e.g., kidney, pancreas, common bile duct, tumor, liver, etc.)) that damages or interferes with the normal function of a cell, tissue, or organ.

By “effective amount” is meant the amount required to ameliorate the symptoms of a disease (e.g., neurological or other symptoms of genetic disease of the target tissue (e.g., kidney, pancreas, common bile duct, tumor, liver, etc.)) relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. The effective amount may also refer to levels of gene expression (e.g., ATP7B mRNA or protein expression) in the appropriate tissues of a patient.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000, 2000, 3000, 4000, 5000 or more nucleotides or amino acids.

A “gene therapy composition” is to be understood as meaning a DNA composition (e.g., including a full length PKD1, PKD2, SLC3A1, SLC7A9 NPHP1-NPHP9, MKS1, SLC12A3, OLRL1, CSNCU1, CFTR, IGF-1, Reg3g, ATP7B, UGT1A1, HFE, OTC, LDLR, ABCB4, PBGD, or VWF nucleotide sequence, or portion thereof) for generating prophylaxis and/or treatment of genetic disease in the target tissue (e.g., kidney, pancreas, common bile duct, tumor, liver, etc.). Accordingly, in certain aspects, gene therapy compositions are medicaments which comprise a full length of PKD1 nucleotide sequence, or portion thereof, and are intended to be used in humans or animals for generating prophylaxis and/or treatment of genetic disease of the kidney. Accordingly, in certain aspects, gene therapy compositions are medicaments which comprise a full length CFTR nucleotide sequence, or portion thereof, and are intended to be used in humans or animals for generating prophylaxis and/or treatment of genetic disease of the pancreas. Accordingly, in certain aspects, gene therapy compositions are medicaments which comprise a full length ATP7B, UGT1A1, HFE, OTC, LDLR, ABCB4, PBGD, or VWF nucleotide sequence, or portion thereof, and are intended to be used in humans or animals for generating prophylaxis and/or treatment of genetic disease of the liver.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid molecule (e.g., a DNA, an mRNA, a cDNA, and the like) that is free of the genes from which, in the naturally-occurring genome of the organism, the nucleic acid molecule of the disclosure is normally associate or derived. The term therefore includes, for example, a recombinant DNA (e.g., including a genomic DNA or cDNA coding for a ATP7B gene, as well as associated regulatory components such as, for example, an enhancer(s), a promoter, 5′ and/or 3′ untranslated regions (UTRs), and the like) that may be incorporated into: a vector, or an autonomously replicating plasmid or virus, or into the genomic DNA of a prokaryote or eukaryote, or into a polynucleotide that exists as a separate molecule (for example, a cDNA or a genomic or cDNA fragment produced by PCR or restriction endonuclease digestion, or a naked DNA construct such as a plasmid or cosmid or linear DNA) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

“Mutation” for the purposes of this disclosure means a DNA sequence found in the gene of a patient that does not correlate with an established wildtype gene sequence, and such mutations may be due to one or more single nucleotide polymorphisms, one or more deletions or insertions of one or more nucleotides, and deletion or insertion of splice site junctions. “Mutation” may also refer to patterns in the sequence of RNA from a patient that are not attributable to expected variations based on known information for the gene and are reasonably considered to be novel variations in, for example, the splicing pattern of the gene of the patient.

Unless specifically stated or obvious from context, as used herein, the term “or” is understood to be inclusive. Unless specifically stated or obvious from context, as used herein, the terms “a,” “an,” and “the” are understood to be singular or plural.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neo-antigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, t toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the pooled tumor specific neo-antigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, refer to reducing the probability of developing a disease or condition (e.g., genetic disease of the target tissue (e.g., kidney, pancreas, common bile duct, tumor, liver, etc.)) in a subject, who does not have, but is at risk of or susceptible to developing the disease or condition (e.g., genetic disease of the target tissue (e.g., kidney, pancreas, common bile duct, tumor, liver, etc.)).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison (e.g., a wildtype ATP7B gene sequence). A reference sequence may be a subset of, or the entirety of, a specified sequence; for example, a segment of a full-length cDNA or genomic sequence, or the complete cDNA or genomic sequence. For polypeptides, the length of the reference polypeptide sequence will generally be at least about 10-5,000 amino acids, 10-4,000 amino acids, 10-3,000 amino acids, 10-2,000 amino acids, 10-1,500 amino acids, 10-1,000 amino acids, 10-500 amino acids, or 10-100 amino acids. Preferably, the length of the reference polypeptide sequence may be at least about 10-50 amino acids, more preferably at least about 10-40 amino acids, and even more preferably about 10-30 amino acids, about 10-20 amino acids, about 15-25 amino acids, or about 20 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

Nucleic acid molecules useful in the methods of the disclosure include any nucleic acid molecule that encodes a polypeptide (e.g., an ATP7B polypeptide) of the disclosure, or a fragment thereof. Such nucleic acid molecules need not be 100% identical with an endogenous nucleic acid sequence, but will typically exhibit substantial identity (e.g., 100%, 99%, 98%, 97%, 96%, 95%, 94%, 93%, 92%, 91%, or 90%). Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., an ATP7B gene described herein), or portions thereof, under various conditions of stringency. (see, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid or nucleotide sequence (for example, any one of the amino acid or nucleotide sequences described herein). Preferably, such a sequence is at least 60%, at least 70%, at least 80%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, or at least 100% identical at the amino acid sequence or nucleic acid sequence used for comparison (e.g., wildtype ATP7B).

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the terms “treat,” “treated,” “treating,” “treatment,” and the like refer to reducing or ameliorating a disorder and/or symptoms associated therewith (e.g., genetic disease of the target tissue (e.g., kidney, pancreas, common bile duct, tumor, liver, etc.)). It will be appreciated that, although not precluded, treating a disorder or condition does not require that the disorder, condition, or symptoms associated therewith be completely eliminated.

The term “therapeutic effect” refers to some extent of relief of one or more of the symptoms (e.g., neurological, kidney-related, liver-related, etc.) of genetic disease of the target tissue (e.g., kidney, pancreas, common bile duct, tumor, liver, etc.) or its associated pathology. “Therapeutically effective amount” as used herein refers to an amount of an agent or combination therapy which is effective, upon single or multiple dose administration to the cell or subject, in prolonging the survivability of the patient with genetic disease of the target tissue, reducing one or more signs or symptoms of genetic disease of the target tissue, preventing or delaying onset of symptoms of genetic disease of the target tissue, and the like, beyond what would be expected in the absence of such treatment. “Therapeutically effective amount” is intended to qualify the amount required to achieve a therapeutic effect. A physician or veterinarian having ordinary skill in the art can readily determine and prescribe the “therapeutically effective amount” agent or combination therapy required.

The pharmaceutical compositions typically should provide a dosage of from about 0.0001 mg to about 200 mg of compound per kilogram of body weight per day. For example, dosages for systemic administration to a human patient can range from 0.01-10 μg/kg, 20-80 μg/kg, 5-50 μg/kg, 75-150 μg/kg, 100-500 μg/kg, 250-750 μg/kg, 500-1000 μg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 1500-2000 mg/kg, 5 mg/kg, 20 mg/kg, 50 mg/kg, 100 mg/kg, of 200 mg/kg. Pharmaceutical dosage unit forms are prepared to provide from about 0.001 mg to about 5000 mg, for example from about 100 to about 2500 mg of the compound or a combination of essential ingredients per dosage unit form.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the disclosure.

These and other embodiments are disclosed and/or encompassed by the following Detailed Description.

### BRIEF DESCRIPTION OF THE DRAWINGS

The above-mentioned and other features and advantages of the present disclosure will be better understood when reading the following detailed description taken together with the following drawings in which:

FIGS. 1A-1D depict a cartoon and three images that show the technique for hydrodynamic injection by accessing the ureter through the bladder, so that injection into the ureter could be used to deliver pDNA into the kidney of pigs. FIG. 1A shows a cartoon diagram of the catheter localization. The blue circle represents where the catheter balloon will be positioned and placed.

FIG. 1B is a cytoscopic image of the bladder that reveals the ureter orifice in close proximity to the urethra. FIG. 1C shows a bulge in the bladder wall, verifying inflation of the balloon after it was inserted at the entrance of the ureter into the bladder. FIG. 1D is a fluoroscopic image of the entire ureter and renal pelvis filled with radiocontrast to approximate the total volume.

FIGS. 2A-2D show images and four photographs of hydrodynamic injection through the ureter that can cause kidney rupture. Hydrodynamic injection of fluid through the ureter was explored by testing both contrast injection, and injection with normal saline solution. FIG. 2A shows the escape of contrast into the subcapsular space after hydrodynamic injection. The image on the left is an earlier time point than the right, only seconds later. Gross examination of kidney post-injection (FIG. 2B) reveals pooled blood and a large hematoma underneath the kidney capsule. Subsequent removal of the kidney capsule (FIG. 2C) reveals an approximate 1.5 cm tear in the kidney surface at the upper pole of the kidney. Dissection of the kidney in FIG. 2D shows that tears start at the base of the medulla and dissect the entire cortex.

FIGS. 3A-3C show a photograph and images from GFP staining that demonstrate that hydrodynamic injection through the ureter can result in gene delivery. The plasmid, pCLucf, encoding firefly luciferase and GFP was injected into the kidney at different injection parameters. FIG. 3A is a photograph that shows kidneys sectioned at the upper, middle, and lower poles, represented by black circles. FIG. 3B is an image at higher magnification, that shows GFP staining in the glomeruli of scattered cells. FIG. 3C depicts representative photos of luciferase staining of the upper, middle and lower poles at the cortex/medulla interface. These are depicted for the kidney injected at 20 mL and 2 mL/sec, which resulted in the delivery of 1.2 mg pDNA. The lower right image shows an un-injected kidney as a control for staining. Pigs injected with other injection parameters (27 mL, 2 mL/sec; 30 mL, 1 mL/sec) showed similar immunohistochemistry (IHC) staining pattern (data not shown).

FIGS. 4A-4C depict four images showing that hydrodynamic injection achieves expression in multiple cell types in the kidney. GFP staining is shown after pCLucf injection in different cell types in the kidney. FIG. 4A shows GFP staining in tubules within the cortex. FIG. 4B shows that GFP staining occurs in endothelial cells, and FIG. 4C shows that GFP staining can be found in small groups of tubules within the kidney medulla.

FIG. 5 shows five photographs that demonstrate that hydrodynamic injection at lower parameters yields a safer injection. Hydrodynamic injection parameters for a ureteral distal injection were undertaken for flow rates ranging from 0.5 mL/sec to 2 mL/sec. In these injections, volumes ranged from 16 mL to 20 mL in total, as indicated. Gross dissection of the kidneys was performed post-injection to look for ruptures. At top left, the kidney injected with 20 mL @ 1 mL/sec showed signs of rupture, and the photo on the right shows bruising when injected with 20 mL @ 1.5 mL/sec. In the lower right photo, rupture occurred when the kidney was injected with 20 mL @ 0.5 mL/sec, but the lower right image shows no injury when 16 mL was injected at 2 mL/sec. The fifth photograph is a control, with no injection. Dissected kidneys are depicted for the different groups, which all show no signs of rupture.

FIG. 6 shows six images of hydrodynamic injection at lower parameters that can still yield gene delivery. Hydrodynamic injection parameters for a ureteral distal injection were undertaken for flow rates ranging from 0.5 mL/sec to 2 mL/sec. Volumes ranged from 16 mL to 20 mL, as indicated, and injected pDNA resulted in the delivery of 2 mg of pCLucf. Representative photos are depicted for firefly luciferase staining within the kidney upper pole for each parameter. Two control tissues are also presented at the bottom that were not injected. Similar expression patterns are observed for the middle and lower poles of the kidney, and when stained with GFP (data not shown).

FIGS. 7A and 7B are images of hydrodynamic injection at the lowest flow rate that yields expression comparable to higher flow rates. Hydrodynamic injection of tissue samples from the kidney at the lowest tested flow rate of 0.5 mL/sec is presented showing that protein expression can still be achieved. FIG. 7A shows that GFP staining demonstrates positive cells in the glomeruli, while a control did not show any staining. FIG. 7B shows the cortex at low magnification that depicted abundant tubules stained for firefly luciferase.

FIGS. 8A-8D are images and a photograph showing that a proximal ureteral injection can cause kidney injury. Hydrodynamic injection was undertaken within the ureter at a location proximal to the kidney pelvis. Injection was then undertaken at 15 mL and 1 mL/sec using contrast as the injection fluid. Fluoroscopy in FIG. 8A demonstrates that a balloon positioned in the pelvis of the kidney fails to prevent antegrade flow of contrast into the ureter. Fluoroscopy in FIG. 8B post-injection shows rupture of the kidney capsule, resulting in contrast escape into the subcapsular space. Hemorrhage and rupture was confirmed in the photo in FIG. 8C upon gross inspection of the kidney at necropsy. FIG. 8D shows no GFP via immunohistochemistry staining from the kidney proximally injected with 15 mL @ 1 mL/sec.

FIG. 9 depicts two images revealing that a proximal ureteral injection can cause kidney injury. Hydrodynamic injection at 15 mL and 1 mL/sec in proximity to the kidney can lead to significant kidney injury. The top image is a tissue section with a large area of tissue injury and lymphocyte infiltration starting at the medulla and extending through the cortex. The bottom image shows high magnification of an area of necrosis, along with areas of neutrophil and lymphocyte infiltration.

FIG. 10 presents eight images that demonstrate gene expression from a proximal ureter injection at different lower injection parameters. To test for safety and gene expression, hydrodynamic injection was assessed from a ureteral proximal location at various lower injection parameters. Plasmid DNA doses of 10 mg pCLucf were delivered, and injection parameters tested were 11 mL at 2 mL/sec, 12 mL at 1.5 mL/sec, and 10 mL injected at 1 mL/sec. Pigs were harvested 3 days post-injection and kidneys sectioned at the upper, middle, and lower poles. IHC staining for GFP was undertaken on the upper, middle, and lower kidney poles in the kidney cortex in the left images, and the medulla in the right images. Representative images of IHC staining for the different parameters evaluated are shown.

FIGS. 11A and 11B are two images and a graph that demonstrate the correlating flow rate to pressure during hydrodynamic injection in the proximal ureter. Pressure was monitored during hydrodynamic injection in the distal ureter. For the experiment, a quadruple lumen catheter was obtained that consisted of a guidewire, balloon, pressure sensor, and injection ports. The catheter tip was put in a proximal position closer to the kidney. FIG. 11A depicts an example of contrast that fills ureter and kidney pelvis for localization. A series of flow rates were tested, and pressure was monitored at the same time. As an example, 10 mL was injected at a flow rate of 1 mL/sec. In FIG. 11B, the pressure tracing graph reveals a peak pressure of 105 mmHg. The pressure rapidly declined, possibly due to kidney rupture, or alternatively balloon failure.

FIGS. 12A-12C show two photographs of a kidney, and a graph that correlates the flow rate to pressure during hydrodynamic injection in the distal ureter. Pressure was monitored during hydrodynamic injection in the distal ureter. For the experiment, a laparotomy was performed, and excision made into the bladder wall. The ureter orifice was visualized, and a catheter inserted into the ureter. The catheter itself was connected to a power injector for delivery of saline solution. A pressure sensor was advanced alongside the catheter into the ureter, being too large in size to fit through the lumen. Both the pressure sensor and catheter were advanced into the distal aspect of the ureter. FIG. 12A shows a surgical tie that was made to prevent antegrade flow during injection into the ureter. A series of different flow rates were tested to evaluate for pressure with a fixed volume of 10 mL. FIG. 12B depicts the results of the pressure tracings with arrows indicating injections. The study observed that pressure is flow rate dependent. Flow rates of 0.5-1 mL/sec yielded pressure of 70-80 mmHg. Flow rates of 1.5-2 mL/sec yield 120-140 mmHg. Additional testing with higher volume injected at a constant flow rate did not further increase pressure (data not shown). Baseline in the ureter was observed to be 5-15 mmHg prior to injection. After the series of injections, the kidney was further dissected. FIG. 12C shows pooling of fluid in the space between the capsule and kidney surface that was observed. Several tears were noted, which have occurred during injections greater than 3 mL/sec.

FIGS. 13A-13D depict a cartoon and three images assessing the pancreatic duct by endoscopic retrograde cholangio-pancreatography (ERCP) for gene delivery in pigs. The pancreatic duct is a conduit for gene delivery via hydrodynamic injection. Pigs are an excellent model for ERCP given that their organs are similarly sized relative to humans, although the anatomy of the pig pancreas differs in lobular structure between the two species. FIG. 13A shows a cartoon model of a catheter placement in a pig pancreas with a depiction of the two separate orifices of the bile and pancreas ducts. FIG. 13B demonstrates canulation of the pancreatic duct in pigs, which differs notably from humans because of the lack of ampulla of Vater. In the pig, the orifice of the pancreatoduodenal junction and the choledochoduodenal junction are separate. FIG. 13C shows an image of a representative fluoroscopy of a pancreatic duct pre-hydrodynamic injection is depicted. FIG. 13D shows an image of a representative fluoroscopy of pancreatic duct post-hydrodynamic injection confirming that there were no signs of rupture post-injection.

FIGS. 14A-14C show images and two graphs that demonstrate the safety of pancreatic hydrodynamic injection. Hydrodynamic injection was conducted into a pig pancreas at injection parameters of 22 mL at 2 mL/sec, which resulted in the delivery of 1.2 mg of pDNA. FIG. 14A shows abdominal CT scans of pancreas organs from two different pigs, which were obtained 1 day post-injection. To measure pancreatic injury, FIG. 14B shows amylase levels at pre-injection, post-injection, day 1, and day 3 time points. To measure for inflammation induced by the injection procedure, FIG. 14C shows the white blood count (WBC) levels at pre-injection, post-injection, and day 1 time points.

FIGS. 15A-15D show a photograph, a Western blot, and images of pancreatic tissue samples that evaluate gene expression resulting from hydrodynamic gene delivery of plasmid DNA into the pancreas. FIG. 15A shows a photograph of a pancreas obtain from necropsy of an injected pig. Gross examination of the pancreas is conducted to look for signs of liver injury. The gross specimen was assessed and no gross pancreatic ductal or parenchymal injury was observed.

FIG. 15B shows a PCR that was performed as a first step to detect the presence of plasmid DNA in the duodenal lobe, the splenic lobe and the connecting lobe of a pig. FIG. 15C shows images of pancreatic tissue from the three lobes that was analyzed by immunohistochemistry (IHC) for the presence of firefly luciferase. FIG. 15D shows an image of pancreatic tissue analyzed by IHC for the presence of firefly luciferase at low-magnification.

FIGS. 16A-16D show images of the expression of reporter genes seen in different pancreatic cell types after hydrodynamic injection. Hydrodynamic gene delivery of plasmid DNA into the pancreas was evaluated for gene expression in different cell types in the pancreas. In FIG. 16A, high-magnification images of pancreatic ductal cells show specific staining of GFP in pancreatic ductal cells (left panel) relative to a saline control (middle panel). Cytokeratin staining confirmed the identity of the ductal cells. High-magnification images in FIG. 16B also show specific staining of luciferase in pancreatic islet cells (left panel) relative to a saline control (middle panel). Synaptophysin staining confirmed the identity of the islet cells. In addition to these cell types, firefly luciferase reporter gene expression was also observed in endothelial cells (FIG. 16C) and neurons (FIG. 16D) in pancreatic tissue.

FIG. 17 shows images that demonstrate that hydrodynamic injection into the pancreas can yield areas of tissue injury and cause immune infiltration. Pancreatic tissue was assessed for signs of tissue damage. In the top two images, areas of immune infiltrate with neutrophils and lymphocytes were seen 4-5 times per tissue section. The bottom image reveals an acellular area of necrosis surrounded by an area of neutrophil and lymphocyte infiltrate.

FIGS. 18A and 18B show an image and four photographs that depict the development of pancreatic caseating necrosis after ductal injury and hydrodynamic injection. Hydrodynamic injection was conducted into a pig pancreas through the ductal system. An 11 mm balloon was inflated prior to the injection to seal the duct. A pig was injected at injection parameters of 22 mL @ 2 mL/sec which resulted in the delivery of 2 mg pDNA of pCLucf. FIG. 18A shows post-injection fluoroscopy. Contrast blush was observed, revealing that contrast injection was leaking from the ductal system and that a rupture of the wall occurred. On day 3 post-injection, the pig was harvested, and the pancreatic tissue was analyzed. Gross examination and dissection of the duodenal lobe of the pancreas depicted in the photographs in FIG. 18B revealed a large area of pale, yellow colored tissue consistent with necrosis and injury from the injection, seen in the photographs on the left, top and bottom. The photographs on the right show a magnified view of the necrotic, injured tissue. Tissue staining through the rest of the organ did not demonstrate any gene delivery (data not shown).

FIGS. 19A-19C show images of gene delivery into the pancreas with minimal injection parameters. Hydrodynamic injection through the ductal system was conducted in a pig model. Decreased injection parameters were tested to evaluate if they can still mediate decreased gene delivery. FIG. 19A depicts a representative picture of IHC staining for firefly luciferase showing an expression inside islet and ductal cells, after a first pig was injected at injection parameters of 20 mL, 1 mL/sec, which resulted in the delivery of 4 mg pDNA (pCLucf). Pre- and post-procedure amylase levels are reported. Gross dissection of the pancreas shows no abnormalities. FIG. 19B shows a representative picture of IHC staining for firefly luciferase, showing an expression inside islets and ductal cells, after a first pig was injected at 15 mL, 2 mL/sec, which resulted in the delivery of 4 mg pDNA (pCLucf). Pre- and post-procedure amylase levels are reported. Gross dissection of the pancreas shows no abnormalities. FIG. 19C compares areas of immune infiltration in a pig injected at injection parameters of 22 mL at 2 mL/sec at 1 mg pDNA and 15 mL at 2 mL/sec at 4 mg pDNA, showing decreased immune infiltrates at lower injection parameters.

FIG. 20 shows a conventional straight plastic biliary stent placed that traverses the papilla and the cystic duct. This type of stent is designed for optimal drainage of bile and has an internal antimigration flare. This is an example of a type of stent that would not facilitate the goal of bypassing the cystic duct orifice due to the relatively narrow caliber stent and the two additional side-holes within the bile duct aspect of the stent towards the liver end of the stent that would result in inadequate diversion of injected fluid away from the cystic duct or between the stent and the bile duct wall.

FIG. 21 shows a relatively magnified view of a conventional straight plastic biliary stent placed that traverses the papilla and the cystic duct. As above, this is an example of a type of stent that would not facilitate the goal of bypassing the cystic duct orifice due to the relatively narrow caliber stent and the two additional side-holes within the bile duct aspect of the stent towards the liver end of the stent that would result in inadequate diversion of injected fluid away from the cystic duct or between the stent and the bile duct wall.

FIG. 22 shows the balloon occlusion catheter placed within the transpapillary straight plastic bile duct stent that traverses the cystic duct. As above, this is an example of a type of stent that would not facilitate the goal of bypassing the cystic duct orifice due to the relatively narrow caliber stent and the additional side-hole within the bile duct aspect of the stent towards the liver end of the stent that would result in inadequate diversion of injected fluid away from the cystic duct despite the presence of an occlusion balloon.

FIG. 23 shows a large bore fully covered metallic biliary stent that would be deployed transpapillary and cross the cystic duct orifice. This stent would near immediately expand to abut the walls of the bile duct creating a seal. A balloon occlusion catheter with injection capabilities would be placed within the stent, the balloon inflated to abut the walls of the stent to create a seal, and the plasmid DNA or medical fluid be injected from within the stent at the level of the common bile duct.

FIG. 24 shows a large bore fully covered metallic biliary stent that would be deployed within the bile duct covering the cystic duct orifice but not crossing the papilla. This stent would near immediately expand to abut the walls of the bile duct creating a seal. A balloon occlusion catheter with injection capabilities could be placed within the stent or in the common bile duct below the level of the distal aspect of the stent. The balloon inflated to abut the walls of the common bile duct to create a seal, and the plasmid DNA or medical fluid be injected from within the stent at the level of the common bile duct. A short tether would be attached from the distal end of the stent and run into the duodenum.

FIG. 25 shows a method of placing the balloon occlusion catheter in the common bile duct and injecting fluid into the liver with the cystic duct occluded by a balloon placed percutaneously in the cystic duct.

FIG. 26 shows a method of placing the balloon occlusion catheter in the common bile duct and injecting fluid into the liver with the cystic duct occluded by an umbrella placed percutaneously in the cystic duct, at the gallbladder neck, or the junction of the cystic duct entry into the bile duct. The figure also shows an illustration of how a balloon could be placed inside the stent, such that it has effective seal across the stent and contrast does not leak out along the sides of the stent. Depictions of the balloon in different areas of the stent (common hepatic duct versus common bile duct) with effective seal of contrast offer confirmation of the flexibility of the approach.

FIGS. 27A-27C show applied implementations including stent, ballon and contrast agent. FIG. 27A shows an image obtained for a stent deployed with balloon inside. FIG. 27B shows an image for a stent deployed with balloon inside at approximately the common hepatic duct level, contrast above balloon. FIG. 27C shows an image for a stent deployed with balloon inside at approximately the common bile duct level, contrast above balloon.

FIGS. 28A-28D show that different flow rates modulate the efficiency of gene expression during biliary hydrodynamic delivery. Four separate flow rates (1 mL/sec, 4 mL/sec, 7 mL/sec, 10 mL/sec) were tested at a fixed volume of 40 mL and fixed DNA dose of 10 milligrams of plasmid DNA, pCLucf. The pigs used in the study were 37.1 to 45.9 kg in size. Immunohistochemistry for firefly luciferase protein was performed on pig liver tissue sections harvested at day 1 post-injection. FIG. 28A shows that the 1 mL/sec flow rate showed minimally apparent bile duct or hepatocyte staining. FIG. 28B shows that the 4 mL/sec flow rate displayed the highest transfection rate of hepatocyte staining, while also demonstrating bile duct and endothelial cell delivery. FIG. 28C shows that the 7 mL/sec in FIG. 28D and 10 mL/sec flow rate in had a similar transfection pattern of positive cells to the 4 mL/sec flow rate, with strong expression appreciated in bile duct and endothelial cells. The amount of the intensity of protein staining in hepatocytes was progressively less, along with mild decreases in transfection area. The most prominent staining in hepatocytes was appreciated at lobular borders and/or near large vessels.

FIGS. 29A-29C demonstrate that different volumes modulate the efficiency of gene expression during biliary hydrodynamic delivery. Three separate volumes (40 mL, 60 mL, 80 mL) were injected at a fixed 2 mL/sec flow rate and pDNA dose of 10 mg pCLucf. Pigs were all 40 kg in size. Immunohistochemistry for firefly luciferase protein was performed on pig liver tissue sections harvested at day 1 post-injection. FIG. 29A shows that the 40 mL volume demonstrated efficient hepatocyte transfection along with expression in bile ducts and endothelial cells. FIG. 29B shows that the 60 mL volume had relatively similar hepatocyte delivery to the 40 mL volume, while maintaining similar delivery into bile duct and endothelial cells. The 80 mL volume in FIG. 29C appeared to decrease the intensity of protein staining in hepatocytes as well as a mild decrease in transfection efficiency. The intensity in bile duct delivery was maintained.

FIG. 30 depicts the improvement of gene delivery with nanoplasmid versus regular plasmid for efficiency of biliary hydrodynamic injection. A novel DNA vector platform was tested against a regular plasmid DNA vector with traditional bacterial backbone to understand its effect on the efficiency of gene delivery during biliary hydrodynamic injection. To test this, 10 mg of a regular plasmid DNA vector and 10 mg of nanoplasmids were injected into pigs at 40 mL and 2 mL/sec. The nanoplasmid platform contains a smaller vector backbone (<500 bp) versus a regular plasmid DNA (>2 kb). Pigs were injected with the same expression cassette of LP1-ATP7B,C9, and stained for the C9-tag in pig liver tissue. Comparing equivalent pDNA doses normalized to DNA size, it was observed that the transfection area with nanoplasmid was significantly higher than the regular plasmid (p=0.0002). Representative photos of the IHC staining area with the nanoplasmid and regular plasmid are depicted. Parametric, t-test used (significant P<0.05).

FIG. 31 shows that the nanoplasmid mediates long-term expression in pigs after biliary hydrodynamic injection. The duration of expression from episomal DNA injected into pigs after biliary hydrodynamic injection was investigated. A nanoplasmid was hypothesized to mediate long-term expression in liver compared to a regular plasmid. Four pigs were injected with 20 mg nanoplasmid LP1-ATP7B, C9 and staining for C9-tag on the liver was used to validate the efficiency. Pigs were euthanized monthly to evaluate the duration and efficiency of expression. Representative images of IHC staining for each pig liver are depicted demonstrating successful expression out to four months. Regular plasmids injected as a control did not demonstrate any expression at one-month post-injection (data not shown).

FIG. 32 shows that biliary hydrodynamic injection mediates efficient expression with transposase that is maintained long-term in pigs. The duration of protein expression after biliary hydrodynamic injection with an integrating system was evaluated. The piggyBac transposon system was used to integrate a transgene cassette from a plasmid DNA injected through the hydrodynamic procedure. Three pigs were injected with 15 mg transposon and 5 mg transposase. Pigs were injected with the same expression cassette of LP1-ATP7B, C9, and stained for the C9-tag in pig liver tissue to evaluate the presence of gene-derived protein and the efficiency of delivery. Core and wedge biopsies were taken in the animals halfway through the experiment at 1.5 months post-injection and abundant expression of protein in canicular and cytoplasmic patterns (data not shown). At euthanasia of the animals 3 months post-injection, there was abundant staining among hepatocytes in all three animals for ATP7B, C9. A representative IHC image is displayed, along with an uninjected control tissue. The estimated transfection efficiency was calculated to be 41.83%-44.50%. This compares favorably to the delivery efficiency at 1-month post-injection for a cohort of pigs injected at the same pDNA combination (42.62%-48.44%; difference not significant). Both cohorts were not significantly different to pigs sampled at day3 post-injection. This indicates that transposons can mediate stable expression over time with the relative transfection efficiency showing no changes. Parametric, t-test used (significant P<0.05)

FIG. 33 demonstrates that biliary hydrodynamic injection can be re-dosed with similar efficiencies achieved with each gene. An unknown of biliary hydrodynamic injection is if pDNA can be successfully re-dosed. This was evaluated through a sequence of injections undertaken 4 weeks apart. Briefly, three pigs were injected with 15 mg transposon and 5 mg transposase encoding the expression cassette of hepatocyte-specific, LP1-ATP7B, C9. Four weeks later, the pigs were injected with a plasmid, pCMV-GFP-ATP7B, with a different protein tag and a ubiquitous promoter. The pigs were harvested three days later and stained for the C9-tag and GFP-tag in pig liver tissue to evaluate the presence of gene-derived protein and the efficiency of delivery. Representative serial sections of a single lobule are depicted on the left, showing that ATP7B, C9 and GFP-ATP7B can be detected in the same lobule and in the same cells. The GFP stain also reveals that other cell types (bile ducts, endothelial cells) can be targeted during a second injection (bottom left). Immunofluorescent staining adds further evidence for co-localization in the same hepatocyte (date not shown). The transfection efficiency by stained lobular area was compared for the first (C9) and second (GFP) injections, showing that both are similar (46.2% vs 50.7%; not significant). Parametric, t-test used (significant P<0.05)

FIG. 34 shows that increasing doses of plasmid DNA mediate higher transfection efficiency after biliary hydrodynamic injection. The influence and ability of progressively higher pDNA doses to increase transfection efficiency has not been tested. For this goal, four different pDNA doses (10 mg, 20 mg, 30 mg, and 40 mg) were studied. The plasmid, pT-LP1-ATP7B,C9, was used in all studies for standardization. Pig livers were harvested on day 3 after injection and were stained for the C9-tag with 1D4 antibody. Representative images of pig livers representing 10 mg, 20 mg, 30 mg, and 40 mg doses are presented. Quantification of the stained area in individual lobules is presented for each pDNA dose for randomly counted lobules in each pig, revealing a significant increase in the efficiency of biliary hydrodynamic injection with increasing pDNA dose. Parametric, t-test used (significant P<0.05)

FIG. 35 demonstrates evaluating the limits of plasmid DNA size that can be delivered by biliary hydrodynamic injection. Biliary hydrodynamic injection can mediate the delivery of naked plasmid DNA inside cells, but it is unclear if there exists an upper size limit of plasmid DNA for the approach. To address this, plasmid DNA of 12 kb and 17 kb in size were injected into the pig liver at injection parameters of 40 mL and 2 mL/sec. Plasmid DNA doses were 10 mg pDNA and 15 mg pDNA respectively. Pigs were harvested on day 3 post-injection. Representative areas of immunostaining for the 12 kb plasmid, pCDNA4/Full length FVIII, are depicted at low and high-power magnification. Images reveal gene delivery into every single lobule in the stained section at low power magnification. As seen at high power magnification, the darkest staining is found around the central vein reflecting increased local pDNA delivery. Quantification of the stained area that 49.48% of hepatocytes express the 12 kb gene, which was not significantly different from 5.5 kb and 8.6 kb plasmid previously injected. Testing of the 17 kb plasmid revealed similar ability of protein expression and relative efficiency (data not shown).

FIG. 36 shows that biliary hydrodynamic injection can achieve expression in vascular endothelial cells and liver sinusoidal endothelial cells. In previous studies, biliary hydrodynamic injection was observed to meet gene expression in endothelial cells and bile duct cells using a ubiquitous promoter. However, immunostaining near vessels or ducts can sometimes be unreliable. To validate the specific delivery of plasmid DNA into these cell types, two different endothelial cell-specific promoters was employed, that would only yield GFP expression specifically inside vascular or liver sinusoidal endothelial cells, respectively. The plasmids, pICAM2-GFP and pCD36p-Luc, were synthesized and injected into the pig liver at 2 mL/sec, 40 mL volume, and 10 mg pDNA dose. The pigs were harvested on day 3 post-injection. Representative IHC staining against GFP and Luciferase are depicted for each plasmid, respectively. For the ICAM-2 promoter, results show strong, clear GFP staining in every arterial endothelial vessel observed across the entire section. Larger venous vessels do not appear to have any staining, and there was no staining along the liver sinusoids. For the CD36 promoter, results show strong clear staining along the liver sinusoids, while portal areas are spared have more minimal staining.

FIG. 37 depicts the validation of volume to liver weight dosing for biliary hydrodynamic injection in pigs. Translation of the volume injection parameter to individuals of different sizes remains uncertain. To address this question, different injection parameters were tested based on a volume to liver weight dosing. These tests include 20 mL/kg, 30 mL/kg, 40 mL/kg, and 55 mL/kg. All injections were carried out at identical flow rates of 2 mL/sec with constant doses of 10 mg pDNA of pCLucf. Immunohistochemical staining results of the injections are demonstrated with IHC staining against firefly luciferase. Example results demonstrate that pigs of 25 kg and 27 kg can successfully express protein across different volume to liver weight doses, as tested 40 mL/kg and 55 mL/kg, respectively. Pigs as small as 5 and 15 kg were separately tested and showed similar results (data not shown).

FIG. 38 shows the evaluation of pressure thresholds for biliary hydrodynamic gene delivery into pigs. The minimum pressure threshold to mediate gene delivery through biliary hydrodynamic injection remains uncertain. To address this, a pig was injected with a constant pressure injection device. The pressure threshold was set for 50 mmHg to be maintained for the entire injection. The total volume injected was 30 mL, while the pDNA dose was 10 mg pCLucf. IHC staining was conducted for Firefly Luciferase on the liver tissue section. A control tissue sample injected with pCLucf at 2 mL/sec is provided for comparison. Results show that the pressure threshold at 50 mmHg was not sufficient for injection with no IHC staining for GFP or luciferase observed. A separate experiment demonstrated that 80 mmHg is sufficient for gene delivery.

FIGS. 39A-39F show ERCP-mediated hydrodynamic injection is feasible in non-human primates. Baboon liver anatomy is depicted (FIG. 39A), possessing a short common hepatic duct (CHD) of ~1.5 cm before the bile duct enters inside the liver (FIG. 39B). A duodenoscope used in humans can be advanced into the small intestine and the biliary orifice of baboons identified (FIG. 39C). Subsequently, the orifice can be successfully canulated to advance into the biliary system (FIG. 39D). Fluoroscopy pre-injection demonstrates the different branches of the biliary system in baboons (FIG. 39E). Post-hydrodynamic injection visualizes bile duct branches again, showing they are intact (FIG. 39F).

FIGS. 40A-40C show evaluation of the safety of biliary hydrodynamic injection in non-human primates. Different common clinical laboratory tests were tested pre-injection and several days post-hydrodynamic injection to monitor potential toxicity of the procedure. The study consisted of four baboons injected at similar parameters, with each baboon (FIG. 40A) A panel of liver function tests consisting of Alanine aminotransferase (ALT), aspartate aminotransferase (AST), lactate dehydrogenase (LDH), gamma-glutamyl transferase (GGT), albumin, and alkaline phosphatase was conducted. (FIG. 40B) A panel of hematology tests consisting of white blood count (WBC), granulocyte count, and hemoglobin was conducted. (FIG. 40C) Post-ERCP pancreatitis was evaluated by monitoring Amylase and Lipase enzymes, which are biomarkers pancreatic injury.

FIG. 41 shows primate bile duct walls have increased elasticity leading to leakage around the balloon. A series of fluoroscopic images are presented of three different baboons. Injection parameters were similar (3-4 mL/sec and 30-40 mL volume). The balloon was placed in the common hepatic duct (CHD) for each injection at a size of 8.5 mm. To help illustrate potential leakage, contrast was placed below the balloon. Pre-injection and post-injection fluoroscopic images are depicted for all three baboons, demonstrating an increase in gall bladder size post-injection reflective of fluid escaping around the balloon in the CHD and entering through the cystic duct into the gall bladder. The black arrow points to the common bile duct, which can be seen cleared of contrast post-injection, reflective of saline solution circumventing the balloon flowing in antegrade flow. The post-injection fluoroscopy image for baboon #3 also demonstrates notable dilation of all the bile ducts compared to the pre-injection, a finding which was observed in other baboons tested.

FIG. 42 shows oversized catheter balloons in the extrahepatic bile ducts can risk rupture. Larger balloon sizes could assure pressure seal and avoid leaks, but put more stress on the bile duct wall. To test this, a timelapse series of fluoroscopic images are presented in single baboon during hydrodynamic injection. Injection parameters were 4 mL/sec and 40 mL volume with saline solution. The pre-injection diameter of the common hepatic duct was approximately 3 mm, and the balloon was inflated to 15 mm. Upon hydrodynamic injection, the already maximally stretch bile duct wall is seen to rupture around the balloon, leading to leakage of contrast in the surrounding tissue. Similar findings can be replicated in pigs at ratios 4-5× balloon to duct size in the extrahepatic system.

FIGS. 43A and 43B show adjustments to catheter balloon location and size leads to successful seal in primate liver. A series of fluoroscopic images are presented of two different baboons. Injection parameters were similar between the two injections. In both baboons, the catheter balloon location is in the intrahepatic location in a left branch of the liver. The balloon was inflated 11.5 mm in size. (FIG. 43A) Pre-injection and post-injection fluoroscopy images are show for baboon #1 and baboon #2. As illustrated, the injected saline cleared intrahepatic ducts with hydrodynamic injection, as expected. Crucially, the gallbladder sizes in both animals were not increased post-injection, while the black arrow indicated that the common bile duct did not show clearance of contrast solution. (FIG. 43B) An image from one baboon is presented, showing hydrodynamic injection of contrast solution into a baboon liver via the biliary route. Contrast is observed entering into the parenchyma of the liver (Acinarization).

FIGS. 44A-44D show pressure monitoring can be used to detect leakage of fluid due to poor seal during biliary hydrodynamic injection in primates. A cohort of baboons had a series of biliary hydrodynamic injections performed on them. A pressure transducer (EchoTip Insight, Cook Medical), sensing a fluid-filled column in the injection port, was connected to the Multi-3V Catheter (Olympus). Injection parameters were used indicated above the graph. The balloon size was 8.5 mm in the common hepatic duct for the injections in (FIG. 44A), which demonstrated progressive fall in pressure during the injection. Faster flow rates and more volume were used to tested if this loss of pressure could be overcome in (FIG. 44B), but the results were the same with progressively loss in pressure. (FIG. 44C) An alternative strategy was employed to vary the flow rates in real-time during the injection. This was effective in stabilizing the pressure and in some cases raising pressure, but the total amplitude of the pressure remained low. (FIG. 44D) Adjustment the placement of the balloon (intrahepatic location, left branch) and size of the balloon to 11.5 mm led to stable plateau pressures during the duration of the injection, without the loss previously observed. Of note, these pressures were achieved at flow rates significantly below the other flow rates tested without good seal.

FIGS. 45A-45D show biliary hydrodynamic injection can mediate gene delivery in non-human primates. A cohort of baboons was obtained to test for expression of a delivered gene. Balloon size, location, and parameters were similar in all groups. (FIG. 45A) Two baboons were subject to repeat injections of DNA vector encoding human FIX (hFIX). The first injection was a 20 mg pDNA dose, while the second injection was 3-fold higher at 60 mg pDNA dose. Both animals displayed a dose-dependent response with 3-fold higher hFIX expression within individual baboons. (FIG. 45B) Gene delivery procedures repeated monthly in an individual baboon achieves similar peak expression of hFIX at day-1 post-injection at similar doses and parameters. (FIG. 45C) An alternative DNA molecule, the nanoplasmid DNA, was compared to a regular plasmid DNA with large bacteria backbone (e.g. antibiotic resistance genes) that is two-fold higher. As illustrated, the nanoplasmid allows for lower DNA doses that can still yield equivalent expression of hFIX levels to a regular pDNA at a much higher DNA dose. (FIG. 45D) In a separate experiment, a baboon was injected by biliary hydrodynamic injection with a GFP/Luciferase reporter DNA and euthanized 24 hours post-injection. Detection of GFP expression by immunohistochemical staining revealed positive gene expression around central veins of hepatic lobules in baboons.

FIG. 46 shows mechanical injuries from the guidewire can cause intrahepatic biliary leaks. A guidewire is used to initially help canulated and access the biliary system, followed by the catheter being driven over the guidewire into the bile ducts. As shown in fluoroscopic images, the advancement of a guidewire into the intrahepatic system is common, with here the guidewire projecting into the deep left ductal system. However, after the catheter was placed into the bile ducts and contrast injected, a small blush of contrast could be observed at the point where the guidewire tip was. During subsequent hydrodynamic injection, the size of the contrast/biliary leak increased, emphasizing that it communicates with the fluid injected and can serve as a sieve and restriction factor for pressure generation.

FIGS. 47A-47D show a stent-based approach can be utilized to do biliary hydrodynamic injections. A series of fluoroscopic images are depicted that demonstrate the utility of using a stent catheter without a balloon for the injection of contrast fluid into the liver. (FIG. 47A) The catheter with undeployed stent is advanced into the common hepatic duct past the cystic duct. (FIG. 47B) The stent within the catheter is 50% deployed with the stent opening up at the distal end, as marked by the black arrow. (FIG. 47C) Contrast was injected through the guidewire channel of the catheter leaving the olive tip of the catheter. No contrast was detected within the stent and no contrast extended around the stent into the cystic duct. (FIG. 47D) Pulling the stent and catheter back into the common bile duct and injection of contrast through the catheter now demonstrate that the cystic duct becomes opacified. Together, the stent deployment was able to block fluid entry into the cystic duct and holds potential for delivery of biliary hydrodynamic injection.

FIGS. 48A-48D show biliary hydrodynamic injection can achieve gene delivery into liver tumors. The Oncopig model has mutations in key tumor suppressor and oncogenes, TP53R167H and KRASG12D, that can be activated by the introduction of Cre recombinase. After Cre introduction by a virus into cells, large tumors can grow within 1-2 weeks in multiple different tissue types. (FIG. 48A) A liver tumor induced in pig liver is depicted (1-2 cm in size), which demonstrates a large, highly necrotic tumor on histology. Pigs with tumors were injected with a GFP/Luciferase reporter DNA by biliary hydrodynamic injection and subjected to immunohistochemical staining for GFP to detect cell types that are positive. (FIG. 48B) Positive cells for GFP were detected in peri-tumoral areas at the borders of the tumor, where normal tissue is infiltrated by tumor tissue. (FIG. 48C) Positive cells for GFP were also observed within the tumor tissue itself. (FIG. 48D) Positive expression for GFP in normal hepatocytes and normal lobules is still observed in the same liver section, indicating the ability to target both cell types.

FIGS. 49A-49C show ductal hydrodynamic injection can achieve gene delivery into pancreatic tumors. The Oncopig model has mutations in key tumor suppressor and oncogenes, TP53R167H and KRASG12D, that can be activated by the introduction of Cre recombinase. After Cre introduction by a virus into cells, large tumors can grow within 1-2 weeks in multiple different tissue types. (FIG. 49A) A pancreatic tumor was induced in pig pancreas. The tumor can be seen on histology in the pancreas section (black arrow). Pigs with tumors were injected with a GFP/Luciferase reporter DNA by pancreatic ductal hydrodynamic injection and subjected to immunohistochemical staining for GFP to detect cell types that are positive. (FIG. 49B) Pancreatic tumor section was obtained and stained for GFP, revealing scattered positive cells of different intensities. (FIG. 49C) A normal pancreatic tissue section on the same slide also reveals gene delivery primarily into ductal cells, as detected by GFP staining. Thus, both the tumor and normal tissue can be targeted for DNA delivery at the same time through a single injection.

### DETAILED DESCRIPTION OF THE DISCLOSURE

The prior art has established embodiments of the biliary hydrodynamic injection focused on flow rate as a key parameter for programming successful gene delivery into liver tissue. The prior art gives recommendations on optimal flow rates for achieving gene delivery from biliary hydrodynamic injection along with recommendations on use of specific flow rate to target specific areas in the liver.

The present disclosure relates to improve methods of hydrodynamic injection and delivery, which provide an advance over prior art approaches. In certain aspects, the present disclosure relates to compositions and methods for treating kidney disease. More particularly, the present disclosure relates to compositions and methods for treating kidney disease by gene therapy. As described in detail below, the present disclosure is based, at least in part, on the surprising discovery that the ureter hydrodynamic injection could mediate rupture of the kidney cortex variety of different flow rates and volumes, such that only limited parameters were observed to be safe. There is likely an absence of a vascular or lymphatic escape, which has previously never been tested before. The discovery was that volume was the key parameter that led to rupture, since lower volumes were better tolerated, regardless of flow rate. Nevertheless, injection parameters were discovered that were safe and could mediate gene delivery, which had never previously been described before, and indeed, it was doubtful that any could be discovered. It was also surprising that the procedure could be performed without the need for fluoroscopy, which has constrained all other gene therapy procedures prior. Additional surprises were the relative sensitivity of the kidney during proximal injections for injury, as well as that relatively small flow rates were capable of large increases in pressure during injection.

### Overview

The kidney is an attractive target for gene therapy. Current therapeutic strategies are ineffective for many patients, who require regular dialysis for chronic kidney disease (CKD). Concerning health care burden, CKD is one of the largest drivers of health care costs in the United States. Any solutions toward treating CKD would be extremely valuable, whether by reversing CKD or preventing its development. Chronic dialysis is a demanding and debilitating procedure for patients. Kidney transplant could be curative, but many patients aren't able to undergo kidney transplant due to limited donor availability. Kidney transplant also has many significant risk factors, particularly with complications of rejection and chronic immunosuppression.

Beyond chronic kidney disease, there are several other important kidney diseases. A relatively common rare disease is adult polycystic kidney disease (PKD), which affects around 500,000 people in the United States. PKD is caused by a mutation in the PKD1 or PKD2 genes. Causative mutations in these genes leads to overactivation of tubules leading to the formation of cysts throughout the kidney. Accumulation of these cysts overtime can later cause chronic kidney disease and need for a transplant. Treatments today are poor and lack significant efficacy. Many individuals require eventual kidney transplant with PKD. Other major diseases are various glomerulus disorders, including Anti-GBM disease and IgA nephropathy. These are typically marked by an immune process affecting these organs that leads to the destruction of the glomerulus through either inflammatory or fibrosis with the thickening of the glomerular basement membrane.

Current treatments for all these indications different kidney diseases are ineffective. Gene therapy is a new therapy which could help solve this clinical gap. However, gene therapy into the kidney is limited by several factors. Many investigators leverage the systemic infusion of viral vectors including adeno-associated virus (AAV). AAV is efficient and delivers genes to numerous organs including the liver. Unfortunately, AAV has very poor transduction into the kidney. Poor transduction is thought to be due to a number of factors, but the size exclusion of the glomerulus is the most problematic when filtering blood. There is no traditional route for viral vectors to migrate from systemic circulation into the kidney. Since systemic injection of viral vectors, is ineffective, other investigators have pursued local administration of AAV into the kidney. Examples of local routes include injecting through the renal artery or vein into the kidney. These routes of administration have shown promise in achieving delivery in mouse models but are still relatively inefficient. Another approach is to infuse vectors through the ureters. This strategy is a relatively non-invasive route not requiring any skin incision. However, the majority of infused viral vectors through ureters escape rapidly from the kidney and back into systemic circulation (Hum Gene Ther. 2019 December; 30 (12): 1559-1571). In these cases, liver transduction is more effective than the kidney.

Non-viral approaches would be attractive for kidney gene delivery. The 30+ million patients with chronic kidney disease in the United States means that the total number of patients far exceed the capacity of viral vector manufacturing. Treatment of any kidney disorder likely needs the ability to be redosed, since kidney cells have a shorter half-life than more stable cells like hepatocytes. Non-viral vectors could address this deficiency because they do not mount an immune response. Unfortunately, the most common strategies for delivering non-viral vectors also suffer from the same limitations as viral vectors. Approaches for delivering nucleic acids use delivery vehicles like lipid nanoparticles, which range in size up to 100 to 200 nanometers. Lipid nanoparticles are very efficient at delivering siRNA or mRNA into different cell types including hepatocytes and macrophages among other cell types. Lipid nanoparticles are especially useful in vaccine applications in combination with messenger RNA. Lipid nanoparticles are less efficient for DNA, since they mediate inadequate delivery into the nucleus.

If a lipid nanoparticle (LNP) could be obtained, and systemically administered through the vascular system, it would face the same size restriction barrier as viral vectors in the kidney glomerulus. Thus, LNPs would not efficiently enter the kidney in order to transfect kidney cells. In previous studies, local administration of nanoparticles into the kidney has not been described.

As an alternative approach, hydrodynamic gene delivery holds great promise. It leverages fluid pressure to physically deliver DNA inside different cell types. This occurs through the transient production of pores in the cell that leads to the entry of naked DNA into these cell types. This strategy is most well-known in mouse models where hydrodynamic tail vein injection can efficiently introduce plasmid DNA directly into the mouse liver. The same hydrodynamic tail vein technique can also deliver into other organs at much lower percentages. Investigators have tried to administer local fluid pressure into these organs in order to drive DNA entry into various other organs. Ultimately, local administration is the only method that can be translated into large animal models.

One study of hydrodynamic injection into the kidney was carried through the renal vein (Mol Ther. 2008 June; 16 (6): 1098-104). The renal vein was exposed in a rat model through a surgical procedure. The peak pressure during injection reported was 100 mmHg and a DNA concentration of 100 μg/mL was injected. For gene delivery into the kidney of a larger pig model, the catheter insertion was made into the right renal vein using an interventional radiology guided fluoroscopic procedure. An inferior vena cava (IVC) occlusion balloon was utilized to block the junction of the renal vein back into IVC, so that fluid during hydrodynamic injection would not leak back into the IVC. Using this strategy, the investigators were able to get gene expression in the pig kidney in the targeted kidney, as measured by firefly luciferase luminescence activity. The procedure appeared safe without any elevation in creatinine, but no immunohistochemical staining data was reported.

While the vascular strategy is promising, an alternative hydrodynamic approach is to inject through the urinary system. A proof of concept to this approach has been studied in mouse models, where hydrodynamic injection procedure was directed into the kidney pelvis (Sci Rep. 2017 Mar. 20; 7:44904). Prior to the injection, the mouse kidney is accessed by surgical opening and exposure of the kidney through the retroperitoneum. A needle was placed into the kidney pelvis and injection proceeded at high fluid pressure. The injected solution contained naked plasmid DNA. The results observed scattered positive cells multiple cell types in the kidney.

Previous studies provide multiple different routes toward administering hydrodynamic pressure into the kidney. A third route that has not been explored in the published literature is hydrodynamic injection through the ureters. The ureters are a unidirectional vessel going from the kidney into bladder, thus it may be easier to drive higher fluid pressure upon retrograde injection. Moreover, it is easy to access the ureters through simple cystoscopy into the bladder.

### Hydrodynamic, Retrograde Ureter Injection

A previous study that focused on retrograde ureter injection presented an idea of how a hydrodynamic procedure through the ureters could take place. This idea is not validated by any experimental evidence, so the designated procedure may or may not be successful in gene delivery. The procedure previously disclosed for hydrodynamic gene delivery into the kidney is summarized as follows:

The first step is to obtain a cystoscope and advance through the urethra into the bladder. Subsequently, the catheter is advanced through the cystoscope and into the right or left ureter. The catheter is then advanced to the distal end of the ureter and entrance to the renal pelvis, preferably using injection of contrast and fluoroscopy to confirm the location of the catheter. Optionally, there could be suction of fluid through the catheter to drain the renal pelvis of urine alleviating potential toxicity once the hydrodynamic injection commences. A balloon would be opened to seal at the ureter, to prevent antegrade flow of solution during hydrodynamic injection. The next step is to inject contrast and image with fluoroscopy to confirm the balloon seal, optionally followed by removal of contrast. The catheter circuit is then primed from power injector to distal tip with DNA solution. Subsequently, the hydrodynamic injection of fluid proceeds containing nucleic acids and/or proteins.

The balloon is then deflated after the cessation of injection. A repeat contrast injection and fluoroscopy may be performed. The catheter and guidewire may be removed from the ureter, and the procedure repeated for the un-injected kidney when desired. When the procedure is completely finished, the catheter is removed from the ureters, bladder, and urethra outside of the patient along with the cystoscope. Post-injection, there are optional steps of monitoring creatinine, blood urea nitrogen, and glomerular filtration rate after injection to monitor damage from hydrodynamic injection.

To aid in the ease of the procedure, a number of additional steps can be incorporated. The use of a guidewire can facilitate canulation of the ureteral orifice, followed by insertion of the catheter. The injection of fluid containing nucleic acids and/or proteins with a power-injector at high speed and high pressure can be monitored with a pressure catheter for quality assurance of injection goals reached Another option is to chase the macromolecule-containing fluid with a non-macromolecule containing solution such as normal saline to ensure complete delivery of solution into the kidney.

There were several hypothetical injection parameters outlined in the study, although no data was provided to support its efficacy, not for its biological rationale. Flow rates of at least 1 mL/second, or at least 2 mL/second, or at least 3 mL/second were proposed. A volume injected up to 10 mL, 20 mL, 30, 40 mL, or 50 mL total was proposed, with the result that volume escapes from the renal pelvis into the parenchymal tissue. It is uncertain how this volume would be affected by changing pig sizes. A proposed pressure achieved during kidney injection is at a minimum of 50 mmHg, or at least 75 mmHg or at least 100 mmHg. It is not known if any of these pressures are efficacious in gene delivery into the kidney.

In other aspects of the procedure, methods of co-administration of various different drug solutions were also presented that would decrease inflammation and/or potential infection from the injection procedure. The best drugs to use in appropriate doses and their efficacy at those doses were not described. Methods of targeting cell-specific targeting were described, by injecting DNA molecules that contain cell-type specific promoters to target expression for specific cell types, but without confirming data

In summary, previous studies do not establish exact injection parameters that would effectively deliver DNA inside cells of the kidney. This ranges from effective flow rates, volumes, and pressures achieved during injection. The relationship of volume injected to the size of the mammal or kidney size is not presented. It is unknown what volumes would be appropriate in order to translate the technique into human patients.

Previous studies do not present what DNA doses would be required in order to achieve gene expression the kidney from the ureter proximal kidney placement. Additionally, no information on the safety of the technique in regard to the injections are reported, concerning proposed injection parameters. Studies so far have not presented a technique for gene therapy that is safe and effective and produces the desired gene therapy outcome.

### DETAILED DESCRIPTION

The instant disclosure also describes details about effective ways to perform hydrodynamic gene delivery through the ureter to target gene expression to the kidney. The present disclosure also subscribes novel ways of doing the procedure to provide more efficacy and safety.

### First Procedure Method to Yield Gene Delivery

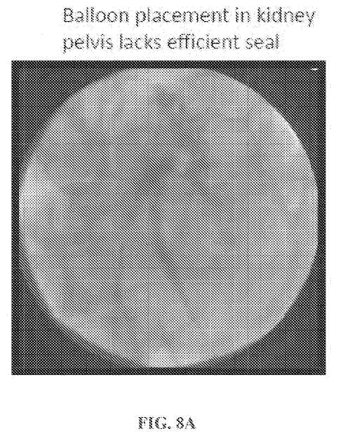

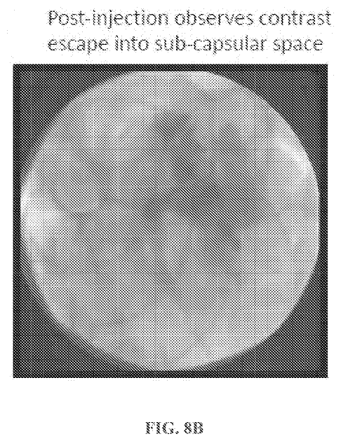

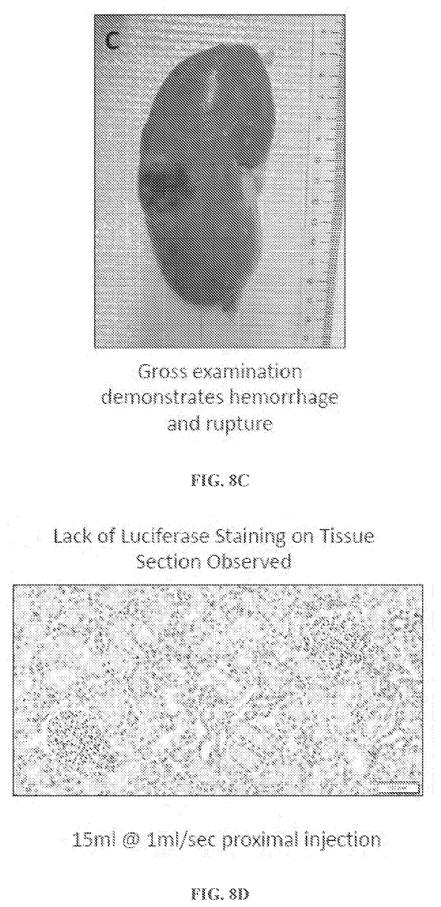

Previous studies disclosed a method of hydrodynamic injection through the ureters, where the catheter balloon is place in the ureter in close proximity to the kidney. Replication of this method was difficult. Placement of the balloon in close proximity to the kidney without the catheter slipping into the renal pelvis was not possible. Placement of the balloon inside the renal pelvis was found to precipitate fluid escaping during hydrodynamic injection due to an ineffective seal (see e.g., FIG. 8A).

To develop a novel method that would be more robust and easier for the practitioner to execute, a novel procedure for hydrodynamic injection through the ureter was designed. The first step in this procedure is to enter a cystoscope into the bladder through the urethra. A catheter could be advanced through the working channel of a cystoscope and into the bladder. Using the camera of the cystoscope to examines the wall of the bladder, the ureter orifice (UO) can be visualized. The catheter would then be advanced toward the ureter orifice to allow for calculation to be achieved.

Once the catheter is into the ureter orifice, it would be advanced and approximately 1, 2, or 3 cm into the ureter. At this point, the balloon on the catheter would be inflated such that it is just at the opening of the ureter orifice into the bladder. The inflated balloon would be seen by a camera with a cystoscope as a bulge in the bladder wall, thereby confirming its localization. The benefit of this strategy is that the balloon placement can be verified with visualization via the cystoscope camera. Thus, no fluoroscopy is required to localize the placement of the catheter. This creates enumerable safety benefits, including a lack of radiation safety required and a lack of expensive equipment including a C-arm being necessary.

Another benefit is that the bladder wall adds reinforcement around the balloon when inflated with its muscular wall, thereby helping to prevent issues of ureter rupture. Furthermore, the ureter orifice can be visually monitored during the infection to see if there is any fluid leakage, giving real-time feedback that the hydrodynamic injection was successful. The major advantage of this procedure and approach is that it can be conducted in facilities without fluoroscopy imaging, but rather in a routine outpatient clinic. Furthermore, the patient and clinical providers would be exposed to less radiation. This new procedure increases the availability of the technique, since bedside cystoscopy is relatively routine and can be conducted in any outpatient facility.

A series of tests were conducted to evaluate hydrodynamic injection from distal ureter balloon emplacement. In all studies, the balloon was able to seal the ureter successfully and block all antegrade hydrodynamic outflow into the bladder.

An important aspect of hydrodynamic gene delivery is that the optimal injection parameters change with slight modifications of the procedure. Given the distal ureter placement of the balloon, the immediate considerations for this catheter location are what volume is needed to fill out the ureter and the renal pelvis, with the ultimate goal of pushing fluid into the kidney parenchyma during hydrodynamic injection. The ureter volume was estimated to be between 4 to 5 mL during empiric testing done prior to the injection. The entire ureter and renal pelvis volume ranged from 6 to 9 mL, when slowly filling it up with radiocontrast solution.

The optimal injection parameters from the distal ureter location were empirically derived. The first step of their testing ascertain what volume and flow rates could be safely injected to the kidney without significant injury. The third parameter was to test which volume of injection fluid could mediate entry of radio-contrast injection to the kidney parenchyma, which in previous studies in the liver was a proxy to DNA entry into tissues. To accomplish this, a series of injections were performed validating safe and efficacious parameters for injection.

The present disclosure also presents injection volumes that avoid rupture of the kidney during hydrodynamic injection. Rupture of the kidney is associated with a physical tear from the renal medulla extending through the rental cortex. As exhibited in the data in the disclosure, the tear usually occurs only at one location in the kidney, approximately 1 cm in size. The kidney rupture causes a small to large amount of hemorrhage into the fibrous capsule surrounding the kidney. Repeated tests of hydrodynamic injection into different kidneys demonstrated that hemorrhage from the induced tear will eventually stop so that no pig suffered significant harm from the kidney injury. However, it's uncertain if this would be a more serious issue if it occurs in human patients. Thus, hydrodynamic injection should be optimized so that the kidney rupture would not occur.

Optimally, injection parameters of less than 20 mL volume and at a flow rate less than or equal to 2 mL/sec are utilized in order to avoid kidney rupture, when the injection occurs near the opening of the ureteral orifice. At volumes exceeding 20 mL, kidney rupture occurred at a high frequency, despite even low flow rates like 0.5 mL/sec used. Flow rates greater than 2 mL/sec should also be avoided at these volumes, since they were found to cause rupture. Flow rates between 0.5 mL/sec to 2 mL/sec intermittently caused kidney rupture in some animals. Larger volumes can be contemplated >20 mL, but require the use of decreased flow rates, ranging from 1 mL/sec to 1.5 mL/sec, and still carry the risk of kidney rupture in some animals.

Concerning the efficacy of gene delivery from these parameters, an optimal injection parameter to mediate gene entry into kidney cells would optimally be a volume between 10-20 mL is injected into a kidney with a mass of 70-80 g. This volume would be injected at 0.5 to 2 mL/sec in order to acquire gene expression, while avoiding deleterious kidney trauma and injury. Gene expression could also be seen at higher flow rates of injection, but this injection comes with aforementioned risk of kidney rupture, which are undesired.

One unexpected result was that relatively low flow rates in the ureter can generate significant pressures. It was observed that flow rates of 0.5-1 mL/sec generated pressure of 80 mmHg. Flow rates of 1.5-2 mL/sec yielded 120-140 mmHg. Consequently, little flow rates are necessary to generate pressure, which explains why even 0.5 mL/sec could yield gene expression and why relatively slow flow rates were still associated with the risk of kidney rupture. Optimally, a pressure of at least 80 mmHg could be targeted during injection in order to achieve gene expression within different cell types in the kidney.

Optimally, the balloon would be deflated after hydrodynamic injection, which can be confirmed on visual camera inspection from the cystoscope. The catheter can be withdrawn from the ureter orifice and into the bladder. The catheter would then be withdrawn through the cystoscope followed by the cystoscope being withdrawn from the patient. Any damage to the kidney could be monitored through serum testing for creatinine and other biomarkers.

### Second Procedure Method to Yield Gene Delivery

In a different example, the procedure proceeds via an alternative method. The cystoscope would be advanced into the bladder through the urethra. A guidewire would then be advanced through the cystoscope and into one of the ureter orifices. The guidewire would be advanced all the way into the kidney pelvis, where it would begin to loop signifying that it is present the kidney, or alternatively resistance would be felt while advancing the guidewire. The cystoscope would be withdrawn from the bladder. The catheter would be subsequently inserted over the guidewire and advanced for the kidney. The advance of the catheter would be monitored with fluoroscopic imaging in the use of a C-arm. Preferably, the catheter has radio-opaque markings to verify its placement while it is being advanced.

In this example, the catheter would be placed in proximity to the kidney pelvis. The catheter would still be located within the ureter. Successful blockage of the ureter would be realized through testing for the blockage of antegrade radiocontrast flow. In one embodiment, the guidewire would remain in the ureter to help verify its position, even after balloon inflation and injection. The guidewire could extend above or below the balloon. In a different example, the guidewire would be removed and the localization of the catheter ascertained with just contrast injection alone.

Preferably, the catheter balloon would be at least 1 cm, 2 cm, or 3 cm below the kidney pelvis to assure that the balloon is able to fully occlude the ureter, which is cylindrical in shape, as opposed to the kidney pelvis, which has a more conical shape. Optimally, a balloon size of 11, 12, 13, 14 or 15 millimeters is used to successfully seal and block the ureter.

For this proximal injection near the kidney pelvis point, the optimal injection parameters disclosed are as follows:

For volumes less than or equal to 15 mL/sec, the flow rate should be less than or equal to 2 mL/sec. Optimally, the total volume injected is equal to or less than 10 mL. A small injection volume is required due to the small volume in the upstream renal pelvis and ureter which is estimated to 3-4 mL total. Moreover, a small injection volume is also used to avoid rupture of the kidney. Preferably, the flow rate is between 0.5 to 1 mL/sec inclusive to mediate gene expression of plasmid DNA inside the kidney.

In another example, a constant pressure injection device could be employed for the injection. This would target pressure to specific settings, decreasing the risk of kidney rupture. Moreover, given that many patients' kidneys would have chronic kidney disease, the relative stiffness of the tissue would be different than a normal kidney. Stiffness would influence the relative resistance of the tissue thereby increasing pressure. For the proximal location, the pressure generated was evaluated at a given parameter. As an example, 10 mL was injected at a flow rate of 1 mL/sec. The pressure tracing reveals a peak pressure of 105 mmHg was achieved. In optimal embodiments of the invention, a pressure of at least 100 mmHg could be targeted for injection from the proximal location. In other embodiments, a pressure of at least 80 mmHg could be targeted for injection from the proximal location.

After successful injection at these parameters, the balloon would be deflated, and the catheter withdrawn from the ureter, bladder and urethra. Subsequently, the guidewire would also be withdrawn from the ureter, bladder, and urethra. The patient would be monitored for any toxicity with follow up urine analysis and serum chemistries including creatinine levels Hydrodynamic, Retrograde Ureteral Injection of Viral Vectors

### Hydrodynamic Injection is Traditionally Used to Deliver Plasmid DNA Inside Cells.

Consequently, it is traditionally thought of as a nonviral modality. However, hydrodynamic injection is a non-specific process that mediates the delivery and entry of any number of DNA RNA proteins or even viruses into cells (J Gene Med. 2006 July; 8 (7): 852-73). Hydrodynamic injection can have multiple different mechanisms in this process from greater intracellular entry into cells, as well as greater penetration of macromolecules into the tissue.

Viral vectors are the traditional backbone of gene delivery. Through millions of years of evolution, viruses have acquired properties to target, internalize into cells, and for DNA viruses, traffic into the nucleus. However, viruses repurposed for gene therapy are not adapted for the high-level delivery efficiency required. For example, viruses only need to target a fraction of cells in a given tissue in order to replicate. Viruses have not readily acquired the ability to permeate through tissues at levels that would be desired for gene therapy, replacing a gene inside every cell for example.

There have been limited studies on the potential synergy between hydrodynamic gene delivery and its use with traditional viral vectors. In one study, vein injection was used to increase the delivery of an SV40 viral vector into the liver (Hum Gene Ther. 2005 March; 16 (3): 361-71). It was noted that expression levels are much higher than the viral actor alone. In this instance, the SV40 vector does not exclusively target liver with natural tropism, such that hydrodynamic delivery helps focus all viral vector delivery into the tissue.

Even for modalities with natural tropism for the liver like adenovirus, hydrodynamic delivery could play a beneficial role. Because hydrodynamic injection is a local procedure that forces the delivery of substances into a given issue, it has the benefit of greatly decreasing viral vector doses required for treatment. This has been seen in an early study in mouse models where hydrodynamic injection of adenoviral vectors increased hepatic transduction while reducing the levels of inflammation normally elicited by the adenovirus (Mol Ther. 2005 July; 12 (1): 99-106). This study was also translated into a nonhuman primate model where injection at higher efficiency also helped reduce the amount of vector doses needed, leading to a longer-term expression than otherwise required (Mol Ther. 2007 April; 15 (4): 732-40). Disease relevant ability of the approached was proven in a rat model of hyperbilirubinemia, wherein lower viral vector doses were used when combined with hydrodynamic gene delivery into liver (Hum Gene Ther. 2011 April; 22(4): 483-8).

As discussed above, there have been a few studies of hydrodynamic gene delivery into the kidney, which have shown only modest efficacy of gene delivery. Viral vectors have been injected through the renal vein, regal artery, subcapsular space, and through the ureters, all only mediating modest transduction of the kidney (Hum Gene Ther. 2019 December; 30(12): 1559-1571). There is a large gap in efficacy currently with the delivery efficiency in order to treat clinically relevant diseases, wherein depending on the disorder, the majority of cells in the glomeruli or tubules would need to be targeted.

One study looked at the delivery of viral vectors in combination with hydrodynamic gene delivery through the renal vein and rats (Mol Ther. 2008 June; 16(6): 1098-104). The authors suggested that there was increased delivery efficiency with the combination of our vectors and hydrodynamic delivery compared to vectors delivered at a normal rate. The authors claimed that minimal-to-no adenoviral transduction occurred with the normal infusion rate, whereas GFP staining only occurred with hydrodynamic delivery in rats. This experiment occurred in a rat model, so that discovery might not occur in large animal models.

The instant disclosure also improves on the previous studies and describes methods to synergistically deliver viral vectors through the ureter system at hydrodynamic pressure. While viral vectors have been infused through the ureter system previously, they have never been infused at hydrodynamic pressure. The instant disclosure combines these two modalities to remedy the previous low efficiency of minimal gene delivery through the ureters. Another objective is how to scale any of these methods into large animals, in order to translate these methods into treatment of human subjects.

The instant disclosure also describes optimal hydrodynamic parameters that can be used for delivery of viral vectors. These hydrodynamic parameters are just below the maximal pressures that cause tissue injury in kidney rupture, as described elsewhere in the invention. The invention teaches that compared to a traditional viral vector infusion into the ureters at a flow rate of less than 0.1 mL/sec, the higher flow rates and volumes used to inject viruses in the procedure are more effective in yielding increased viral vector penetration into the tissue and resulting viral transduction. The mechanism of higher transduction is caused by greater permeability between tissues via higher fluid pressures at injection.

Exemplary viral vectors that can be used in the invention include adeno-associated virus, adenovirus, lentivirus, retrovirus, baculovirus, anellovirus, and Sindbis virus. The invention is not specific to a virus type or viral serotype, since the hydrodynamic gene delivery process is particle nonspecific, such that all viral vector particles could stand to benefit from this combination.

The present disclosure also relates to a redosable viral vector technique. The hydrodynamic injection procedure can be modified to push through a non-viral vector solution first, such that it would clear any antibodies present. Subsequently, viral vector containing solution is rapidly infused, such that the hydrodynamic fluid pressure immediately pushes viral vectors into the tissue and target cells. This obviates any antibodies present, such that viral vectors can be reliably dosed into the kidney.

The synergy of these two modalities has not been described in previous studies, so it is uncertain if there would be any improved transduction with a retrograde ureter hydrodynamic injection of viral vectors. In particular, previous evidence suggests that viral vectors could readily be ejected out of the kidney through a retrograde ureter injection, therefore it's uncertain if the additional pressure would facilitate that process, rather than leading to enhanced gene delivery (Hum Gene Ther. 2019 December; 30(12): 1559-1571).

The ultimate goal of hydrodynamic injection is the generation of pressure, which serves to create pores in the cell membrane to allow for gene delivery. A previous publication discovered that flow rate is key parameter that determines pressure in the biliary system during hydrodynamic injection (Huang, PLOS One 2021). However, a challenge with the prior art is that flow rate may be not be the optimal injection parameter for hydrodynamic injection. The actual pressure achieved during hydrodynamic injection depends on many different factors including the bile duct diameter, liver stiffness, liver volume, liver size, and viscosity of the injection substance formed. The injection parameters in the prior art were all formulated around liver sizes that were roughly equivalent within a narrow range (800 to 1000 grams), and with injection solutions of similar viscosity. Moreover, all the livers were healthy in animals that lacked any disease. More importantly, pig liver at the cellular level has significantly more fibrosis at baseline versus a human liver. Specifically, this is seen in prominent fibrotic tissue in between every pig lobule. The different composition of the pig liver could influence the injection resistance in this system, which would ultimately influence the pressure achieved versus the flow rate injected. These differences together make translating the injection parameters from pigs into humans a challenge, despite humans and pigs possessing similar organ sizes. Thus, the prior art may be challenged in enabling the exact gene delivery efficiency achieved in the pig studies.

Another challenge is that flow rates achieved during biliary hydrodynamic injection may not be translated into expected pressures readily. More specifically, a challenge in translating flow rate into pressure during biliary hydrodynamic injection, however, is that it does not appear to yield reliable translation, according to the prior art. Both 1 mL/sec and 2 mL/sec both appear to generate 80 mmHg in pressure during injection, for example (Huang, PLOS One 2021). Thus, a physician is left uncertain how to program a power injector for gene delivery if guided by a given pressure threshold to reach.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

The current disclosure describes and teaches solutions for that challenge. The disclosure teaches a method of injecting at defined pressure perimeters instead of defined flow rate parameters to be executed during biliary hydrodynamic injection. This is because the flow rate needed for gene delivery could be different between patients for the factors defined above. A prominent example of this would be a patient with cirrhosis or non-alcoholic steatohepatitis. The defined pressure parameters in the disclosure are such that these thresholds are sufficient to achieve gene delivery into the pig liver or another mammalian liver. Furthermore, the disclosure teaches for pressure thresholds, which should not be exceeded because of a loss of efficient gene delivery. The disclosure also teaches that given pressure thresholds that will cause tissue damage and injury.

In order to achieve a pressure-directed biliary hydrodynamic injection, the first step is to acquire a balloon catheter, which contains a lumen that can fit a pressure sensor or be connected to transducer. This pressure sensor should be able to monitor and reflect the pressure within the biliary system. The pressure sensor will be connected to a device, which can give real-time pressure results and/or graph the pressure achieved during injection in real-time.

The biliary hydrodynamic injection procedure will commence similar to the established protocol in the prior art, with the advancement of the balloon catheter to the common hepatic duct. In other embodiments, the catheter could be placed within the common bile duct. After the catheter is localized in the common hepatic duct, a pressure sensor will be attached or will be inserted into the catheter through a dedicated lumen. In some embodiments, the pressure sensor is already present in the catheter. A baseline reading of the pressure in the biliary system will be conducted without the balloon inflated, followed by a pressure measurement with the balloon inflated. A test solution not containing the plasmid DNA or other nucleic acids will be injected into the bile duct. This test solution is meant to have the same osmolarity, osmolality, and viscosity as the DNA injection solution. Beyond DNA or other nucleic acids, it should not contain any other active drug substances that may be included in the therapeutic DNA solution. A power injector will be loaded with this test solution substance.

The balloon on the catheter will next be inflated. Subsequently the test solution will be injected into the biliary system while pressure is monitored. The goal pressure to be achieved is greater than 80 mmHg. Initial injection parameters for the test injection should be at 3 mL/sec. The total volume for the test injection should be at most 15 mL, 10 mL, or 5 mL. In other cases, the total volume for the test injection should be at most 40 mL, 30 mL or 20 mL. This volume should be sufficient to measure the column of total fluid resistance in the entire circuit and gauge if sufficient pressure will be achieved. The prior publication states that a steady state pressure is achieved within the first second of the injection supporting that only a short injection time in total injected volume is needed for a test injection (Huang, et al. PLOS One 2021).

Depending on the results of the test injection, the flow rate will be adjusted. If the flow rate yields a pressure above 80 mmHg, then the flow rate will be chosen at that threshold for the injection and injection of DNA solution will proceed. If the pressure exceeds 250 mmHg, then the flow rate will be reduced below 3 mL/sec. If the flow rate was not above 80 mmHg, and the flow rate will be adjusted to a higher speed. For any adjustments that would occur, a second test injection will occur. In general, the flow rate could be adjusted up or down 1 mL/sec in order to achieve the desired outcome, whether increasing the pressure or decreasing the pressure experience. In other embodiments, shorter increments of 0.5 mL/sec could be utilized to adjust the flow rate. Once the correct pressure level is achieved on test injection, the DNA solution injection will then commence. The DNA solution injection will be programmed at the flow rate establish that will yield the desired pressure.

In other embodiments of the invention, a flow rate series is conducted during one single test injection, wherein multiple flow rates are tested. In this embodiment, at least two or more test flow rates will be tested within a single test injection. For example, an injection could commence at 1 mL/sec and then progressed to 2 mL/sec, 3 mL/sec, and 4 mL/sec. During this series of test injections, the pressure will be monitored throughout, creating a curve of multiple different pressures. This pressure curve may have many different forms, including a stepwise function. From this test injection, one could precisely determine within the range of flow rates tested which would be the optimal and achieves the desired pressure for the injection. In order to conduct a test series, it is envisioned that the total test volume would need to be larger than a single test injection. In these embodiments, the total injected volume may be at most 40 mL, 30 mL or 20 mL in total volume in order to test all of the different injection parameters.

In preferred embodiments of the invention, the minimum pressure for efficient hydrodynamic gene delivery would be greater than 50 mmHg, or greater than 80 mmHg, or greater than 100 mmHg. The maximum pressure for efficient hydrodynamic gene delivery would be less than 200 mmHg or less than 250 mmHg.

The prior art teaches that “pressure in the biliary during hydrodynamic gene delivery will be equal to or greater than 40 mmHg, 50 mmHg or more. In at least some aspects, an upper limit may be 200 mmHg, although in certain systems higher pressures may be employed.” However, the prior art only teaches pressure levels that could be achievable by a power injector, based on its technical specifications. Moreover, while the power injector could theoretically achieve these different pressures, the prior art does not tie any of these results to actual gene delivery. Thus, there is an unknown of what actual gene delivery parameters would be sufficient for gene injection. The current disclosure remedies the prior art by supplying data on what injection pressure which achieve a given gene delivery parameter.

For example, the current disclosure teaches that pressure below 50 mmHg for hydrodynamic pressure only achieves minimal gene delivery through the biliary system, as measured by immunohistochemical staining (<10%) for reporter genes, firefly luciferase and green fluorescent protein. Only at pressures of 80 mmHg or above could efficient immunohistochemical staining for reporter genes for reporter genes be observed (>20%). Moreover, at pressures ranging from 100-150 mmHg was the transfection area was increased even more compared to lower pressures.

The disclosure is advantaged in not being tied to any given species, allowing empiric measuring and tailoring of the injection parameters for that species during that procedure. The disclosure is also advantaged in deciphering the inherit differences between subjects within a species, allowing for customized injection parameters to be deciphered.

The disclosure is made possible by the rapid accumulation of pressure within the biliary system, such that only minimal fluid volumes are required for injection in order to ascertain what flow rates cause what pressure levels.

The present disclosure relates to compositions and methods for treating diseases of the pancreas. More particularly, the present disclosure relates to compositions and methods for treating diseases of the pancreas by gene therapy. As described in detail below, the present disclosure is based, at least in part, on the surprising discovery that the volume of fluid required for sufficient gene expression is lower than 20 mL, as previously published to be sufficient. The injected volume can be as low as 5 to 15 mL and still achievement effective gene delivery. The flow rate required to transfect pancreatic cells is lower than 2 mL/s as previously reported to be required. The flow rate sufficient to mediate gene delivery is at least 1 mL/sec. In other studies, it was at least 0.5 mL/sec. These two findings were in conjunction with the discovery that the tissue injury with hydrodynamic injection into the pancreas could be removed if these lower injection parameters were utilized, making a significant difference in immune infiltration and necrosis compared to the parameters outline in the prior art. The present disclosure also outlines optimal occlusion balloon sizes that are smaller than the listed prior art, being discovered that larger balloon sizes can rupture pancreatic ducts, but smaller balloon sizes don't do this and still can have sufficient seal for injection. In particular, 8 mm balloons are sufficient. As this is translated to patients, one will likely inflate balloons to diameters that are 1, 2, 3, 4, 5 mm or a maximum of 5 mm larger than the diameter of the pancreatic duct. The current disclosure also rectifies a gap in the prior art, teaching that pressure required to transfect pancreatic cells is lower than 75 mmHg above baseline pancreatic duct pressure. It is likely between 30-75 mmHg.

### Overview

Gene therapy of the pancreas is a potential modality for a variety of diseases from autoimmune diabetes to genetic chronic pancreatitis to pancreatic cancer. Proof-of-concept studies employing viral vectors have achieved modest efficiency in rodents. Importantly, gene delivery into the pancreas of a human-sized, large animal model has not been achieved, representing a major gap in the field. Herein is demonstrated proof-of-concept that an endoscopic procedure accessing the pancreatic duct by endoscopic retrograde cholangio-pancreatography (ERCP) allows for non-viral hydrodynamic delivery of naked plasmid DNA solution, mediating efficient delivery into a variety of cell types in the pancreas of adult pigs, including almost 100% transfection of islet cells, paving the way for future therapeutic development.

The pancreas is an important exocrine and endocrine organ of the body that plays an important role in food digestion and energy storage. Disruption of pancreatic function can lead to a wide variety of disorders. Pancreatic digestive enzyme secretion through the pancreatic duct leads to disorders of pancreatic insufficiency. Chronic enzyme secretion of this kind is seen in cystic fibrosis. Chronic pancreatitis is marked by inflammation, scarring of the pancreas, and can also cause pancreatic insufficiency, a condition which occurs when the pancreas does not make enough digestive enzyme to break down food and absorb nutrients. Hereditary pancreatitis is an inherited disease mutation where patients suffer from pancreatic insufficiency and/or chronic damage at a very young age.

The pancreas is a vital organ in the pathogenesis of a variety of different diseases, such as Type 1 and Type 2 diabetes that are caused by a disruption or dysregulation of insulin production. The beta islet cells of the pancreas secrete and regulate insulin, modulating total body stores of fat and protein. In type 1 diabetes, insulin-producing beta islet cells are destroyed by autoreactive T cells, leading to absolute insulin insufficiency and hyperglycemia. Type 2 diabetes causes systemic insulin resistance and relative insulin deficiency. Pancreatic cancer is another devastating disease with a poor survival rate. One strategy to treat disorders of the pancreas is gene therapy, which could deliver genes that are found to be deficient in pancreatic cells, or by transforming pancreatic cells. Gene therapy could also be used to deliver proteins that could fight against genetic diseases, cancer, or autoimmune diseases.

Various strategies have been attempted for gene therapy of the pancreas. Viral strategies have employed systemic or targeted therapy through vascular routes (see e.g., Griffin, M A et al. “A novel gene delivery method transduces porcine pancreatic duct epithelial cells.” Gene therapy vol. 21, 2(2014): 123-30. doi: 10.1038/gt.2013.62) while other studies have shown the feasibility of injecting virus through the pancreatic duct (see e.g., Wang, Yuhan et al. “Long-Term Correction of Diabetes in Mice by In Vivo Reprogramming of Pancreatic Ducts.” Molecular therapy: the journal of the American Society of Gene Therapy vol. 26, 5(2018): 1327-1342.) Non-viral strategies have also been employed leveraging hydrodynamic injection through the pancreatic artery in rats (see e.g., Ogawa, Kohei et al. “Efficacy and Safety of Pancreas-Targeted Hydrodynamic Gene Delivery in Rats.” Molecular therapy. Nucleic acids vol. 9 (2017): 80-88) as well as via the pancreatic duct in mice Yamada Y, et al. In Vivo Transgene Expression in the Pancreas by the Intraductal Injection of Naked Plasmid DNA. Journal of Pharmaceutical Sciences. 2018 February; 107(2): 647-653). However, these non-viral strategies have not been translated into large animal models, and there is only one published report of viral vectors in neonatal pigs at birth (~1.5 kg), wherein AAV was delivered via the celiac artery in the pig, disseminating the vector into the left gastric and splenic arteries which service the pancreas (see e.g., Griffin, M A et al.). If translatable pancreatic gene therapy strategies can be achieved in adult animals, other important factors would be to find out how efficiently gene delivery could be delivered into the pancreas and what cell types should be efficiently targeted.

In the present disclosure, endoscopic retrograde cholangio-pancreatography (ERCP) was used to mediate non-viral hydrodynamic gene delivery through the pancreatic duct in pigs. Non-viral gene therapy was used because its significantly lower cost may allow for routine use in metabolic conditions such as diabetes. Previously, ERCP has been used to deliver plasmid DNA efficiently into hepatocytes (see e.g., Kumbhari V, et al. Successful liver-directed gene delivery by ERCP-guided hydrodynamic injection (with videos). Gastrointest Endosc 2018; 88:755-763.e5).

Very few studies have been conducted for pancreatic diseases even though there is significant potential for gene therapy to make a meaningful impact on pancreatic disease. One approach to gene therapy is to administer viral vectors or nanoparticles, but they do not accumulate to the extent needed in the pancreas. Approaches that been more successful target local administration of viral vectors of nanoparticles to the pancreas, such as injection through the pancreatic artery or vein. Intraductal delivery is another technique used. One drawback to early investigations is the use of mouse and rat models because they respond to treatment with very low efficiency. More research is needed to ascertain whether viral vectors are an efficient way to deliver gene therapy.

Hydrodynamic injection is a technique that mediates non-viral delivery of nucleic acids including DNA and RNA. Studies in mouse models conducted by various groups show that hydrodynamic injection can mediate a limited amount of gene delivery through venous or ductal routes Resulting data from rodent studies is not robust, and there have been few follow-up studies.

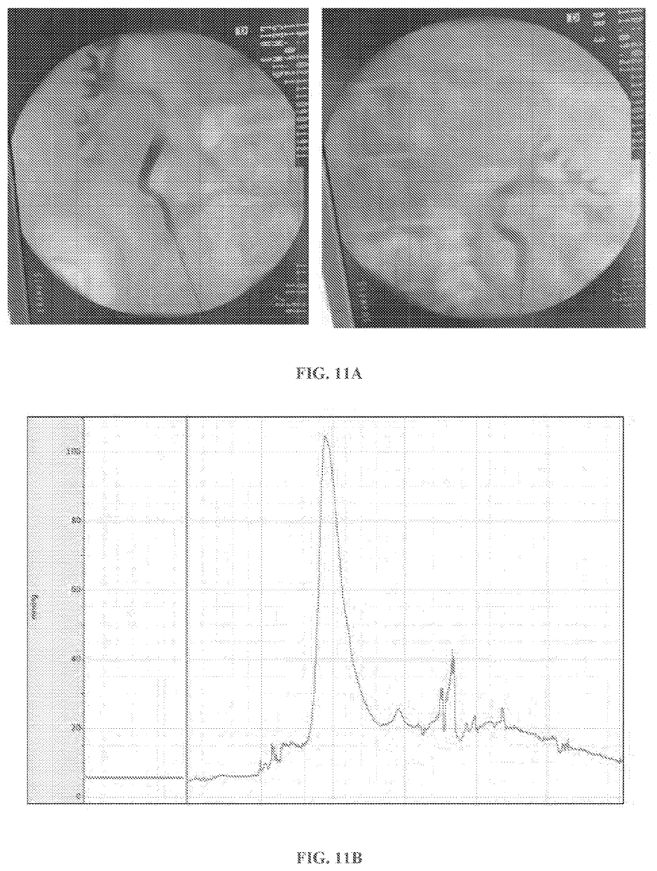

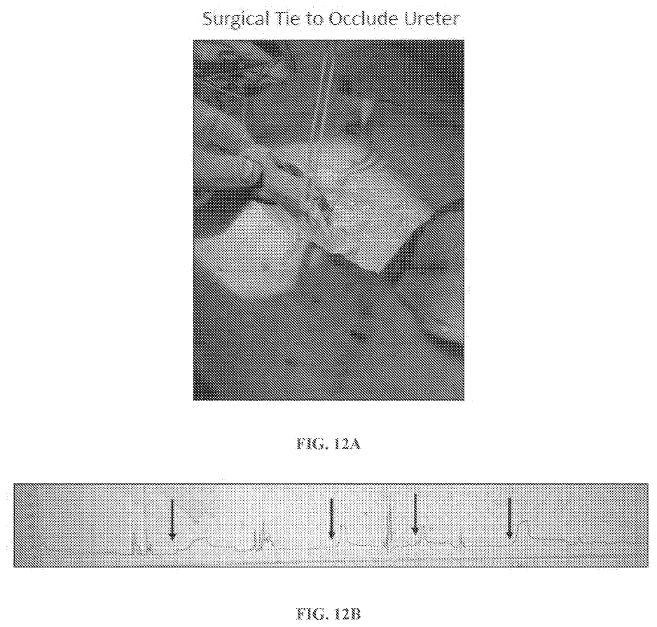

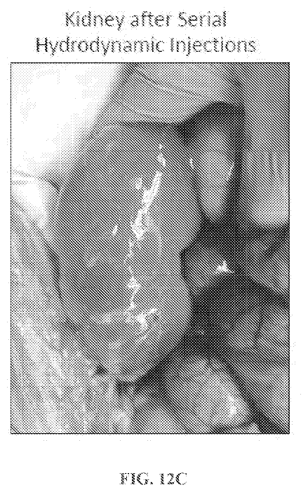

Hydrodynamic delivery in large animal models is a new technique that produces more efficient and robust delivery of gene therapy. The ductal system was chosen for hydrodynamic injections since it reaches the entire pancreatic tissue directly through one network (FIG. 13). Furthermore, the ductal system is unidirectional so that retrograde injection of fluid at high pressure directly enters into the pancreatic tissue.

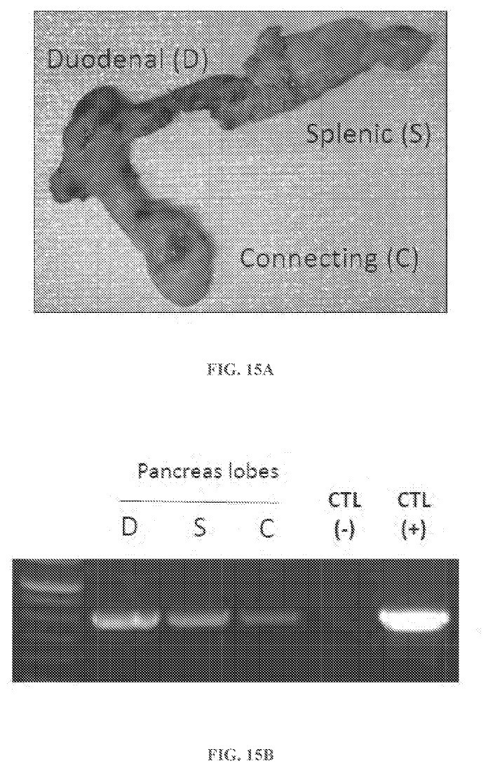

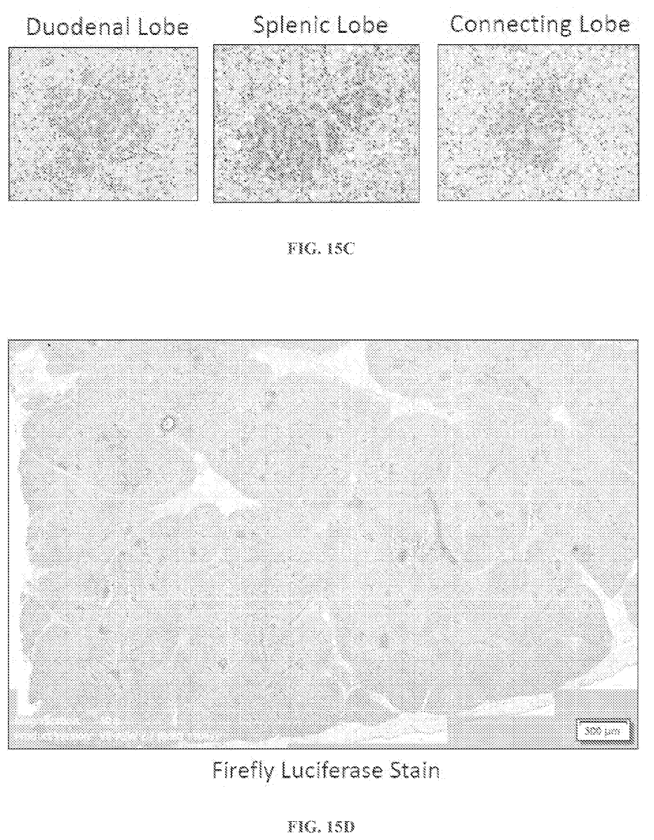

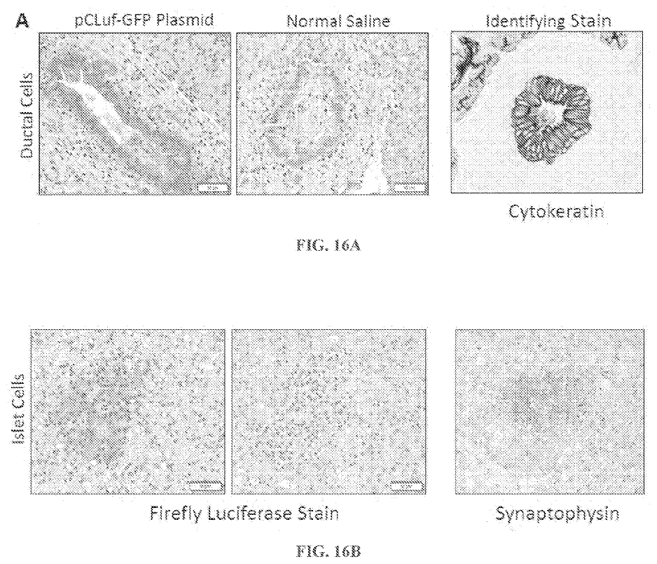

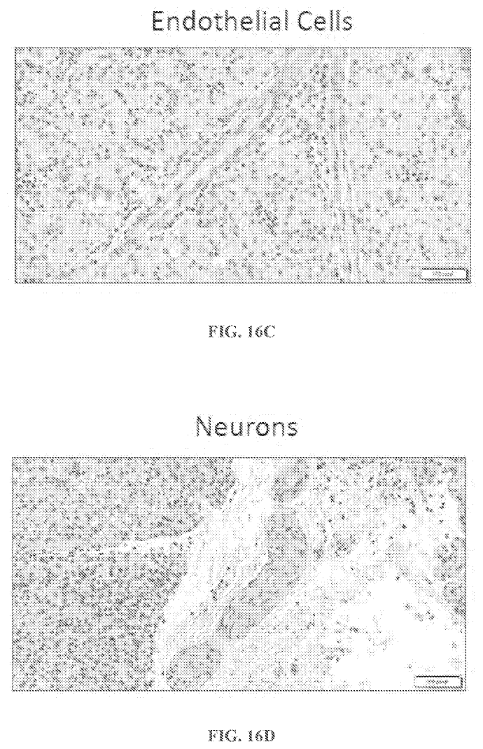

Previous investigations discovered that ductal hydrodynamic injection is efficacious in mediating efficient delivery into multiple different pancreatic cell types. These include islet cells, ductal cells, endothelial cells, and neurons of the pancreas (FIG. 15, FIG. 16). This was accomplished by injecting ubiquitous promoters that could tag multiple cell types.

Previous techniques used for ductal hydrodynamic gene delivery include:
(1) placing a catheter through a major duodenal papilla into a main pancreatic duct, distal to a portion of the pancreatic duct that fuses with a common bile duct, or into a minor duodenal papilla in an accessory or a dorsal pancreatic duct, optionally advancing the catheter farther into the main pancreatic duct;
(2) optionally removing fluid residing in the pancreatic duct to remove digestive enzymes from the ductal lumen;
(3) injecting a contrast agent into the pancreatic duct to confirm correct placement of the catheter;
(4) inflating a balloon in the catheter near an entrance to the pancreatic duct past the common bile duct to prevent retrograde flow of a fluid;
(5) injecting at a flow rate of at least 2 mL/sec and a volume at least 20 mL of a solution comprising at least 1 mg DNA,
(6) wherein the flow rate, volume, and DNA dose are sufficient to mediate gene expression in all pancreatic lobes and multiple pancreatic cell types.

Pancreatic tissue injury monitoring is done by serum testing for amylase and lipase levels. To reduce pancreatic injury, previous studies suggest the administration of different drugs administered during or after hydrodynamic injection, although the best combinations and concentrations of drugs to be employed are not described.

The designated injection parameters previously described are:
(1) the volume injected into the pancreas is at minimum 20 mL, or can exceed 30 or 40 mL in volume, such that volume escapes from the pancreatic ducts into the parenchymal tissue.
(2) the flow rate for the procedure exceeds 2 mL/sec and in other embodiments 3 mL/sec, or 4 mL/sec.
(3) optimal ductal pressure for pancreatic gene delivery is greater than 50 mmHg, greater than 75 mmHg, greater than 100 mmHg, greater than 150 mmHg, or greater than 200 mmHg.

Additional methods described in previous studies include a strategy for changing the promoter specificity in order to target specific cell types in the pancreas. This would allow targeting of exclusive expression in alpha-islet cells, beta-islet cells, acinar cells, or ductal cells, for example. Successful gene delivery to the pancreas only occurred when parameters of 2 mL/sec and 20 mL of volume or greater for gene delivery were used. It is uncertain whether other injection parameters would be successful for gene delivery.

A successful method of gene delivery into the pancreas by hydrodynamic injection into a large animal such as the pig is a large step forward, but several challenges and limitations with the technique remain.

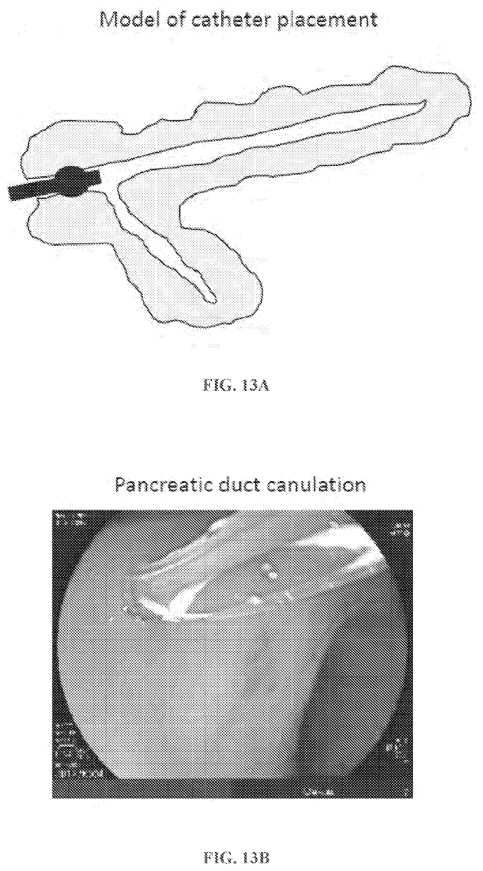

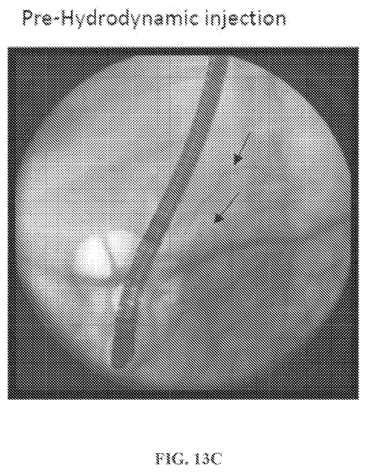

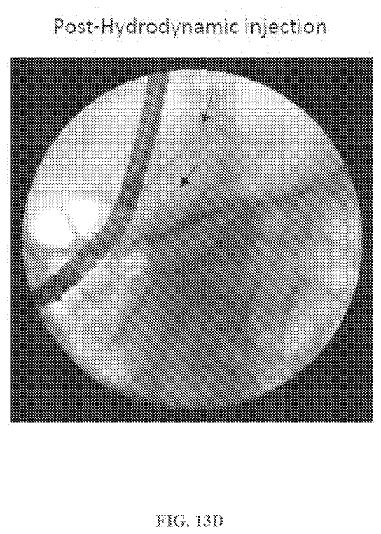

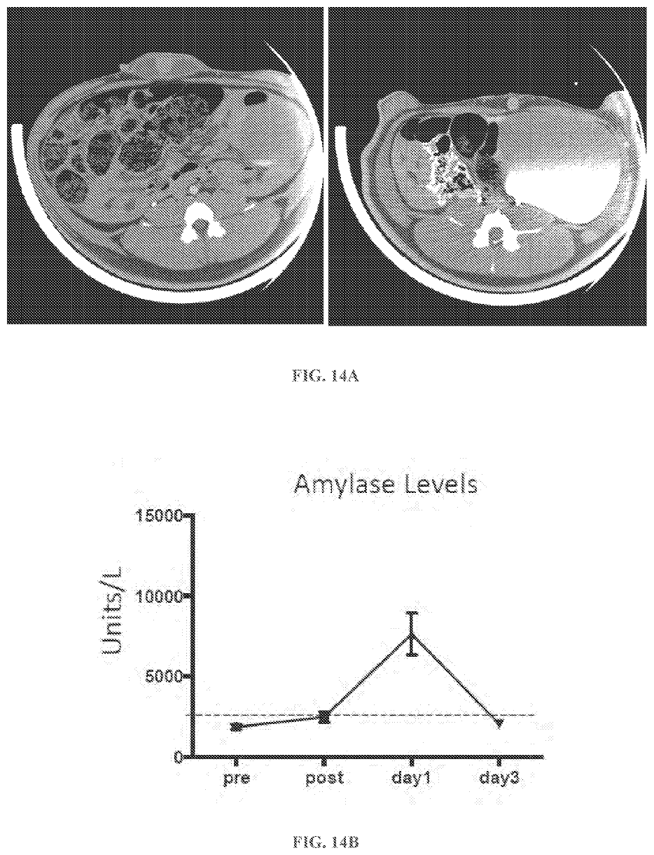

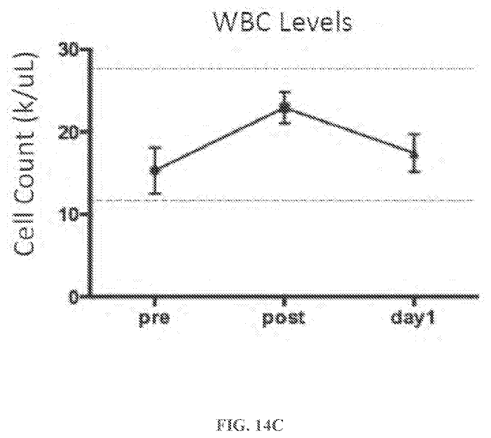

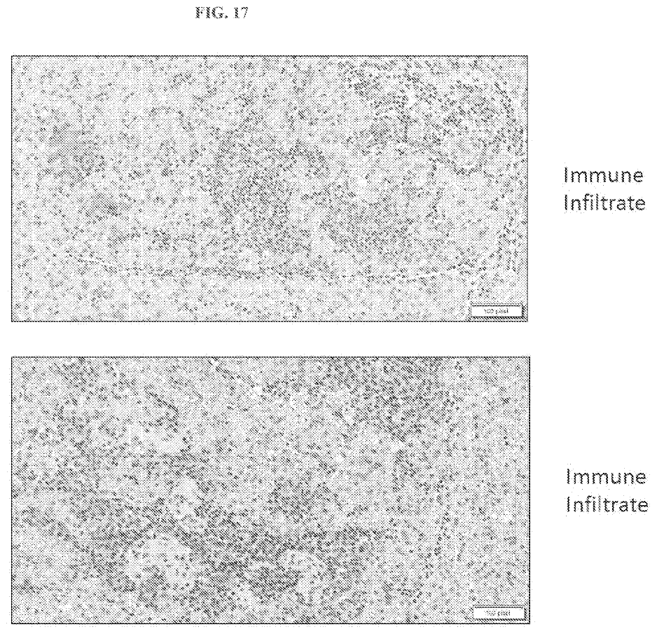

The first drawback is that the injection itself appeared to cause significant tissue injury. While most of the tissues appear to be normal, there were numerous areas on every tissue section showing necrosis (FIG. 17). The corresponding histological image showed small areas where there was significant lymphocyte and neutrophil infiltrate occurred. These areas were not seen in an un-injected pig pancreas. It is uncertain if necrotic areas would develop into full blown pancreatitis at longer time points. At the short time intervals of this study, it did appear that amylase levels normalized (FIG. 14), but necrotic areas were still observed.

While the pig pancreas may be tolerant to pancreatic injury, it is well known that the pancreas from primates including humans is extremely sensitive to any disturbance. To introduce areas of necrosis may be risky or unacceptable for treating human patients. Thus, methods and strategies to reduce pancreatic injury is an important advancement and improvement for this technique.

A potential strategy toward lessening pancreatic injury would be to modulate the parameters so that less force and stress is put on that issue. One way to change the parameters is by decreasing the flow rate, lessening the peak pressure experienced by the. The second way is by decreasing the volume such that the duration of pressure on the pancreas is less.

An important factor in pancreatic gene delivery is that the total volume of the pancreatic duct is only 2-3 mL. Previous studies injected from 4 to 6 times the volume than normal into the pancreatic ducts. There was no rupture of the pancreatic tissue from this additional volume, suggesting that the additional volume can be absorbed into the vasculature or surrounding retro-peritoneal cavity. However, lower volumes injected could be tested and suffice for efficient gene delivery.

Given the small size of the pancreatic duct volume, it is possible that a lower flow rate could create in the ductal lumen a similar threshold pressure in order to push plasmid DNA directly into the surrounding tissues. The lowest tested flow rates that created gene expression in the previous study was 2 mL/sec. It is possible that lower flow rates could achieve gene expression.

Previous studies do not define the target volume for pancreatic injection. Parameters are defined for pigs of a certain weights (40-54 kg), but they are not modulated for pigs of different sizes. This is important because the volume parameter is an essential factor for clinical translation into humans that vary in size. An assumption in the previous studies was that adult human pancreas and the pig pancreas' tested are similar sizes, such that the parameters, including DNA dosing, could be translated directly

### Hydrodynamic Injection in the Pancreas

The use of endoscopic retrograde cholangio-pancreatography (ERCP) to access either the main pancreatic duct or the pancreatic accessory duct for hydrodynamic injection improves the efficiency and safety of the procedure. The catheter can either be placed in the pancreatic head or the pancreatic tail. Additional modifications for the new method and improved technique of gene delivery are described below:

### Safe Balloon Size

The first modification to the procedure is to define a maximal balloon inflation size for hydrodynamic injection that avoids pancreatic duct injury and damage of surrounding pancreatic tissue. It was observed that inflation of the balloon to diameters of 11 millimeters or higher could lead to damage of the pancreatic wall (FIG. 18), that can lead to escape of fluid into the surrounding tissue when the hydrodynamic injection starts.

Generally, the pancreatic duct is a little smaller than the bile duct. The average diameter of the human bile duct is 4 mm on average, which can be dilated up to 6-8 mm in some individuals. However, some patient bile ducts are only 2-3 mm in diameter. The pancreatic duct by comparison, is 3.5 mm in the head of the pancreas, and 2.5 mm in the tail of the pancreas, and the ureter is 6 to 8 mm.

When inflating the balloon in the catheter, the bile duct has more plasticity. It can accommodate more dilation and remain intact with maximal balloon sizes. By comparison, the pancreatic duct does not possess enough plasticity to accommodate larger balloon sizes. The pancreatic tissue in general is more sensitive and friable leading to ease of injury.

The Multi-3 V Plus, Triple-lumen, single-use stone extraction balloon catheter (Olympus Medical) has been utilized in studies of the liver and pancreas. The balloon on the catheter can be inflated to three different sizes (8.5, 11 and 15 mm), and has withstood hydrodynamic pressure. The 11 mm balloon size was observed to cause injury, so future testing would use balloon sizes less than 11 mm, preferably less than 10 mm or 9 mm in size.

### Injection Volume Calculation

A second modification to the injection procedure clarifies and expands on the exact volume to be dosed into the pancreas for hydrodynamic injection. Previously, a volume greater than 20 mL was injected into the pancreas for successful gene delivery. However, this specification does not account for differences in the size of individuals or how to adjust the volume for translation into human patients. For instance, the pig pancreas has two different wings composed of three different lobes, while the human pancreas is a single wing or tissue mass composed of two main lobes. The relative mass of both pig and human pancreas is comparable (discussed in further detail below). In theory, the data on parameters in pigs, should be translatable to adult human patients. Previous studies have only disclosed strategies of injection in pigs of ~40-50 kilograms in size, so it is uncertain how the injection volume would change for individuals significantly larger or smaller in human patients. Previous studies provide instruction for dosing by pancreas mass, but this does not show how to use the information for translation into the proper volume for hydrodynamic injection into the pancreas.

The current disclosure remedies the deficiency by describing in detail how to calculate a safe and effective injection volume to be dosed into the pancreas. As shown in Table 1, the weights of each of the four pigs and their respective pancreas weights were listed as follows, along with the injection parameters for those injections. Of note, some of the pancreas dissections in the early pigs studied 1 include fat tissue around the pancreas organ, making these measurements inaccurate in deciphering weight-based dosing recommendations.

TABLE 1
Injected Gene Delivery

| Animal ID# | Animal Weight | | Pancreas Weight | | Injection Parameters |
|---|---|---|---|---|---|
| Pig #1 | 54 | kg | 130 | grams | 22 mL @ 2 ml/sec |
| Pig #2 | 49.3 | kg | 112 | grams | 23 mL @ 2 mL/sec |
| Pig #3: | 41.3 | kg | v167.2 | grams | 25 mL @ 2 mL/sec |
| Pig #4 | 33.3 | kg t | 75 | gram | 22 mL @ 2 ml/sec |

Although the weight distribution of a pig is different than a human being, the internal organ sizes can be similar. The masses of the pig pancreases from these studies are similar to the human pancreas mass data as shown in Table 2.

TABLE 2
Non-injected with DNA

| | | |
|---|---|---|
| Animal ID# | Animal Weight | Pancreas Weight |

| | | | | |
|---|---|---|---|---|
| Pig #5 | 34.7 | kg | 73.59 | g |
| Pig #6 | 40 | kg | 80.1 | g |
| Pig #7 | 36.4 | kg | 78.2 | g |
| Pig #8 | 37.7 | kg | 64.62 | g |

One method to address this disparity is to weigh the pancreas prior to the injection. The weight determines how much injection volume is appropriate. This avoids the injection of too much fluid into the pancreas, which would risk damaging the organ. While the data from the pig pancreases is similar to human adults, it is more precise to calculate the pancreas based on its weight.

Examples exist from the literature to determine how to estimate the pancreas weight in individual human. An early study in the journal, The Anatomical Record, published in 1926 and summarized known data at the time from autopsies on the weight of the pancreas across adult males and females (The normal weight of the pancreas in the adult human being: A biometric study—https://doi.org/10.1002/ar.1090320204).

The studies summarized in this paper state that the normal weight of the pancreas of an adult man is 60 to 100 grams. The average weight is 80 grams with extremes of 60-100 grams, 70 to 108 grams, or 70 to 90 grams. Additional studies reported that the average weight of a normal pancreas in males is 70 grams, while the average weight in females is 66 grams.

In another study, thirty cadaver pancreas specimens were dissected and carefully measured. The weights were recorded and averaged 91.8 g (range: 40.9 to 182 g). (Am J Surg. 1994 February; 167 (2): 261-3)

Another study shows that the average age of patients was 47.9±17.8 years (between the ages 25 and 88), height 172.2±7.5 cm (between 145 and 190 cm), body weight 78.1±15.2 kg (between 42 and 120 kg), BMI average 26.2±4.7 kg/m2 (between 17 and 38 kg/m2) and BSA 1.9±0.2 m² (Forensic Medicine and Anatomy Research, Vol. 02 No. 03 (2014)). The average pancreas weight was reported as 87.3±30.6 grams.

Another study of pancreas weight in cadavers found significant differences between normal individuals and those with type 1 diabetes, the latter having an atrophy in pancreas size (JAMA. 2012; 308 (22): 2337-2339). This is an important consideration for dosing hydrodynamic gene delivery in this patient population. The investigators found the mean weight of pancreases from those without diabetes (controls) was 81.4 g (95% CI, 73.0-89.8 g) compared with 61.3 g (95% CI, 46.8 g-75.8 g; P=0.02) from the group positive for a single autoantibody only and 44.9 g (95% CI, 36.0 g-53.9 g; P<001) from the TID group.

An alternative method for dosing would be to calculate the pancreas volume alone, either directly from imaging studies or from calculations. The density of the pancreas has been estimated to be 1.1 grams/mL (Cellular Transplantation, 2007). Thus, a volume could be converted into a mass for proper dosage according to the guidelines here. Similarly, the mass-based guidelines here could be converted into volume-based guidelines by one skilled in the art using routine dimensional analysis. An example of a method to calculate pancreas volume based on MRI is provided here as an example of one of many studies that have studied this technique (PLOS One. 2014; 9 (3): e92263).

While the average anatomical studies of pancreases are useful, it is important for the clinical translation of pancreatic gene delivery to have more precise estimations of pancreatic weight. Toward this goal, a previous study provides a solution for how to calculate pancreas weight prior to hydrodynamic injection.

In this study, the pancreas weight from 354 cadaveric donors with respect to gender, age, body weight, body height, body mass index (BMI), and body surface area (BSA) was studied (Cell Transplant. 2006; 15(2): 181-5). The investigators created a mathematical formula to predict pancreas weight from patient-specific factors.

“In younger donors (<40 years old), body weight and age were the major predictors of pancreas weight [pancreas weight (g)=4.355+0.742×body weight (kg)+0.837×age (years) (R2=0.564, p<0.001)].”

“The pancreas weight of elderly donors (>40 years old) was best predicted by BSA and gender [pancreas weight (g)=−17.624+60.036×BSA (m2)-7.152×gender (R2=0.372, p<0.001; “gender”: 1=female, 0=male)].”

By utilizing the mass of the pancreas, the current technology describes how to dose injected volume based on pancreas weight.

In one embodiment of the current invention, the volume for the hydrodynamic injection can be calculated by multiplying the weight of the pancreas by 0.30 mL/g, or 0.35 mL/g or 0.40 mL/g or more wherein the mass in the denominator is the pancreas weight. The ratio multipliers would reflect yielding volumes greater than 22 mL.

### Reduced Volume for Injection

In the current disclosure, new volume targets are disclosed to decrease toxicity from the pancreatic ductal hydrodynamic injection. These volume targets address the drawbacks of previous methods, wherein damage was noted with microscopic lesions of necrosis, along with amylase elevations. These new volume targets are the way to finding the most minimal, yet efficacious gene delivery parameters for hydrodynamic gene delivery.

In one instance, the volume for hydrodynamic injection should be 0.15 mL/g or 0.20 mL/g of pancreatic tissue weight to decrease the amount of tissue damage observed. These calculations aim to represent total volume injected at below 20 mL for a pig of ~40 kg, as was tested previously, or in an average adult human. In more preferred embodiments, the injected volume is 15 mL, or 10 mL in volume total.

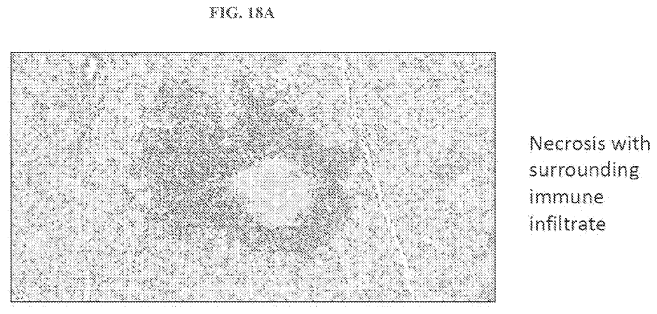

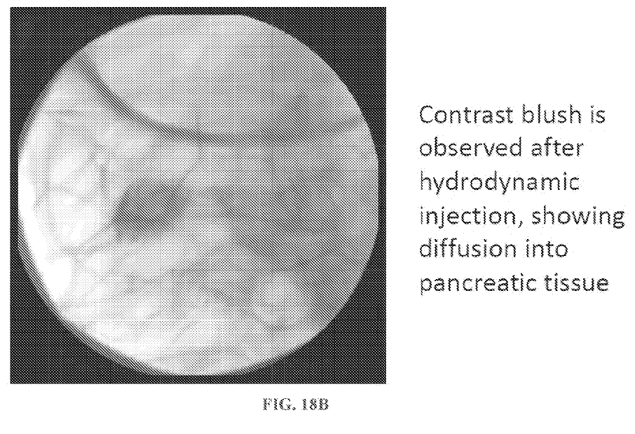

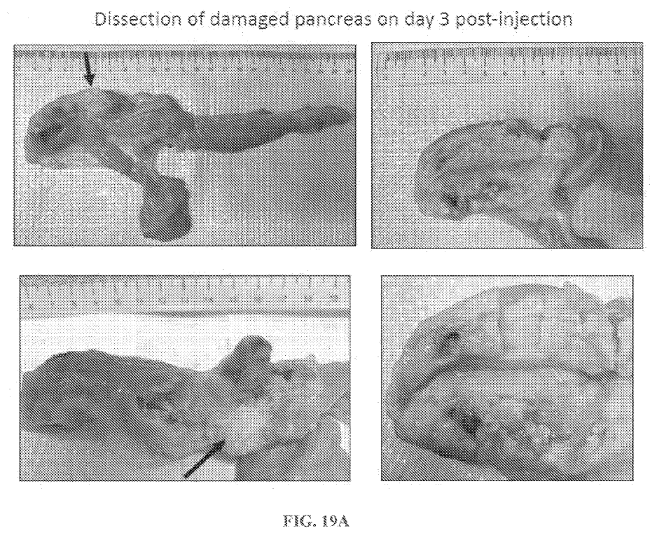

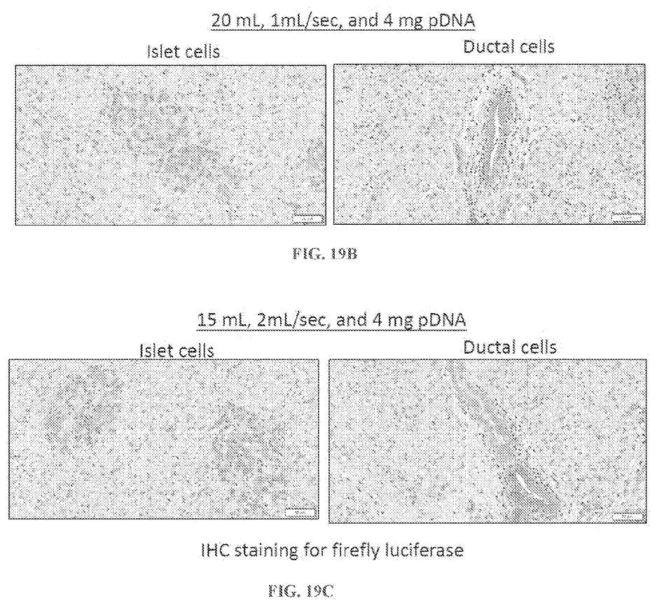

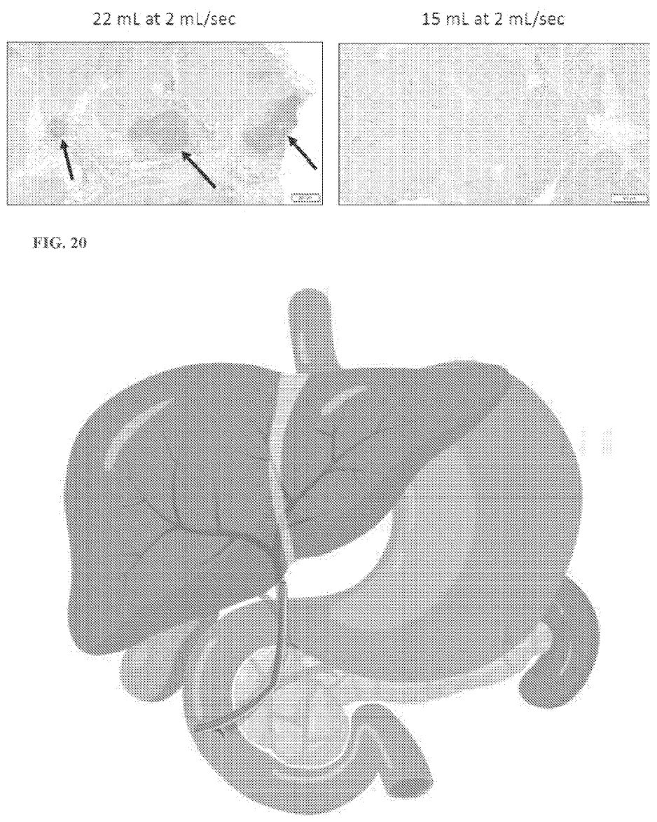

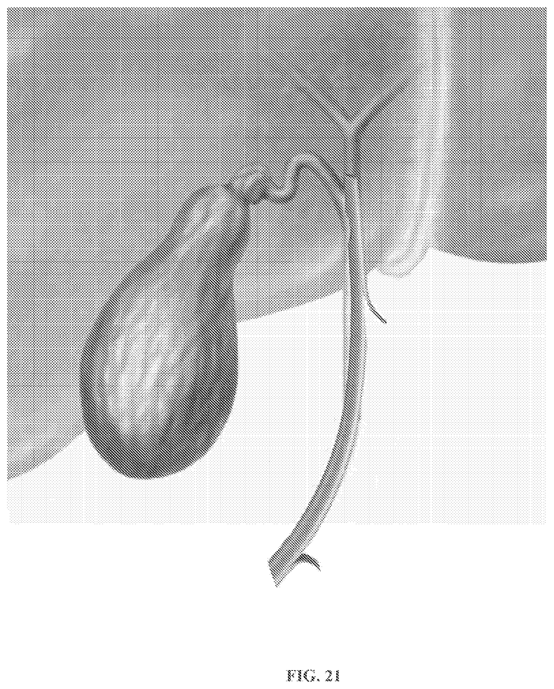

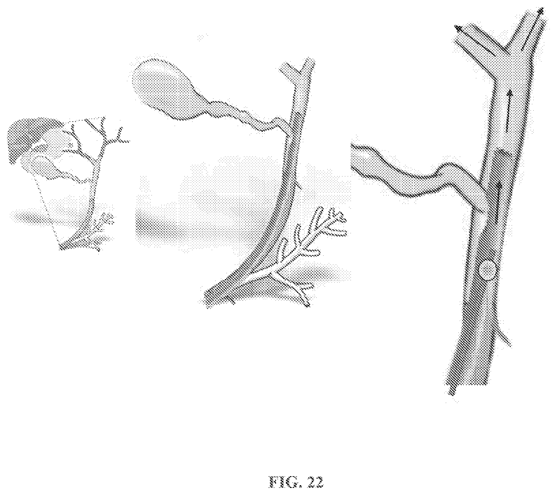

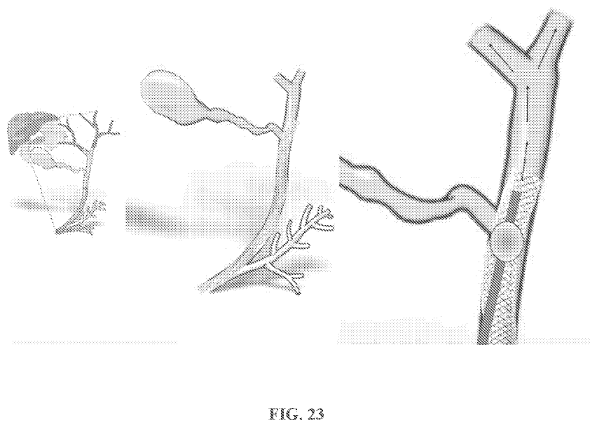

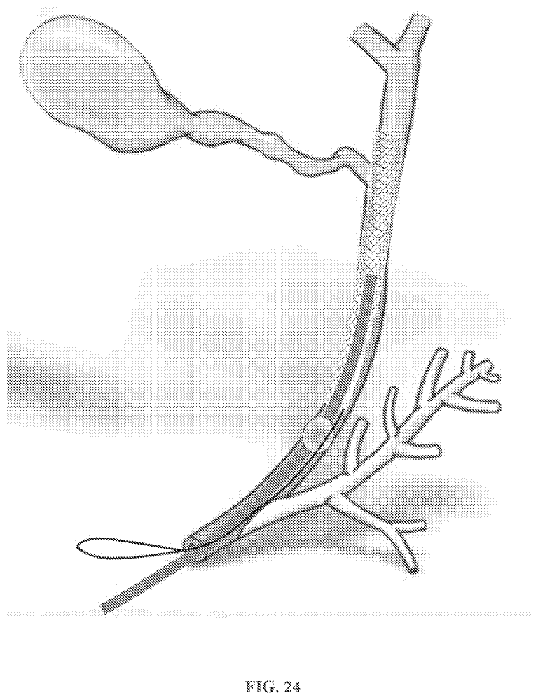

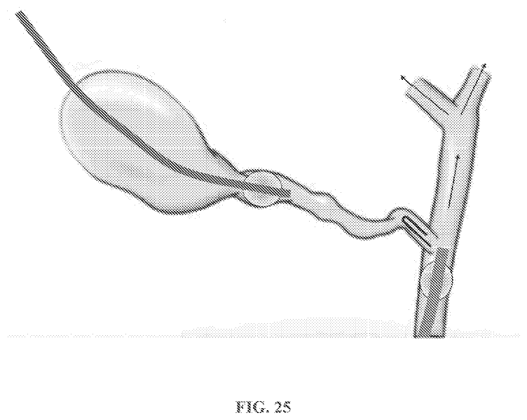

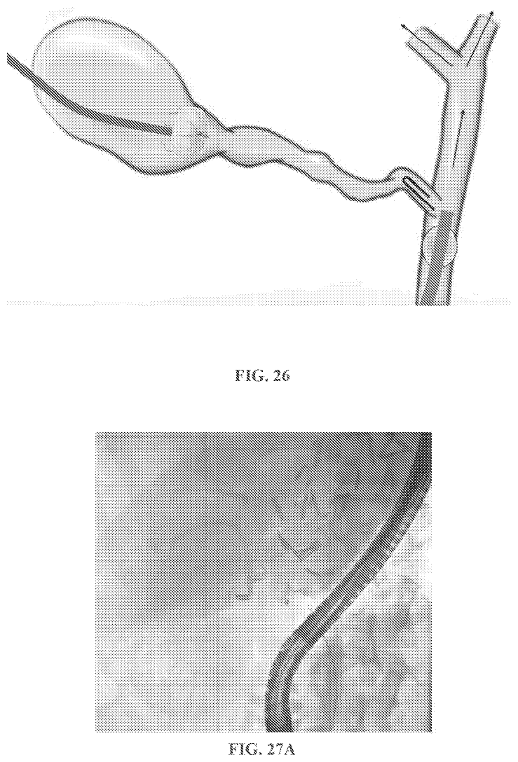

New data supporting the instant disclosure shows that a decreased volume of 15 mL still yields sufficient hydrodynamic gene delivery when combined with a 2 mL/sec flow rate. Gene expression was observed in multiple different pancreatic cell types, with similar efficiency to the prior art disclosed with larger injection volumes (FIG. 19A, FIG. 19B). The goal of decreasing the volume is to decrease the tissue injury during the procedure. This was validated with decrease areas of necrosis observed on histology, as well as decreased amylase increase post-injection (FIG. 19C). The total volume of the pancreatic duct is only 4-5 mL total, which suggests that more minimal injection volumes could yield DNA solution entry into the pancreatic tissue.

A summary of pancreas injection experiments is provided in Table 3 below, documenting different pig and pancreas weights, along with calculated parameters used for those injections.

Guidelines for dosing in the current invention are based on a synthesis of best practices from the previous experiments, accounting for gene delivery efficiency and safety observed.

TABLE 3
Pancreas Injection Experiments

| Pig | DNA | | Flow | Pancreas | | DNA | |
|---|---|---|---|---|---|---|---|
| Weight | dose | Volume | Rate | weight | Volume | dose | Pancreas/body |
| (kg) | (mg) | (mL) | (mL/sec) | (g) | (mL/g) | (ug/g) | (g/kg) |
| 54 | 1 | 22 | 2 | 130 | 0.17 | 7.69 | 2.41 |
| 49.3 | 1 | 23 | 2 | 112 | 0.21 | 8.93 | 2.27 |
| 34.7 | | | | 73.59 | | 0.00 | |
| 41.3 | 1 | 25 | 2 | | | | |
| 40 | 1 | 25 | 2 | 80.1 | 0.31 | 12.48 | 2.00 |
| 36.4 | | | | 78.2 | | 0.00 | |
| 33.3 | 2 | 22 | 2 | 75 | 0.29 | 26.67 | 2.25 |
| 36 | 4 | 20 | 1 | 87.76 | 0.23 | 45.58 | 2.44 |
| 37.2 | 4 | 15 | 2 | 57.78 | 0.26 | 69.23 | 1.55 |
| 37.7 | | | | 64.62 | | | 1.71 |

### Reduction in Flow Rate

Concerning the other injection parameter, the considerations of flow rate for the current technology also differs from previous methods. It was established that volumes greater than 20 mL at a flow rate of 2 mL/sec are sufficient to yield gene expression within the pancreas and multiple cell types.

In the instant disclosure, the preferred total volume injected is under 20 mL, which presents the question of what flow rate would be the best or sufficient for gene delivery. Previously, a flow rate of 2 mL/sec or less was determined to be preferred and sufficient for gene expression.

The flow rate of 1 mL/sec was found to be sufficient for gene expression in studies with a total volume of 20 mL. Thus, preferred flow rates for the invention range are equal to or between 1 to 2 mL/sec. Higher flow rates above 2 mL/sec could be considered, but they are increasingly associated with more pancreatic tissue damage and should be avoided.

### Use of Multiple Flow Rates During Injection

The flow rate can be further modulated by incorporating different flow rates during the injection to further decrease pancreatic injury while maintaining gene delivery and expression. This strategy requires the use of power injectors that can be programmed with multiple injection parameters during a given injection.

An initial flow rate between 1-1.5 mL/sec is used for less than 50% of the injected volume, and the flow rate is between 1.5-2 mL/sec for the remaining injected volume. This reduces the time the main of peak fluid pressure so that it occurs when DNA solution has filled the ductal system to completion.

Another technique is to use an initial flow rate between 0.5-1 mL/sec for less than 50% of the injected volume, and the flow rate is set between 1-2 mL/sec for the remaining injected volume. This reduces the time of the peak fluid pressure so that it occurs when the DNA solution has filled the ductal system to completion.

This strategy was successful in maintaining efficient gene delivery into all relevant cell types (data not shown). Additionally, the number of tissue necrotic areas was decreased, and amylase elevation peak was reduced as well.

Hydrodynamic injection through the liver represents an exciting new gene therapy modality. It is advantageous by being non-viral and thus is highly scalable and safer than viral approaches. Hydrodynamic injection can deliver naked DNA alone representing the simplest gene therapy modality possible. The technique is known to be very efficient in mouse models but traditionally has not scaled well into large animal models.

Several groups have investigated the ability to scale hydrodynamic injection to large animal models over the years. An inherent challenge in developing new technologies for hydrodynamic injection is the challenge in translating the technique between animals. New methods must be adapted and optimized for each animal and organ, empirically testing which strategies will work. The pig is a convenient animal model for testing, since its liver size can be equivalent to an adult-aged humans depending on the age of the pig. Thus, hydrodynamic techniques established in pigs are likely to be translatable into humans. There remain several caveats with this translation, though, including the pig's liver have interlobular fibrosis between lobes that could create some uncertainty.

In that context, the work in mouse, rat, and rabbit models in hydrodynamic gene delivery are certainly exciting proof of concept work, but unfortunately do not educate on how the procedure would be conducted in human patients, and how the gene delivery efficiency could be optimized. A simple example of this is that many groups isolate a specific lobe of the liver and inject through the hepatic vein servicing that lobe. This technique was first described in a paper in rabbits in Human Gene Therapy 2002 Nov. 20; 13(17): 2065-77. Very similar techniques have been used in pigs since then, applying the same strategy of isolating an individual lobe with a balloon occlusion catheter to into that lobe with hydrodynamic injection (Mol Ther. 2009 March; 17(3): 491-9). However, the flow rates and volumes employed in these later studies to mediate gene delivery in pigs could not have been predicted from the rabbit study, since unlike traditional pharmaceuticals or biologics, there is no simple weight-based scaling to the hydrodynamic injection procedure. This finding is even seen with simple hydrodynamic tail vein injection in mice and rats, wherein the volume and flow rate employed in mice does not translate simply into rates when adjusted for their larger size.

Another example of variability is that the vessel or duct chosen for hydrodynamic injection changes the property of the injection greatly, along with the result gene delivery result. An example of this has been seen in pig models, where single lobe injection appears to be more efficient than strategies injecting through the hepatic vein to reach the entire liver, versus strategies clamping the inferior vein cava and portal vein to isolate blood flow there. The subtle differences to these different vessels are not obvious until one does the experiment.

In a similar way, investigators have also studied the biliary system as a different route for hydrodynamic gene delivery. The biliary system is unique in possessing a total volume that is much smaller than the vasculature feeding the liver. This comparison is ~30 mL for the biliary system volume versus around ~600 mL for blood volume in a human adult. The biliary system also uniquely is unidirectional, so injected fluid doesn't simultaneously escape through an unsealed end. This un-directionality of the biliary system is particularly advantageous in maintaining appropriate fluid pressure during the injection and making sure the DNA solution at high pressure is ejected into the surrounding liver tissue, rather than blasting outward through another liver vessel.

While the hypothesis of liver-directed gene delivery has existed for over 20 years now, the practicality of how to do the procedure efficiently and what the outcomes of such a procedure would be in a large animal model remained uncertain.

Concerning the history of biliary gene delivery development, the first description of hydrodynamic gene delivery through the bile ducts was based on experiments in dogs. The group used a surgical procedure, an injection rate (~1 mL/sec), and a suture to prevent antegrade flow within the biliary system (Hum Gene Ther. 1997 Oct. 10; 8(15): 1763-72). Crucially, the investigators injected into the common bile duct, such that fluid was also injected into the gallbladder in addition to the liver. The investigators could only detect a small level of luciferase activity but did not observe any protein detection by histochemical analysis of the liver, so it is likely an undetectable percentage of the pDNA got into hepatocytes. Moreover, no protein was detected on Western blot.

### Parameters:

Plasmid DNA dose: 10-20 mg
Occlusion: suture instead was used in bile duct for occlusion to prevent antegrade flow
Volume: 200-400 mL
Weight, dogs: 4.2 kg to 10.8 kg
Flow rate: Up to 1.66 mL/sec
Pressure: Provided for mice, not for dogs
Other: optional occlusion of the IVC to enhance effect

Toward improving that result, another group described the use of ERCP to mediate gene delivery into the liver into dogs and pigs (GIE 2005, T1249 Abstract). The ERCP aspect represents an improvement in translation of the strategy over surgery. Other innovations of the method of the procedure using by investigators were to use a balloon to prevent antegrade flow during hydrodynamic injection, thereby increasing the portion of fluid with DNA solution that enters into the liver. The group used an injection rate of 5 mL/min for their procedure and continued to inject through the common bile duct, thus simultaneously injecting into the liver and gallbladder. The investigators did not report any gene delivery efficiency into the liver, as reflected by protein expression detectable on IHC, IF, or Western blot. There was a report of detection of a protein in systemic circulation, however. The efficiency and utility of the procedure remains uncertain as applied to liver diseases.

### Parameters:

Volume: 3-10 ml/kg (body weight)
Flow rate: 5 ml/min
Pressure: 40-47 mmHg
Weight: not provided
DNA dose: not provided
Occlusion: Balloon catheter

At the same time, another group reported that injection of pDNA through bile ducts via a surgical approach in rats could mediate gene delivery (Gut. 2005 October; 54 (10): 1473-1479), but as discussed above, hydrodynamic parameters in rodent models can inherently not be scaled up to larger mammals such as dogs, pigs, and humans. As an example, the flow rate employed by the group was 0.54 mL/min and surgical ties helped seal the bile ducts. The relatively efficiency of pDNA delivery into hepatocytes by histochemical staining was only ~1% of hepatocytes, which is significantly less efficient than the comparable hydrodynamic tail vein approach.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

Previous studies have investigated hydrodynamic injection through the biliary tree. The original studies injected through the common bile duct. The common bile duct connects to both the gallbladder through the cystic duct and the liver through the intrahepatic ducts, respectively. The investigators tested hydrodynamic injection through the common bile duct in dog and pig models. The investigators reported protein expression could be achieved by luciferase assay, as well as systemic protein expression, although no liver tissue delivery was shown as represented by immunostaining.

The current disclosure seeks to improve upon strategies of hydrodynamic injection through the common bile duct. Strategy for injections of the common bile duct includes inflating a balloon within the common bile duct to block antegrade flow into the small intestine. The balloon could be inflated anywhere along the common bile duct. In an alternative strategy, the balloon would be inflated such that the balloon is inflated at the junction of the common bile duct and common hepatic duct such that it occludes the orifice of the cystic duct.

The balloon could be 8 mm in diameter or largest width (depending on the shape of the balloon) or larger depending on the luminal caliber appreciated. To achieve adequate seal during injection, the balloon would need to be at a minimum 1.5× the width/diameter of the bile duct in this location though more likely 2× or 3× or more than greater the width/diameter of the bile duct to provide confidence of an adequate seal. The width/diameter could be fixed at least 4 mm greater or it could be at least 50% greater. Alternatively, the width/diameter could be fixed at least 5 mm, 6 mm, 7 mm, or 8 mm greater or it could be at least 60%, 70%, 80%, 90%, 100% or greater. Too large a width/diameter could cause stretching of the bile duct and subsequent bile duct injury or perforation, most likely to occur during the process of hydrodynamic injection.

Confirmation of an adequate bile duct seal would be ascertained by either measuring the pressure (resting or during a test injection) of the bile duct upstream of the balloon. If the pressure readout suggests an inadequate seal, the balloon would be further inflated. Another method to confirm an adequate seal would be inject contrast through the catheter upstream or downstream of the balloon to confirm the position is distal to the hepatic hilum and occluding at the level of or upstream of the cystic duct take-off. Pressure could also be measured from a lumen that has an orifice that opens proximal/downstream to the balloon.

The previous parameters explored include a volume-based injection ranging from 200 mL to 400 milliliters. In a different study, the injection volume range from 3 to 10 mL per kilogram of subject weight.

The required DNA doses range from 10 to 20 milligrams of DNA into a 5 to 10 kg dog. The other study did not report the DNA doses employed. Concerning the flow rate, one of the studies targeted a flow rate of at most 1 mL per second. The other study preceded with a flow rate of 5 mL per minute. One of the prior art references teaches optimal injection pressure between 40 to 50 mmHg for the injection.

The current disclosure seeks to improve upon the prior art and teaches methods of hydrodynamic injection through the common bile duct in order to deliver genes into the gallbladder and the liver at the same time. In other embodiments, only the liver will be targeted. The disclosure teaches upon appropriate injection parameters and DNA doses necessary to achieve hydrodynamic injection through the common bile duct.

Injection within the Common Bile Duct

The disclosure comprises several steps to achieve gene delivery in the gallbladder and/or liver. The first is to access the common bile duct. The common bile duct can be accessed through endoscopic retrograde cholangiopancreatography, or ERCP, which is a routine clinical procedure wherein a catheter is inserted from the small intestine into the biliary system, conventionally through the major papilla. Alternatively, the common bile duct can be accessed through endoscopic ultrasound guidance, or EUS, wherein a needle is inserted through the duodenal wall and into the common bile duct directly.

Once a catheter is in place within the column bile duct, the catheter would preferably have a balloon that could be inflated to seal the common bile duct entirely. Common bile duct just relatively wide into diameter, such that relatively large balloon size such as 8 millimeters, 11 millimeters, 13 millimeters, or 15 millimeters or greater may be necessary to completely seal the common bile duct. After sealing the common bile duct, in some embodiments, bile could be withdrawn through the catheter by suction in order to remove as much volume as possible from the common bile duct, common hepatic duct, and gallbladder. In other embodiments, catheter may be advanced through the cystic duct and bile withdrawn from the gallbladder similarly in order to remove it prior to injection. In addition to aspiration of bile alone, a lavage of the bile duct with a neutral fluid, such as normal saline, could be performed such that a proportion of the biliary fluid volume of the bile duct is replaced by this neutral fluid.

In other embodiments of the disclosure, the bile may be left in place prior to injection, since the substance fills the gallbladder prior to injection. In other strategies of the disclosure, the gallbladder is filled with fluid or gel like substances prior to the hydrodynamic injection of DNA solution. The purpose of these strategies is to fill the gallbladder such that there is no remaining volume for additional fluid to enter during injection. In certain embodiments of the disclosure, a saline solution may be preloaded into the gallbladder for this purpose. The preloaded saline solution may be between 40, 50 or 60, or greater mL in total volume. In other embodiments, the saline solution may contain contrast such that the filling of the gallbladder it could be monitored in real time.

In other embodiments the disclosure, a gelatinous-like substance could be injection to the gallbladder to fill the gallbladder space. The polymer substance would subsequently be degraded within hours. And some examples of the disclosure, Poly(lactic-co-glycolic acid) (PLGA) powder could be injected into the gallbladder in order to fill room prior to injection.

In optimal embodiments of the disclosure, the injection would proceed at a volume of 50 mL/kg, or 100 mL/kg of liver weight. This volume accounts for the total volume of the gallbladder, the biliary system, and intra-hepatic ductal system. In other embodiments, the injection volume is at least 150 mL/kg liver weight, or at least 200 mL/kg of liver weight.

In certain embodiments of the disclosure, the DNA dose is at least 20 milligrams per kilogram of liver weight. In other embodiments, the DNA dose is least 40 milligrams per kilogram of liver weight.

In other embodiments of the disclosure, the minimum DNA concentration for the injection is at least 0.2 mg/mL of volume. In other embodiments, a minimum DNA concentration of at least 0.5 mg/mL of injection volume.

The optimal flow rate taught by the current disclosure differs markedly from the prior art. The current disclosure teaches that a flow rate of at least 2 mL/sec, or 5 mL/sec is required. In other embodiments, the flow rate of at least 10 mL/sec is required during any injection.

An optimal pressure during the injection is also established by the current disclosure. The current disclosure teaches that minimal pressures of at least 50 mmHg is necessary for efficient gene delivery. In other embodiments, the minimal pressure is at least 80 mmHg, or 120 mmHg for injection. The pressure during gene delivery could be achieved in some examples by a constant pressure injection device, which would monitor pressure and adjust did in real time.

As an example of the efficacy of this gene delivery strategy, efficient gene expression is achieved within the bile duct walls, the common bile duct, and common hepatic duct. This also demonstrates reporter gene expression within the walls of the gallbladder. The injection strategy at the designated injection parameters also shows applications in mediating gene delivery into a hepatocytes.

In some embodiments of the disclosure, antibiotics will be mixed in with the DNA injection solution, since injection lower in the biliary system is associated with more propensity for the presence of gut bacteria, which can lead to cholangitis. In another embodiment, antibiotics would be mixed with the fluid used to lavage the biliary tree after bile aspiration occurred and not with the DNA solution itself.

In summary, presented herein is a novel discovery of efficient gene delivery through the common bile duct. The current strategy remedies previous attempts through executing injection at higher flow rates and pressure levels in order to drive more expression within host hepatocytes. The instant disclosure also teaches gene delivery into the extrahepatic biliary system, which was not reported in previous studies. The disclosure expands the versatility of using biliary system as a route for gene therapy strategies.

Strategies to injection through common bile duct and/or common bile duct and cystic duct junction

The current disclosure describes a method of executing hydrodynamic injection through the biliary system. The injection describes methods to execute injection through the common bile duct, while bypassing the cystic duct to allow for all fluid to enter into the common hepatic duct and subsequently into the liver.

The method describes a new strategy where a stent is placed within the extrahepatic bile duct, such that the stent extends over the cystic duct orifice and into the common hepatic duct. The stent would optimally have a diameter that is greater than the bile duct in order to assure its stability and that no fluid flows around the duct, but rather all solution flows through the interior of the stent. This would effectively block any injection solution from entering into the cystic duct or the gallbladder. An optimal stent would be made of a solid material or have a covering without perforations in the wall such that the fluid cannot permeate through the walls of the stent itself. The stent could be deployed such that it would cross the major papilla into the duodenum for easy removal. The stent could also be deployed such that it is fully within the biliary tree only but a string or tether is attached to the stent such that when deployed it can be grasped with easy and removed.

In an optimal embodiment of the disclosure, the catheter would be placed in several different locations. In one embodiment, the injection could take place downstream of the stent in the common bile duct. A balloon will be inflated within the common bile duct to prevent antegrade flow into the intestine. Injection solution with DNA will proceed retrograde through the stent into the common hepatic duct and then subsequently enter into the liver.

In another embodiment of the disclosure, the catheter is localized within the stent in the bile duct. Inflating the balloon within the stent will allow the balloon to safely inflate such that its shape will morph from spherical to cylindrical within the stent, without injuring the biliary tree as the stent will bear the forces of the balloon as opposed to the bile duct wall directly (FIG. 27A). The exact location of the catheter could either be within the common hepatic duct aspect of the stent (FIG. 27B), the common bile duct aspect of the stent (FIG. 27C), or at the cystic duct junction of the stent. The balloon would be inflated inside the stent to allow for adequate sealing and prevention of antegrade flow of the solution. Moreover, it is expected that the pressure from the fluid injection would firmly press the stent against the walls of the bile duct further improving the seal. Finally, it is expected with an inflated balloon inside, the catheter could be used to keep the stent secure during the hydrodynamic injection.

After the injection is over, regardless of the catheter location, the balloon is deflated and the stent removed with the catheter and the bile duct.

Optimal injection parameters for injection through the common bile duct and stent would follow along with previously described injection parameters for hydrodynamic injection at the common hepatic duct. The main difference would be an additional 5-10 mL of volume being added to the calculated volume to account for the additional volume of the common bile duct and common hepatic duct lumen. The calculated volume would utilize published strategies for the common hepatic duct.

Optimal embodiments of this strategy would inject contrast into the biliary system in order to verify a lack of opacification of the gallbladder. This would effectively confirm that the stent is blocking the cystic duct preventing any entry of DNA solution during injection.

This strategy would find use in scenarios where the common hepatic duct is extremely short or damaged. The strategy may also find use in smaller subjects, such as neonates, where in the comment hepatic duct itself is extremely short.

In optimal embodiments of the disclosure, the following parameters could be used for the injection. The parameters are similar to strategies employed by common hepatic duct injection, given only the limited additional volume involved.
Preferred flow rates during the injection are at minimum 1 ml/sec, or 2 mL/sec.
Preferred injection pressures are at least 50 mmHg, in other embodiments at least 80 mmHg.
Preferred injection volumes are at least 30, 40, 50, or 60 mL per kilogram of liver weight. Optionally, an additional 5 mL of injection solution is advised to account for the additional lumen within the biliary system.
Preferred DNA doses are at least 20 mg per kg liver weight, or larger.
Preferred DNA concentration is at least 0.5 mg/mL DNA or more.
Preferred use of hepatocyte-specific promoters to increase hepatocyte transfected area.
   Occlusion of the Cystic Duct Through a Secondary Approach Combined with a Common Bile Duct Strategy

One strategy to negate loss of plasmid DNA solution is to occlude the cystic duct. This can be performed temporarily or permanently and would allow for the injection through the balloon catheter to occur at the level of the common bile duct. One example would be to have the patient undergo a cholecystectomy. Another example would be a patient having a choledoenterostomy (gallbladder to small intestine anastomosis) created (surgically, percutaneously via interventional radiology, or endoscopically via EUS) and a catheter with an occlusion balloon attached being placed from the gallbladder to occlude the cystic duct. In the aforementioned example, as an alternative to using a balloon to occlude the cystic duct, one could use a catheter which allows the release of an umbrella like cover from its tip which could be placed over the cystic duct take-off from the bile duct or within the cystic duct itself.

### Intended Uses of Common Bile Duct Strategy

The current disclosure may find several uses. Unconventional anatomy of the biliary tree where the cystic duct take-off is not downstream from the hepatic hilum not infrequently encountered during biliary tract imaging (Sarawagi et al, Pol J Radiol. 2016; 81:250-255). At times, the cystic duct orifice is upstream of the hepatic hilum (i.e coming off the left or right intrahepatic bile ducts). This would significantly complicate an injection strategy that bypasses the cystic duct and gallbladder. In this instance, injection from the common bile duct would favorable.

Strategy could also find use for veterinary settings, wherein the biliary anatomy is different than humans. In some animals, there is not a common hepatic duct or traditional cystic duct. The strategy is also useful in settings where one seeks to treat extra-hepatic biliary disease, such as when treating cholangiocarcinoma or fibrotic narrowing of the biliary duct. Strategy may also be useful in treating certain gallbladder disorders that may be treated with a genetic intervention. The strategy is also useful in gene delivery into the liver wherein the common hepatic duct may be too small to be adequately localized. In these instances, and injection from the common bile duct would be the only feasible solution for gene delivery into liver. An example would be certain injections into neonates.

Gene therapy against cancer is attractive idea. There are many ways to fight cancer with gene therapy. One of the attractive ways is to try to deliver genes directly into the tumor microenvironment. These genes could encode a host of therapeutic proteins including ones to modulate and recreate the immune system. In other cases, the genes could be delivered directly into the tumor cells. In this case, suicide genes could be encoded that would immediately kill the tumor.

There have been several gene therapy strategies previously published. Previous tumor delivery strategies include using viral vectors, oncolytic viruses, which have replication and capacity in the tumor, and different non-viral strategies. Non-viral strategies include direct dosing DNA or RNA into the tumor with a local delivery strategy. Another common strategy is to use lipid or polymers to deliver plasmid DNA directly into the tumor. For these strategies, tumor-specific uptake can be enhanced through having specific tumor binding ligands on the nanoparticle surface, or alternatively using polymers or lipids with natural affinity for specific types of tumors.

The disadvantage of all these strategies is the inherent difficulty of penetrating the gene therapy vector into the tumor microenvironment of the cancer cells themselves. Most strategies rely on systemic administration of the gene therapy vector. In these instances, it is necessary for the gene therapy vector to crossover endothelial barriers and deep into tissues. Many tumor types are well vascularized around the rim of the tumor, but not toward the inside of the tumor. Because of this, the interior tumor is very hard to penetrate from vascular administration. Other tumor types have a lot of fibrosis within the tumor, such that any nanoparticle or viral vector has a hard time penetrating the tumor.

One potential strategy for non-viral gene delivery is hydrodynamic delivery.

Hydrodynamic delivery leverage is fluid pressure to create pores in the cell membranes to allow DNA to enter inside cells. Hydrodynamic delivery is a very efficient gene delivery modality into the mouse liver when delivered through the mouse tail vein. During tail vein injection, fluid is rapidly injected into the mouse liver approximately 10% of body volume weight, over a period of five to 7 seconds. The fluid injection into the tail vein and IVC backs up from the right heart and into the mouse liver causing severe fluid congestion. The fluid pressure creates pores in the mouse liver yielding efficient gene expression.

Although hydrodynamic tail vein injection is a very common technique, it is largely unexplored whether hydrodynamic injection can mediate efficient delivery into tumors inside the liver. A major obstacle in even testing this hypothesis is to try to generate a local tumor within the mouse liver. The majority of mouse models develop multifocal development of large tumors over time through germline mutations.

The only study that has ever evaluated hydrodynamic injunction for gene delivery into a hepatocellular carcinoma (HCC) was performed in rats (J Gene Med. 2006 August; 8(8): 1018-26). Rats can similarly be subjected to hydrodynamic injection. The rats in this study were treated with a chemical, diethylnitrosamine, that causes mutations and the rat liver. These mutations overtime can eventually lead to the development of how to say like carcinoma. The investigators found that hydrodynamic tail vein injection was very poor and mediating any delivery to the tumor with almost no expression seen. Expression could only be detected in the tumor when the DNA vector was administered through the hepatic artery. Thus, it appears that the efficiency of delivery is highly dependent on the route of administration.

A central challenge with hydrodynamic injection is the uncertainty and unpredictability of how to scale up of the technique from rodents to large animals. When pondering how to scale hydrodynamic injection, the volume and flow rate increases by an order of magnitude compared to what parameters are used in rodents. Furthermore, the routes of entry and the methods to access those routes differ markedly in large animal models versus what is executed it and rodent models.

Without any data supporting the actual delivery into liver and pancreatic tissue in a large animal, it is currently unpredictable whether gene delivery by hydrodynamic injection would be efficacious. In particular, it's unpredictable what areas of the liver, if any, would express the gene being injected. Another important unknown is whether different tumor types within the liver, all having different types of tissue architecture, would up take any DNA vector differently when delivered by hydrodynamic injection. Thus, it's uncertain if tumors could be reached by hydrodynamic injection.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

The disclosure describes methods of gene delivery by hydrodynamic injection into tumors of the liver. The method leverages the biliary route to deliver fluid pressure into the tumor microenvironment. The biliary system is a dynamic network a branching architecture that contacts all hepatocytes. However, previous studies prior to the disclosure have taught that the biliary network does not contact the tumor microenvironment. This includes primary liver tumors and metastatic liver tumors. Therefore, the prior art to this study would have speculated that this method would have failed. The current disclosure teaches that the biliary route is efficacious in mediating gene delivery into different aspects of the tumor. The current disclosure also teaches that multiple tumor types, and not just a hepatocytes can be engaged through the biliary system for gene delivery.

The first step of the method is to perform endoscopy. ERCP is used to access the common hepatic duct in the biliary system. Once in the common hepatic duct, a balloon will be inflated to seal the duct. At the same time, a DNA solution will be prepared wherein the DNA encodes a therapeutic protein of interest to treat the tumor. In other embodiments, the DNA of interest may encode a diagnostic protein to help recognize the tumor. The DNA solution will be loaded into the power injector. In preferred embodiments, the solution volume loaded will be at least 30 mL per kilogram of liver weight. In other embodiments, the solution volume loaded will be at least 40 mL per kilogram of liver weight. The flow rate will be designated to be at least two mL per second in preferred embodiments. In other embodiments the flow rate is at least 4 mL per second. The technology has previously been optimized for delivery into normal hepatocytes. The delivery into normal hepatocytes has been taught in the prior art at a flow rate of minimum of 2 mL per second. For intra-tumoral delivery, the inventor teaches that higher flow rates are more preferable to allow for deeper penetration tumor microenvironment. In preferred embodiments, flow rates up to 10 mL per second may be utilized to increase the expression of DNA within the tumor.

The minimal DNA dose for efficient delivery inside the tumor is a DNA dose of at least 1 milligram of DNA per kg liver weight. In other embodiments, a minimal DNA dose of 5 mg per kg liver weight. In other preferred embodiments, a DNA dose of 10 milligrams per kg liver weight may be preferable. In general, higher DNA doses are required for this specific technique of tumor targeting since the hydrodynamic approach delivers DNA into the entire liver, such that only a small amount of DNA will end up within the tumor microenvironment. The amount of DNA in the tumor influences the degree of transfection appreciated.

In some embodiments of the disclosure, methods of addressing this limitation are to localize the catheter not in the common hepatic duct, but in the right or left hepatic duct of the bilary system. This would facilitate DNA delivery in only the right or left side liver. If the tumor were only localized in the right or left hepatic ducts, then this strategy could be used to increase the amount of DNA delivered to that specific tumor. In other embodiments, wherein multiple liver tumors or present and/or metastases are present throughout the liver, injection from the common hepatic duct is preferred.

In some embodiments of the disclosure, the DNA molecule could be further formulated. The DNA molecule could be formulated with a variety of polymers, peptides, or lipids that have been reported to facilitate greater uptake into tumors. It is envisioned that the hydrodynamic injection method could synergize with these delivery reagents to facilitate even more efficient delivery of these nanoparticles into the tumor. Hydrodynamic injection would particularly help with their penetration to reach into the inside of the tumor. Given that tumor cells are dividing, the nuclear envelope breaks down more frequently, allowing more non-viral DNA to access the nucleus for expression.

Gene delivery into the pancreas proceeds with similar parameters, except ERCP accesses the pancreatic duct, and the dosing is per estimated pancreas weight. Most tumors are located in the head of the pancreas, such that the tumor should be downstream of it in order to deliver DNA under pressure into the tumor.

Monogenic hereditary liver diseases encompass a spectrum of disorders ranging from metabolic diseases to clotting disorders, including hemophilia A and B, alpha-1 antitrypsin deficiency, familial hypercholesteremia, Wilson's disease, Crigler-Najjor syndrome, methylmalonic acidemia, ornithine transcarboxylase deficiency and more. These diseases cause significant morbidities in patients even with current modern therapies. The curative therapy in these disorders is liver transplant, but the availability of livers is limited. Moreover, transplant itself requires lifelong immunosuppression leading to the risk of infection and drug side effects.

As an alternative to liver transplantation, gene therapy has been explored, modifying the patient's own tissue with the missing or dysfunction gene. The success of adeno-associated virus (AAVs) in preclinical models has been translated into clinical trials, but has revealed several limitations, including many patients have preexisting neutralizing antibodies against AAVs; high levels of AAVs infusion triggers immune response and there after clearance of AAVs; limited gene packaging size to about 4.8 kb, etc.

In order to solve these problems, the inventor has developed a non-viral gene therapy using hydrodynamic injection via endoscopic route. a gene was successfully delivered in a pilot study using endoscopic retrograde cholangio-pancreatography (ERCP) into pigs liver, achieved 30-50% transfection efficiency which can potentially meet the need to cure many monogenic liver diseases. The previous work in ERCP gene therapy for hemophilia B received R21 funding from NHLBI/NIH to future explore this gene therapy research in nonhuman primates.

The current work will explore optimal injection methods into primate liver that can be translated into human patients. The current work also describes optimal methods and compositions for hemophilia B gene therapy with hydrodynamic injection.

Rare individuals have mutations in their genes that can disrupt basic processes in the liver, such as metabolism or clotting. The disruption in metabolism or clotting can lead to various issues, including blood vessels thickening with cholesterol, metals such as copper building up to toxic levels in tissues, or persons continually bleeding without stopping.

A possible cure for these approaches would be to insert a functional copy of the gene into the persons liver, thereby reversing these processes. This research will explore this gene therapy, using a common medical procedure where a tube is stuck down the throat, through the stomach and into the intestines. A small wire can then be guided to the liver and target DNA delivery directly. This research will explore an approach that does not use viruses as the delivery vehicle, which will improve safety. The potential impact could be cures for these patients, saving them from frequent infusions of other medications, and hundreds of thousands of dollars in drug costs per year.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

The disclosure describes a series of steps toward the effective delivery of DNA into the liver of primates for the purpose of gene therapy.

The first step consists of accessing the biliary system for gene delivery and injection. In preferred embodiments, the procedure of endoscopic retrograde cholangio-pancreatography (ERCP) will be executed in order to place a balloon catheter inside the common hepatic duct of the baboons. In preferred embodiments, the ampulla of Vater may be cut to increase the size of the opening and the ease of canulation of the duct.

Using radiocontrast solution, the catheter is localized in the common hepatic duct past the cystic duct to avoid injection to the gallbladder. Contrast injection will also be used to verify that that balloon seals the duct during the injection, and that the right and left hepatic ducts are visualized.

Subsequently, the injection protocol will begin. The injection proceeds with the use of a power injector loaded with a DNA solution of interest. Tubing connects the end of the power injector and to the biliary catheter in order to inject DNA solution through one of the lumens.

The DNA solution in preferred embodiments is a normal saline solution with pure recombinant DNA dissolved in the solution. The DNA may be a number of types, including plasmid DNA or minicircle DNA. In other embodiments, linear closed-ended DNA could be utilized.

The injection shall proceed designated parameters that mediate efficient gene delivery. The volume parameter will be set at 30 milliliters per kilogram of liver weight. The volume can also be 40/mL/kg or greater.

The flow rate parameter in the preferred embodiment shall be at least 2 mL per second. In other embodiments, the flow rate can be at least 3 mL/sec or greater.

The pressure parameter, when using a specialized constant pressure injection device, will be at least 50 mmHg. In other embodiments, higher pressure will be employed, including greater than 80 mmHg, or greater than 120 mmHg.

The DNA dose will be at least 10, 20, 30, 40, or 50 milligrams per kilogram of liver weight in certain embodiments. The goal of higher DNA doses is to yield sufficiently high serum or plasma concentration in order to yield efficient levels of human Factor IX to treat hemophilia B.

The DNA vector composition will employ a hepatocyte-specific promoter. The vector in preferred embodiments should also have one or more hepatocyte-specific enhancers to drive even higher levels of transcription. A codon optimized gene cassette with codons selected for abundance in the hepatocytes will be selected. This can be applied in preferred embodiments toward the expression of human Factor IX. A strong polyadenylation sequence will also be utilized.

Injection of primate liver will also seek to monitor for signs of liver injury, which can be accomplished through a common liver panel of chemistry tests including transaminase function.

The balloon is deflated post-injection, where after the catheter withdrawn from the bile duct. The injection may be repeated within the same procedure or on a different procedure day as indicated.

The present disclosure relates to compositions and methods for hydrodynamic gene delivery into the liver through the biliary system. More particularly, the present disclosure relates to compositions and methods for increasing the efficiency of hydrodynamic gene delivery through the biliary system. As described in detail below, the present disclosure is based, at least in part, on the surprising discovery that modifying the DNA vector to substantially reduce the bacterial sequences on the plasmid DNA to less than 500 basepairs, substantially increased the transfected area of hepatocytes observed to over 70% of hepatocytes, an increase from under 50% with a regular plasmid. The present disclosure also elucidates the surprising finding that the efficiency of gene delivery greatly increases at doses equal to or above 20 mg DNA per kg per liver weight, and subsequently saturates thereafter. Such a non-linear relationship was not anticipated, but is greatly useful in designing therapeutic dosing schemes. The invention also makes the surprising finding that a high-level transfection of an integrated reporter gene above 40% does not show any loss in levels over three months, countering previous reports with transposons and the first to show immune tolerance at this threshold of antigen expression. The disclosure also makes the surprising finding that episomal vectors delivered by hydrodynamic injection can be expressed for 4 months, the longest duration of expression ever observed, and that they survive a major dilutional loss of liver up to 50% increase in size. The disclosure also reports the surprising finding that very large DNA vector sizes can by delivered into large animals, exceeding 12 kb. Even more surprising, the transfection efficiency did not appear to decrease, in spite of progressively larger DNA vector sizes. This counters against every published report with non-viral DNA vectors. The disclosure also makes the discovery that a gene injection can be repeated again with successful gene expression at equivalent efficiencies to the first injection, such that both proteins are detected and found in the same cells. It was uncertain whether hydrodynamic injection from the first injection would disrupt the liver architecture to prevent a second injection. The disclosure also makes the discovery that high DNA vector doses up to 40 mg per kg liver weight show no toxicity, which had never been tested before, and more importantly that DNA concentration in the injection fluid makes a major determinant on observed delivery efficiency. The disclosure also elucidates new discoveries related to the efficiency of gene expression in regards to flow rate, and how to optimize injection with appropriate volume doses. Both of these had never been reported previously. Finally, the disclosure reports that liver sinusoidal endothelial cells can be targeted for gene delivery, which had not been shown in the previous publication.

### Overview

Hydrodynamic injection through the liver represents an exciting new gene therapy modality. It is advantageous because it is non-viral and thus is highly scalable and safer than viral approaches. Hydrodynamic injection can deliver naked DNA alone representing the simplest gene therapy modality possible. The technique is known to be very efficient in mouse models but traditionally has not scaled well into large animal models.

Several groups have investigated the ability to scale hydrodynamic injection to large animal models over the years. An inherent challenge in developing new technologies for hydrodynamic injection is the challenge in translating the technique between animals. New methods must be adapted and optimized for each animal and organ, empirically testing which strategies will work. The pig is a convenient animal model for testing, since its liver size can be equivalent to an adult-aged humans depending on the age of the pig. Thus, hydrodynamic techniques established in pigs are likely to be translatable into humans. There remain several caveats with this translation. For instance, pig's livers have interlobular fibrosis between lobes that could create some uncertainty.

The work in mouse, rat, and rabbit models in hydrodynamic gene delivery is solid proof of concept work, but unfortunately does not inform how the procedure would be conducted in human patients, and how the gene delivery efficiency could be optimized. One example of this is that many groups isolate a specific lobe of the liver and inject through the hepatic vein servicing that lobe. This technique was first described in a paper using a rabbit model in Human Gene Therapy 2002 Nov. 20; 13(17): 2065-77.

Similar techniques have been used in pigs since then, applying the same strategy of isolating an individual lobe with a balloon occlusion catheter into the lobe with hydrodynamic injection (Mol Ther. 2009 March; 17(3): 491-9). However, the flow rates and volumes employed in these later studies mediating gene delivery in pigs could not have been predicted from the rabbit study, since unlike traditional pharmaceuticals or biologics, there is no simple weight-based scaling to the hydrodynamic injection procedure. This finding is seen with simple hydrodynamic tail vein injection in mice and rats, wherein the volume and flow rate employed in mice does not translate easily into predictable rates when adjusted for their larger size.

Another example of variability is that the vessel or duct chosen for hydrodynamic injection makes the parameters of the injection change, along with how the gene delivery works and the results thereof. An example of this has been observed in pig models, where single lobe injection is more efficient than strategies that inject through the hepatic vein to reach the entire liver, compared to strategies clamping the inferior vein cava and portal vein to isolate blood flow there. The subtle differences between these different vessels are not predictable until the experiment is performed.

Similarly, the biliary system as a different route for hydrodynamic gene delivery has been studied. The biliary system is unique in possessing a total volume that is much smaller than the vasculature feeding the liver. This comparison is ~15 to 30 mL for the biliary system volume compared to around ~600 mL for blood volume in a human adult. The biliary system is also unidirectional, so injected fluid doesn't simultaneously escape through an unsealed end. This unidirectional feature of the biliary system is advantageous in maintaining appropriate fluid pressure during the injection and making sure the DNA solution at high pressure is ejected into the surrounding liver tissue, rather than blasting outward through another liver vessel.

Even though a hypothesis of liver-directed gene delivery has existed for over 20 years, specific details about how to do the procedure efficiently in a large animal model and what the outcomes might be have eluded investigators. The first description of hydrodynamic gene delivery through the bile ducts was based on experiments in dogs. The group used a surgical procedure, an injection rate (~1 mL/sec), and a suture to prevent antegrade flow within the biliary system (Hum Gene Ther. 1997 Oct. 10; 8(15): 1763-72). The investigators injected into the common bile duct, such that fluid was also injected into the gallbladder in addition to the liver. The investigators could only detect a small level of luciferase activity, but did not observe any protein detection by histochemical analysis of the liver, so it is likely that an undetectable percentage of the pDNA got into hepatocytes.

Another group applied the use of ERCP to mediate gene delivery into the liver into dogs and pigs (GIE 2005, T1249 Abstract). ERCP was an improvement in technique over surgery. Other changes to the procedure used by investigators was to use a balloon to prevent antegrade flow during hydrodynamic injection. thereby increasing the amount of fluid with DNA solution that enters into the liver. The group used an injection rate of 5 mL/min for their procedure and continued to inject through the common bile duct, thus simultaneously injecting into the liver and gallbladder. The investigators did not report any gene delivery efficiency into the liver, as reflected by protein expression detectable on IHC, IF, or Western blot. There was a report of detection of a protein in systemic circulation, however. The efficiency and utility of the procedure remains uncertain as applied to liver diseases.

At the same time, another group reported that injection of pDNA through bile ducts via a surgical in rats could mediate gene delivery (Gut. 2005 October; 54 (10): 1473-1479), but as discussed above, hydrodynamic parameters in rodent models can not be scaled up to larger mammals such as dogs, pigs, and humans. As an example, the flow rate employed by the group was 0.54 mL/min and surgical ties helped seal the bile ducts. The relatively efficiency of pDNA delivery into hepatocytes by histochemical staining was only ~1% of hepatocytes, which is significantly less efficient than the comparable hydrodynamic tail vein approach.

Improved techniques for biliary hydrodynamic injection were explored. In a first study, mediating gene delivery into pigs via hydrodynamic injection and observing detection by immunostaining was investigated (Kumbhari, G I E 2018). New injection parameters that could be tolerated by the pig's biliary system were studied. The pig's bile ducts ruptured at parameters higher than 2 mL/sec and 30 mL per second, so those parameters were chosen for subsequent work. The pig's biliary system was accessed by ERCP and the balloon was placed in the common hepatic duct to avoid the cystic duct and gallbladder injection. This was a key innovation compared to previous studies, since avoiding gallbladder injection would allow for more pressure to be generated in the liver during injection and thus more efficient gene delivery. The balloon was kept inflated after the injection for 1 minute to increase more fluid delivery.

The result was that the presence of genes in the liver expressed from pDNA was verified for the first time with experimental data (Kumbhari, G I E 2018). PCR showed the presence of pDNA in all lobes and aspects of the liver. Western blot confirmed that the delivered protein was expressed in all liver tissues. Immunofluorescent staining for the delivered protein was also observed, although the delivery efficiency was <1% of hepatocytes. The expression of protein appeared to be stable with a Sleeping Beauty transposon effect out to 60 days.

While this previous study was a significant advancement, the delivery efficiency was not sufficient to treat any clinical disease, which would require a significant percentage of the liver exceeding 10-20% of hepatocytes to express the transgene of interest. Improvements were needed over the previous study that were discussed in detail in published data (Kruse, G I E 2021).

First improvements focused on better vector composition that was injected into the liver. A hepatocyte-specific promoter was used to drive expression, along with codon-optimization of the transgene, and 3′ UTR stabilization. Higher doses of plasmid were introduced, and a more active transposase system in piggyBac. Together, these changes to the composition, pDNA doses, and transposase afforded a full magnitude increase in transfection efficiency, exceeding 30% of hepatocytes expressing the transgenes. These improvements were all described in Kruse, G I E 2021.

Additional efforts to improve the technique involved targeting multiple cell types in the liver via ubiquitous promoters. This included bile duct cells, endothelial cells, and neurons. Vector composition refinement demonstrated the capacity for cell-specific delivery, showing that only hepatocytes or endothelial cells could express the desired transgene when respective hepatocyte or vascular endothelial cell promoters were utilized. Additional new discoveries included the ability to change the flow rate to target zone 3 instead of zone 1 of the hepatic lobule. The flow rates causing transaminase elevation and liver injury were defined. Alternative strategies of accessing the biliary system beyond ERCP to conduct biliary hydrodynamic injection were described for the first time.

The tolerance of the bile ducts to higher flow rates and volumes without rupture was understood more completely. The pressure relationship with the injection parameters was defined, as well as identification of pressures reached during biliary hydrodynamic injection that are sufficient to mediate gene delivery. These findings were described in Huang, PLOS One 2021.

Another group has subsequently contributed to biliary hydrodynamic gene delivery (Mol Ther Methods Clin Dev. 2022 Jan. 19; 24:268-279). The group studied the hydrodynamic gene delivery technique in infant pigs weighing between 4-6 kg. The methods and technique employed by this group were different than the published results by Kumbhari and Kruse; consequently their efficacy was different. For their protocol, they accessed the biliary system by a surgical procedure, rather than ERCP. They did not use a balloon catheter, but rather leveraged different surgical clamps over the cystic duct and multiple different veins. A single lumen catheter was used for the injection. No clamp was placed over the bile duct itself, likely to avoid breaking the catheter or, if the catheter was in place, the surgical clamp would never seal. The injection itself occurred in the common hepatic duct, but no antegrade flow was blocked into the common bile duct, allowing for pressure decrease and DNA solution to escape into the intestine.

The injection parameters were significantly higher than what was employed in the previous Kruse and Kumbhari studies with targets of 10 mL/sec and 100 mL of injected volume. The group was able to detect the presence of DNA by PCR in all lobes of the pig liver. Similarly, luciferase enzyme activity was also detected in all lobes. Unfortunately, very little protein expression could be detected upon immunostaining of the liver, with only rare cells detected. Another important factor was that while protein expression could be detected initially, it diminished almost completely by 10 days post-injection, the investigators used episomal minicircle and nanovector/nanoplasmid vectors as the DNA construct. These vectors are reported to be superior to regulator plasmid DNA, but they were inadequate to increase expression levels on IHC or mediate long-term expression. Together, the results suggested limited efficacy of their method/technology and it is doubtful whether these alternative vectors can improve expression from the biliary hydrodynamic technology.

The instant disclosure seeks to provide improved techniques, methods, and compositions to increase the gene delivery efficiency after biliary hydrodynamic injection in large animals.

Additional improvements include new safety strategies and methods of targeting gene expression for specific cell types. Other improvements include redosing of a gene vector as well as strategies to achieve long-term expression. Size limitations that are possible with biliary hydrodynamic injection were also described.

### Increasing Transfection Area

A key goal of gene therapy techniques is to deliver DNA inside as many target cells as possible and have the DNA reach the nucleus such that protein expression can be detected in as many target cells as possible. This is key whether for viral vectors or non-viral strategies. Previously, expression was achieved in 30% to 50% of hepatocytes in pigs while differing on the DNA dose. However, the expression in each lobe could be notably variable. Furthermore, only one plasmid DNA composition and only one transgene were described for this result, which could bias the efficiency of this technique when it is applied to other vector sizes and proteins.

To current delivery of DNA to more hepatocytes, further modifications to the hydrodynamically injected vector composition were initiated. A modified vector composition could synergize with the procedure parameters to yield a better result. Modification of the plasmid DNA by reducing or removing the bacterial backbone could greatly increase the observed transfected area in pig liver sections to over 50% of hepatocytes. In certain sections of the transfected area, 60%, 70%, 80% or even 90% of hepatocytes within the lobules expressed the gene of interest. On average, 72% of hepatocytes were staining positive for the gene of interest. The stained area was notably darker, which indicates higher intrinsic protein expression within the cells.

This improved transfection efficiency was achieved by injecting a vector that is a circular DNA molecule that lacks most bacterial sequences. The bacterial sequences present are less than one kilobase in length or in some cases less than 500 base pairs. One example is the nanovector or nanoplasmid, which has a reduced origin of replication and bacterial selection region. The nanoplasmid has a R6K conditional replication origin, which requires pir+ E. coli host cells for propagation. Selection is accomplished by the RNA-OUT system, which downregulates the SacB gene. In another example, the vector is pFAR, which has a smaller bacterial backbone under 1000 base pairs. The pCOR backbone consists of three bacterial elements: the 0.4 kb R6K γ conditional origin of replication (ori γ), which requires a functional R6K π initiator protein, a 0.2 kb selectable tRNA suppressor gene (sup Phe), and a 0.4 kb cer (ColE1 resolution) fragment to resolve pCOR oligomers, totaling under 1000 base pairs. Any bacterial backbone of different construction under 1000 base pairs, preferably under 500 bp's would be suitable for this approach. Another option is the use of minicircle vectors, a circular DNA molecule that has no bacterial elements present. Minicircle vectors are generated by the recombination of two different sites on the plasmid DNA. Recombination of these sequences removes the bacterial sequences on the plasmid DNA, leaving only the mammalian sequences and a short remnant of the recombination site.

Another DNA vector composition that produces a higher transfection area after biliary hydrodynamic injection is a linear DNA molecule. In preferred embodiments, the linear DNA vector is a close ended linear DNA molecule (PLOS One. 2013 Aug. 1; 8(8): e69879), also known as a ministring DNA molecule (Mol Ther Nucleic Acids. 2014 June; 3(6): e165), or doggybone DNA (Hum Vaccin Immunother. 2015 August; 11(8): 1972-1982). These linear DNA molecules lack any bacterial sequence and have covalently closed ends. As a result they do not trigger DNA damage responses and will not integrate into the genome. These linear DNA molecules also have move more freely in solution which aids in their translocation during hydrodynamic injection into the cell nucleus. The linear DNA molecules have small residual sequences (up to 50 bp on each end) that are remnants of viral or bacterial excision sites for production, but otherwise contain no other bacterial sequences.

These vector compositions serve to synergize with biliary hydrodynamic injection because the lack of bacterial elements gives them a better expression profile. Another hypothesis is that the smaller size may help the translocation to the nucleus during hydrodynamic injection. The result is unexpected since these types of DNA molecules do not notably improve the transfected area in mouse studies and most their effects are thought to be cell specific for expression. It is not known whether these molecules improve the transfection area during hydrodynamic injection. In one study of biliary hydrodynamic injection, a reduced bacterial backbone (minicircles, nanoplasmid) failed to improve the procedure (Mol Ther Methods Clin Dev. 2022 Jan. 19; 24:268-279). However, for gene delivery using biliary hydrodynamic injection, the vector compositions described above should be used to maximize the amount of protein expression in hepatocytes.

### Improvement in Expression Longevity

An important aspect of gene therapy is the length of expression. Length of expression is influenced by several different factors including intrinsic silencing of the gene vector, dilutional loss of the episomal DNA construct, death of the cell receiving the gene, and adaptive immune responses against the gene product. These can be greatly influenced by the delivery vehicle and/or the procedure employed to deliver the gene.

In the case of hydrodynamic gene delivery, the procedure itself can cause traumatic injury via the high-pressure injection of fluid into the tissue. Tissue transiently swells with fluid, which can cause cell death in multiple different cell types. This is particularly seen in mouse models after hydrodynamic injection with necrosis of some tissue, as well as transient inflammatory responses with immune infiltrate. In a study in canines using vascular hydrodynamic gene delivery, it was observed that the designated hydrodynamic injection parameters were associated with early loss of transgene expression. It was thought that this occurred because they primed immune response to the transgene product. The transgene product being expressed in the inflammatory milieu of hydrodynamic injection can have vaccine-like effects. Therefore, it is not certain whether any hydrodynamic procedure would have long-term expression, whether from DNA intrinsic reasons, or from an immune response against the transgene product.

The potential for hydrodynamic injection to lead to short-term expression of delivered transgenes is consistent with observations in mouse models where hydrodynamic injection of a plasmid encoding hepatitis B virus antigens leads to acute clearance. In comparison, adeno-associated virus (AAV)-mediated introduction of the hepatitis B virus antigens leads to long-term expression of viral proteins with a lack of inflammation. Thus, it can be uncertain if hydrodynamic injection will lead to short-term or long-term expression, particularly in comparison to other modes of gene delivery.

It is unknown how the biliary hydrodynamic injection technique will modulate or interact with the host immune system to allow for long-term expression, since the technique is intrinsically different from other hydrodynamic strategies. Published data showed that a transposon strategy with biliary hydrodynamic injection could express the genes for up to two months (Kumbhari, G I E 2018). However, the amount of protein produced in that study was miniscule with few cells (<1%) expressing the delivered genes. There was not enough protein from a clinically relevant transfection rate for the immune system to recognize and respond to. It was unpredictable to know whether or not the immune system would mount an adaptive immune response after high-level transfection based on prior studies. Considering the subsequent improvements to the biliary hydrodynamic technique, it is unpredictable to know if the additional protein would be more visible to the immune system, resulting in its eventual elimination. Published data showed that expression was maintained for three weeks, but adaptive immunity can take up to two months to develop, so three weeks is a very early time point for consideration. A longer time course of expression is required in order to know if the integrated transgene would express the genes for the life of the animal.

The present disclosure demonstrates that the combination of an optimized expression cassette with hepatocyte-specific promoter and piggyBac transposon is successful at mediating transgene expression in pig hepatocytes for up to three months. The relative transfection area of hepatocytes expressing protein showed no significant difference compared to the transfection percentage at day 3 and at 1 month. This verifies that stable expression occurred, and no immune responses occurred. The designated parameter techniques as well as this vector composition are the best options to achieve goals of three months or more duration of expression.

A major challenge of gene therapy is the potential for genotoxicity from the integration of DNA into the host chromosome causing cancerous transformation. Consequently, many regulatory agencies and physicians prefer strategies wherein episomal DNA is delivered into cells. Episomal DNA does not integrate into the host genome and is expected to have minimal to no effect on host cell viability. The challenge with episomal DNA strategies is that they can often lose their potency of expression over time, due to a variety of reasons including. gene silencing of the episome and dilutional loss of the plasmid DNA with cell division. Improvements are still needed to develop the best practices, methods and vector compositions in order to yield long term expression of episomal DNA after biliary hydrodynamic injection.

Previous studies of hydrodynamic injection through vascular routes in pigs observed episomal pDNA expression for up to two months post-injection time, although the transfected area declined by approximately 50% in that time (Mol Ther Nucleic Acids. 2013 October; 2 (10): e128).

Literature from mouse studies illustrates that a large portion of plasmid DNA molecules injected will silence expression after one month, so it is unknown how long expression time would last with a non-viral construct. Even for viral vectors such as AAV, a substantial loss of expression from episomal DNA occurs after weeks to 2-3 months post-injection (bioRxiv 2022.03.24.485675). While the previous study of vascular hydrodynamic injection is encouraging, it is unknown if a biliary approach could achieve similar duration of expression or even longer, particularly in growing pig with progressive pDNA silencing. There are no studies describing expression of pDNA from biliary injection in large mammals beyond 1 week.

The present disclosure addresses these limitations by describing a method for achieving episomal DNA expression for up to four months with the use of a vector composition that lacks a large bacterial DNA backbone sequence. In the previous vascular hydrodynamic studies, this approach was either not pursued or it failed to achieve long-term expression with reduced bacterial backbone vectors. This modified vector DNA backbone must be delivered into cells using a hydrodynamic biliary approach at set parameters such that the immune response will not be triggered and that proper translocation of the pDNA into the nucleus will occur. The present disclosure is an improvement because it achieves expression of pDNA from biliary hydrodynamic injection for the longest duration to date. Additionally, the techniques herein also overcome prior obstacles in the gene delivery of episomal vectors, wherein during the current experiment, the pig liver increased 50% in size from the time of DNA administration to the time of liver harvest at the end of the four month experiment. Based on the literature of episomal vectors, this would have normally caused a significant dilutional loss of vector genome, which should drive down the transfected area of hepatocytes observed (Hum Gene Ther. 2012 May; 23(5): 533-539). By contrast, the transfected area of hepatocytes in our study remained relatively similar throughout the duration of the study, suggesting special, unexpected features of the biliary hydrodynamic gene delivery approach and the vector composition with reduced bacterial backbone size.

### Redosing of Gene Expression Between Procedures

A current limitation of all gene therapy approaches is the inability to redose the gene therapy vector. This is primarily due to viral vectors yielding immune responses against the viral capsids such that a potent immune response will prevent free dosing of the vector during a second administration. Hydrodynamic injection may be a method of solving this limitation since it is non-viral and lacks any protein components that could generate an immune response. In mouse models, hydrodynamic injection in mouse models has been able to be redosed with the result that the second injection yields similar protein expression to the original injection. Hydrodynamic injection in mice has not been shown to be additive to the original injection, however, likely because pDNA gene silencing made this experiment difficult to prove.

While hydrodynamic injection in certain mouse models has been shown to be redosable, studies in large animal models have not succeeded. A redosing study in a dog model with hydrodynamic injection failed to achieve any expression with the second dose (Hum Gene Ther. 2017 July; 28(7): 551-564) despite the administration of immunosuppression drugs. This was thought to be caused by a profound immune response to the transgene during hydrodynamic injection, which can be restimulated with each subsequent injection. In another study in pigs, redosing was achieved with a second administration of human alpha-1 antitrypsin, but the contribution of the first and second doses toward the expression observed is difficult to determine. As a result, previous studies do not teach whether hydrodynamic injection in large animal models can be redosed, or if two different genes could be redosed in succession without inhibition of the expression of the first gene dosed, and if the same cells within the liver could be targeted for expression with a second dose. Previous studies do not determine whether redosing of gene therapy is feasible from a gene expression and immune response perspective in the biliary system. There is no existing data from the biliary system to support the potential for redosing. It was previously published that the procedure itself could be repeated a second time in the pig without rupture of the bile duct or substantial injury to the liver, but this does not address whether gene expression using the procedure is possible. More importantly, it does not establish whether or not the second injection would have the same gene delivery efficiency as the first injection.

To address the challenges of redosing, the present disclosure describes an approach to achieve redosing of non-viral gene therapy through the biliary system. One approach is using a vector composition with a cell-specific or ubiquitous promoter for the first injection, using the established biliary injection parameters optimized. This second injection procedure can be accomplished at least one week, two weeks, three weeks, 4 weeks or later after the first injection, thereby achieving significant gene expression. The injection procedure techniques do not need to be changed to achieve this result. The second injection does not result in the loss of expression from the first injection, and individual cells in the liver can express DNA from both injections, with the result that the second injection does not cause translocation or silencing of gene expression of DNA from the first injection. The transfection efficiency does not diminish or change with the second injection. Depending on the promoter used during the second injection, the second injection can have DNA targeted into all cell types, including bile duct cells and endothelial cells, targeted during the first injection without interference.

Redosing of Gene Expression within a Single Procedure

It was previously published that it is technically feasible and well tolerated by the pig to have multiple hydrodynamic injections through the biliary tract within a single bile duct canulation in one pig. What remains unclear is to whether one could inject DNA in multiple injections during a single pig procedure to yield expression from different plasmid DNA molecules. There are reasons to suspect that this may not technically work and may have deleterious effects on the pig liver. It was previously demonstrated that within 15 minutes after biliary injection at high flow rates, that fluid fill vesicles can accumulate inside the cytoplasm of the pig (Huang, PLOS One 2021). If these vesicles were to accumulate, it is uncertain if the hepatocytes could tolerate additional vesicles and/or if the vesicles would disrupt the transport of the second DNA through the cytoplasm into the nucleus. Moreover, while multiple injections were tolerated in a single day, it is unclear if the long-term effects on the pig would be toxic to the liver and if potential liver toxicity would increase at longer time points post-injection leading to ablation of protein expression.

The major clinical reason to conduct multiple injections in a single procedure is two-fold. In one example, the patient requires treatment with different DNA constructs encoding different proteins to treat their disease. The cost and logistics of compounding these DNA constructs into a single volume may be unpractical or change the total fluid volume jeopardizing the efficacy of hydrodynamic delivery. More specifically, combining the volume and/or increasing the DNA concentration into a single injection may alter the optimized parameters of that gene delivery procedure. Therefore, as an alternative to this approach, the same pig could be injected a second time in order to target gene delivery into the liver again.

The second reason to have multiple hydrodynamic injections within a single ERCP procedure is in case of a technical difficulty and/or technical failure, wherein the injection fails because of the balloon sliding out of place, the balloon deflating, the power injector failing, or the Luer lock connections within the catheter to power injector tubing failing. In these circumstances, a second injection could immediately be repeated with the full DNA dose. An important reason to try the second injection at that time rather than reschedule to a different day is that each ERCP procedure carries some risk of pancreatitis and thus the number of total ERCP procedures should be reduced. Moreover, if a second DNA solution is available, it would be relatively easy to load the power injector again with the catheter already in place and proceed forward with the injection.

Previous studies have not demonstrated that redosing of gene within a single biliary procedure is technically feasible. The current technology method shows that it is feasible and practical in human patients. In the present disclosure, the second gene injection can proceed within 5 minutes of the first injection. In other examples, the gene injection can proceed within 10 minutes, 15 minutes, or 20 minutes, or longer after the first injection. The current technology method shows that after the second DNA treatment, the two proteins expressed from these DNA molecules are able to be detected alongside each other within the same liver, despite being injected at separate time points. The current technology method also shows that proteins from the DNA delivered can be found co-localized within the same liver cells. For this method, the invention shows that injection parameters do not have to be modulated from their intended use as single injections. However, flow rates above 5 mL/sec are avoided during the first injection in order to prevent significant fluid vesicle development that would inhibit the second injection.

Mixing of pDNA Vectors During a Single Injection for Co-Delivery into the Same Cells

In some instances, it is preferable to deliver multiple different genes inside cells at the same time. In prior studies, this can be accomplished by placing multiple gene cassettes on the same plasmid DNA. However, the disadvantage of this approach is that the larger size of the plasmid DNA may decrease the translocation the plasmid DNA inside the nucleus. Furthermore, for manufacturing schemes that already produce both plasmid DNA molecules and separately, it would be preferable in many cases for the plasmid DNA molecules to be combined into a single injection volume rather than two different injections. In these cases, cloning of the plasmid DNA constructs into a single plasmid becomes unfeasible and costly. Moreover, the larger plasmid DNA may have reduced delivery efficacy.

Previous studies have never demonstrated that two different plasmid DNA's can be delivered at the same time such that gene expression from both plasmid DNA is achieved. It is uncertain whether both plasmid DNAs would be equally expressed within different cells, or if one plasmid would be dominant over the other for gene delivery efficiency. It is additionally unclear if both plasmids would colocalize within certain cell types, which would be necessary for efficacy of certain disease indications.

The current technology method teaches that biliary hydrodynamic delivery of two different plasmid DNAs at the same time is feasible and that the gene expression can be co-localized in the same cells. Through the biliary hydrodynamic approach, almost all cells seem to have colocalization of both proteins within them after gene delivery. This suggests that the same liver cells are receiving the same amount of fluid pressure at the same time thus leading to co-delivery of plasmid DNA.

### DNA Size Limitation

The size limitation of DNA vectors for a given gene therapy method is crucial when defining the potential applications of gene therapy. Current vector systems including adeno-associated virus (AAV, 4.8 kb genome size) are very restricted in their DNA cargo sizes, which limits their ability to deliver fully genes for several rare diseases (VWD, hemophilia A), amongst other therapeutic possibilities. Viral vectors are intrinsically limited in packaging size since they must encapsulate their entire genome into a capsid defined geometry. Non-viral strategies lack this limitation in general, since they are not packaged into a viral capsid. However, all non-viral strategies have been observed to have different transfection efficiencies depending on the plasmid DNA size used. For example, some transfection reagents do not transfect larger pDNA constructs as well as smaller pDNA sizes. In mouse models, it is known that hydrodynamic injection can deliver DNA of substantial sizes inside cells. However, it has never been investigated whether this fact extends into hydrodynamic strategies in large animals. Additionally, it has never been demonstrated whether or not biliary hydrodynamic injection can deliver plasmid DNA molecules of substantial sizes, greater than 8 kb, 9 kb, or 10 kb in size.

Previous studies show that a plasmid DNA molecule of the size 5.5 KB could be delivered into pig liver by biliary hydrodynamic injection at high efficiencies. There was no further data published on what happens with plasmids or larger sizes. In the present disclosure, strategies of delivering DNA molecules exceeding 12 kilobases in size are described, as well as, delivery of DNA molecules greater than 15 kilobases, and 20 kilobases in size through the biliary approach. These strategies include the delivery of DNA into multiple cell types including hepatocytes, bile duct cells and endothelial cells. Obtaining plasmid DNA in excess of 10 kb of size and leveraging higher DNA mass doses to maintain equivalent per molecule DNA dose are described. Biliary hydrodynamic injection using injection parameters of volume >30 mL/kg and greater than 2 mL/sec being sufficient for delivery of large DNA constructs into the cell types is described. Expression is achievable with ubiquitous promoters such as cytomegalovirus promoter. Cells can also be targeted with specific promoters that could be utilized for increased expression.

The newly discovered ability to deliver larger DNA sizes helps to define guidelines for appropriate dosing. Transfection efficiency can be maintained despite larger plasmid size if the proportion of molecules to vector is the same. The first step is to normalize all DNA weight-based dosing (mg DNA/kg liver) by the index plasmid size to achieve the transfection threshold of the index plasmid. For example, in this case the index plasmid used to establish the transfection efficiencies has a size of 8.6 kb, with known goals of ~10 mg/kg dose for 50% and ~20 mg/kg dose for 70%. Within those parameters, multiplying the weight-based dose times the plasmid DNA size (in kb) divided by the 8.6 kb index plasmid size would yield a plasmid DNA dose that should yield similar injection results.

To simplify the clinical application, the following formula is used to calculate the DNA dose for any given liver weight (kg) and plasmid DNA size (kb) as summarized in Table 4.

TABLE 4
Formula for DNA dose

| | |
|---|---|
| DNA dose(mg)/Liver (kg)/plasmid DNA(kb) | % Delivery |

| | | |
|---|---|---|
| 1 | mg/kg/kb | 50% |
| 2.5 | mg/kg/kb | >70% |
| 3.5 | mg/kg/kb | >70% |
| 5 | mg/kg/kb | >70% |

Slight deviations in this formula within 0.5 mg/kg/kb will not affect the percentage greatly and produce similar dosing efficacy.

This calculation should not be used for a nanoplasmid or other reduced bacterial backbone size vector, since it was observed in experiments described above that even when accounting for different plasmid DNA sizes, nanoplasmids or other vectors with reduced backbone size had intrinsically better properties for mediating higher transfection efficiency. Consequently, only like vector types should be compared when using the formula. Similar calculations could be made in a nanoplasmid dosing experiment to elucidate proper dose.

Summarizing the above experiment, a 5.8 kb nanoplasmid was found to achieve transfection ~70%. The formula for 70% would have calculated 14.5 mg pDNA, when only 10 mg pDNA was needed. Thus, the nanoplasmid itself has more intrinsic activity. In order to account for this activity, a 30% reduction in DNA dose could be applied to account for higher nanoplasmid activity. Alternatively, a similar empiric series of doses could be calculated, or the current dosing formula could be used, with the caveat that doses higher than 2.5 mg/kg/kb are unnecessary.

### Escalating DNA Doses

Another avenue of research is to determine if increasing doses of plasmid DNA could improve on the biliary hydrodynamic injection. A prior published report only tested pDNA doses of 3 mg and 5.5 mg pDNA, which showed a higher transfected area with the higher dose of pDNA (Kruse, G I E 2021). It would be useful to find out if increasing the dose incrementally might keep increasing the transfection area, and if a different pDNA molecule would work. This information would be invaluable for guiding the proper amount of clinical dosing for gene therapy procedures. While the intent of higher pDNA dosing would be to secure more gene delivery into hepatocytes and thus increase the transfected area observed, a secondary concern with escalating pDNA doses is whether any toxicity would occur. Naked plasmid DNA can be recognized by innate immune receptors inside cells causing inflammation. In general, dose dependent toxicity is a major issue for all gene therapy vectors today, which has been observed in gene therapy trials with AAV and adenovirus. Dose limiting toxicities in clinical trials can also be observed with lipid nanoparticle vectors delivering mRNA or siRNA, or with naked oligonucleotide conjugates. There exists a possibility that a given concentration or amount of DNA that could be hydrodynamically injected into the liver of a large mammal could cause significant toxicity. Toxicity could also represent itself as the paradoxical decrease in transgene expression from cell-based death, or from triggering innate immunity to decrease expression from pDNA. Such knowledge would inform practitioners on proper dosage levels for this biliary hydrodynamic delivery technology.

The question of toxicity was investigated empirically to refine the technological approach. Four different concentrations of the plasmid, pT-LP1-ATP7B,C9 were injected into pigs ranging from 36 kg to 41 kg in weight. The concentrations were ~10 mg, ~20 mg, ~30 mg, and ~40 mg pDNA in size. The flow rate was kept the same at 2 mL/sec in all studies. It was observed that pDNA continues to increase in transfected area with increased doses up to 20 mg pDNA. Higher doses of 30 mg or 40 mg pDNA did not show any significant difference from 20 mg pDNA. This discovery avoids wasted pDNA doses, so that more patients can be treated. There was no difference in underlying toxicity from the injection with higher pDNA doses, because there were no changes in vital signs and no elevations in transaminases post-injection.

Higher doses of pDNA can be incorporated to achieve higher transfection area, beyond the 5 mg pDNA previously described. Doses up of 20 mg, 30 mg, or 40 mg are well tolerated. The present disclosure shows that DNA doses equal to or more than 20 mg pDNA can increase the transfection area up to 70% of total hepatocytes. This represents an unexpected increase in efficiency, given that 5.5 mg and 10 mg pDNA only amounted to 50%. Moreover, it was also unexpected that the effect would saturate at around 20 mg/kg, and that larger plasmid DNA doses for a given plasmid DNA size are unnecessary because they do not increase the transfected area. In addition, DNA doses at least up to 40 mg pDNA do not cause cell-mediated toxicity that would decrease transgene expression. Higher doses can be utilized safely through biliary hydrodynamic injection and can be considered when applying the technique for multiple different therapeutic applications.

### Injection Parameters

Previous studies reveal a combination of vector composition and procedural technique that yields efficiency gene delivery achieving a transfected area of tissue expressing at high efficiency. The portion of the technology that was undefined is how different flow rates and volumes impact gene expression, and how to improve gene delivery.

While all components of the technological method are important, determining the optimal injection force is crucial for delivering more pDNA inside cells, while avoiding potential toxicity.

Previous studies describe injection parameters of 2 mL/sec to 4 mL/sec used in gene delivery strategies. Volumes tested range from 30 mL to 40 mL in total. The studies also describe that the biliary system can tolerate a wide range of injection flow rates, up to 10 mL/sec tested. The highest volumes also tested were 140 mL going into the liver. However, while it has been shown that these parameters can be tolerated from a technical perspective without bile duct rupture or serious liver injury, it does not show how they would impact the delivery of DNA, and what parameters would be optimal to use.

A series of studies were conducted to understand how higher and lower flow rates influence gene delivery efficiency of the biliary procedure, as evaluated by transfected area of immunohistochemical staining. Another set of studies tested what the influence of volume is on these parameters. All studies kept the other variables the same, including plasmid DNA construct and plasmid DNA dose.

### Low Flow Rates

Lower flow rates are attractive because they could induce less tissue damage and inflammation from the injection and may be safer for patients. However, the techniques herein provide that flow rates of 1 mL/sec yield minimal-to-no gene expression in the liver combination with the procedural technique employed. The vector composition in this experiment has ubiquitous promoters of CMV and SV40 driving reporter gene expression. Therefore, flow rates at or below this threshold should be avoided, unless the purpose is to load DNA solution into the liver before a faster injection.

### High Flow Rates

Concerning the effect of elevating flow rates, the hypothesis was that the higher flow rates might mediate more pDNA entry into cells, with more hepatocytes transfected. However, the present disclosure shows that using significantly higher flow rates is not optimal. The flow rate of 4 mL/sec reflected previous studies with similar staining of hepatocytes and non-hepatocyte cells like bile ducts. The flow rate of 4 mL/sec could bias more expression to peripheral regions of the lobule, although central delivery could still be observed. Higher flow rates of 7 mL/sec and 10 mL/sec surprisingly decreased transfection efficiency into hepatocytes, with progressively fewer hepatocytes expressing the gene. Delivery into non-hepatocyte cell types remained strong at higher flow rates, with prominent biliary staining.

The current technology shows that flow rates for efficient gene delivery are optimally greater than 1 mL/sec and less than 7 mL/sec. Optimally, the flow rate is at least 2 mL/sec and no more than 4 mL/sec for hepatocyte-targeted delivery. Alternatively, bile duct cells can be preferentially targeted by increasing flow rates greater than 4 mL/sec. In other embodiments, the flow rates to target gene delivery into bile duct cells are greater than 7 mL/sec or greater than 10 mL/sec to drive more DNA into this cell type.

### Volume

Concerning the effect of increasing volume, it is unknown if delivering a larger fluid volume with a set plasmid DNA (pDNA) concentration into the liver would actually increase the expression observed. One hypothesis is that a larger pDNA volume would increase the saturation of DNA solution into the bile ducts and ensure more pDNA volume enters into cells. On the other hand, more volume could also serve to dilate the biliary system and tight junctions surrounding it, thereby allowing more fluid to escape into the vasculature. pDNA delivery into hepatocytes would not occur. A related consideration is that only a given portion of the DNA solution volume is “active”, such that a lower net concentration of pDNA in that active portion would actually be harmful. Moreover, a more dilute DNA solution could also mean that less effective DNA gets into individual cells, which would lower transfection efficiency.

An investigation was conducted by injecting 10 mg of pDNA and diluting it into 40 mL, 60 mL, or 80 mL of normal saline solution. The flow rate was kept at 2 mL/sec for all injections, which would push pDNA fluid solution into hepatic lobules.

The results of the investigations of the delivery technology found that higher volumes at a constant flow rate and set amount of pDNA concentration decreased gene expression. There was very little immunohistochemical staining among hepatocytes in the pigs injected with 80 mL volume, while 40 mL and 60 mL volume doses were more similar in efficiency.

Preferably, the volume the pDNA solution should be less than 80 mL. In other embodiments, the pDNA solution should be less than 60 mL. These volumes should be adjusted based on the weight of the target liver, which in these studies was approximately 1 kg.

Translating this information into a liver weight dosing strategy, optimally a flow rate of 20 mL/kg, 30 mL/kg, 40 mL/kg, 50 ml/kg, or 60 mL/kg is applied, wherein the kg represents the liver weight, which can be determined through a variety of different methods in the literature.

### DNA Dosing Guidelines

Previous studies specified that nucleic acid comprises DNA administered in an amount of at least 1 mg of DNA per kilogram of the subject's total liver tissue weight. However, there was no information on optimal concentrations of DNA solution to prepare. The current studies showed that large volumes can have a deleterious impact on transfection efficiency, but that this could be countered by increasing the concentration of DNA injected at large volumes.

Herein, DNA concentrations (mg/mL) should optimally be greater than 0.30, 0.40, 0.50, or preferably more than 0.60 mg/mL could be used. For simplicity, DNA dose per kg liver weight could be used as a paradigm, in which case doses of 10 mg/kg, 20 mg/kg, 30 mg/kg, or 40 mg/kg or more could be used. The volume should be adjusted to whatever DNA dose is selected, so that these parameters are realized.

### Increasing Transfection Rate Through Flow Rate Modulation

The injection parameters are a crucial part of achieving successful gene delivery. Regardless of the route of hydrodynamic administration, it has been observed in all animal models that slight modifications in the injection parameters greatly influence the outcome of the gene expression observed.

Previous studies have shown that biliary hydrodynamic injection uses set flow rates to achieve gene expression. All experiments are conducted at a single flow rate which is advised to mediate efficiency expression. Additionally, the distribution of expression can be slightly modulated from zone 3 to zone 1 of the hepatic lobule with increasing flow rate.

Previous studies do not present methods of utilizing multiple different flow rates during the procedure. While the idea has been contemplated previously, there are no details of which combinations of flow rates would achieve the highest-level expression. The current technology method presented suggests modifications of the gene delivery method that would yield even higher transfected area of hepatocytes beyond what was achieved with the current testing paradigm.

The present disclosure shows that transfection area in the liver can be further increased by using at least 2 different flow rates during the injection. In other instances at least three different flow rates are used. Previous studies state that this is technically possible but does not present specifics on what combinations of flow rates and time would be optimal to increase transfection area.

The present disclosure shows that it is optimal to start the initial hydrodynamic injection with a slower injection flow rate to avoid bile ducts and canaliculi from being dilated too soon with higher pressure thereby allowing more vascular fluid escape. Furthermore, the injection protocol teaches that it is best to fill the biliary system with fluid first before escalating into higher pressures.

For example, the initial injection proceeds at 2 mL/sec, followed by an increase in the injection rate to 4 mL/sec. This serves to first target gene expression into zone 3 at the center of the hepatic lobule, before targeting gene expression to the perimeter of the lobule with a faster flow rate. The net result of designing the injection parameters in this way would be to cover the entire lobule more efficiently, thereby increasing the number of hepatocytes that express the gene of interest. Optimally, the slow phase of the injection will proceed for half of the fluid volume. In another example, the slow phase will proceed for two-thirds of the injection volume. The fast phase is kept minimal since the increased force can be instantly communicated to the distal fluid and does not need to be experienced for longer periods of time. It is preferable to avoid the faster flow rate dilating the ducts and sinusoids too soon, so that the later flow rate achieves less pressure.

In another example, the first fluid phase proceeds at 1 mL/sec until the total volume reaches the estimated volume of the large bile ducts in the liver, followed by proceeding with one or more faster volumes to lead to increase expression of pDNA. Alternatively, the fast flow rate occurs first followed by a slower flow rate to introduce plasmid DNA into the center of the lobule. In this example, the initial fast injection would occur for the first half of the total volume, followed by the slow fluid volume that results in a shorter period of time. Optimally, the total volume is used to calculate how much time is needed for each fluid injection phase. Time is not the most important factor, since the volume injected can differ among many different procedures and this depends on the size of the large mammal injected.

In another example, the flow rate is increased in a step-wise function, such that three or more flow rates are used. In one example, the flow rate of 2 mL/sec is used first for 33% of volume injection, 3 mL/sec is used second 33% of the volume, followed by 4 mL/sec for the remaining injection volume.

Scaling the Procedure into Smaller and Larger Livers

Previous studies show that pigs the size of 40 kilograms and larger can tolerate the injection through the biliary system using the set parameters and balloon inflation. However, they do not show whether smaller pigs could tolerate such a procedure, or whether their smaller bile ducts would burst, or their livers suffer significant injury.

One study previously reported that pigs approximately 5 kilograms in weight could tolerate injection through the biliary system at parameters of 100 mL and 10 mL/sec. This study did not use a balloon catheter to secure pressure during injection, so it is uncertain what the effective pressure was inside the biliary system. Moreover, there are several other details of the procedure that are different, including the use of surgery. Consequently, although the study shows that the procedure is tolerated in small animals, it does not address the concerns that arise when the procedure is performed on large animals. This means that the method used in the study cannot be transferred to large animals without further testing.

To ensure that the biliary hydrodynamic injection can be scaled down into smaller animals, the current technology method shows that pigs smaller in size down to at least 25 kilograms and down to at least 15 kilograms tolerate hydrodynamic injection well, without rupture in the biliary system. Moreover, smaller volumes injected into these pigs still resulted in robust gene expression as measured by immunohistochemistry.

The present disclosure demonstrates methods of scaling the injection volume down to specific animals. Previous studies specified volume parameters for hydrodynamic injection and stated a goal of volume to liver weight dosing, but the exact specifics were limited to 30 ml/kg liver weight and 100 mL/kg liver weight. The present disclosure remedies these deficiencies with additional recommendations for weight-based dosing.

Optimally, the volume injected during hydrodynamic injection through the biliary system is 40 mL/kg of liver tissue weight. In another example, the volume is 50 mL/kg of liver tissue weight. In other instances, the volume is 60 mL/kg of liver tissue weight. The liver tissue weight was calculated through available calculations in the liver or through direct imaging modalities, prior to biliary hydrodynamic injection. The present disclosure shows that it is best not to exceed 70 mL/kg in total volume, since this is associated with lower efficiency. In optimal instances, the injection volume is between 30-70 mL/kg, or more preferably 40-60 mL/kg.

These new dosages for volume per kilogram of liver tissue are supported by data from additional pig experiments, showing that they are efficacious in mediating gene delivery that results in reporter gene expression in the liver.

### Pressure-Mediated Injection

Previous studies disclose pressures for effective gene delivery between 50 mmHg to 150 mmHg. The present disclosure seeks to further delineate which pressure yields the most efficient gene delivery within this range and in a higher-pressure range. Series of tests were conducted using a constant pressure injection device, which takes real-time pressure monitoring and uses it to adjust the injection real time to maintain a given pressure.

Using this device, a constant pressure injection at about 50 mmHg was tested. It was found that injection at this pressure threshold was largely inefficient, with limited transfection of hepatocytes observed. Using a higher-pressure threshold of 80 mmHg, it was observed that a higher transfected portion of a hepatocytes expressed the gene of interest. Consequently, a pressure of at least 80 mmHg would be preferable for hydrodynamic injection. Pressures above 150 mmHg were further tested to determine whether they would be effective in hydrodynamic gene delivery. It was observed that pressures of 175 mmHg and 200 mmHg could still yield effective gene delivery, with efficiency similar to injections at lower pressures below 150 mmHg. Injections that pressure higher than 200 mmHg began to yield less efficient transfection compared to these lower pressure thresholds.

### Targeting Liver Sinusoidal Endothelial Cells

Previous studies have disclosed that biliary hydrodynamic injection can mediate gene delivery into multiple different cell types in the liver. However, they do not show whether liver sinusoidal endothelial cells (LSEC) can be targeted. This cell type has very specialized functions bordering the sinusoids and space of Disse helping to regulate access to hepatocytes. Given that the sinusoids are next to these endothelial cells, however, it is possible that pressure would not build up along these cells to allow for a transfection. Alternatively, it is possible that the injection technique could still reach these cells. In order to ascertain if biliary hydrodynamic injection could reach LSECs, a cell specific promoter for liver sinusoidal endothelial cells was realized. This cell specific promoter, CD36, is expected to have expression in all LSECs throughout the entire hepatic lobule.

It was observed that hydrodynamic injection into the biliary tree could efficiently yield expression into LSECs, being most concentrated around zone 3, but also extending outward to zone one. Consequently, a technique toward targeting the LSEC population can be demonstrated.

The injection parameters to be used for LSEC delivery are similar to gene delivery into other liver cell types. The volume and flow rate parameters established can be utilized for LSECs then. Different DNA doses can be contemplated, starting at DNA doses of 5 milligrams per kilogram (liver weight) or larger.

### EXAMPLES

The present disclosure is further illustrated by the following examples, which should not be construed as limiting. The contents of all references, GenBank Accession and Gene numbers, and published patents and patent applications cited throughout the application are hereby incorporated by reference. Those skilled in the art will recognize that the disclosure may be practiced with variations on the disclosed structures, materials, compositions and methods, and such variations are regarded as within the scope of the disclosure.

### Example 1. Proof of Concept Study for Hydrodynamic Delivery from Retrograde Ureters

The kidney could be an important target for gene therapy. There are many different diseases in the kidney that could be treated, from chronic kidney disease (CKD) to polycystic kidney disease to different glomerular disorders. Gene therapy has not been successful for the kidney because systemically administered viral vectors into the kidney has not been well targeted, since the glomerulus prevents viral vectors from further entry into the kidney due to size exclusion. This is primarily caused by poor targeting of systemically administered viral vectors into the kidney, which is thought to be due to size exclusion of viral vectors in the glomerulus preventing their further entry into the kidney.

Different strategies of local administration could be pursued for viral vectors, which has shown some efficacy in delivery into the kidney. Non-viral strategies are an exciting alternative to viral vectors which could dramatically reduce costs and allow for redosing. The latter could be important since the half-life of some cell types in the kidney could be relatively short.

Hydrodynamic injection into the kidney has been pursued through high pressure injection through the renal vein. This has been tested in both rat and pigs demonstrating delivery of firefly luciferase by luminescence activity. An alternative strategy for hydrodynamic injection is to inject into the kidney pelvis. This has been demonstrated in mouse models, wherein scattered cells were positive for staining amongst different kidney types following injection.

The ureter can be easily accessed through routine cystoscopy conducted in outpatient clinics. The ureter provides convenient access to the kidney through a uni-directional vessel, wherein urine only flows from the kidney in one direction toward the bladder. This uni-directional vessel resembles the uni-directional nature of the biliary ducts of the liver and pancreatic ducts, wherein bile and pancreatic digestive enzymes, respectively, travel in one direction toward a defined outlet.

High pressure fluid injection through the ureter would lead to accumulation of pressure within the kidney, facilitating delivery of plasmid DNA inside cells. To support this hypothesis, different catheter placements within the ureter were tested, along with different injection parameters (flow rate, volume) to see if any combination would lead to gene delivery into the kidney. Safety measures were also investigated.

### Methods:

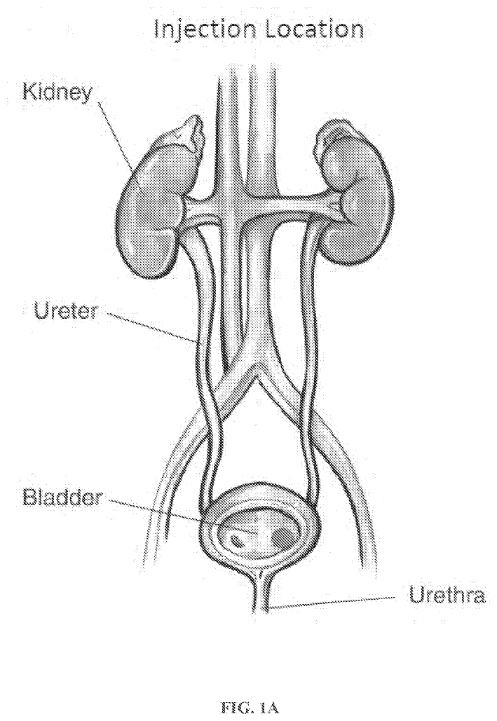

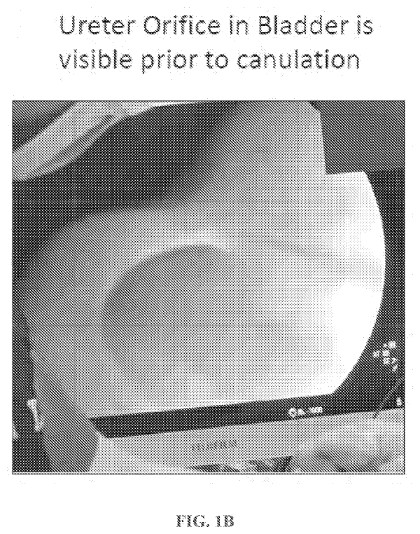

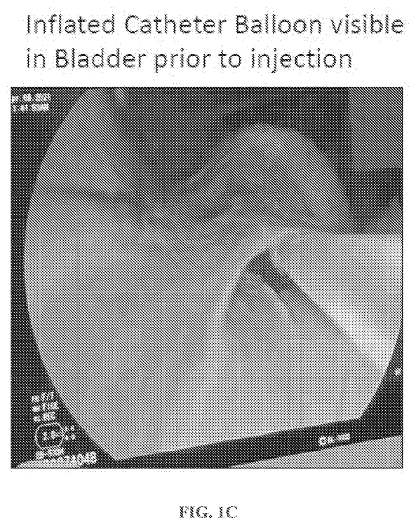

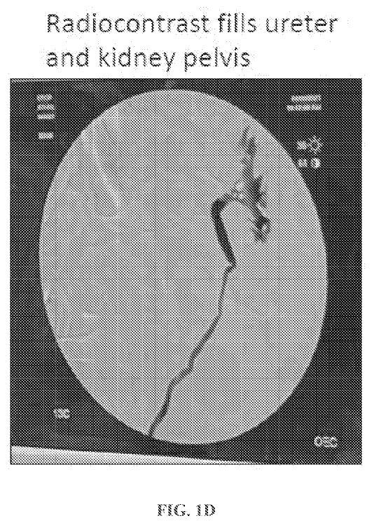

Three pigs were obtained for this experiment. Since each pig has two kidneys, each kidney can be used as a separate experiment with different injection parameters utilized. For each kidney, the strategy was to enter the bladder through cystoscopy. Once in the bladder, a catheter could be inserted into the cystoscope and navigated into the ureter orifice (see e.g., FIG. 1). The catheter was just inserted a short distance into ureter orifice (UO), whereafter the balloon was inflated 8.5 mm (Boston Scientific balloon with 2 ports (balloon inflation and wire (0.018 inch)/fluid) OD 26Fr or 8.5 mm, catheter width 5Fr or 1.7 mm, length 65 cm). The balloon inflation could be seen visually through the cystoscope, as a bulge of the bladder wall (see e.g., FIG. 1). During hydrodynamic injection, the UO was monitored to see if there was any leakage around the balloon.

For all experiments, the plasmid, pCLucf was used as a reporter pDNA. The plasmid contains two different gene expression cassettes: cytomegalovirus (CMV) promoter-firefly luciferase and SV40 promoter-green fluorescent protein (GFP). Both of these promoters are expected to confer ubiquitous expression of the transgenes in multiple cell types.

### Pig #1, 55096-30.1 kg

The right kidney was targeted first. It was used for learning about the volume to be used for the injection and testing possible injection parameters. Contrast only was injected for this purpose, so that the injected could be monitored by fluoroscopy in real-time.

When slowly injecting contrast to fill the ureter system, it was discovered that 5-8 mL of contrast was needed to opacify and fill the ureters and renal pelvis when the balloon was placed at the far distal end of the ureter.

Attempting hydrodynamic injection, 10 mL contrast injected at 2 mL/sec from the balloon set at the UO. This injection only seemed to further fill the ureter and pelvis. No contrast appeared to enter into the kidney parenchyma, which is a surrogate for DNA entry into cells of the kidney.

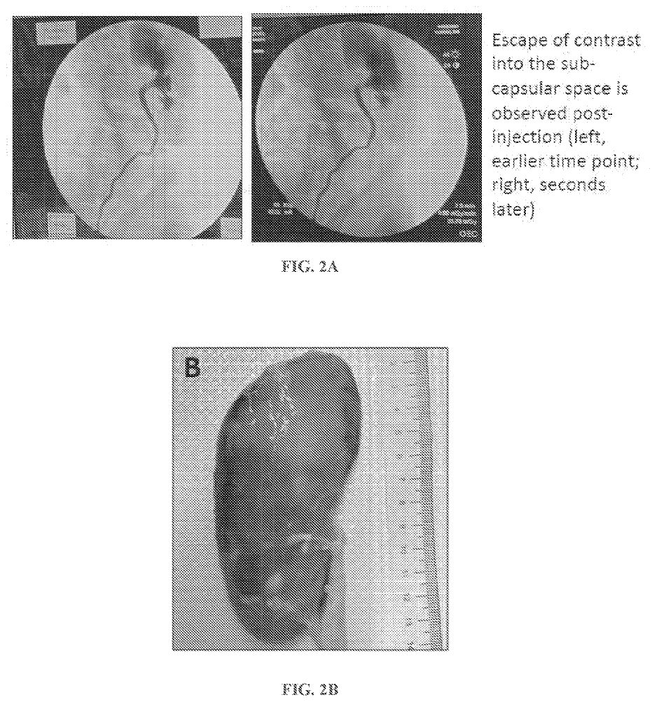

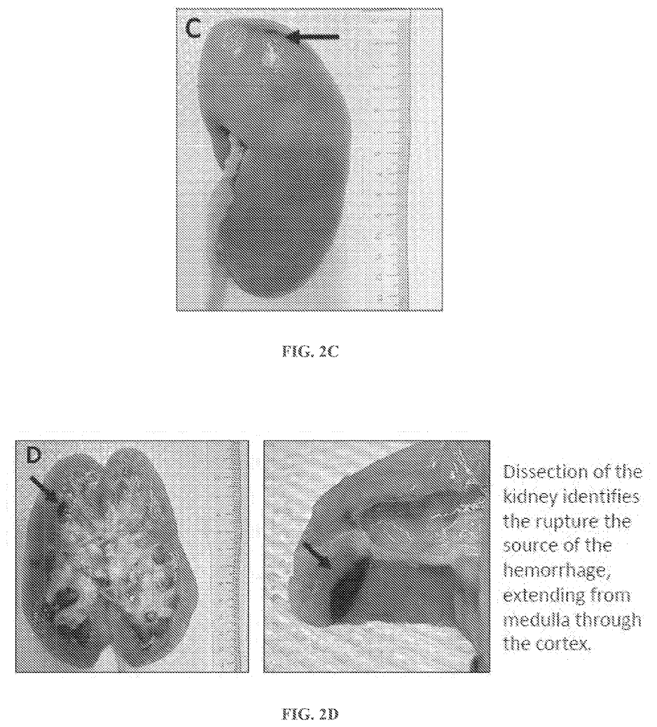

A second injection within the same kidney was undertaken at 10 mL and 3 mL/sec injection parameters in order to try to get contrast to enter kidney parenchyma. Contrast was not removed from the ureter prior to this injection. With this injection, opacification of the kidney parenchyma was observed, although this was mostly at the super pole. The fluoroscopy later showed that all the radiocontrast pooled between the kidney and kidney capsule (see e.g., FIG. 2). Upon necropsy of the pig after the experiment, it was confirmed that this fluoroscopic finding represented a rupture of the kidney cortex. The kidney weight was 82.8 g.

The left kidney was next targeted. This kidney was used for a DNA injection. A flow rate above 3 mL/sec was avoided given the previous rupture of the kidney pole. A larger volume was chosen to try to attempt to build more pressure and drive pDNA into the parenchyma. Parameters of 22 mL @ 2 mL/sec were used. The total DNA dose was 1.2 mg pDNA. The kidney weight was 83.8 g.

### Pig #2, 55098-31.7 kg

The right kidney was first targeted. It was injected from the distal ureter at injection parameters of 27 mL at 2 mL/sec. It was a perfect injection of pDNA with no leak at the UO as monitored by visualization of the camera. There was no ureter rupture by post-injection fluoroscopy. There was also no blood noted post injection coming out of the UO post-injection. The pDNA dose was 1.2 mg for the injection. The kidney weight was 73.2 g.

The left kidney was next targeted. An injection was planned with contrast to see if acinarization of the radiocontrast fluid could occur with a higher injection volume. The injection parameters were 20 mL and 2 mL/sec with contrast as the injected fluid. The contrast was at 100% concentration with no dilution. Acinarization was noted with contrast entry into the kidney parenchyma with this injection. There was blood coming out of the left orifice after the injection, which accumulated into the bladder. The kidney weight was 110.1 g, larger due to bleeding experienced.

### Pig #3, 55097-27.7 kg

The right kidney was first targeted for injection. The injected proceed from the distal ureter. The injection parameters were 20 mL volume at 2 mL/sec flow rate. The rationale was to inject a lower volume that would repeat the acinarization seen with the contrast injection at that parameter but would not cause any kidney rupture since the viscosity is normal saline solution. The pDNA dose for this injection was 1.2 mg. The injection proceeded without incident or leakage of the UO, and no blood was noted from the UO after the injection. The right kidney mass was 72.5 grams.

The left kidney was injected from the distal ureter. The injection parameters were 30 ml @ 1 ml/sec. The rationale was to increase the volume from the previous parameter to a larger amount to see if this could create more pressure. At the same time, a lower flow rate was used, knowing that there was a risk of rupture if injection proceeded too long. The pDNA dose was 1.2 mg injected. The hydrodynamic injection proceeded without incident. The left kidney mass was 74.8 grams.

All pigs survived for 72 hours. Pigs displayed normal behavior in the three days post-injection. Before kidney harvest, a blood specimen was collected prior to euthanasia to assess for toxicity. All kidneys were processed that same day for tissue analysis.

### Results:

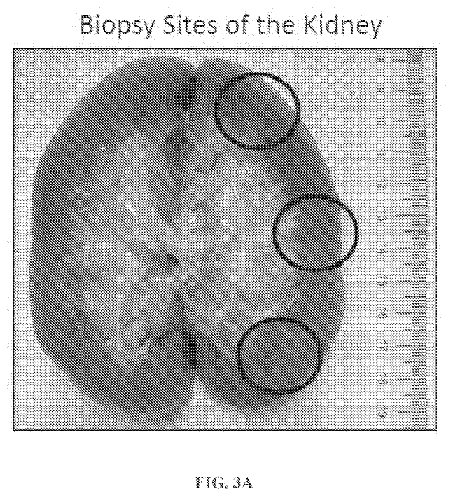

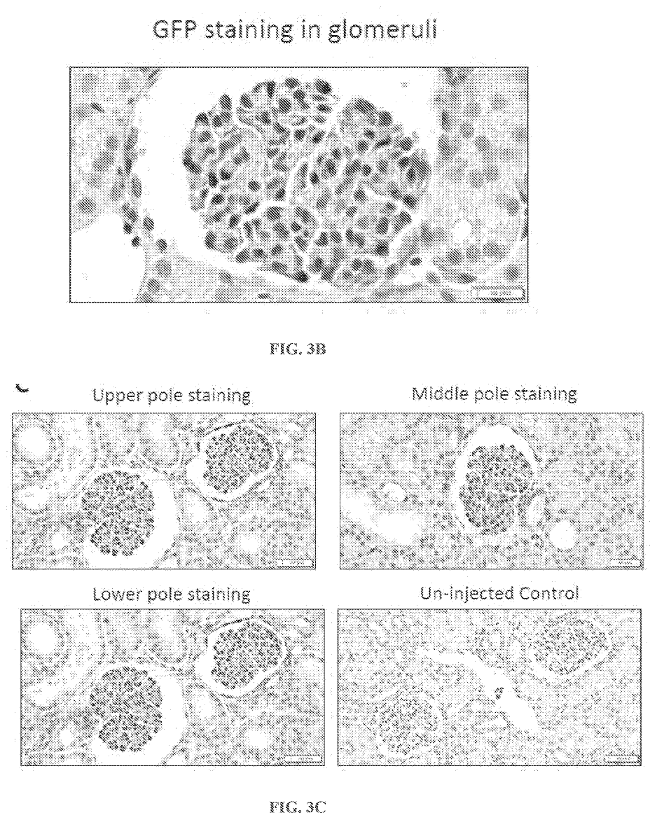

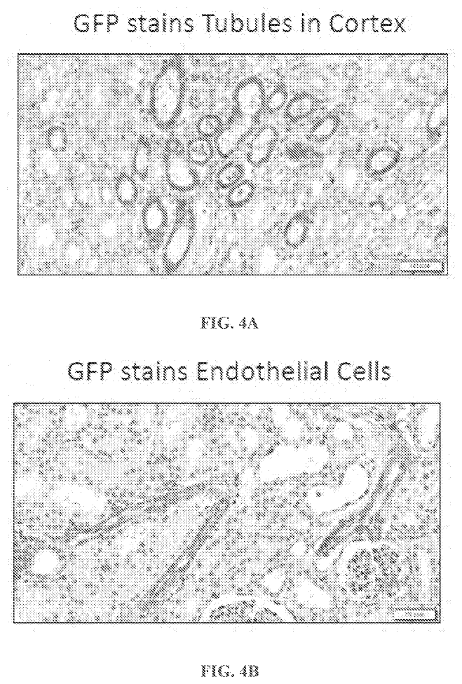

A total of four kidneys were injected with equivalent doses of 1.2 mg of plasmid DNA. The plasmid DNA encoded for GFP and firefly luciferase. Sections of the kidneys from me upper middle and lower poles were sent sampling the border of the renal cortex and the renal medulla. Immunohistochemistry of these sections for the reporter genes was conducted. Studies revealed that the different injection parameters mediated positive expression of reporter proteins (FIG. 3). Specifically, a portion of cells in the glomerulus were positive for GFP. A portion of individual tubules were found to be positive for firefly luciferase. Most expression was in the cortex, although some occurred in the medulla. Other cell types were also noted to stain positive for reporter genes including endothelial cells (GFP) and small tubules in the medulla (GFP) (FIG. 4).

Upon the necropsy, there were several 1 cm tears through the renal cortex and multiple different kidneys leading to a small collection of hemorrhage between the kidney and the capsule. A lack of kidney tear and injury was noted for pig number 3, left kidney. While the pigs did not appear to suffer any clinical abnormalities in their health, there was a notable drop and hematocrit rate in some of the pigs it did experience the bleeding. There was no elevation in creatinine levels and any of the pigs.

### Conclusions:

Hydrodynamic injection from the distal ureter is feasible. The bladder wall provides sufficient strength for balloon inflation without any concern for ureter rupture. The ureter can withstand high fluid pressure without rupture. There seems to be an accumulation of pressure within the kidney, which can be observed as physical tears of the kidney cortex beneath the capsule. Both on fluoroscopy and on the necropsy, most of the kidneys appeared to have tears in the upper poles, which may follow the direction of fluid force ascending from the bladder. Occasional tears are, however, seen in lower poles. Future testing will need to produce strategies to limit the rupture of the kidney, while maintaining to optimize the ability to have gene delivery.

Concerning gene delivery, it was observed that kidney cells were positive for expression of the GFP and luciferase transgenes. There is not a clear difference apparent from injection parameters yet, so this will have to be evaluated in future studies.

### Example 2: Testing Different Injection Parameters for Hydrodynamic Kidney Delivery Through the Ureters

The previous experiment represented the first time that hydrodynamic injection had been attempted through the ureters in the prior art. As such, important anatomical findings concerning injection volumes were learned. Validation that the ureter could withstand hydrodynamic pressure at given fluid volumes and flow rates was established. New unexpected toxicities were also established including the rupture of the kidney cortex from fluid pressure.

The studies also showed that potential combinations of injection parameters might be able to mediate the entry of contrast and DNA inside cells. In these preliminary studies, several injection parameters proved to be promising and showed scattered expression inside different types of kidney cells, including glomeruli and tubules. Further work is necessary to better define what injection parameters are actionable and sufficient for gene delivery.

To understand which parameters are tolerated by the kidney during injection, an experiment was set up. The flow rates to be tested included: 0.5 mL/sec, 1 mL/sec, 1.5 mL/sec, and 2 mL/sec. The volumes were kept constant at 20 mL total, which is a little over double the volume of the ureter and renal pelvis based on the previous work. The parameters of 20 mL volume and at 2 mL/sec did not appear to rupture the kidney pelvis in the previous study, so it was felt that this was a maximum volume that could be built upon. Given that the 1 mL/sec flow rate also appeared to mediate some level of gene expression, it was attempted to test delivery parameters between these two flow rates. In parallel to the flow rate studies, an experiment was set up to see if a lower volume (16 mL) at 2 mL/sec would be well tolerated (no kidney tear or bleeding) while also mediating gene delivery.

In summary, the goal of the study is to determine if there is some minimum flow rate for kidney transduction, and how the flow rate influences firefly luciferase expression in the kidney.

### Methods:

For all kidney injections with pDNA, 2 mg pCLucf was injected. This plasmid encodes firefly luciferase and GFP on the same plasmid from two separate promoters, which offer relatively ubiquitous expression in multiple cell types. Three pigs were tested, using each kidney as a separate experiment. The injection parameters and data collected during the procedure are reported below.

### Pig #1, 55212-36.4 kg

The left kidney was injected from the distal ureter at parameters of 16 mL and at 2 mL/sec. The rationale was to try to find lower volume that would not risk any kidney injury. The injection was perfect with no blood at the UO detected.

The right kidney was injected from distal ureter at parameters of 20 mL and at 1.5 mL/sec. The rationale was to decrease the flow rate while keeping the volume the same to see if equivalent pressure would lead to gene expression. The balloon moved back 1 cm, but the seal in the ureter was maintained.

ig #2, 55253-33.3 kg

The left kidney was first targeted. The procedure was conducted at the distal ureter at the junction of the UO and bladder as had been done previously. The kidney was injected at parameters of 20 mL at 1 mL/sec. The injection had no issues, and no bleeding was noted from the UO after the injection.

The right kidney was next targeted. The right ureter was successfully canulated without difficulty and the balloon placed in the UO entrance. While visualizing the ureter and kidney pelvis, the right ureter was noted to have a twisted path to the kidney on fluoroscopy. The injection was carried out at injection parameters of 20 mL and 0.5 mL/sec. The purpose was to see if there was lower threshold where no gene transfer occurred. The injection was completed without incident.

### Pig #3, 55210-34.3 kg

The left kidney was canulated and the catheter advanced into a proximal location to the renal pelvis. The balloon was placed inside the renal pelvis and contrast slowly injecting. It was noted that the balloon failed to prevent contrast from leaking behind.

The catheter was moved backwards to a location in the ureter pelvis where the balloon could fully seal the ureter. The estimated location in the ureter was 2-3 cm from the renal pelvis. A hydrodynamic injection occurred from this location with pressure sealing by the balloon. The injection parameters were 15 mL at 1 mL/sec of contrast. The volume was reduced since the catheter was closer to the kidney, although the flow rate was also reduced in conjunction given this volume is high for a limited space. After the injection, the fluoroscopic imaging showed pooling of contrast between the kidney and capsule, indicating rupture (FIG. 8). Furthermore, there was blood noted in the bladder to support rupture of the kidney.

The right kidney was not targeted for injection, and the kidney was harvested at necropsy for control pig kidney tissue.

All pigs were survived for 72 hours. Pigs displayed normal behavior in the three days post-injection. Before kidney harvest, a blood specimen was collected prior to euthanasia to assess for toxicity. All kidneys were processed that same day for tissue analysis.

### Results:

Further analysis of the kidneys injected were conducted analyzing the kidneys at the necropsy for any ruptures or tears in the kidney poles, as well as taking tissue sections at the upper middle and lower pole of the kidney to determine if any gene delivery had occurred. The rationale for sampling the kidney at three different sites was to see if there was a difference in the pressure felt by the kidney, which would then influence the gene expression pattern observed.

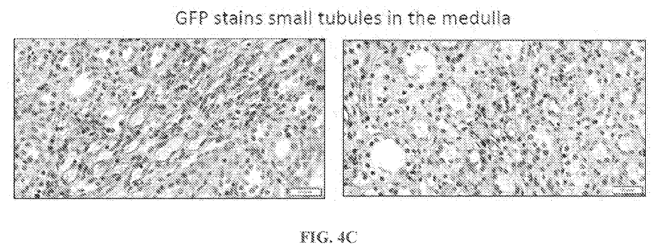

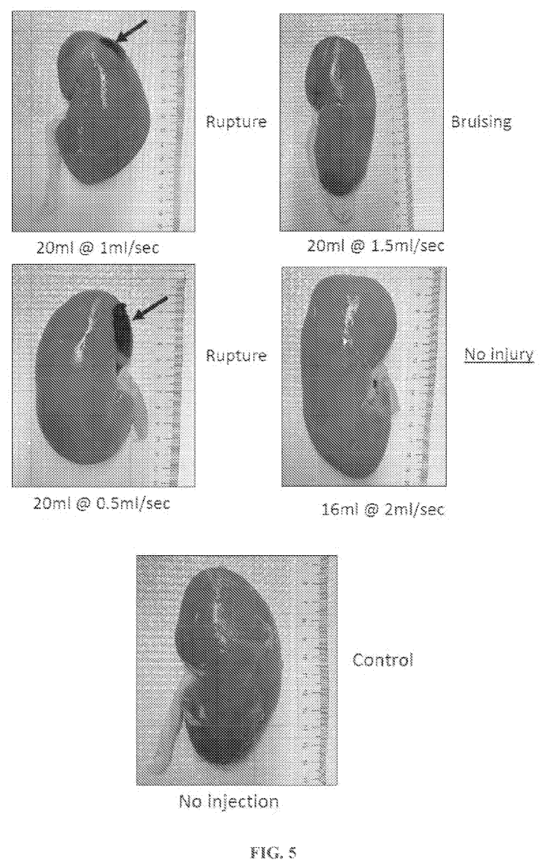

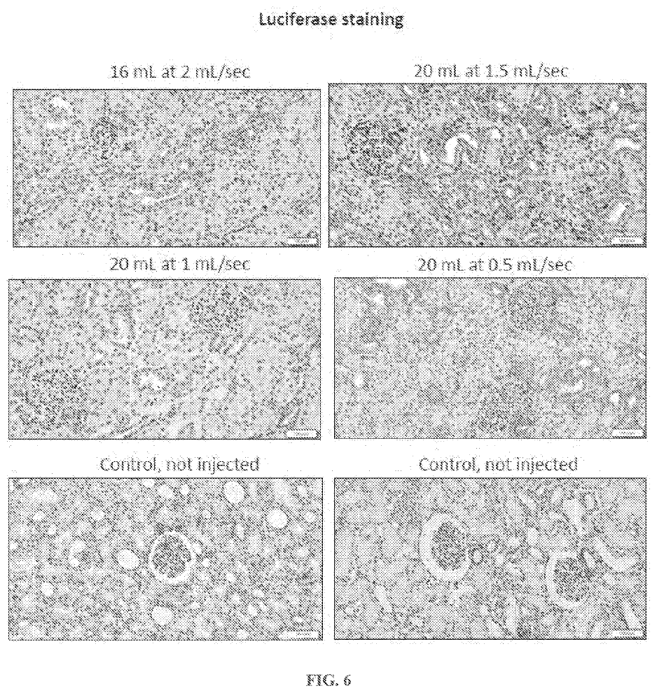

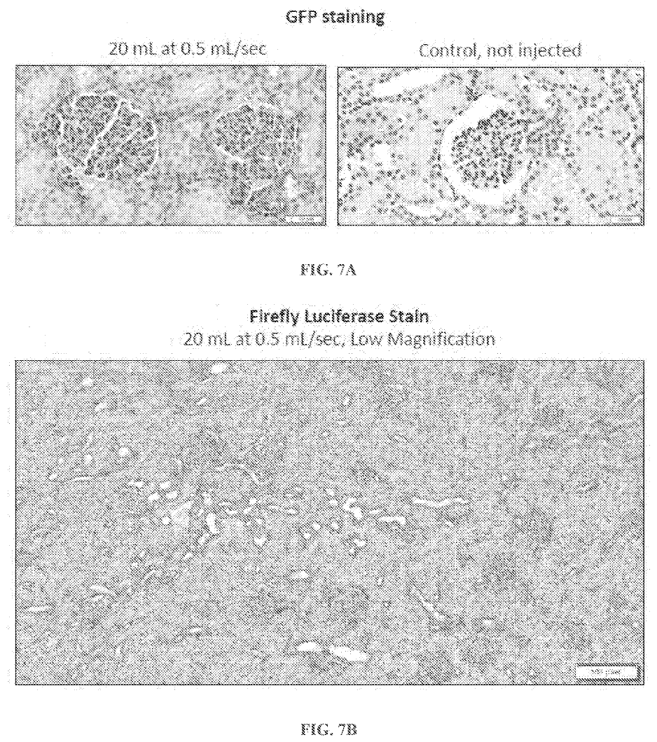

For the experiment, it was observed that the flow rate below 2 mL/sec was well tolerated during the experiment. However, kidney ruptures or damage was still noted for these injection parameters, except for the 16 mL at 2 mL/sec parameter (see e.g., FIG. 5). This indicates that the total volume influences the rupture of the kidney. Looking at the flow rates tested, the flow rate of 0.5 mL/sec surprisingly had the best gene expression with stronger staining for luciferase in the tubules (FIG. 6). The 1 mL/sec, 1.5 mL/sec, and 2 mL/sec had positive cells for firefly luciferase and GFP but were less intense in relative staining versus the lower flow rate. At the condition where a lower volume was tested, the 2 mL/sec flowrate was successful at 16 mL of volume for mediating expression of positive cells staining positive for luciferase and GFP. The ability of the lower flow rate (0.5 mL/sec) was further examined confirming that GFP staining was seen in the glomeruli (see e.g., FIG. 7).

For the kidney injected with contrast, the necropsy of the kidney injected at the proximal location confirmed the rupture in the cortex that was seen on fluoroscopy during the hydrodynamic injection (see e.g., FIG. 8). Placement of the balloon in the pelvis of the kidney was ineffective in promoting seal for injection. A lack of gene delivery was observed on tissue staining, possibly reflecting instant rupture and escape of fluid. The damage of the proximal injection at 15 mL and 1 mL/sec was further observed in the necropsy tissue, wherein a large immune extended from the medulla into the cortex with areas of necrosis observed (FIG. 9).

### Conclusion:

It was observed that a balloon inflated inside the renal pelvis does not effectively inhibit antegrade contrast flow and is a poor location for future injections (FIG. 8A). A balloon can be placed in the most distal aspect of the ureter just inside the UO and have a good seal without rupture of the ureter during injection. Inflation of the balloon and injection in the proximal region of the ureter closer to the kidney may be associated with kidney rupture with a higher incidence of bleeding (FIG. 8). This is surprising since a larger volume at similar flow rate of 1 mL/s was well tolerated from the distal aspect of the ureter. There may be some equilibration of fluid escape, which occurs within the kidney at the distal injection point. Some pigs were noted to have tortuous ureters, which inhibit easy catheter advancement into the kidney. It would be interesting to understand how often this occurs in human patients. The volume from the UO to fill the ureters and renal pelvis is only up to 9 mL, representing a relatively small total volume.

Gene expression was best at 0.5 mL/sec flow rate, suggesting that pressure can effectively be generated at lower flow rates. Surprisingly, the 0.5 mL/sec still mediated rupture of the kidney. Given the other results, decreasing the volume may be the best strategy to avoid any kidney rupture.

### Example 3: Testing of Injection of a Proximal Location to the Kidney Pelvis

### Background:

The initial results for kidney hydrodynamic injection through the ureters were promising for gene delivery, as measured by immunohistochemical staining for gene expression. Subsequent follow up studies suggested that the amount of gene delivery observed may be significantly less efficient.

To continue to validate and test for optimal parameters for conducting gene delivery studies through the kidney, additional studies were conducted. There were several hypotheses to be tested. One of the hypotheses was that the pDNA dose used in previous studies was too low to mediate efficient gene expression. Another hypothesis was that the injection location could still be further modulated in order to achieve a positive effect, which was in part necessitated by the catheter size as explained below.

In addition to testing new injection parameters, a new catheter was also tested to mediate injection into the kidney. One of the challenges of hydrodynamic injection into the kidney is that many of the working channels of cystoscopes are too small to fit most suitable catheters that would be used in the procedure. Suitable catheters in this case would have 3 or 4 lumens, which would allow for simultaneous monitoring of pressure, guidewire placement, and contrast/DNA injection. However, the diameter required of the catheter to fit all those lumens is generally larger than the size most cystoscopes can manage. Herein, an alternative method for executing the procedure that overcomes this challenge is described.

### Methods:

The hydrodynamic injection procedure into the kidney was conducted in a novel format. For this procedure, a cystoscope was inserted through the urethra and into the bladder. A guidewire was inserted through the cystoscope and into the bladder. The guidewire was then inserted through the ureter orifice. The guidewire was advanced up the ureters until reaching the kidney pelvis. The exact position of the guidewire was confirmed by C-arm.

The cystoscope was subsequently removed from the bladder leaving the guidewire in place. A custom, quadruple lumen balloon catheter was then used for the experiment. This catheter consisted of lumens for the air balloon, guidewire, pressure sensor, and contrast/DNA injection. The quadruple lumen catheter was then advanced over the guidewire and up toward the kidney. The exact position of the catheter was confirmed by C-arm. The catheter balloon was inflated in a proximal location in the ureter next to kidney pelvis. Balloon seal was confirmed with slow contrast injection by hand push (FIG. 11A). The hydrodynamic injection was then conducted as the designated parameters outlined below. In general, higher pDNA doses of pCLucf were utilized than the previous study, since there was an idea that even higher gene expression could be obtained with a higher pDNA dose.

### Pig #1, 673-50 kg

The left kidney was targeted for the first injection. A preparation of pDNA solution was made. It was 5 mg pDNA into 15 mL total DNA solution. The dead space of the tubing was estimated at 5 mL total, with 3 mL in the tubing and 2 mL in the catheter. The injection proceeded at 10 mL injected at a 1 mL/sec parameter. The injection was very close to the kidney in proximity to the kidney pelvis.

The right kidney was next targeted using the same procedure. For this injection, 10 mg pDNA was able to be prepared. The injected proceeded at 10 mL and at 1 mL/sec without issue. Using contrast solution, the upstream volume was estimated at 3 mL in total.

### Pig #2, 676-55 kg

The right kidney was targeted first. For this injection, 15 mg pDNA was planned for the injection with that intent of dissolving in 15 mL total volume, knowing that 10 mg pDNA would ultimately enter into the kidney. It was observed that a total of 3 mL of contrast was injected into the kidney calyx at the proximal balloon location. The injection parameter of 10 mL injected at 1 mL/sec was utilized. The injected proceeded without incident.

The left kidney was targeted for injection next. It was noted that the baseline pressure in the ureter with the catheter placed was ~7 mmHg. Into this kidney, 50% saline and 50% contrast was injected. An injection was delivered at 10 mL and 1 mL/sec. The pressure achieved during the injection was up to 50 mmHg. No apparent acinarization of the kidney parenchyma was noted.

### Pig #3, 679-55 kg

The right kidney was targeted for the first injection. Catheter was successful localized using the established procedure. The injection parameters of 11 mL at 2 mL/sec were utilized. The flow rate was increased here to try to augment potential gene delivery observed, since the 1 mL/sec flow rates did not mediate apparent acinarization. The plasmid DNA dose utilized was 10 mg pDNA in total. The pressure reading topped out at 66 mmHg injection, that subsequently fell. There was a concern that the balloon may have failed during the injection. Testing ex vivo, the balloon seems to be inflated, but still concern there was a hole or failure.

The left kidney was targeted next. A different quadruple lumen catheter was utilized. The pDNA dose was 10 mg pDNA of pCLucf. The injection parameters were set at 12 mL at 1.5 mL/sec. The power injector performed as instructed with the injection to completion. While the balloon appeared working on the catheter prior to insertion in the pig, the balloon failed after it was subsequently removed. Furthermore, the pressure sensor failed to detect elevated pressure during the injection.

After the procedure, the pigs were all sent for necropsy. Kidney survey for rupture or tear in the renal cortex was not performed due to outsourcing to the veterinary staff. The kidney organs were isolated and tissue samples taken from the upper middle and lower poles. The kidneys that were injected with pDNA were subject to immunohistochemical analysis for reporter protein expression.

### Results:

The kidneys injected with pDNA were evaluated for gene delivery and expression after injection at the proximal kidney location. The kidneys injected at 1 mL/sec only showed that scattered positive cells, largely reflecting a similar pattern to the previous experiment with some expression appreciated in the glomeruli and were observed (FIG. 10). Gene expression was relatively fainter than the previous study, but different IHC conditions were utilized.

In addition to gene expression, the pressure generated during the proximal injection was evaluated. In a separate naïve pig, parameters of 10 mL at a 1 mL/sec injection rate were executed (FIG. 11B). Pressure after injection peaked at 105 mmHg, before rapidly declining. Since the injection should have lasted 10 seconds with a plateau pressure maintained, this likely indicated that a balloon failure or kidney rupture occurred.

### Conclusion:

Relevant pressures for a given flow rate were identified really first time leveraging the use of a pressure sensor with approximately 100 mmHg identified in relation to 1 mL/sec in the proximal location. Moreover, a new method for executing hydrodynamic kidney was established and proved to be efficient and easy to execute. Gene delivery efficiency remains relatively low from the proximal injection site employed in this experiment, and mimic results from the distal ureter injection site leveraged in previous experiments, and possibly was worse.

### Example 4: Testing of Pressures Generated During Distal Ureter Injection into the Kidney

Previous studies of the proximal injection location at the junction of the bladder and ureter did not evaluate the pressure generated during the injection. In the current study, further investigations were undertaken to understand the pressures achieved from this location at different flow rates. Such knowledge could help guide future pressure-directed strategies of injection into the ureter for kidney gene delivery.

### Methods:

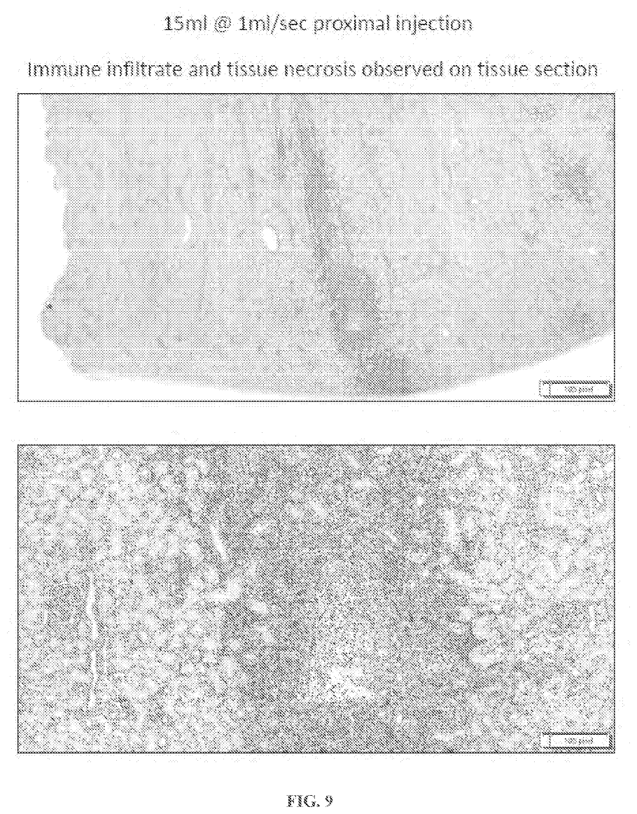

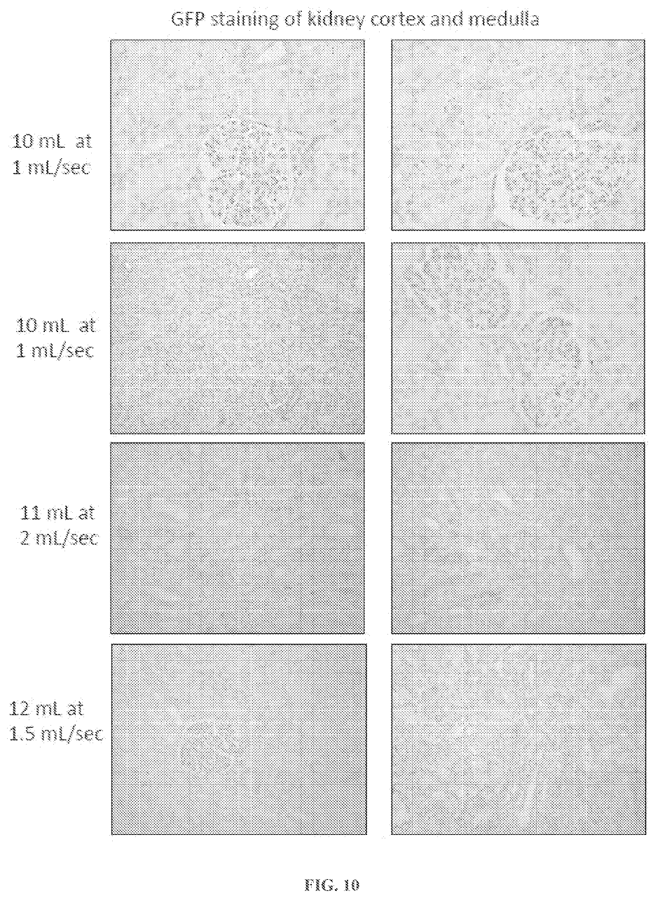

For the experiment, a pig was obtained that was scheduled to be euthanized. A laparotomy was performed, and excision made into the bladder wall. The ureter orifice was visualized, and a catheter inserted into the ureter. The catheter itself was connected to a power injector for delivery of saline solution. A pressure sensor was advanced alongside the catheter into the ureter, being too large in diameter to fit through the lumen of the catheter. Both the pressure sensor and catheter were advanced into the distal aspect of the ureter side by side. A surgical tie was made to prevent antegrade flow during injection into the ureter (FIG. 12). A series of different flow rates were tested to evaluate for pressure with a fixed volume of 10 mL.

### Results:

Results of the pressure tracings are depicted in FIG. 12B with arrows indicating injections. The study observed that pressure is flow rate dependent. Flow rates of 0.5-1 mL/sec yielded pressure of 70-80 mmHg. Flow rates of 1.5-2 mL/sec yield 120-140 mmHg. Additional testing with higher volume injected at a constant flow rate did not further increase pressure (data not shown). Baseline in the ureter was observed to be 5-15 mmHg prior to injection. After the series of injections, the kidney was further dissected (FIG. 12C). Pooling of fluid in the space between the capsule and kidney surface was observed. There were several tears were noted, which may have occurred during injections greater than 3 mL/sec.

### Conclusion:

Relatively flow rates were able to generate effective pressure in the ureter system, with 0.5-1 mL/sec yielding pressure of 70-80 mmHg. By comparison, biliary hydrodynamic injection requires a flow rate of 2 mL/sec to generate an intraductal pressure of around 80 mmHg. This observation speaks to the inherent differences in tissue anatomy between the two organs. It also suggests, in part, a reason for the ruptures observed with greater pressures being readily achieved at low flow rates.

### Example 5: Hydrodynamic Delivery Through Endoscopic Retrograde Cholangio-Pancreatography (ERCP) Does Not Cause Pancreatitis 1 Day Post-Injection

The pancreas is an intriguing organ for gene therapy. There are numerous important diseases that have their etiology within the organ. Traditional gene therapies do not traffic well to the organ after intravenous injection. Local injection strategies have been employed to direct viruses into the pancreas. Although viruses are efficient delivery vehicles, non-viral gene delivery has significant advantages.

The techniques herein provide the use of hydrodynamic injection to mediate nonviral delivery of plasmid DNA directly into cells of the pancreas. The challenge with hydrodynamic delivery into the pancreas is the potential for causing pancreatitis. Injury of acinar cells leads to the leakage of digestive enzymes contained within acinar cells. This leakage causes autodigestion of the pancreas tissue, which feeds forward on itself causing more digestion and inflammation. Nevertheless, there have been pilot studies in rodent models of hydrodynamic injection, which have shown that any toxicity is only transient to the pancreas. One promising study has delivered pDNA by hydrodynamic injection through the superior mesenteric vein with demonstrated gene expression by luciferase assay along patchy staining in acinar cells (Mol Ther Nucleic Acids. 2017 Dec. 15; 9:80-88). Another study explored intraductal hydrodynamic injection in mice and report it to be safe without development of pancreatitis (Journal of Pharmaceutical Sciences, 107(2), 647-653). Gene expression was found to occur by firefly luciferase assay. Unfortunately, no results on tissue staining within the pancreas itself were published, which suggests the technique was likely inefficient. Importantly, no studies of pancreatic gene delivery through ductal or vascular systems have ever been translated into large animal models.

The techniques herein apply a ductal hydrodynamic injection approach to the pancreas, which can be accessed by ERCP. Gene delivery into the pancreas has similar rationale to the biliary system, wherein a single ductal system feeds into small intestine, with a lack of communication or circulation into other tissues. Thus, the fluid pressure would directly build up and spill into the surrounding pancreatic tissue to mediate gene delivery.

### Methods:

To assess the ability to deliver target nucleic acids to the pancreas, pCLucf plasmids, which contain a cytomegalovirus (CMV)-promoter driving expression of firefly luciferase and an SV40 promoter driving expression of green fluorescent protein (GFP), were used in a pancreas-specific hydrodynamic injection protocol. Both the CMV and SV40 promoter are active in most cell types, and thus could reasonably be active in most cell types in the pancreas.

The pCLucf plasmid was expanded with gigaprep kits and dissolved in normal saline solution prior to the experiment. To assess what injection parameters would yield successful injection in the pancreas. Initially, a flow rate of 2 mL/sec was used in the pancreas specific hydrodynamic injection protocol was reduced and values above 20 mL were chosen in order to assure entry of DNA solution into the surrounding tissue. The results are summarized in Table 5.

TABLE 5

| Animal ID# | Animal | | Pancreas | | Injection Parameters |
|---|---|---|---|---|---|
| (DNA Payload) | Weight | | Weight | | (Comments) |
| Pig #2241 | 54 | kg | 130 | g | 22 ml @ 2 ml/sec |
| (GFP-Luc) | | | | | |
| Pig #2256 | 49.3 | kg | 112 | g | 23 ml @ 2 ml/sec |
| (GFP-Luc) | | | | | |
| Pig #54643 | 41.3 | kg | 167.2 | g | 25 ml @ 2 ml/sec |
| (GFP-Luc) | | | | | |
| Pig #54644 | 40 | kg | 80.1 | g | (Injection: Could not |

| | |
|---|---|
| (GFP-Luc) | localize the duct for |
| | injection) |

Pigs had blood draws performed pre- and post-injection, and 24 hours after injection prior to euthanasia. The pancreas was collected from the pig at day 1 after injection. Amylase levels were monitored as a sign of pancreatitis. Of note, the pancreatic duct in pigs is separate from the ampulla of Vater, and can be very difficult to localize from the small intestinal wall. Thus, it may not technically reliable to inject every pig into the pancreas every procedure.

The pancreatic ductal hydrodynamic procedure could be performed with ease through ERCP. Results are presented which show the feasibility of canulation of the pancreatic duct and pigs as well fluoroscope he pre- and post-procedure (FIG. 13). The post-procedure fluoroscopy, in particular, shows that the pancreatic duct is intact.

Immunohistochemical staining for GFP and luciferase revealed abundant staining throughout the tissue in multiple cell types (FIG. 15). Prominent staining was observed in islet cells and ductal cells, wherein essentially 100% of these cell types were positive (FIG. 16). Curiously, acinar cells did not stain strongly positive, which may be due to the inefficiency of the promoter, or an intrinsic resistance of acinar cells to the expression of exogenous DNA. Expression was observed in all three pancreatic lobes, and expression was positive in both pigs emphasizing the reproducibility of the technique (FIG. 15).

The pigs did not exhibit any short-term physiological changes in vital signs during the injection. Amylase levels on Day 1 post-injection were 3-4-fold above baseline, while other labs including white blood cell count were normal (FIG. 14). Small micro-regions of necrosis were observed within the pancreas, suggesting some tissue damage from the procedure (FIG. 17). This tissue damage is not apparent gross dissection of the organ, where color, and firmness are indistinguishable from wildtype pig pancreas. These small necrotic regions have heavy inflammatory infiltrates of lymphocytes and neutrophils (FIG. 17). The significance of these damaged areas is uncertain, and whether or not they would result in pancreatitis if left to develop for several days.

Hydrodynamic injection through the ductal system of the pancreas in pig models is a feasible way to deliver DNA into multiple cell types of the pancreas with very high efficiency of delivery. Practically all islet cells and ductal cells were positive for expression. Preliminary investigations show the procedure may be safe, although elevations in amylase are noted at day 1 post-injection.

### Example 6: Hydrodynamic Delivery Through Endoscopic Retrograde Cholangio-Pancreatography (ERCP) Does Not Cause Pancreatitis 3 Days Post-Injection

The previous Example tested for pancreatitis at day 1 post-injection. The results demonstrated a 3-4-fold increase in amylase levels at this timepoint post-injection. Amylase elevation is a sign of pancreatitis in human patients, and this early amylase elevation could be a sign of pancreatitis that could become clinical worse over time with more tissue inflammation and destruction. Alternatively, amylase levels could be a sign of leakage of amylase from cells without frank injury. In order to investigate the significance of this result, additional pigs were injected at the same injection parameters as the previous experiments.

### Methods

Two pigs were obtained and ERCP was performed. The details of the pig weight, pancreas weight, pDNA delivered, and pDNA employed are provided below in Table 5. The injection parameters are listed.

TABLE 5

| Animal ID# | | | | Injection Parameters |
|---|---|---|---|---|
| (DNA Payload) | Animal Weight | Pancreas Weight | | (DNA Dose) [Comments] |
| Pig #55212 | Weight: 36.4 kg | 78.2 | g | (Could not canulate |
| (GFP-Luc) | | | | pancreatic duct. No |
| | | | | injection occurred.) |
| Pig #55253 | Weight: 33.3 kg | 75 | g | 22 mL @ 2 mL/sec |

| | |
|---|---|
| (GFP-Luc) | (2 mg pDNA) |

The pig was bled for chemistry testing pre-injection, post-injection, and on day 3 post-injection. The pig was euthanized on day 3 post-injection and pancreas sampled that same day. All three lobes were sampled.

The pancreatic duct for pig #55253 was successfully cannulated by ERCP. The pancreatic duct was intact as the guidewire and subsequent contrast injections helped localized the catheter inside the duct. The ideal location is the duct before the bifurcation of the duct into the different pancreatic lobes. Injection parameters of 22 mL of volume at 2 mL/sec were programmed into the power injector. Prior to injection, the balloon was inflated to 11 mm, matching sizes used in the bile duct. The injection proceeded without incident. Repeat fluoroscopy, however, demonstrated diffusion of radiocontrast into the surrounding tissue space, in close proximity to the catheter tip (FIG. 18).

There are several interpretations of what occurred. The first is that the stress of the hydrodynamic injection resulted in rupture of the pancreatic duct. While this is possible, it would be surprising since the previous pigs tolerated the injection into their pancreas' without incident. The second possibility is that the duct was already traumatized before the hydrodynamic injection, such that the high-pressure injection further served to open and enter into the damaged ductal wall. Regarding this, the hypothesis was raised that the balloon was too large and could have caused trauma to the pancreatic duct wall. Another hypothesis that could have synergized with this mechanism is that the contrast injection in this catheter is side-facing, such that any pressure from the hand-push of the catheter would go directly onto the pancreatic duct.

Despite this trauma from the injection, the pig was kept alive to study what may happen from such an event. Much like the other pigs, the pig experienced a transient increase in amylase on day 1 post-injection. Surprisingly, by day 3 post-injection, amylase levels were back within normal range. The gross dissection of the pancreas from the pig did exhibit evidence of caseating necrosis in the duodenal lobe where the damage occurred on fluoroscopy (FIG. 18). However, the rest of the pancreas was healthy and intact.

The pancreas was sampled for tissue processing and immunohistochemical analysis for gene expression. Positive staining for firefly luciferase and GFP was observed in islet cells and ductal cells in similar abundance to the previously injected pigs (data not shown). This suggests that the ductal wall injury did not significantly impair the fluid pressure generated, since sufficient pressure was enough to enable gene delivery.

### Conclusion

The hydrodynamic injection procedure through the main pancreatic duct pancreas not without risk. Careful consideration in improvements in the procedure can be made including two use smaller balloon sizes through the pancreatic duct in future experiments. A catheter would be optimally used that could have a forward-facing guidewire and forward-facing injection lumen in order to assure that pressure is avoided on the sidewall the catheter.

It is encouraging that the pig did not suffer severe pancreatitis from this injury. It was even surprising that the amylase level was normalized by day 3 post-injection. The latter finding could be clinically relevant to pancreatic hydrodynamic injection in general, since the fluid pressure observed in rodent studies only transiently increase markers of tissue injury and by several days post-injection these markers normalize. That said, a similar injury occurring on the duct wall and humans would likely lead to severe pancreatitis and hospital admission for a patient. Therefore, it should not be inferred from this study that such a complication will be tolerated in human patients.

ERCP to target the pancreas of pigs is extremely difficult since the orifice in pigs is not readily apparent on the duodenal wall, and the pancreatic duct was not successfully cannulated in every pig. It is separate from the muscular bile duct of the ampulla of Vater that can be readily canulated in pigs and human patients. Another factor could be the size of the pigs employed. These pigs were relatively smaller (33-36 kg) versus the previous pigs employed (40-54 kg). Future tests may need to be conducted with larger pigs, 40-50 kg, which could be for easier pancreatic duct localization with larger orifices. It is contemplated within the scope of the disclosure that pharmacologic agents may be used to cause secretion of pancreatic juices for easier visualization, since the fluid would be flowing into the duodenum.

### Example 7: Testing Whether Lower Injection Parameters Could Still Mediate Gene Delivery into the Pancreas

As described herein, gene delivery into the pancreas may occur by ductal hydrodynamic injection. The procedure appeared to be very efficient in delivery into islet and ductal cell types. It was noted that amylase levels were elevated at day 1 post-procedure, as well as potential concern for damage to pancreatic tissue on histology.

The human pancreas may be more susceptible to damage than the pig pancreas. Because of this, it would be important to understand what the minimal injection parameters are that still mediate efficient gene delivery, with the hypothesis being less pressure would mean less tissue damage and less likely to develop pancreatitis.

### Methods

The previous parameters employed all had the volume higher than 20 mL. The flow rates were also all 2 mL/sec or higher.

Toward reducing the injection parameters, there are two different strategies. One is to reduce the volume, such that the pressure would be felt by the tissue for a shorter time period. Given that the pancreatic duct likely has a volume of 3-4 mL, an excess of volume was likely used in these previous studies. The other strategy would be to decrease the flow rate, such that the pressure would be reduced during the injection. This would require the reduced flow rate pressure to still be sufficient for gene delivery.

Three pigs were selected for this experiment. Their total weight, pancreas weight after necropsy, the DNA dose, and the injection parameters provided are listed below in Table 6.

TABLE 6

| Animal ID# | Animal | | Pancreas | | Injection Parameters |
|---|---|---|---|---|---|
| (DNA Payload) | Weight | | Weight | | (DNA Dose) [Comments] |
| Pig #1 | 36 | kg | 87.76 | g | 20 ml @ 1 ml/sec |
| (GFP-Luc) | | | | | (4 mg pDNA) |
| Pig #2 | 37.2 | kg | 57.78 | g | 15 ml @ 2 ml/sec |
| (GFP-Luc) | | | | | (4 mg pDNA) |
| Pig #3 | 37.7 | kg | 64.62 | g | [No injection was |

performed, unable to
canulate duct]

Pre- and post-procedure samples were collected. The day of euthanasia and pancreas harvest was on day 3 post-injection. Pancreas tissue was carefully dissected away from the other intestinal organs.

Both pigs only exhibited limited amylase elevations, which were lower than the three prior pigs who underwent ductal hydrodynamic injection. The gross organs for the pigs were intact with no abnormalities noted. Immunohistochemical staining for luciferase and GFP was conducted on all lobes in both pigs. Expression of both proteins was observed in the islet cells and ductal cells in a similar pattern previous seen in the prior pigs' injection at faster speeds and larger volume (FIG. 19A). Micro-necrosis areas within the pancreas on H&E were rare and/or absent on most tissue sections (FIG. 19B).

### Conclusion

The lower injection parameters were sufficient for gene delivery to the pancreas. Because of their potential to mediate less pressure and tissue injury, these new parameters have exciting potential to be useful in future gene therapy studies of the pancreas. Given the concerns over pancreatitis in the clinical application, the signs of tissue damage were less using these promising new injection parameters. This could lead to a safer procedure in humans, where pancreatitis is easier to induce through ERCP.

### Example 8: Methods and Materials

### Plasmid DNA

pCLucf was a gift from John Schiller (Addgene plasmid #37328 http://n2t.net/addgene: 37328; RRID: Addgene_37328).6 pCLucf encodes for firefly luciferase under a CMV promoter and GFP under an SV40 promoter. The plasmid was prepared for injection using a gigaprep kit from ZymoResearch. One milligram of pCLucf plasmid DNA was diluted into sterile normal saline prior to injection.

### Pancreatic Injection Procedure

Endoscopy was performed in four pigs under anesthesia. All pigs were weighed before to determine anesthesia drug testing, and a pre-procedure blood draw (red and purple top tubes) was obtained. The endoscope (therapeutic video duodenoscope, ED-580XT, FUJIFILM Medical Systems U.S.A) was advanced into the duodenum and the pancreatic orifice was visualized. The pancreatic orifice was cannulated by sphincterotome (CleverCut 3V, Olympus Medical), and passed Guidewire (VisiGlide, Olympus Medical) and subsequently, catheter (Multi-SV Plus, Olympus Medical) was advanced into the pancreatic duct. Balloon placement was just inside the pancreatic duct in the duodenal lobe of the pig pancreas before the branching of the pancreatic duct into a superior and inferior terminus, covering the duodenal and splenic lobes and the connecting lobes respectively. Contrast (Omnipaque, 350 mg/ml; GE Health Co.) injection confirmed proper placement of the catheter as seen by visualization of both branches. The balloon was next inflated in the duodenal lobe of the pancreas, to prevent retrograde motion of any injected fluid at high pressure. Hydrodynamic injection was commenced using a power injector (MedRAD Mark V Arterion, Bayer) under settings of 22-25 mL of DNA solution injected at 2 ml per second. 25 ml of the saline solution was loaded into the power injector prior with 5 ml of DNA solution priming the circuit from the power injector to the distal end of the catheter. Repeat fluoroscopy was performed with the contrast post-procedure to evaluate the integrity of the pancreatic duct. 15 minutes post-injection, a second blood draw was obtained (red and purple top tubes). A CT scan was performed on each pig 24 hours post-injection

### Tissue Analysis

Pigs were harvested 24 hours post-injection. The injected pancreas was surgically dissected in isolation from duodenum and stomach, weighed, and each of the three lobes sampled for tissue analysis. Control, non-injected pig pancreas tissue was also obtained separately and a similar analysis was performed.

Tissue samples were alternatively fixed in 10% formaldehyde or snap-frozen in liquid nitrogen. PCR was performed on frozen tissue from different samples to confirm the presence of pCLucf DNA. Immunohistochemistry was performed by VitroVivo (Rockville, MD) using polyclonal firefly luciferase antibodies (Promega) and polyclonal GFP antibodies (Genscript), respectively.

### Blood Analysis

Serum chemistries and hematology were performed by the Johns Hopkins Phenotyping Core on Diasys Respons®910 chemistry analyzer and Procyte automated analyzer, respectively. (edit, copied) For plasmid DNA detection, DNA was isolated from serum using the QIAgen DNeasy Blood & Tissue kit, and then subjected to PCR (Dream Taq, ThermoFisher).

### Statistics

Statistical analysis was performed with GraphPad Prism 7 software (GraphPad Software), which also serviced to generate graphs. Data are presented as mean±standard error of mean (SEM). Unpaired, parametric, two-tailed t-tests were used to test mean differences. The significance level used was P<0.05.

### Results

The techniques herein enable pigs to be injected via the ductal system of the pancreas locating the catheter within the entrance lobe of the pancreas so that all three lobes could receive fluid pressure to allow for gene delivery (FIG. 13A). Five pigs weighing 40-54 kg were subjected them to ERCP to access their pancreatic ducts. The pancreatic orifice was readily visualized during the procedure (FIG. 13B), approximately 5 cm downstream of the ampulla of Vater. Fluoroscopy prior to injection confirmed that both branches of the pancreatic duct were accessible reaching the entire organ (FIG. 13C). Hydrodynamic injection proceeded using a power injector, injecting 22-25 mL of saline solution containing 1 mg pClucf DNA at 2 mL/second. Repeat fluoroscopy demonstrated patent pancreatic ducts, indicating no injury from the hydrodynamic injection (FIG. 13D).

Abdominal CT imaging at day 1 post-injection revealed no acute signs suggestive of pancreatitis (FIG. 14A). To further investigate toxicity, blood was sampled prior to the procedure, and then 15 minutes post-injection, 24 hours post-injection, and at the harvest of pig pancreas. Data showed that amylase was transiently elevated after pancreatic injection (FIG. 14B), with no other abnormalities in serum chemistry (data no shown). There was no elevation in leukocyte count after injection as well (FIG. 14C). Vital signs (heart rate, respiratory rate, pulse oximetry) were stable throughout the injection, indicating high tolerance from the procedure in pigs (data not shown).

The pigs were harvested at day 1 post-injection to evaluate for gene expression, avoiding variables of plasmid DNA expression stability/silencing. Pig pancreas tissue was carefully dissected revealing the characteristic three-lobe structure (FIG. 15A), lacking the rotation and fusion of the connecting and duodenal lobes seen in human patients, which forms the head of the pancreas. Pancreas weights across all pigs was 112.3□36.3 g (n=4), correlating with pig weight. Tissue was sampled in all three pancreatic lobes, with catheter placement as noted above in the duodenal lobe.

The pCLucf plasmid encodes separate expression cassettes for firefly luciferase and GFP under CMV and SV40 promoters, respectively. This should yield expression in a variety of cell types. PCR demonstrated the presence of pCLucf DNA in all three pancreatic lobes (FIG. 15B). Immunohistochemistry for FLuc and GFP yielded detectable expression in all three liver lobes, represented by positive islets appreciated in all three lobes (FIG. 15C). Positive cells were ubiquitous across the entire tissue section, emphasizing the high efficiency of the approached (FIG. 15D). Analyzing the cell specificity of gene delivery, there was gene expression primarily in islet cells and ductal cells (FIG. 16A, FIG. 16B). The identity of these cell types was confirmed using pan-cytokeratin and synaptophysin antibodies, which are known to bind to ductal cells and islet cells, respectively (FIG. 16A, FIG. 16B). Immunofluorescent co-staining of specific pancreatic cell types confirmed these findings (data not shown). Beyond islet cells and ductal cells, it was also noted gene expression in endothelial cells (FIG. 16C) and neurons (FIG. 16D) respectively. Levels of FLuc protein were quantified in the different pancreatic lobes using a luminescence assay, finding similar levels of FLuc protein, regardless of proximity to the injection site (data not shown).

It was noted that the presence of damage to the pancreas, which could be observed across all tissue sections to varying degrees. Small areas of necrosis were observed, while areas of immune infiltrates comprising neutrophis and lymphocytes were more common (FIG. 17). The significance of this areas is uncertain, since they did not appear to lead to pancreatitis by day 3, since amylase levels were normal at that time point (FIG. 14B).

### Discussion

The techniques herein provide for the first time non-viral gene delivery into the pancreas of a large animal model, and the first publication to gene delivery into the pancreas of a human-sized animal model (50 kg vs 1.5 kg in previous AAV report). It was demonstrated that ERCP can readily access the pancreatic duct, whereafter hydrodynamic delivery can directly deliver DNA into multiple cell types. DNA was successfully delivered into almost 100% of pancreatic ductal and islet cells in all lobes of the pancreas. Protein expression was confirmed using two different reporter proteins, GFP and luciferase, which both demonstrated positive staining in both cell types. The luciferase staining is stronger, likely due to the CMV promoter being significantly stronger than the SV40 promoter driving GFP expression. Importantly, only a very small level of DNA, 1 mg, was injected into the pancreas in these proof-of-concept experiments, such that increasing the dose in the future may yield higher levels of protein expression. Further optimization of the injection parameters may also yield higher transfection efficiency.

A major concern of this approach is the potential for causing pancreatitis in human patients, which can occur during routine clinical ERCP procedures secondary to cannulation of the pancreatic duct and contrast injection (Clin Exp Gastroenterol. 2021 Feb. 2; 14:27-32). These data in pigs shows that the procedure was well-tolerated with only minimal elevation in amylase levels, and no other biochemistry hematological or imaging abnormalities. There were some histological signs of injury, including areas of immune infiltrate and small areas of necrosis. They likely represent the death of some acinar cells accompanied by release of digestive enzymes. In the time frame of 3 days, no pancreatitis developed, so it's uncertain what clinical significance these areas may have. Even if these injuries are well tolerated in pigs, the physiology and anatomy between pigs and humans are different, such that future studies will have to evaluate tolerability in other animal models. Dogs are a useful model for studying pancreatitis, which has been induced in other studies by large volume contrast injection through ERCP (Comp Med. 2009 February; 59 (1): 78-82). Additional studies will determine whether there are any long-term side effects from the pancreatic ductal hydrodynamic injection, as well as confirm the stability of gene expression. Moreover, it will be important to test different pancreatic cell-specific promoters in order to explore targeting gene expression in intended cell types.

In conclusion, the techniques herein present data demonstrating that gene delivery is possible and efficient into the pancreas of large animals, leveraging a common clinical procedure in ERCP routine today, as well as a power injector used in clinical practice. Given the employment of plasmid DNA as the gene vector, this greatly reduces the cost of treatment and helps obviate potential immune responses to gene therapy. The scalability of plasmid DNA manufacturing may make pancreatic gene therapy possible for the million-plus patients with diabetes, cystic fibrosis, and other pancreatic diseases. Future studies will continue exploring the safety and persistence of gene expression, as well as the delivery of therapeutic genes in specific pig models of pancreatic disease. The demonstration of gene delivery in a human-sized animal model suggests the potential for clinical gene therapy of pancreatic diseases.

### Example 9: Testing for Gene Delivery in Oncopig Model

This project intends to model the creation of primary and metastatic tumors in the liver in a large animal model. Among metastatic tumors of the liver, the project will study colon and pancreatic cancer. The project will also study formation of tumors in the pancreas of large animals. The OncoPig model will be leveraged, which has tumor suppressor (p53) and driver (KRAS) gene under the control of the Cre/LoxP system. A secondary goal of the project will be to test for the possibility of gene delivery into these tumors using a non-viral endoscopic technique. The conclusion of the study will establish the creation of a large animal model of primary and metastatic liver cancer and pancreatic cancer and demonstrate the potential application of gene therapy as a modality for these diseases.

Liver and Pancreatic cancers are devastating diagnoses. Liver cancer can originate either from the liver or from the spread of tumors from other organs. Most pancreatic cancers original within the pancreas. The prognosis for liver and pancreatic cancer is poor and thus, better therapies are needed. Most research is conducted in mouse models, but mice can't readily model the types of interventional procedures that human patients receive. To better model procedures and patience, a pig model will be used to study liver and pancreatic cancer. OncoPig develops liver cancer through a gene switch. The delivery a potential DNA therapy through endoscopy will be tested, where patients get a camera inserted through their mouth into their intestines to access the tumors.

The established OncoPig will be utilized to model liver and pancreatic cancer in a porcine model. Liver cancer will be modeled as a primary hepatocellular carcinoma, as well as metastatic lesions including pancreatic cancer and colon cancer. Pancreatic cancer will be established in the head of the pancreas, where it is localized in human patients as well. The OncoPig contains two cre-inducible mutations, KRASG12D and p53R172H, both of which in combination promote cancer growth. Given that these genes are present in all pig cells, introduction of Cre recombinase can trigger a variety of different tumor types depending on which tissue it is introduced. Established protocols will be used to create tumors in the liver (PMID: 33500185) and pancreas (PMID: 32956389) in OncoPigs.

Once the tumor is formed, endoscopic retrograde cholangiopancreatography (ERCP)-mediated hydrodynamic injection methodology will be utilized to deliver reporter genes into the liver and pancreas tumors. Before and after tumor induction, samples of blood, stool, and saliva will be obtained to aid in the discovery of early biomarkers of pancreatic and liver disease.

### Example 10: Induction of Primary and Metastatic Liver Tumors and Pancreas Tumors in Pigs

Established, published protocols will be followed for the induction of cancer in the liver and pancreas of pigs. In brief, the protocols consist of a core tissue biopsy, and incubation of the tissue for 20 minutes with a solution of Ad-Cre. The core tissue biopsy is then reinserted into the pig for tumor development. Multiple tumors can be initiated within a single pig liver and that paradigm will be followed to gain additional data from the approach. A single tumor will be initiated in the duodenal lobe of the pig pancreas, which corresponds to the head of human pancreas. An additional tumor will be initiated in the splenic lob, which corresponds to the tail of the pancreas.

To expand on the previous work in the liver, it was noted that the phenotype of the induced tumors tended to have an inflammatory component. It is hypothesized that non-hepatocyte cell types may be driving this phenotype. To recapitulate tumor development that resembles hepatocellular carcinoma, an adenovirus will be used that has Cre recombinase under a hepatocyte-specific promoter. For this experiment, the albumin promoter is envisioned to be used.

The prior literature is also limited into exclusively studying the development of primary liver tumors. However, most liver tumors are metastatic, originating from other organs. These metastatic tumors have unique architecture and interactions with the liver tissue around them. In order to study metastatic liver tumors in a large animal for the first time, a biopsy of tissue from the colon and pancreas will be performed. Tissues from these organs will be incubated in vitro with Ad-Cre for 20 minutes, similar to the protocol for the liver biopsy. For this experiment, Ad-CMV-Cre will be utilized, which would express Cre recombinase under a ubiquitous so would express in multiple different cell types in the colon and pancreas tissue.

To achieve tissue collection of these internal organs, endoscopy ultrasound (EUS) will be used to biopsy the pancreas, with a needle protracted from the endoscope and inserted into the target tissue. A proctoscopy will be performed and pinch biopsies will be taken from the rectum. Tissue from the pancreas and colon will be withdrawn from the endoscope and incubated for 20 minutes with Ad-CMV-Cre, like the other tissues. After incubation time, the core tissue biopsies from the pancreas and colon will be inserted into the liver via an ultrasound guided percutaneous injection to see their tumor formation. Previous publication has established that tumors will be 3 cm in size at 1 to 2 weeks post-injection. Ultrasound on the tumor will be used to confirm the tumor size.

To initiate the pancreatic tumor, the same tissue sample harvested from the pancreas by EUS will be re-inserted into the pancreas after Ad-Cre incubation with EUS again. In this manner, a pancreatic tumor can be triggered and formed. Previous publication with this protocol indicates a 3 cm tumor formation at 1-week post-injection (PMID: 32956389).

Ad-Alb-Cre and Ad-CMV-Cre will be purchased from a vendor for the experiment. OncoPigs will be obtained from the National Swine Resource and Research Center (NSRRC) in Columbia, Missouri. OncoPigs will be shipped post-weaning to Rochester, Minnesota for further experiments.

Prior to tumor induction, samples will be collected from all pigs, including from blood, stool, and saliva. These samples will be frozen for later testing of tumor molecular markers with these samples serving as a pre-tumor control.

The biopsy procedure will then proceed forward for the designated tissues of liver, colon, and pancreas. All biopsies will be incubated with Ad-Cre for 20 minutes, and then be re-implanted into the liver or pancreas, respectively. Tissue will be reimplanted with gel to promote tumor formation. Up to 4 liver tissue sites will be used, to represent 4 tumors formed in the liver per pig. Each implantation site will be at least 5 cm apart in the liver.

After the cancer induction procedure, pigs will be monitored for 2 weeks with ultrasound imaging. Blood draws, stool and saliva will be sampled at the time of tumor being 3 cm in size. These samples will be used for cancer and biomarker analysis. A panel of common tumor markers will be tested to check for any trends in several assays including protein markers by ELISA, as well as miRNA or cell-free DNA. In partnership with other labs, it may be possible to find a combination of biomarkers that could be predictive.

At the time of 3 cm tumor size, pigs will be enrolled in experiment #2. Should any pigs with tumors not be enrolled in experiment #2, they will proceed to be euthanized. Tissue will be harvested and analyzed by H&E staining and immunohistochemistry for relevant tumor markers both in the tumor and the tumor microenvironment.

### Groups:

Liver
A. HCC induction (n=4 pigs)
B. Metastatic induction
   i. Colon (n=2 pigs)
   ii. Pancreas (n=2 pigs)
Pancreas
A. Head (duodenal) and Tail (splenic) tumor induction (n=8 pigs)

### Timeline for Experiment #1:

1. Day-7 (pre-procedure): The pigs will acclimate for a minimum of 5 days prior to diet protocol and the procedure begins.
2. Day-1 (pre-procedure): Pig will be fed a full liquid diet (boost or ensure) the night before the procedure, and they will have access to water up until the time of the procedure. Collect the pigs' stool and blood as pre-procedure controls.
3. Day 0 (Procedure): Biopsy procedures, endoscopic and percutaneous, will be performed into the liver, colon, and pancreas tissues. After incubation of tumors with Ad-Cre, tissue will be re-injected with gel into the liver.
4. Day 1-14: Observation and ultrasound on Day 3, Day 7, Day 10, and Day 14. Stool and blood will be obtained at the time of transfer into Experiment #2, or at euthanasia.

Note: Should any pigs fail to harbor any tumors after the procedure, they will be extended another 2 weeks for monitoring, past the original 2 week intended. If still not tumors at that time point, the biopsy procedure will be repeated, with tissue being harvested from the liver.

### Detailed Protocol:

### Liver Tumor Induction:

Tumor induction will be performed when the pigs were 8-16 weeks old. An 18 gauge core biopsy of the liver will be obtained under ultrasound guidance (1 or 2 cm core length, Temno Evolution, Merit Medical, South Jordan, UT), using co-axial technique. TP53R167H and KRASG12D expression will be induced by incubating the core biopsy with an adenoviral vector carrying the Cre recombinase gene (109 pfu Ad5CMVCre-eGFP for colon and pancreas, Ad5-Alb-Cre for liver, commercial vendor) for 20 minutes at room temperature, in phosphate buffered saline containing 15 mM calcium chloride (total fluid volume of 1 ml). Gelatin sponge (Gelfoam, Pfizer) will then be added using a 3-way stopcock, and the mixture (virus, core biopsy, gelatin) will be injected percutaneously back into the pig's liver through the biopsy needle, which will be kept in place. At least four sites will be inoculated in each liver. Inoculation sites will be selected to be far apart as possible, easy and safe to access, and deep enough to avoid leakage of injected material into the peritoneum.

Endoscopic ultrasound guidance will be used to biopsy pancreas tissue. To biopsy colon tissue, an endoscope will be advanced into the rectum of the pig. Pigs will be NPO the night prior and given Colace to promote ejection of all stool from their intestines. Rectal tissue will biopsied, and incubated according the protocol above. Pancreas tissue will be biopsied by EUS through the protocol listed below. For both tissues, the incubated tissue in the gelatin mixture will be inserted back into the liver.

### Pancreas Tumor Induction:

Tumor induction will be performed when the pigs were 8-16 weeks old. A19 gauge core biopsy of the pancreas will be obtained under endoscopic ultrasound guidance. TP53R167H and KRASG12D expression will be induced by incubating the core biopsy with an adenoviral vector carrying the Cre recombinase gene (109 pfu Ad5-CMV-Cre-eGFP, commercial vendor) for 20 minutes at room temperature, in phosphate-buffered saline containing 15 mM calcium chloride (total fluid volume of 1 ml). Gelatin sponge (Gelfoam, Pfizer) will then be added using a 3-way stopcock, and the mixture (virus, core biopsy, gelatin) will be injected through the endoscope back into the duodenal or splenic lobe of the pancreas, through the biopsy needle. Note that pigs have a ring-shaped pancreas with 3 lobes: duodenal, splenic, and connecting. For simplicity, the duodenal lobe is a surrogate to the “head” of the pancreas, and the splenic lobe as the “tail.”

### Example 11: Gene Delivery into a Porcine Model of Cancer

All pigs with confirmed tumors will be injected via ERCP-mediated hydrodynamic delivery approach into the liver.

Whether it is feasible to achieve gene deliver into the tumor, or around the tumor. Among the variables are the interaction of the biliary system with the different types of tumors, and the structure of the tumor itself with the underlying liver tissue.

Toward testing these questions, plasmid DNA will be injected encoding a reporter protein, GFP, under a ubiquitous promoter. This may pick up multiple different cell types. The plasmid will also encode a firefly luciferase protein under the control of a ubiquitous promoter.

After endoscopic hydrodynamic injection, pigs will be euthanized 1-day post injection and tissue harvested. IHC sections will be stained for the GFP and assessed for their presence in and around the tumor. Co-staining will be performed with different antibodies against tumor and cell-specific markers to appropriately identify tumor cell types. The bile duct system will also be stained for and elucidated.

At the same time of euthanasia, blood, stool, and saliva will be collected from all pigs to analyze them for the presence. As stated above, a panel of common tumor markers will be tested to check for any trends in several assays including protein markers by ELISA, as well as miRNA or cell-free DNA. In partnership with other labs, a combination of biomarkers may be discovered that could be predictive.

### Timeline for Experiment #2:

1. Day −3 to −7. Pigs will be identified as having 3 cm tumors in their liver by ultrasound imaging. Plans will be made to do the gene delivery procedure.
2. Day −1 (pre-procedure): The pigs will be fed a full liquid diet (boost or ensure) the night before the procedure, and will be kept nothing per mouth (NPO) overnight prior to the procedure. They will have access to water up until the time of the procedure. Collect the pigs' stool and blood as pre-procedure controls.
3. Day 0 (Procedure): ERCP-mediated hydrodynamic injection will be performed to deliver plasmid DNA solution into cells of the liver. Blood draws will be performed pre-procedure and 15 minutes post-procedure to test from toxicity from the gene injection procedure.
4. Day 1: Observation and blood draw on Day 1. Pigs will be euthanized and necropsy will be performed. Liver will be harvested and gross photos taken of the tumor tissue. Tumor tissue sections will be taken, both on the margins of the tumor, and segments within the tumor.

### Detailed Protocol for ERCP-Mediated Gene Delivery:

Pigs will be sedated, transported to the OR, placed under intubated general anesthesia in dorsal recumbency. Vital parameters will be monitored during anesthesia/procedures. A duodenoscope will be passed per oral to reach duodenum. A commercial biliary catheter used in human patients will be inserted through the working channel of the duodenoscope. With the endoscope placed in the duodenal bulb en face to the biliary orifice, the bile duct will be cannulated with the biliary catheter preloaded with a 0.025 inch hydrophilic guidewire. The catheter will be exchanged for a biliary stone extraction balloon. The stone extraction balloon will be inserted over the wire into the common hepatic duct, followed by inflation of the balloon of the catheter to occlude the common hepatic duct. Fluoroscopic evaluation of placement will be performed by several slow injections of contrast dye (Omnipaque, GE healthcare) to determine the volume and effective distribution of the fluid injection throughout the liver, and the proper placement of the balloon. This will be followed by the injection of plasmid DNA encoding a reporter protein, GFP under a ubiquitous promoter mixed with normal saline at 2 ml/s at a total of 50 ml using a power injector which is usually used for contrast injection (MedRad, Bayer). Post injection, repeat injection of diluted contrast under fluoroscopy will be performed to evaluate the intact of the biliary system. Afterwards, the biliary catheter and om insertion of the endoscope through injection and removal of the endoscope. It is expected that the total procedure time will be 30 minutes.

The procedure will then be repeated for the pancreas. The pancreatic ductal orifice will be identified in the duodenum. The catheter will be inserted into the pancreatic duct. Fluoroscopic evaluation of placement will be performed by several slow injections of contrast dye (Omnipaque, GE healthcare) to determine the architecture and placement of the catheter in the pancreas. The balloon will be inflated and the injection of the DNA construct at a designated parameters (1-2 ml/s with a total volume of 20-40 ml) using a power injector which is usually used for contrast injection (MedRad, Bayer). Post-injection, repeat injection of diluted contrast and fluoroscopy will be performed to evaluate the intact of the ductal system. Afterwards, the catheter and duodenoscope will be removed from the animal.

Tissue will be harvested from the liver and pancreas after euthanasia and evaluated for reporter gene expression. Testing results indicated that reporter genes could be found in different regions inside liver tumors and pancreatic tumors for the first time. This forms the framework for future cancer therapy strategies.

### Example 12: Testing of Gene Delivery in Baboons

### Modeling Biliary Hydrodynamic Injection for Gene Delivery in a Non-Human Primate:

In order to assess levels of reporter transgenes after hydrodynamic gene therapy in an animal model with full secretion of human proteins, translate optimized biliary hydrodynamic injection parameters will be translated into a non-human primate model, baboons, which are the main research primate available that has adequate size to model the human procedures. This primate model will also serve to confirm the translatability and safety of the hydrodynamic approach in a model with liver histology closer to humans. All safety parameters will be monitored. Identical commercially available endoscopy equipment will be used, that was previously used in the pigs and inject at the volume, flow rate and DNA doses that yielded highest reporter gene levels in pigs, as based on tests with human FIX, human ATP7B, firefly luciferase, and green fluorescent protein among others. Baboons (n=5) will be injected with plasmid DNA encoding reporter genes and expression of those genes in the serum or plasma will be followed for up to 6 months. Liver biopsy will be performed at designated intervals to measure protein expression and transfection efficiency. Liver biopsies will be taken on days 14, 90, and 180 to look for gene expression in the liver.

Testing of Repeated Hydrodynamic Injection of Reporter Gene pDNA Vector Dosing to Increase reporter gene levels:

The same baboons (n=5) from the previous experiment will have a second gene injection at least 1 month and up to 6 months after the first gene injection in order to test for the feasibility, safety, and increase in reporter gene levels. A third ERCP gene injection procedure may be performed as necessary if gene expression levels are not achieved to satisfactory levels. This will also conserve and streamline animal resources. In preliminary studies, biliary hydrodynamic injection could be repeated in pigs three weeks later with no adverse events detected. During repeated injection, for reporter gene antibody and T cell responses will be monitored, which could represent priming of the immune response secondary to potential inflammation/injury from hydrodynamic injection and reporter gene expression. Alternatively, potential immune tolerance to reporter genes could be achieved with this non-viral strategy. T cell response will be measured against reporter genes by cytokine release. Beyond IHC and western blot, mass spectrometry will be used to compare the level of reporter genes in baboon tissue samples.

It is expected that this procedure will achieve reporter gene expression in baboons, based on preliminary results in pigs observing high-level reporter gene expression among hepatocytes. These preliminary results in pigs compare favorably to AAV vector transduction efficiency in rhesus macaques. Higher transfection efficiency was observed in a study (32.7-51.9%) compared to ssAAV8, which yielded an average 17% transduced area. Since humans can tolerate repeat ERCP procedures, issues in repeat doing in baboons are not expected, although pancreatitis is a consideration with any ERCP procedure. A relatively low pDNA dose (<10 mg) could be sufficient to achieve detectable reporter gene levels, although it is possible that significantly higher DNA doses may be necessary. It is expected that the liver transfection parameters may be similar between pigs and primates, but differences in liver architecture between species influence transfection efficiency.

### Animal Size:

If possible, animal numbers may be determined based on comparable studies for which the desired effect sizes were shown to be statistically significant.

Endoscopic gene therapy in the liver has been previously reported by the group. In this study, proof of concept work will be done to evaluate the feasibility of hydrodynamic gene delivery via ERCP into the liver to treat hemophilia B, phenylketonuria, hereditary tyrosinemia type 1, Wilson's disease, von Willebrand disease, cystic fibrosis, and familial hypercholesterolemia among other rare disease disorders.

Previous study on the parameters of biliary hydrodynamic gene delivery via ERCP was published in PLOS One in April 2021 has a similar design. Similarly, the team also performed study to deliver human factor IX into the pig's liver which was published in Gastrointestinal Endoscopy in July 2021. A preliminary study got funded to further investigate the parameter and efficiency on biliary hydrodynamic gene therapy. An N=2-3 is being used in most groups which is the minimal number to achieve statistical significance were applied in most of the study groups.

The safe injection parameters for the liver to achieve optimized gene expression efficiency will be determined. The justification for the number of baboons used in the study are in line with prior literature such as the study outlined above.

### Manipulations Done to the Animal:

5 Adult baboons will be hydrodynamically injected via ERCP with reporter gene plasmid DNA on day 0. Serum or Plasma levels from the baboons will be followed for 6 months with blood collections taken on days 7, 14, 30, 60, 90, 120, 150, and 180 to examine expression of proteins in the blood along with other biochemical markers of safety. Liver biopsies will be taken on days 14, 90, and 180 to look for gene expression in the liver.

A repeat procedure on the same baboons will be performed at least one month after the first injection, depending on the initial expression of reporter genes. A third ERCP gene injection procedure may be performed as necessary if gene expression levels are not achieved to satisfactory levels. After these second or third injections, blood samples will be taken weekly for the first two weeks then monthly from day 30 to day 180 when the animals will be euthanized. Liver biopsies will be taken on days 14, 90, and 180 to look for gene expression in the liver.

Blood samples will be processed and sent to the sponsor for T cell analysis and reporter gene expression.

Reporter genes to be considered for testing include human or baboon Factor IX, Beta-hCG, ATP7B, Factor VIII, LDLR, PAH, ABCB4, A1AT, FAH, HFE, UGT1A1, and VWF. Other reporter genes include antibody genes, firefly luciferase, Gaussia luciferase, green fluorescent protein, secreted alkaline phosphatase, and baboon beta-chorionic gonadotropin (beta-CG). Reporter genes employed may different between animals, and different reporter genes may be employed during subsequent redosing in baboons. All these reporter genes are not expected to exert any physiologic effect on the host hepatocytes expressing the proteins. In certain experiments, the reporter genes may be integrated into the host genome using a transposon/transposase system, such as piggyBac.

### Timeline for the Experiment:

Experimental Plan for ERCP-Mediated Gene Delivery of Reporter Gene Plasmid DNA

### Adverse Events:

Since humans can tolerate repeat ERCP procedures, no issues in repeat ERCP procedures in baboons are anticipated, although pancreatitis is Ia consideration with any ERCP procedure. Liver biopsies can result in internal bleeding resulting in pain lasting for up to a few hours.

### Results:

ERCP mediated gene therapy was successfully executed in five different primates. A cohort of two baboons were first injected with beta-hCG, while three other baboons were injected with human FIX. Within a week after injection, serum testing by ELISA could detect the presence of human FIX and beta-hCG. Serum levels of both proteins were well over 1000 nanograms per milliliter. The expression levels stayed at constant levels for at least two months post-injection. Subsequently, a redosing study was performed where plasmid for beta-hCG was injected into the human factor IX expressing baboons and human FIX was injected into the beta-hCG expressing baboons. The serum ELISA tests were repeated and both types of proteins could be simultaneously detected, indicating successful redosing.

Upon sacrifice of the baboons, immunohistochemistry for beta-hCG revealed the abundant presence of beta-hCG in the baboon liver tissue. Normal liver tissue had minimal immunohistochemical staining. RNA ISH for human Factor IX similarly revealed immunostaining of approximately 40% of hepatocytes.

Serum chemistries were monitored during the injections. Post-injection on day 1 found a four-fold increase in ALT and AST. Subsequently, the ALT and AST levels dropped back to baseline within a week post-injection.

These studies also evaluated pressures achieved within the biliary system in primates. It was observed that a flow rate of 1 mL/sec second yields a pressure of at least 50 mmHg. A flow rate of 2 mL/sec and yields a pressure of 80 mmHg. In a flow rate of 3 mL/sec yields pressure of 120 mmHg. Overall, the parameters for efficacious gene delivery in primates is similar to pigs, although the toxicity profile differed as well as some of the DNA dose relationships and the protein levels observed. Studies here are useful in providing new parameters for DNA directed dosing in primates, as well as injection parameters yielding efficient and safe gene delivery.

### Example 13: Testing of Biliary Hydrodynamic Injection at Defined Pressure Thresholds

The minimum pressure threshold to mediate gene delivery through biliary hydrodynamic injection remains uncertain. To address this, a pig was injected with a constant pressure injection device. Previous studies in the literature have used constant pressure injection devices to interrogate hydrodynamic delivery through the vasculature (Suda, et al. Mol Ther 2008). The use of constant pressure injection devices to inject into the biliary system has never been reported until now.

In order for a constant pressure injection device to work, the ductal pressure must be monitored in real time, and then fed in a loop to the injection device to adjust the push of the syringe pump in real time in order to maintain the efficacious pressure of interest. This real-time feedback loop system can be executed in multiple different ways (see e.g., Suda, et al. Mol Ther 2008). The techniques herein provide that an electrical motor may be utilized in connection with a commercial FISO pressure sensor.

### Methods

The pressure threshold was set for a goal of 50 mmHg to be maintained for the entire injection. The total volume injected was 30 mL, while the pDNA dose was 10 mg pCLucf. The injection maintained a pressure from 45-50 mmHg throughout the entire injection. This equated to a flow rate of approximately 0.80-1 mL/sec. Pigs were approximately 35-40 kg in weight in size for the study.

### Results

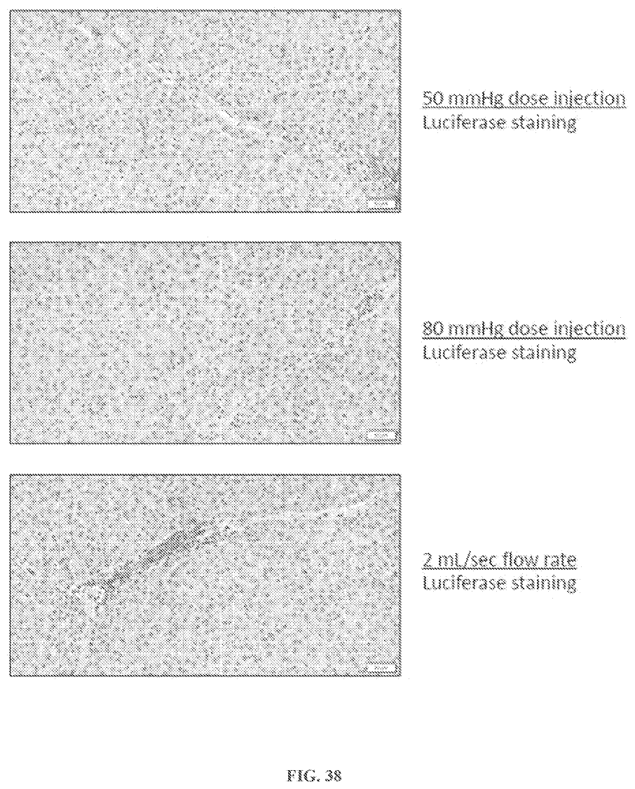

Pigs were evaluated for gene delivery by IHC staining for Firefly Luciferase on the liver tissue section (FIG. 38). A control tissue sample injected with pCLucf at 2 mL/sec is provided for comparison from prior studies. Results show that the pressure threshold was 50 mmHg was not sufficient for injection with no IHC staining for GFP or luciferase observed (FIG. 38). A separate experiment demonstrated that 80 mmHg is sufficient for gene delivery with GFP and luciferase staining observed (data not shown).

### Conclusion

Pressure-directed injection is a feasible strategy for biliary hydrodynamic injection, with real-time ductal pressure able to be monitored. The results were able to show for the first time that 50 mmHg represents a lower threshold for biliary hydrodynamic injection that would not be sufficient to mediate efficient gene delivery.

### Example 14: Testing of Hydrodynamic Gene Injection in Smaller Pigs

Previous experiments have not validated if the designated injection parameters tested in pigs 35 to 50 kilograms would also be relevant to smaller pigs. This is particularly an important issue due to the need to translate the gene delivery procedure from adult patients into pediatric patients. In particular, it remained unknown whether smaller pigs' bile duct lumens could withstand similar flow rates and pressures achieved, and what volume would be tolerated by the pigs during the procedure.

Previous studies have investigated hydrodynamic retrograde intrabiliary injection (HRII), in pigs 4 to 6 kg in weight (see e.g., Mol Ther Methods Clin Dev. 2022 Jan. 19; 24:268-279). However, these studies did not pursue an endoscopy strategy to canulate the bile duct, so it is important to find out if ERCP would be feasible in pigs this size. While the studies did inject 100 mL at 10 mL/sec into the biliary tree, they did not seal the common hepatic duct or common bile duct with a balloon, which would isolate the pressure in the system. Thus, it is uncertain if the ductal systems of these smaller pigs would truly tolerate these higher pressures that occur during hydrodynamic injection.

With the goal of defining the broader applicability of the hydrodynamic injection technique, tests were conducted to determine whether gene delivery could be accomplished in a smaller pig size with similar efficacy. When testing a smaller pig, It is essential to find out how challenging the various parameters might affect gene delivery. Previous studies did not address modifying the volume parameter into smaller subjects. All pigs were injected at about the same weight at 40 kg, so it could be shown that 30 mL volume would be sufficient for these pig sizes. However, whether or not 30 mL should be the volume dose for all sizes of animals is not addressed.

To begin to develop clinical guidelines for dosing, a volume per liver weight parameter was developed. Liver weight was chosen since DNA is only injected into the liver and would not distribute into all tissues including adipose. Moreover, a person's ideal liver weight should be used, which would ignore adipose tissue accumulating in different parts of their body.

### Methods

A 25 kg pig was obtained which is especially smaller than the previous big tested. Injection parameters of 2 mL per second and 25 mL of total volume were chosen. This corresponds to a 40 mL/kg (liver) dose. The volume was reduced with the idea that the pig's liver was lower in volume such that plus volume is needed to sufficiently reach all parts of the liver. The experiment injected 10 milligrams of the plasmid, pCLucf, which encodes a GFP luciferase reporter. The plasmid DNA was diluted into 30 mL of normal saline solution, and an excess solution accounting for the dead-space during injection.

A second pig was obtained which was 13.1 kg in size. This pig was scheduled to have similar injection parameters to the 25 kg pig above with a reduction down to 20 mL in volume. With an estimated liver weight of 450 grams. This equates to a 44 mL/kg dose of volume.

A third pig weighing 27.7 kg was also obtained. It was tested at a higher volume/kg threshold, to gauge the flexibility of the designated parameters. The pig was injected at a dose of 55 mL/kg.

### Results

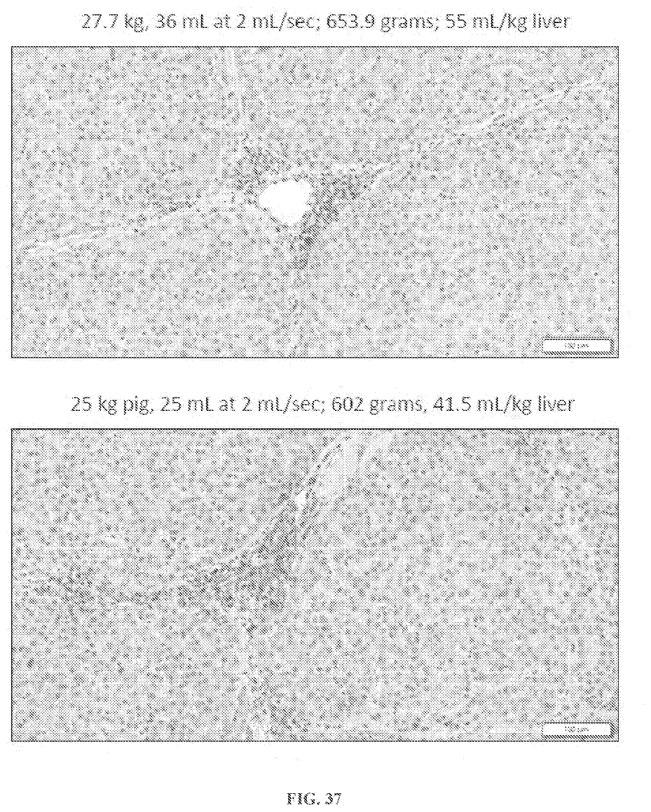

ERCP was able to access the common hepatic duct with success for the 25 kg pig. The small intestine and ampulla of Vater and were still large enough in the 25 kg pig to maneuver the endoscope and catheter, respectively. The injection proceeded without difficulty into the 25 kg pig. Post-ERCP fluoroscopy did not show any rupture of the biliary duct. Immunohistochemistry for GFP and firefly luciferase was able to detect the presence of both proteins in the liver tissue (FIG. 37). The distribution of both proteins within the liver lobule was similar to the previous experiments in larger pigs, with a focus around the central vein of the lobule.

For the 13.1 kg pig, the endoscope was advanced into the small intestine without difficulty. The ampulla of Vater was visualized and was successfully canulated. The catheter was advanced into the bile duct. The sphinctertome was exchanged over the guidewire for the stone extraction balloon. After the stone extraction balloon was being guided into the common hepatic duct versus the cystic duct, it was noticed that a bile duct perforation had occurred. The experiment was subsequently concluded without hydrodynamic injection of DNA being executed, given that the bile duct had ruptured.

For the 27.7 kg pig, the procedure was executed without difficulty. Injection at 36 mL and 2 mL/sec was executed without issue. The liver weight post-procedure was 653.9 grams. Abundant gene delivery with IHC staining was assessed post-injection, with efficiency like other biliary injection studies (FIG. 37). The calculated dose was 55 mL/kg as stated above.

### Conclusion

ERCP is feasible in pigs of smaller size, as low as 25 kg, and hydrodynamic injection through the biliary system is well tolerated. Careful care and consideration needs to be undertaken for pigs of even smaller (15 kg) sizes. Volume dose ranges of 40 mL/kg to 55 mL/kg appear to be equally efficacious.

An important element for the application of biliary hydrodynamic injection will be informed strategy for implementing dosing in a wide range of subjects, from infants to adults. The techniques herein summarize studies from a variety of different biliary hydrodynamic injection procedures in pigs.

The techniques herein have shown that gene delivery can be observed from 22.8 mL/kg (liver weight) to 77.9 ml/kg for volume injected. However, these two extremes were both associated with relatively worse outcomes for transfection efficiency. Volumes from 30 mL/kg to 60 mL/kg exhibited relatively similar outcomes regarding transfection efficiency. The DNA concentration within the injected volume also impacts what is observed. A volume of 68 mL/kg with 43 mg pDNA injected exhibited a >70% transfection, while a 77 mL/kg with 10 mg only much fewer transfection. Thus, DNA concentrations (mg/mL) should optimally be greater than 0.30, 0.40, 0.50, or preferably more than 0.60 mg/mL could be used. For simplicity, one could use DNA dose per kg liver weight as a paradigm, in which case doses of 10 mg/kg, 20 mg/kg, 30 mg/kg, or 40 mg/kg or more could be used.

TABLE 7
Summary of Volume and DNA parameters
during biliary hydrodynamic injection

| Pig | Volume | Liver | DNA | Volume/liver | | |
|---|---|---|---|---|---|---|
| Weight | injected | weight | dose | weight | DNA | DNA |
| (kg) | (mL) | (g) | (mg) | (mL/kg) | (mg/kg) | (mg/mL) |
| 54 | 30 | 1313.5 | | 22.84 | | |
| 34.3 | 31 | 1067 | 5 | 29.05 | 4.69 | 0.16 |
| 41.3 | 33 | 1071.3 | 9.8 | 30.80 | 9.15 | 0.30 |
| 42.5 | 31 | 998 | 6.04 | 31.06 | 6.05 | 0.19 |
| 33.3 | 32 | 992.4 | 5 | 32.25 | 5.04 | 0.16 |
| 36.4 | 29 | 854.41 | 5 | 33.94 | 5.85 | 0.17 |
| 42.9 | 36 | 1000 | 10 | 36.00 | 10.00 | 0.28 |
| 36.4 | 34 | 938 | 13.353 | 36.25 | 14.24 | 0.39 |
| 40 | 36 | 983.5 | | 36.60 | | |
| 49.3 | 40 | 1085.6 | 10.9 | 36.85 | 10.04 | 0.27 |
| 37.1 | 33 | 879 | 10 | 37.54 | 11.38 | 0.30 |
| 31.7 | 32 | 782.9 | | 40.87 | | |
| 25 | 25 | 600 | 10 | 41.67 | 16.67 | 0.40 |
| 55.4 | 60 | 1400 | 10 | 42.86 | 7.14 | 0.17 |
| 30.1 | 40 | 889.6 | | 44.96 | | |
| 36 | 56 | 1046.15 | 19.5 | 53.53 | 18.64 | 0.35 |
| 27.7 | 36 | 653.9 | | 55.05 | | |
| 37.2 | 59 | 1047.21 | 31.5 | 56.34 | 30.08 | 0.53 |
| 37.7 | 66 | 970.18 | 42 | 68.03 | 43.29 | 0.64 |
| 47.8 | 81 | 1040 | 10 | 77.88 | 9.62 | 0.12 |

### Example 15: Effect of Different Injection Parameters on Outcome of Biliary Hydrodynamic Gene Delivery

Only limited investigations were previously conducted to understand how flow rate and volume influence gene delivery through the biliary system. It was observed that increasing flow rate from 2 mL/sec to 4 mL/sec could help bias the distribution from Zone 3 to Zone 1 in the lobule. The potential changes at higher or lower flow rates remain uncertain.

Furthermore, the volume distribution has been kept between 30-40 mL in previous studies, but it remains uncertain whether leveraging larger injection volumes would be beneficial toward injection.

### Methods

To answer these questions, pigs were injected with a reporter plasmid, pCLucf, which encodes reporter genes for firefly luciferase and GFP. Plasmid doses were kept standard at 10 mg for each pig in order to allow for stable comparisons as the injection parameters were changed.

To test flow rates, the following parameters were tested:
33.4 ml @ 1 ml/sec
36 ml @ 4 ml/sec
35 ml @, 7 ml/sec
37 ml @ 10 ml/sec

To test volumes, the following parameters were tested:
36 ml @ 4 ml/sec
60 ml @ 4 ml/sec
81 ml @, 4 ml/sec

### Results

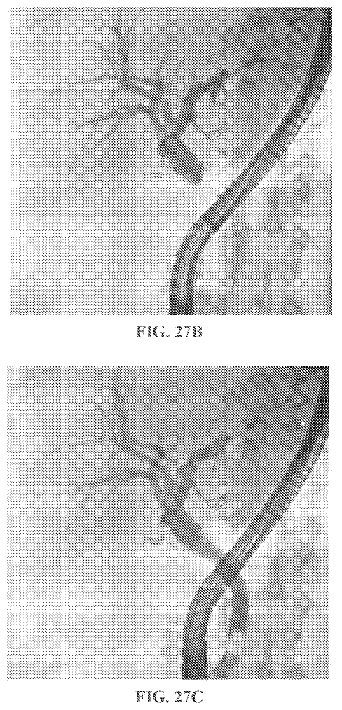

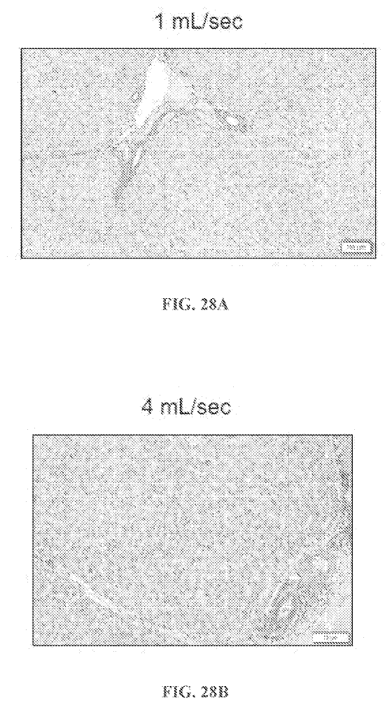

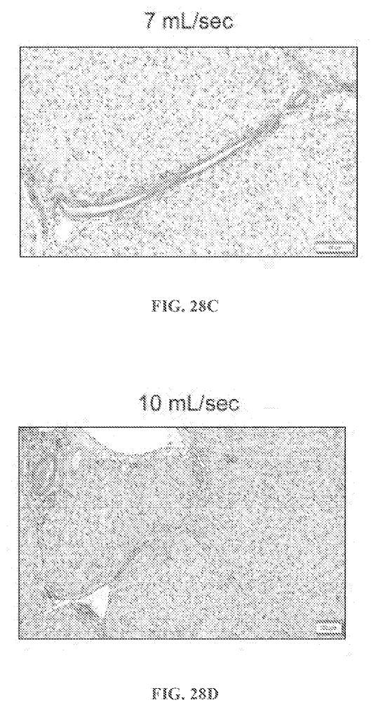

Concerning flow rates, the techniques herein discovered that 1 mL/sec yields almost no detectable immunohistochemical staining for GFP or luciferase (see e.g., FIG. 28). Thus, there is a flow rate threshold for which not enough pressure is generated to yield gene delivery. The 4 mL/sec flow rate yielded similar results to past testing, where there was a mixture of entry into lobule as well as higher percentage around portal areas. At the higher flow rates tested, 7 mL/sec and 10 mL/sec, the pigs tolerated the injections well with no rupture in the bile ducts (see e.g., FIG. 28). Immunohistochemical staining of the tissue revealed gene delivery mostly into bile ducts and endothelial cells, with hepatocytes showing lighter and fewer staining for GFP or luciferase expression. This was particularly true within zone 3 of the lobules, near the central vein. This suggests that the high flow rates may not be more effective in getting DNA into a higher percentage of hepatocytes. Paradoxically, the higher flow rates may actually help push DNA solution faster from the bile ducts into the vascular spaces in the portal triad. Higher flow rates could be particularly effective for more targeted expression into bile ducts without the need for transcriptional targeting with promoters.

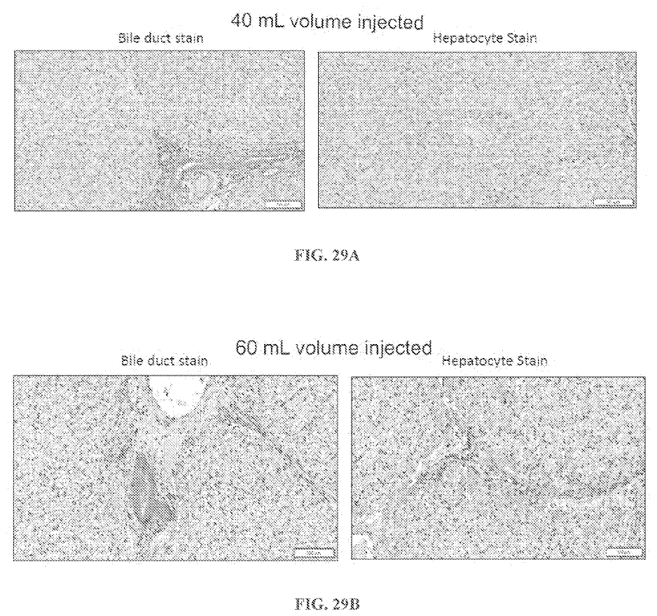

A general decrease in hepatocyte transfection area was observed with larger fluid volumes injected. The volume of 60 mL was largely similar to 40 mL, but 80 mL volume was noticeably less efficient (see e.g., FIG. 29). By liver weight dosing, this corresponds to higher efficiency for 36 mL/kg and 42 mL/kg dosing, compared to 77.8 mL/kg dosing of volume. The 80 mL volume corresponds to the 77 mL/kg dose. This shows that there is better efficacy of smaller DNA solution volumes for gene delivery into hepatocytes of the liver.

An additional factor that influences the lower efficacy of larger volumes is the more dilute DNA concentration. Notably, a separate experiment with 68 mL/kg in volume yielded abundant hepatocyte expression, but the concentration of DNA was 0.64 mg/mL. This is compared to 0.12 mg/mL DNA concentration at the 80 mL volume dose here. Thus, larger volumes for injection should only be contemplated if a threshold of DNA concentration is maintained.

### Conclusion

Optimal flow rates for targeting hepatocytes exist between 2 to 4 mL/sec. It is likely a threshold for fluid to still enter the canaliculi but it possesses significant pressure for gene delivery. The flow rate of 7 mL/sec was somewhat similar to 4 mL/sec, but had lighter staining and relatively fewer hepatocytes. Moreover, the higher flow rates appear to bias gene delivery into bile duct cells, since those cell types remained strongly positive. Lower flow rates down to 1 mL/sec were not enough to mediate any significant gene delivery. Larger volumes are also associated with a general trend toward less efficient gene delivery, particularly at >70 mL/kg threshold.

### Example 16: Efficacy of Nanoplasmid Versus Standard Plasmid in Biliary Hydrodynamic Gene Delivery

Previous testing with biliary hydrodynamic injection demonstrated promising levels of transfection efficiency. The combination of catheter placement, balloon inflation in the bile duct, injection at 2 mL/sec flow rate, pDNA dose >3 mg, and liver-specific promoter synergized to yield transfection areas in the liver exceeding 30%. Although this is an optimal result, further improvements are needed in transfection efficiency to improve the technique.

To identify other areas in which to improve efficiency of hydrodynamic injection, alternative DNA vectors that might enhance transfection and/or expression were evaluated. One of the choices is minicircle vectors, which have the entire bacterial backbone removed. Investigators have previously reported that the bacterial backbone causes transgene silencing from plasmids in gene therapy, such that the use of minicircles greatly improves the longevity and magnitude of expression after gene delivery. The limitation of minicircles is their cost and low yield during manufacturing. An alternative vector system is nanoplasmids, which have many of the same benefits as minicircle vectors. Nanoplasmids have a reduced bacterial backbone size under 500 bp, which is below the threshold of 1 kb thought to cause plasmid DNA silencing.

It was hypothesized that the use of a nanoplasmid, harboring a bacterial backbone less than 500 bp's, would yield greater observed transfection after biliary hydrodynamic injection. Among the possible mechanisms would be smaller DNA entering nuclei better, the nanoplasmid being more transcriptionally active with a smaller bacterial backbone, or both factors working together. On the other hand, the hydrodynamic force could simply introduce more pDNA into the same hepatocytes, or perhaps these factors might have limited utility in large animal livers.

Another group previously employed nanoplasmids to try to enhance their intrabiliary hydrodynamic approach (Mol Ther Methods Clin Dev. 2022 Mar. 10; 24:268-279). The use of nanoplasmids improved detection of luciferase activity at 6 hours post-injection, but by 10 days post-injection, almost all luciferase activity was abolished at almost all tissue samples points. Notably, no protein could be detected by immunohistochemistry after nanoplasmid injection. Therefore, it is uncertain whether the nanoplasmid would significantly improve the hydrodynamic procedure through the biliary system.

### Methods

In order to test this hypothesis, the plasmid, pT-LP1-hATP7B,C9, which has a traditional bacterial plasmid backbone of ~2.6 kb in size containing the pUC origin of replication with the ampicillin resistance gene was tested. The total plasmid size of pT-LP1-hATP7B,C9 is 8.6 kb. The Nanoplasmid with LP1-hATP7B,C9 is 5.85 kb in size by comparison with a bacterial backbone of 428 bp's. The bacterial backbone is comprised of the R6K origin and RNA-OUT selection marker.

For the experiment, 10 mg of NP-LP1-ATP7B was injected by hydrodynamic injection at injection parameters of 40 mL volume and 2 mL/sec. The pig liver was harvested 3 days later and sections were taken in the proximal and distal aspects of the right and left lateral and medial lobes. The quadrate lobe was also sampled. For comparison, a different pig was injected with 10 mg of pT-LP1-hATP7B,C9, and injected the same injection parameters. The liver was also harvested in a similar manner.

### Results

Upon harvesting the pig liver, the NP-LP1-ATP7B demonstrated a profound and unexpected positive rate across all lobes (see e.g., FIG. 30). The average area positive for ATP7B staining exceeded 73.31% in tissue sections. This compared to an average of 47.62% in the ATP7B sections with the pT-LP1-ATP7B containing the bacterial backbone. This difference was highly significant (p=0.0002).

### Conclusion

DNA vectors with significantly reduced or absent bacterial regions, such as the nanoplasmid, can greatly increase the observed transfection efficiency from biliary hydrodynamic injection, when combined with the other optimized procedural methods and promoter. This result is both surprising and unexpected since a recent study with the nanoplasmid and biliary hydrodynamic injection found that the plasmid could not yield any immunohistochemical staining. Moreover, this result was found to be true even when normalizing the per DNA molecule dose administered into the liver, with both plasmids harboring the same gene cassette region. Thus, the nanoplasmid has intrinsically more efficiency at entering into more hepatocytes in the setting of biliary hydrodynamic injection than was expected based on results from other hydrodynamic investigations.

### Example 17: Long-Term Expression of Episomal DNA in Pig Liver

A major challenge of gene therapy is ensuring the longevity of vector expression. For many different gene therapy modalities, expression is often short-lived, which decreases its clinical utility in the patient. For episomal vector strategies, wherein the introduced DNA does not integrate into host chromosomes, it is particularly challenging. Episomal vectors do not divide with the host chromosomes, so are subject to dilutional effects over time. More importantly, episomal vectors are subject to epigenetic silencing overtime, losing expression. This is witnessed by many gene therapy studies in different animal models struggling to achieve long-term expression.

Concerning hydrodynamic injection studies in large animal models, one publication observed expression up to two months, which required redosing at the midpoint. A recent publication with a nanoplasmid injected through the biliary system by hydrodynamic injection did not achieve any expression past two weeks. Thus, it remains uncertain if plasmid DNA introduced by hydrodynamic injection can mediate long-term expression in large animals. The failure of long-term expression, particularly by episomal DNA, would severely limit the clinical utility of the approach.

These challenges were tested in a pig model, where it was possible to assess silencing of the plasmid DNA, while also checking for episomal loss in rapidly growing pig with increasing liver sizes.

### Methods

Four different pigs were injected with 20 mg of nanoplasmid DNA encoding ATP7B, C9 under the control of the liver specific promoter, LP1. The nanoplasmid was used since it should be subject to less silencing potential than regular plasmids containing large bacterial backbones.

TABLE 8
Pigs injected with nanoplasmid

| | | | |
|---|---|---|---|
| Pig #1 | 44.8 kg | NP-ATP7b 20 mg pDNA | 33 ml @ 2 ml/sec |
| Pig #2 | 43.4 kg | NP-ATP7b 20 mg pDNA | 37 ml @ 2 ml/sec |
| Pig #3 | 42.5 kg | NP-ATP7b 20 mg pDNA | 37 ml @ 2 ml/sec |
| Pig #4 | 41.7 kg | NP-ATP7b 20 mg pDNA | 36 ml @ 2 ml/sec |

Injection parameters for all pigs were kept similar to avoid any differences and ensure roughly equivalent transfection efficiency. To gauge the transfection efficiency and gene delivery into the pig liver, each pig was progressively harvested at one month intervals, with the last pig being harvested at 4 months post-injection. Pig sections were harvested sections in the proximal and distal aspects of the right and left lateral and medial lobes.

### Results

Immunohistochemical staining for the C9-tag on human ATP7B was able to detect protein expression in the pigs after every month interval, including the fourth month at the conclusion of the study (FIG. 31). The stain was intermittently either cytoplasmic, or along the cell surface depending on the quality of the section. The expression level was high throughout the study. This was despite the fact that the pigs kept on growing during those four months, with a final weight of 70 kg, representing an almost a 50% increase in total weight. Thus, the nanoplasmid was surprising in maintaining transfection efficiency in a large animal with an increasing liver size. Comparable gene therapy experiments in neonatal mice using episomal vectors have generally lost expression within the same context of liver growth.

### Conclusion

The techniques described herein can yield episomal expression of DNA encoding ATP7B as a reporter over 4 months in pigs. Without being bound by theory, it is believed that the nanoplasmid likely aided in this process. The transfection efficiency appeared to stabilize, suggesting that this expression could extend for much longer in the pig at almost indefinite periods of time. Moreover, there was immune tolerance to the foreign human protein in the pig observed out to 4 months.

### Example 18: Redosing Experiment Through Biliary Hydrodynamic Injection

A key limitation of current gene therapy technologies is the inability to conduct redosing. Current gene therapies rely on different viral vectors, that elicit potent immune responses after the first administration. These immune responses consist of both neutralizing antibodies as well as T cell responses against vector capsids. It is known that most gene therapies based on viral vectors then cannot be redosed due to the vigorous immune response induced after the first administration that would prevent effective gene expression.

Redosing is crucial for gene therapy, however, given the loss of vector expression that can occur over time. This is caused by a variety of different factors from gradual dilution of that DNA vector through cell division as well as gradual silencing of the transgene. Recent studies with human factor 8 for hemophilia treated with gene therapy emphasizes this problem with a gradual declines in factor VIII levels over time. Beyond rare disease, there are also other applications such as cancer, wherein when applying gene therapy to the disease, it could be advantageous to introduce one therapeutic gene at one time, and then subsequently inject a different therapeutic gene as the cancer course changes.

To solve these problems, non-viral strategies for gene delivery are attractive since they do not introduce viral proteinaceous components that would trigger adaptive immune responses. These should in theory allow for multiple doses. This has already been observed through the delivery of mRNA via lipid nanoparticles, which can be administered to a patient multiple times.

Among different non-viral gene delivery strategies, hydrodynamic injection is particularly appealing since it leverages the ability to delivery of naked DNA through fluid pressure, thereby containing no protein components. Hydrodynamic gene delivery is very efficient in mouse models but to date it has not been scaled to work in larger animals. However, in mouse models it has been demonstrated that hydrodynamic injection can be repeated in mice, with the second injection yielding equivalent protein expression.

It remains uncertain whether hydrodynamic injection could be redosed in large animals, and how the route of delivery would influence the outcome of redosing. In a previous study, pigs were injected through a vascular route, wherein each liver lobe is injected individually with DNA solution (Mol Ther Nucleic Acids. 2013 October; 2(10): e128). In one set of experiments, the pigs were injected again by the same procedure and with the same plasmids a second time approximately 40 days after the original injection. The pigs were able to recover completely from both the 1st and 2nd injections with no toxicity effects. The pigs were subsequently sacrificed after these second injections at three week and nine-week time points, respectively. It was noted that the percentage of hAAT-expressing hepatocytes slowly decreased to 8.0, 7.9, 5.6, and 7.8% in the RLL, RML, LML, and LLL, respectively, at the 9-week time point after the second gene delivery. This compares to the higher levels of gene expression at the pigs harvested at the earlier 3-weeks after the second gene delivery, where the gene-injected liver lobes showed 15.3, 14.6, 11.6, and 14.2% positively stained cells in the RLL, RML, LLL, and LML. Thus, the total area transfected decreased rapidly after hydrodynamic injection in their protocol.

Even though this study had some promising results, there are still questions about the relative contributions made by the first and second gene therapy doses because the same plasmid and gene was used for both. Since the entire contribution of the immunohistochemical stain could be from the second injection, it cannot be determined whether redosing occurred.

Even more importantly, these studies were not conducted through the biliary system, which differs greatly in the structure of the ductal system along with potential toxicity and immunological properties. It was previously demonstrated that repeating the ERCP procedure twice in pigs was feasible, with both procedures separated by 3-4 weeks. Pig liver enzyme levels were normalized between procedures. Since it could not be demonstrated that genes could be redosed between procedures, more testing is needed to resolve whether or not this could be achieved.

There are several reasons why redosing could fail. The second hydrodynamic injection could induce loss of expression of the first gene, whether by silencing the expression through primed innate immune responses or extrusion of the first plasmid DNA out of the nucleus. There is also the possibility that the hepatocytes themselves may still be damaged from the first gene injection, such that experiencing a second gene injection would cause toxicity and loss of the hepatocyte and the gene being expressed. As explained above, this latter factor would not be immediately apparent if the second gene was the same as the first gene injected.

To address the issue of whether biliary hydrodynamic injection could be successfully redosed, experiments were conducted in which the same gene with two different protein tags would be injected over two procedures, so that the first injection could be clearly differentiated from the second gene injection.

### Methods

For the first gene injection, the plasmid pT-LP1-ATP7B,C9 was injected along with pCMV-hyPBase, the latter of which would serve to facilitate integration of the transposon into the host pig chromosomes. This would ensure the stability of the first gene injection expression until the second gene injection could proceed.

The injection parameters were 36 mL at 2 mL/second. The pDNA dose injected was 15 mg in total for pT-LP1-ATP7B,C9, and 5 mg for pCMV-hyPBase.

A second series of injections were conducted on Sep. 17, 2021. For these injections, the plasmid, pCMV-GFP-ATP7B was utilized, wherein the n-terminus of the ATP7B protein has a GFP tag on it. The DNA dose injected was 10 mg of pCMV-GFP-ATP7B.

In total, 3 pigs were enrolled in this study, receiving a first injection with a C9-tagged human ATP7B, followed by a GFP-tagged human ATP7B. The interval period between the injections was approximately 4 weeks.

The pig liver was harvested 3 days later after the second injection and sections were taken in the proximal and distal aspects of the right and left lateral and medial lobes. The quadrate lobe was also sampled. Immunohistochemistry proceeded with antibodies against the C9-tag and GFP marker on the same sections.

### Results

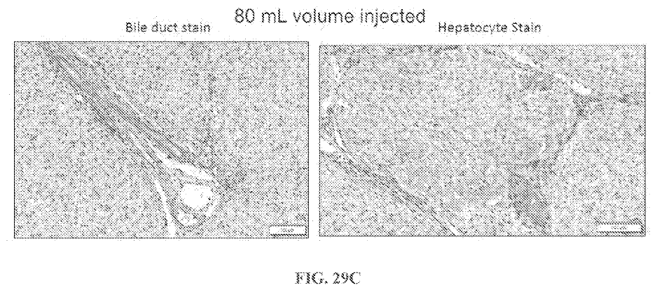

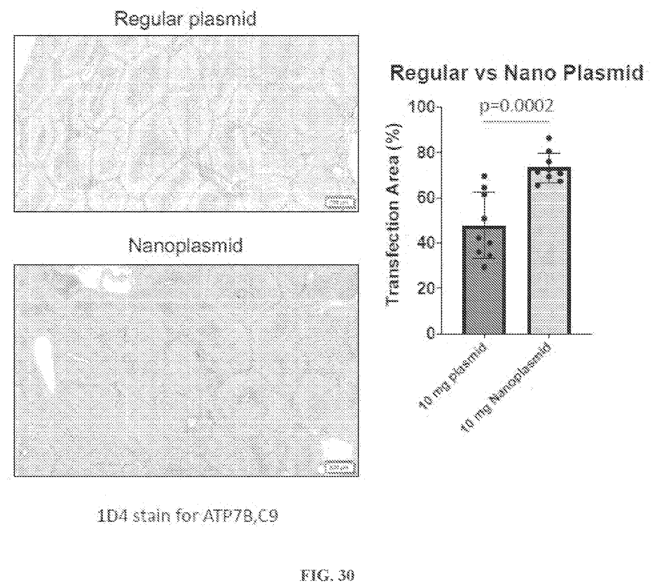

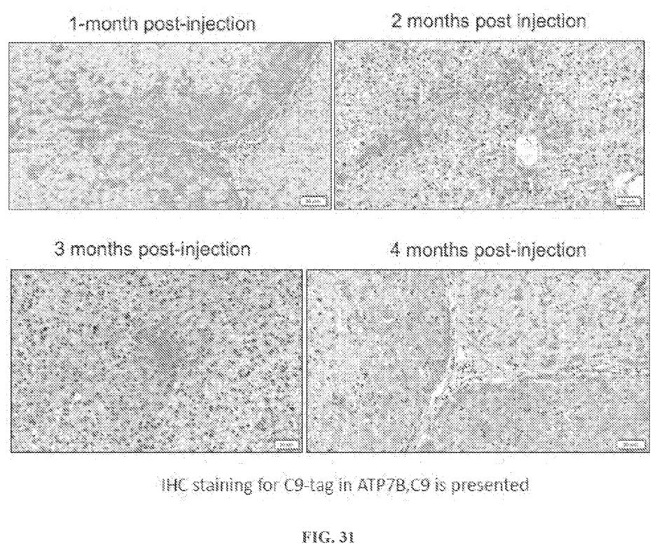

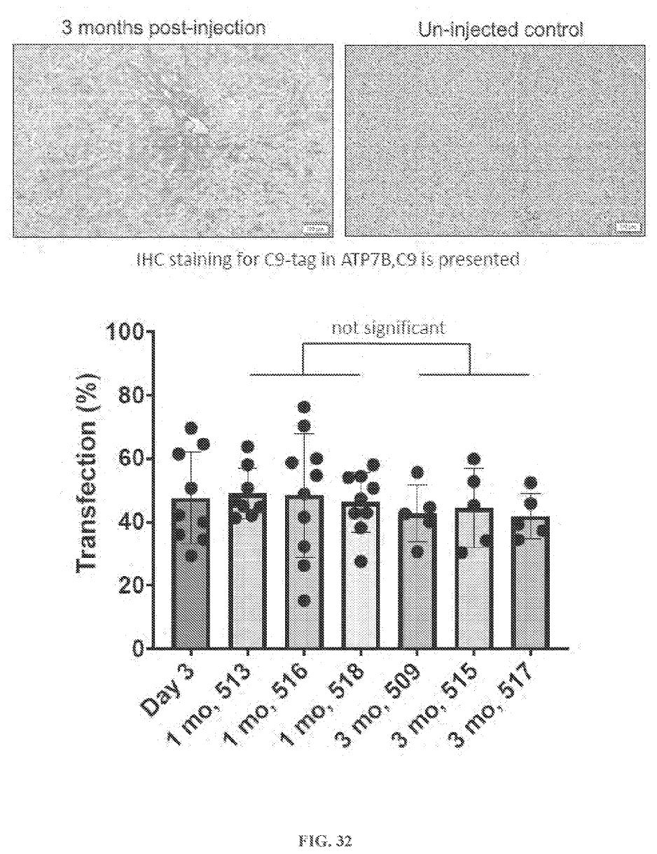

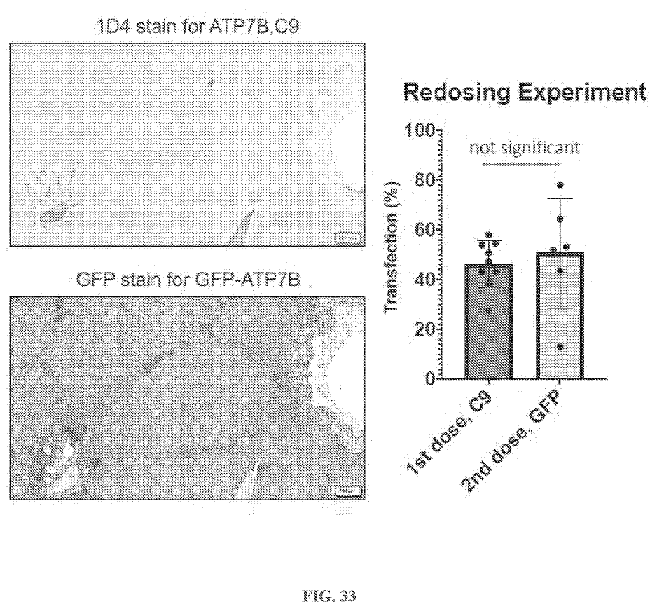

It was observed that GFP-ATP7B and ATP7B-C9 could both be detected by IHC on the same sections in all three pigs (see e.g., FIG. 33). This shows that the result can be reproduced. Furthermore, both stains could be found within the same lobules and even the same cells, the latter suggesting the second injection is not toxic to the first hepatocytes and that repeat dosing into the same cells is feasible. Equally important, the transfection efficiency of the first and second doses was similar (46.2% vs 50.7%; not significant), indicating no impact of the first dose on the tissue architecture to allow subsequent redosing (see e.g., FIG. 33).

Clinical chemistry analysis of the pigs prior to and subsequent to both injections did not show any abnormalities. Thus, the procedure was well tolerated. Moreover, there was no rupture of the bile ducts after the second injection, which suggests that no additional strain or permanent weakening of the ductal walls occurred.

### Conclusion

The present disclosure shows that a secondary gene treatment form of gene therapy by biliary hydrodynamic injection in a large animal model is feasible. This is the first time any group has demonstrated the ability to detect two different genes after a secondary gene treatment of gene therapy in pigs. This finding adds further support for the clinical viability of biliary hydrodynamic injection. No group has previously shown that biliary hydrodynamic injection can maintain equal efficiency upon redosing in large animals as well.

### Example 19: Testing of Delivery of Different Reporter Genes and Plasmid Sizes by Biliary Hydrodynamic Injection

Biliary hydrodynamic injection was previously validated with the plasmid, pT-LP1-hFIX, which demonstrated efficient expression in pig hepatocytes (Kruse, G I E 2021). A challenge of the prior study, however, was the low-level cross-reactivity of the anti-human FIX antibody against pig FIX, which made consistently clear IHC staining across sections difficult due to low-level background staining. Moreover, there is always the risk of individual antibodies targeted at antigens having unforeseen background effects. The easiest way to solve this problem is to validate gene therapy with multiple different gene cargo, including proteins that would have no homologous antigen within the pigs for cross-reactivity.

Another question from the previous studies is what the potential efficiency of gene delivery for plasmids of different sizes is. The previous plasmid DNA employed was pT-LP1-hFIX, which is 5564 bp in total size. This pDNA is relatively compact, due to the transgene, human FIX, being only 1383 bp in size. Many therapeutic proteins are significantly larger, including several over 5 kb. This also excludes therapeutic scenarios where dual expression of different genes from different promoters would be optimal, which would require even larger plasmid sizes and payloads.

### Method

In order to address these questions, a series of different plasmids and transgenes were tested. Descriptions of the genes are described further below. All pigs were injected by biliary hydrodynamic injection. Pigs were harvested on day 3 post-injection. Sections were taken of the proximal and distal aspects of the right and left lateral and medial lobes for all pigs. The quadrate lobe was also sampled. Additional serum samples were obtained where indicated.
1. pBa-LSS-GFP-LDLR wt: Plasmid size: 9336 bp's
Gene: Human Low Density Lipoprotein Receptor (LDLR): 2580 bps
N-terminally tagged with Green Fluorescent Protein.
Promoter: Chicken Beta Actin

The CAG promoter would be expected to yield expression in multiple cell types. The coding sequence for GFP-LDLR still uses the native human LDLR signal sequence, which is cleaved leaving the GFP attached to the mature LDLR protein. The goal was to detect the LDLR gene against the N-terminal GFP tag, using a GFP antibody for this purpose.

The LDLR gene was injected into a pig that weight 38.6 kg at injection parameters of 42 mL at 2 mL/sec. The plasmid DNA dose was 12.89 mg.
2. pCDNA4/Full length FVIII Plasmid size: 12,086 bp
Promoter: CMV
Gene: Full length human FVIII, 7065 bp

It would be expected that the CMV promoter would yield expression in multiple cell types. The coding sequence for human FVIII is the full-length protein over 7 kb, which greatly exceeds the native AAV packaging limit. To address that issue, investigators and companies have created B-domain deleted (BDD)-FVIII constructs, since that particular domain is not essential for clotting function. The BDD-FVIII genes end up being around 4374 bp in size. This allows the gene to just barely fit inside AAV vectors. Since hydrodynamic injection lacks any DNA size restriction, it would be suitable to deliver the full-length human FVIII protein, which may still provide better regulated function. Furthermore, hydrodynamic injection could also deliver the gene into endothelial cells, which is the native cell type where FVIII is made, not inside hepatocytes.

For the current experiment, the plasmid, pCDNA4/Full length FVIII, contains a C-terminal myc-tag at the end of the FVIII gene. The goal of the experiment then was to inject the FVIII-myc into the liver, and then detect the human FVIII gene with the c-terminal myc tag by immunohistochemistry, using a myc antibody for this purpose.

The human FVIII gene was injected into a pig that weight 35.4 kg at injection parameters of 42 mL at 2 mL/sec. The plasmid DNA dose was 9.26 mg.

### Results

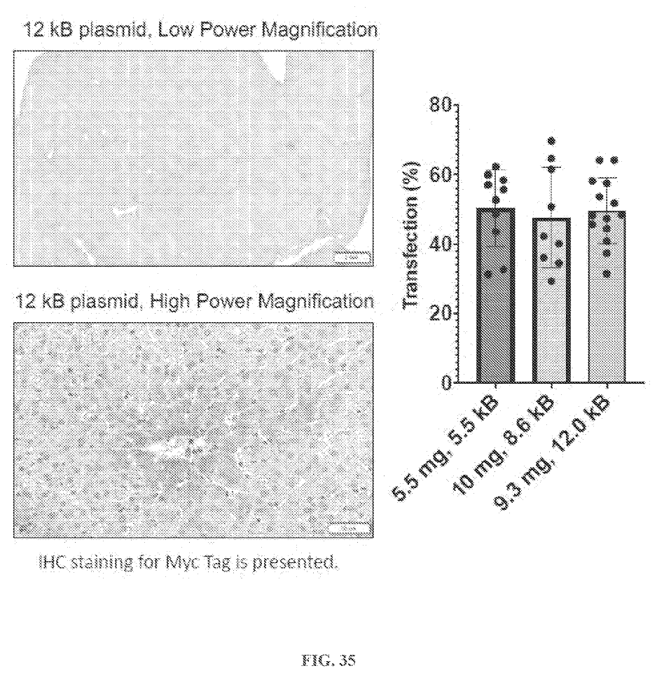

All pigs underwent successful hydrodynamic injections. The ERCP procedure in each animal was unremarkable, with no rupture of the biliary ducts noted. Chemistry analysis did not detect any increase in transaminase levels pre- and post-injection. Pigs were harvested at day 3 post-injection. Results of gene expression for each animal are summarized below.
1. Immunohistochemical staining for GFP in the pig injected with GFP-LDLR was able to detect efficient staining in hepatocytes, predominately in zone 1 and 3 regions of the hepatic lobule, as had previously been published. Immunostaining could be seen in every hepatic lobule.
2. Immunohistochemical staining for myc in the pig injected with hFVIII was able to detect efficiency staining hepatocytes, predominately radiating out of the zone 3 region of the hepatic lobule, had been previously published (see e.g., FIG. 35). Immunostaining could be seen in every hepatic lobule. The stained area quantified to be 49.48% and compared to past experiments using different plasmid DNA sizes at different doses, with all corresponding to 0.78-1.0 mg per kB of pDNA (see e.g., FIG. 35). There was no significant difference in the percentage of stained area between groups, which all consisted of different IHC antibodies (5.5 kb: hFIX; 8.6 kb: ATP7B, C9; 12.0 kb: FVII-myc).

The transfection efficiency can be maintained using larger DNA sizes, as long as DNA dosage per kb guidelines are maintained. The simplest example would be to normalize all DNA weight-based dosing (mg DNA/kg liver) by the index plasmid size to achieve the transfection threshold of the index plasmid. In this case, the index plasmid has a size of 8.6 kb, with known goals of ~10 mg/kg dose for 50% and ~20 mg/kg dose for 70%. Within those bounds, multiplying the weight-based dose times the plasmid DNA size (in kb) divided by the 8.6 kb index plasmid size would yield a plasmid DNA dose that should yield similar injection results.

In another example, the following formula could be used to calculate the DNA dose for any given liver weight and plasmid DNA size. The formula is provided below, based on the current results and the results of plasmid DNA dosing reported separately.

TABLE 9
Formula for DNA dosing

| | |
|---|---|
| DNA dose(mg)/Liver (kg)/plasmid DNA(kb) | % Delivery |

| | | |
|---|---|---|
| 1 | mg/kg/kb | 50% |
| 2.5 | mg/kg/kb | >70% |
| 3.5 | mg/kg/kb | >70% |
| 5 | mg/kg/kb | >70% |

The formula outlined in Table 9 should be accurate for dosing DNA. Depending on the availability of DNA dose, slight deviations of this formula are acceptable within plus or minus 0.25 mg/kg/kb, 0.5 mg/kg/kb, 0.75 mg/kg/kb, or 1 mg/kg/kb, such that these differences will not affect the percentage greatly and will produce similar dosing efficacy.

### Conclusion

The previous results of IHC staining with human FIX in pig liver could be reproduced with antibodies against GFP and myc. In addition, separate studies have also demonstrated IHC staining against C9-tag and firefly luciferase. For all different gene and antibody combinations, the IHC pattern was very similar, showing that the mechanism was robust and validating the efficacy of biliary hydrodynamic injection in efficiently delivered DNA inside hepatocytes. The study success utilizing the largest plasmid DNA size ever demonstrated by biliary hydrodynamic injection. This may be the largest non-viral plasmid DNA vector ever delivered into a large animal of any size by hydrodynamic injection, and represents a new avenue for the clinical potential of this approach.

### Example 20: Cell-Specific Targeting into Endothelial Cells of the Liver

The techniques herein have also conducted experiments using ubiquitous promoters like cytomegalovirus promoter to direct expression of reporter genes inside pig liver. During these experiments, it was noted gene expression occurred in other cell types beyond hepatocytes. These cell types included reporter gene stains for bile duct cells, endothelial cells, neurons, and some light staining in fibrous tissue. Without being bound by theory, it is believed that the DNA solution escapes the biliary system and ends up bathing multiple different cell types with DNA, as pressure opened up borders between these different cell types to allow DNA to enter inside the cells throughout the liver. A previous experiment explored using two different promoters and two different reporter genes to interrogate this phenomenon. Both of these ubiquitous promoters, cytomegalovirus and SV40 promoter, were both able to drive expression in multiple cell types in the liver.

Given the potential for gene delivery into multiple cell types, it would be useful to target gene expression into specific-cell types. Hydrodynamic gene delivery is unique in not having a receptor-mediated entry process, such that DNA enters into multiple different cell types. Because of that, the key method to tailor gene expression into specific cell types is to alter the promoter being utilized.

The ability to modify the promoter for a specific cell type was tested in order to demonstrate that cell-specific expression could be obtained. Vascular endothelial cells were tested as the first cell type as a proof of concept, given that a well characterized vascular endothelial cell-specific promoter exists. Furthermore, vascular endothelial cells are a good target since they do not directly border bile ducts or bile canaliculi, and thus, the DNA solution must pass through different intercellular spaces in order to reach the endothelial cells. Therefore, it would be a significant, surprising, and unexpected discovery for endothelial cells to be able to be targeted by biliary hydrodynamic injection.

Endothelial cells have multiple different phenotypes. Within the liver, there are vascular endothelial cells which border arteries and large veins, and liver sinusoidal endothelial cells (LSEC), which form along sinusoids in close proximity to hepatocytes, with a narrow Space of Disse between the cell types. LSEC have been known to have specialized functions in sorting nutrients and antigens perfusing through the liver.

### Methods

The plasmid, pICAM2-GFP, was synthesized for endothelial cell targeting. The ICAM2 promoter has previously been characterized in the literature to be endothelial cell-specific, and the sequence was obtained from Invivogen. A codon-optimized green fluorescent protein (GFP) for pig was synthesized downstream of the promoter. GFP was chosen since the protein is well characterized in the field and importantly, and no homologous, cross-reacting sequences in the pig.

The ICAM2 promoter was previously used in the creation of a transgenic pig model to direct endothelial cell expression (Xenotransplantation. 2003 May; 10 (3): 223-31). However, only two of out of 57 transgenic pigs showed expression, which appeared to be restricted to vascular endothelium in heart and kidney but was markedly weaker than in transgenic mice produced with the same batch of DNA. Thus, in this case, promoter performance in mice and pigs was not equivalent. The weak expression driven by the human ICAM-2 promoter in pigs relative to mice suggests the need for additional regulatory elements to achieve species-specific gene expression in pigs.

Given this literature, the techniques herein sought to identify an alternative promoter that could specifically seek expression inside LSECs. LSECs are of particular interest since they are the cell type that mediates expression of FVIII important in hemophilia A disease. LSECs have unique phenotype and cell markers compared to other endothelial cells. Among the different cell markers, CD36 shows uniform expression in all LSECs across the different lobular zones. Consequently, a CD36 promoter was utilized to drive expression of a reporter protein, firefly luciferase in order to determine cell-specific expression inside LSECs in pig liver.

For the experiment, a pig with the weight of 34.0 kg was selected for injection. Injection was undertaken at 41 mL of volume and 2 mL/sec for flow rate. ERCP was used to access the common hepatic duct of the pig, and hydrodynamic injection into the liver proceeded without incident. Pre- and post-injection serum samples was obtained. Pigs were euthanized on day 3 post-injection. The liver was collected and samples from the right and left medial and lateral lobes of the liver were obtained. Within each lobe, proximal and distal aspects to the injection point were obtained. A GFP antibody was used to stain all liver tissue by IHC.

### Results

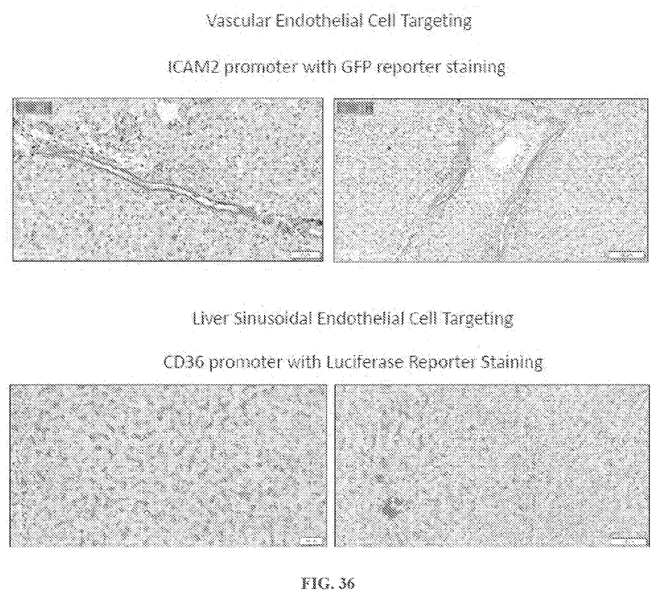

Prominent immunohistochemical staining was observed in endothelial cells around arteries with the pICAM2-GFP vector (see e.g., FIG. 36). This staining pattern accumulated in the arteries and arterioles found in portal triads. No endothelial cells stained positive in larger veins in portal triads. There was no endothelial staining among sinusoidal endothelial cells (LSEC) bordering hepatocytes. The immunostaining was extremely cell-specific, with all hepatocytes, bile duct cells, and other cell types demonstrating no brown staining at all.

Using the pCD36-Luc vector, it was observed that prominent and specific immunohistochemical staining was observed along the borders of sinusoids, corresponding to where LSEC are found (see e.g., FIG. 36). No expression could be observed in large veins or large arteries in portal triad regions. Furthermore, hepatocytes and bile duct cells were also stained negative.

When considering the efficiency of the procedure, all visible endothelial cells bordering arteries were stained positive in multiple tissue sections analyzed, across all visible portal triads. This equates to essentially a 100% transfection percentage into the vascular endothelial cell types. Similarly, it was estimated that 70-80% of sinusoids stained positive, with the highest concentration occurring around the central vein within the hepatic lobule. This corresponds to the area of most efficient hepatocyte delivery.

### Conclusion

Vascular endothelial cells and liver sinusoidal endothelial cells can be targeted with different cell-specific promoters after biliary hydrodynamic injection. The efficiency of delivery into endothelial cells bordering arteries appears to be very high. This strategy could be useful for address disorders such von Willebrand's disease, where the cognate proteins VWF are produced naturally by endothelial cells, being packaged into special vesicles before release. The strategy could also be useful in treating hepatic artery stenosis. Of note, no expression was observed in liver sinusoidal endothelial cells, which may reflect a lack of ICAM-2 promoter activity in these cell types, or alternatively no pressure being achieved on the surface of these cells. Expression in LSECs could find use for expression of FVIII to treat hemophilia A.

### Example 21: Long-Term Expression after Biliary Hydrodynamic Delivery when Using Integrating Systems

Length of expression after biliary hurting make injection is crucial when appreciating the potential clinical applications. Expression only lasted a very short time, everybody limited diseases that could be treated by the procedure. Even if the procedure could be repeated it would not be clinically feasible to have so many repeat procedures in order to maintain gene expression.

Previous studies only investigated very short-term time points of gene expression after pig injection ranging at most up to three weeks post-injection (Kruse, G I E 2021). This time point is after the typical period of time where an adaptive immune response would have occur to remove foreign protein expression. Although promising, this time point is still relatively short to assess what levels of DNA silencing would occur long term.

In previous studies, it has been noted that transposon systems are needed for stable long-term expression for the lifetime of the mouse after hydrodynamic tail vein injection. This is true whether this system is from Sleeping Beauty or piggyBac transposon. It remains an open question how long hydrodynamic injection could mediate expression in large animals, which in general are resistant to gene therapy across all modalities. Concerning the prior literature on hydrodynamic injection, two informative studies have been employed.

In these studies, balloon catheters were used to deliver DNA solution by hydrodynamic injection into dog liver through vascular routes (Hum Gene Ther. 2017 Jul. 1; 28 (7): 541-550). The investigators observed the ability of canine liver to secrete reporter proteins that were detectable in the serum. However, expression lasted only up to six weeks with secreted alkaline phosphatase (SEAP) and only up to 10 days with canine erythropoietin. This was in spite of using Sleeping Beauty transposon to mediate in theory long term expression.

A separate study by the same group sought to extend the expression time of the hydrodynamically injected genes beyond six weeks (Hum Gene Ther. 2017 July; 28(7): 551-564). In order to achieve long-term expression, immunosuppression of dogs with gadolinium chloride (GdC13) was required to achieve the presence of cSEAP in the circulation up to 5.5 months after a single vector infusion. Using potential therapeutic genes, activities of transgenic IDUA and GUSB in plasma peaked at 50-350% of wildtype, but in the absence of immunosuppression lasted only a few days. Importantly, even with immune suppression, transgene expression declined gradually but appeared to stabilize after about 2 months at approximately fourfold baseline level.

Notably, the durability of transgenic protein expression in the plasma was inversely associated with transient increase of liver enzymes alanine transaminase (ALT) and aspartate transaminase (AST) in response to the plasmid delivery procedure, which suggests a damaging effect of hepatocellular toxicity on transgene expression. GdC13 treatment was ineffective for repeat vector infusions, suggesting that hydrodynamic injection might not be able to be redosed.

Previous studies also investigated duration of expression after biliary hydrodynamic injection. In a published study, they observed <1% transfection efficiency using human oncogenes as the delivered gene with Sleeping Beauty transposon mediated integration (Kumbhari, G I E 2018). Positive immunofluorescent staining for a reporter gene could be observed up to 2 months. However, given that the genes themselves promoted oncogenesis of the target cells, this could have facilitated maintaining this gene expression. Therefore, it is uncertain what the duration would be for a normal therapeutic gene after hydrodynamic injection, whether integrated or episomal. Furthermore, the transfection efficiency was very low, so it was doubtful that any immune response would have developed to the foreign protein. However, it is uncertain what would happen when an abundant foreign protein is expressed in the liver, if the immune system would fully recognize it for adaptive immune clearance. In summation, given that therapeutic application of biliary hydrodynamic injection will not use either oncogene and requires a very high transfection percentage, this study does not inform on the potential for long-term expression of therapeutic genes at clinically relevant transfection percentages.

To find out the long-term durability of gene expression from transposons after hydrodynamic injection, it was necessary to form a similar experiment to the previous publication (Kruse, G I E 2021) using a transposon system to mediate integration into the host pig chromosome. Monitoring expression over several months was performed to observe if the percentage of cells expressing the transgene changed over time. A transposon system was chosen since it closely resembled previous work. It has been noted previously that episomal DNA can be prone to silencing. One of the other major risk is that the pig liver is still growing overtime which would further dilute any episomal DNA. In order to remove those possibilities, the gene was integrated into the genome. This provide an important study for future applications where an integrating system might be chosen for a potential simple gene therapy to treat a rare disease.

### Methods:

Three pigs with weights of 42.2 kg, 40.7 kg, and 41.8 kg were selected for the experiment, respectively. Plasmid DNA doses were 15 mg pT-LP1-ATP7B,C9 and 5 mg pCMV-hyPBase.

Plasmid DNA was diluted in normal saline solution prior to the injection to the total designated volume below. ERCP was used to access the common hepatic duct of the biliary system in all three pigs without difficulty. Hydrodynamic injection proceeded at parameters of 36-43 mL and 2 mL/sec in all three pigs.

Approximately halfway through the experiment, at 1.5 months after the first hydrodynamic injection, all three pigs had a liver biopsy. Two pigs at percutaneous core liver biopsies. A third pig had an open surgical incision and a small wedge biopsy, due to complications during anesthesia that prevented obtaining a core biopsy. All biopsies were sent for histology and immunohistochemical staining to verify the presence of ATP7B, C9 with 1D4 antibody against C9-tag. This step was used for an assessment that gene expression was present in all pigs at this time point, such that it would be worthy to continue the experiment out to 3 months. The biopsies also helped assess the relative efficiency of gene delivery, although the limited sampling makes any determination of efficiency prone to inaccuracy.

Pigs were sacrificed at 3 months post-injection, which was determined to be a timepoint based on other published studies where gene therapy expression has stabilized indefinitely. All four of the main liver lobes were sampled, including the right and left medial and lateral liver lobes. These tissue samples were submitted for formalin fixation and immunohistochemical staining with 1D4 antibody. Comparisons to the efficiency of transfection at this timepoint were made to previous studies of biliary hydrodynamic injection of the same plasmid on shorter Day 3 timepoints.

### Results:

All three pigs demonstrated immunostaining for ATP7B, C9 at the three-month time point, reflective of stable expression and immune tolerance to the human protein in fully wild-type, immune competent pigs (see e.g., FIG. 32). Immunostaining at the interim biopsy points at 1.5 months also showed positive expression of ATP7B,C9 (data not shown). The pattern of immune staining was similar to short-term studies that were conducted, where staining accumulated predominately around zone 3 within the hepatic lobule. Comparing the transfection efficiency to pigs previously injected and harvested at earlier time points, it was observed that the pattern and efficiency of transfection were similar and there was no significant difference from pigs injected with equivalent doses at day 3- and 1-month post-injection (see e.g., FIG. 32). This reflects that the transposon system should stable integrate and maintain relative transfection efficiency, even if the pig livers grow in time.

Data was collected on each pig, finding the liver weights at 3 months were 1692.8 g, 1399.4 g, and 2324.9 g. These liver weights are on average 60-70% larger than the comparative liver weights when the pigs were injected, reflecting a substantial increase in liver size that should exhibit dilutional loss of any unintegrated plasmid DNA. By comparison, the stable transfection area suggests sufficient integration of the delivered transposon.

### Conclusion:

Long-term expression of integrated DNA extending to 3 months tested can be achieved after biliary hydrodynamic injection. The genes injected did not undergo silencing and do not trigger any immune responses that would clear the foreign transgene.

### Example 22: Plasmid DNA Dosing Series with Biliary Hydrodynamic Injection

The vector dose influences the outcome of gene delivery into the tissue and it is a major factor in gene therapy technology. It follows that higher levels of the vector dose leads to more delivery across the tissue, although this effect can be limited, as in seen with small molecule drugs and biologic agents. Higher doses of gene therapy vectors can be associated with toxicity. High doses of AAV have been associated with hepatotoxicity in patients and even death. Moreover, higher vector doses can stimulate to the immune system leading to clearance of transgene.

Considering hydrodynamic injection, a wide range of plasmid DNA doses have been applied in mouse models, exceeding 50 μg for a 25 g mouse. There is no trend in toxicity of plasmid DNA dose in the mouse with changes in gene delivery outcome, although no investigations have studied this in detail.

The relationship of DNA dose to the outcome of hydrodynamic injection in large animal models remains uncertain. Vascular routes of injection have been more extensively described in the literature, but because that route of delivery contacts deliver surface differently than biliary system. It is uncertain if those results on plasmid DNA doses would truly educate liver toxicity. In more detail, the vascular space contacts sinusoidal endothelial cells and Kupffer cells in the Space of Disse. The direct contact with immune cells offered by vascular injection threatens to lead to more inflammatory reactions, as compared to the biliary system which lacks direct contact with Kupffer cells.

Previously published work only studied DNA doses up to 5.5 mg pDNA through the biliary system (Kruse, G I E 2021). This study did find that apparent transfection area increased from the smaller 3 mg pDNA dose. The transfected area between these doses ranged between 30-50%. It remains to be seen how this would translate into other therapeutic genes, and more importantly, if the transfected area could continually get higher with progressive DNA dosing.

Of note, one other group has studied hydrodynamic injection through the biliary system (Mol Ther Methods Clin Dev. 2022 Mar. 10; 24:268-279). The group injected 2 mg and 12 mg into pigs weight 4-6 kg in size. Relative to total pig weight, these doses ranged from 2-3 mg/kg. The approach and published data suggest a lack of toxicity with injecting DNA solution, however, the numerous differences between their method and the one described in the current patent, ranging from surgical technique used, to lack balloon seen in the common hepatic duct (or even common bile duct), and lack of detected protein expression upon immunohistochemical staining suggest a lack of efficient DNA entry into cells. This is supported by the fact that the DNA presence detectable by PCR drop decreased significantly from 6 hours to 3 days to 10 days post-injection, which would be reflective of extracellular DNA being digested that is not internalized within cells. Importantly, previous studies were accomplished with doses of 0.1-0.3 mg/kg pDNA with much better gene expression achieved, emphasizing the differences in the techniques.

It was determined that it was essential to resolve these outstanding questions and understand the toxicity of higher pDNA doses as well as the potential for higher transfection percentage to be achieved with the progressively increasing doses.

### Methods:

The plasmid, pT-LP1-ATP7B, C9 was selected for the dosing series, since it has generated clear staining with the 1D4 antibody by immunohistochemistry after biliary hydrodynamic injection in pigs in other pilot experiments. The 1D4 antibody reacts with the C9-tag, a sequence only otherwise found in retinal cells and not expected to have cross-reactivity anywhere else in the liver. The plasmids were prepped with gigaprep maxiprep rates

TABLE 10

| Animal ID# | | |
|---|---|---|
| (DNA Payload) | Animal Weight | Liver Weight |

| | | | | |
|---|---|---|---|---|
| Pig #1 | 36 | kg | 1045.15 | g |
| (pDNA dose: 19.5 mg) | | | | |
| Pig #2 | 37.2 | kg | 1047.21 | g |
| (pDNA dose: 31.5 mg) | | | | |
| Pig #3 | 37.7 | kg | 970.18 | g |
| (pDNA dose: 42 mg) | | | | |

All three pigs were injected at parameters of 65 mL of volume at 2 mL/sec flow rate. A higher volume was utilized to accommodate gigapreg kits with higher pDNA yield, but elution into larger volumes. This variable adds some differences to the previous pDNA studies, but within this self-contained study was kept the same among all pig groups.

### Results:

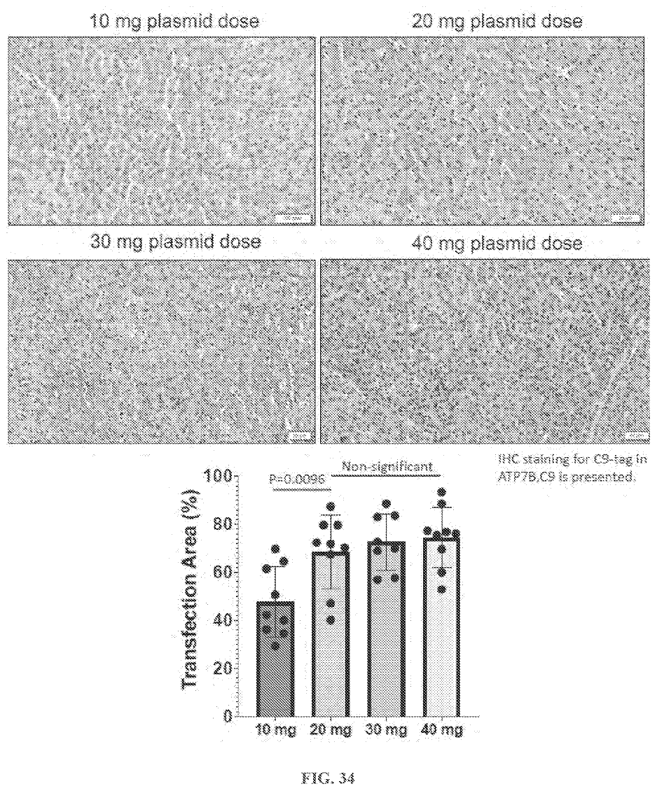

Gene delivery was achieved in all three pigs (FIG. 34). Expression could be detected on immunohistochemistry with the 1D4 antibody. The 20 mg dose exhibited significantly gene expression compared to historical comparisons of 10 mg doses plasmid DNA (47.62% vs. 68.42%, p=0.0096). There were slight increases with the 30 mg plasmid DNA dose (72.65%), but the 40 mg dose (74.43%), although these differences were not significant compared to the 20 mg dose. This suggests an unexpected saturating effect is achieved, where in additional plasmid DNA dose simply goes to the same cells experiencing hydrodynamic pressure.

Looking at the chemistry studies of all the animals after injection, no notable increases and transaminases post-injection were observed. The pigs did not have any notable physiologic changes during injection, including heart rate or blood pressure (data not shown). Furthermore, histology using H&E stain do not show any distorted liver architecture or inflammation. This suggests that the higher plasmid DNA doses were well tolerated.

### Conclusion:

Higher plasmid DNA doses are well tolerated from a toxicity perspective. The higher plasmid DNA doses mediated unexpected levels of increased transfected immunostained area, although this effect is saturable at the highest doses. The saturation level had not been previous reported with biliary hydrodynamic injection, which previously only reported doses of 3 mg and 5.5 mg. The diminishing returns may be due to the physics of the biliary hydrodynamic injection procedure only targeting certain regions. It was noted that optimal transfection from biliary hydrodynamic injection occurs with DNA doses of 20 mg/kg or more. Higher plasmid DNA doses may yield more protein expression within individuals cell, however. This question remains to be resolved in future studies.

### Example 23: Testing Different Flow Rates to Quantify Protein Expression Differences

A previous study using optimized DNA vectors for gene expression (Kruse, G I E 2021) had validated biliary hydrodynamic injection using similar injection parameters to the original published study, namely 30 mL volume at 2 ml/sec flow rate (Kumbhari, G I E 2018). Subsequently, it was found that the biliary system could tolerate significantly higher volume and flow rates than what was previously described (PLOS One. 2021 Apr. 28; 16(4): e0249931). Pressure testing indicated that the flow rate was the predominant driver of pressure with the volume contributing no major role to the pressure achieved within the biliary system.

With that knowledge, high flow rates were tested to determine if they could mediate increased protein expression within cells if possible, along with whether a higher transfected area was appreciated. Without being bound by theory, it is believed that higher flow rates create higher pressure, which would then create more pores within the cell membrane to allow either more DNA to enter inside liver cells, or to allow DNA to enter inside more liver cells, or both.

### Methods:

In order to test the influence of flow rates on the outcome of biliary hydrodynamic injection, it was important to deliver reporter genes that could be readily quantified. Secreted alkaline phosphatase (SEAP) is one example of a reporter gene, which is secreted into serum. The gene is resistant to heat activation, which otherwise kills the endogenous alkaline phosphatase. Upon incubation with a reporter chemical, light is released which can be quantified, the amount being proportional to the concentration of SEAP in the serum. To gauge intracellular reporters, the genes for GFP and firefly luciferase were also introduced. GFP is a convenient marker for IHC detection, while firefly luciferase enzyme concentration levels can also be quantified according to a chemical reaction with luciferin, similar to the SEAP enzyme.

Three pigs were obtained for the study. The information on those pigs is listed below, including weight, DNA dose, and injection parameters utilized.

TABLE 11
Influence of Flow Rates

| Animal ID# | | |
|---|---|---|
| (DNA Payload) | Animal Weight | Injection Parameters |
| Pig #1 (pig SEAP 3.5 mg + | 36.4 kg | 29 ml @ 2 ml/sec |
| GFP-Luc 5 mg) | | |
| Pig #2 (pig SEAP 3.5 mg + | 33.3 kg | 32 ml @ 6 ml/sec |
| GFP-Luc 5 mg) | | |
| Pig #3 (pig SEAP 3.5 mg + | 34.3 kg | 31 ml @ 10 ml/sec |
| GFP-Luc 5 mg) | | |

The pigs were sacrificed three days after the injection, after which serum was obtained as well as tissue samples from all four of the major liver lobes at proximal and distal locations from the point of hydrodynamic injection.

### Results:

During the injection in Pig #3, the balloon slipped backwards during the injection slide from the common hepatic duct and into the common bile duct, allowing the injected solution to pass into the cystic duct. This resulted in the loss of pressure and a failed hydrodynamic injection. This animal was therefore not taken for any further analysis.

Concerning the other two pigs, serum testing for SEAP was undetectable in both pigs. This suggests expression was not at sufficient levels for detection, or that secretion of the protein did not occur. The latter would be somewhat surprising since an endogenous pig sequence was utilized.

For tissue expression, GFP and firefly luciferase could be detected by immunohistochemical stains. The pattern of expression was similar to a previously published study with prominence in zone 3 of hepatic lobules. The firefly luciferase concentrations were tested by protein expression and luciferase assay. Higher levels of firefly luciferase activity were observed at the 6 mL/sec flow rate versus the 2 mL/sec flow rate.

### Conclusion:

Higher flow rates may contribute to more total protein production, but this needs to be verified with more animals and analyzing other flow rates. Secreted reporter proteins continue to struggle to be detected, which could be due to insufficient protein expression

### Example 24: Validation of Novel Reporter Genes in Pig Liver After Biliary Hydrodynamic Injection

Previous studies validated biliary hydrodynamic gene delivery using human FIX as reporter genes (Kruse, G I E 2021). The challenge with human FIX as a reporter gene, however, is the potential for endogenous cross-reactivity with endogenous pig FIX. This makes detection by immunohistochemistry challenging. An additional problem was that human FIX, despite being a secreted protein, could not be detected in the pig plasma. It was hypothesized that inter-species differences could be the limitation.

To solve these two issues it was necessary to test the delivery of new reporter genes by biliary hydrodynamic injection. To address the issue of more specific IHC staining, proteins such as GFP and firefly luciferase were tested that are not naturally expressed in pigs. There should be, optimally no cross-reactivity with endogenous pig proteins, so that the staining could be more accurate. In a separate experiment, a potential human therapeutic protein was synthesized, but appended two different protein tags to aid in specific detection. Specifically, the human PAH gene was chosen for study, which is deficient in the disorder, phenylketonuria. An n-terminal His-tag was appended to allow for efficient protein purification and attached a c-terminal C9-tag to aid in IHC staining.

While IHC staining can give an idea of delivery efficiency into different cells of the liver, a secreted reporter gene would be ideal, since it could be readily quantified in the pig serum or plasma. This would allow for long term studies of gene expression in pigs, as well as quantification of gene delivery efficiency when different injection parameters are modulated.

With the goal of finding secreted reporters, three different reporter proteins were tested. The first is a Gaussia luciferase, which is naturally secreted and very compact in size. Gaussia luciferase furthermore exits the cell via a non-traditional pathway, which could be less influenced by the endogenous pig mechanisms. Furthermore, Gaussia luciferase is not glycosylated, so any differences between species in ER and Golgi apparatus processing should also not affect it. Gaussia luciferase also has the advantage as well of being the most sensitive reporter protein, approximately at least 100 to 1000 times more sensitive than secreted alkaline phosphatase.

The secreted alkaline phosphatase (SEAP) reporter gene has the advantage of the sequence being able to be generated from a pig, by finding a protein homologous to human placental alkaline phosphatase, which is what the original SEAP reporter is based on. The SEAP reporter is similarly quantitative and secreted in the serum like Gaussia luciferase but would have the advantage of being more amenable for long term studies of gene expression, since there would be no immune response to it. A similar dog version of SEAP, canine SEAP, was produced previously to monitor gene expression in that animal model long-term (Hum Gene Ther. 2017 Jul. 1; 28 (7): 541-550.)

As mentioned above, previous work attempted delivery of human factor IX into pig liver (Kruse, G I E 2021). human factor IX expression was observed throughout the pig liver, at relatively high levels of delivery efficiency into many hepatocytes. However, human factor IX in the pig plasma was undetectable. This is unusual, since so many hepatocytes appeared to be expressing human factor IX in the pig liver. With the goal of creating a quantitative reporter and gaining insight if the levels of human factor IX would be sufficient for clinical relevance in hemophilia B patients, it the overexpression of a protein that would be very similar to the endogenous pig factor IX was explored. The strategy was to create a pig factor IX sequence and append a c-terminal tag and express the gene inside pig hepatocytes. The pig hepatocyte should be able to express and secrete pig factor IX with tag, since that is the cell-type that normally expresses pig FIX. For this strategy, a his-tag to the C-terminus of pig Factor IX was appended. His-tags have previously been used to help purify recombinant factor IX protein without affecting its function, so shouldn't interfere with function or secretion. Conveniently, there are his-tag ELISA assays available for the detection of his-tagged proteins secreted into the supernatant. In this way, pig factor IX levels produced by gene therapy could be indirectly measured. Usefully, there are human Factor IX recombinant proteins with His-tags that could be used as standards for even more accurate quantification.

### Methods:

Towards validating the different reporter genes, four pigs were obtained. The size of the pigs, their respective liver weights, and the injection parameters are provided below. The DNA dose is provided next to the gene name.

TABLE 12
Validating Reporter Genes

Animal ID#

| (DNA Payload) | Animal Weight | | Liver Weight | | Injection Parameters |
|---|---|---|---|---|---|
| Pig #1 | 30.1 | kg | 889.6 | grams | 40 ml @ 2 ml/sec |
| (Gaussia Luc-IRES- | | | | | |
| GFP, 10 mg pDNA) | | | | | |
| Pig #2 | 27.7 | kg | 653.9 | grams | 36 ml @ 2 mL/sec |
| (Human PAH, 10 mg | | | | | |
| pDNA) | | | | | |
| Pig #3 | 31.7 | kg | 782.9 | g | 32 ml @ 2 ml/sec |
| (pig SEAP, 10 mg | | | | | |
| pDNA) | | | | | |
| Pig #4 | 40 | kg | 983.5 | g | 36 ml @ 2 ml/sec |
| (pig FIX-His + GFP- | | | | | |
| Luc, 6.5 mg pDNA) | | | | | |
| Pig #5 | 54 | kg | 1313.5 | g | 30 ml @ 4 ml/sec |

(pClucf, 10 mg pDNA)

For all pigs the ERCP procedure was executed without incident. The power injector was able to inject the full dose DNA at the designated parameter without issue.

### Results:

The serum of the pig injected with pig secreted alkaline phosphatase (SEAP) had no detection of this protein by chemiluminescence assay. This represents a failed experiment. Of note, pig SEAP was readily detected after hydrodynamic injection in mouse liver.

The ELISA for the His-tag on pig Factor IX also failed to detect the presence of any protein in the pig serum. This represents a failed experiment. Of note, previous studies from in vitro cell culture also failed to detect this protein, so it may also be an issue of the assay.

The Gaussia luciferase assay also failed to text any luminescence activity within the pig serum. Control serum from mouse injected with Gaussia luciferase was successful in detecting the enzyme. Suggests that expression levels were below the detection limit in pigs.

The PAH protein was successfully detected on immunohistochemistry staining of the pig liver using the 1D4 antibody, which targets a C9-tag added to the human PAH gene. Interestingly, the IHC staining pattern was relatively punctate within hepatocytes for the PAH enzyme, which is different than the staining pattern appreciated with the same antibody. It's uncertain if this localization is natural for the PAH protein, or if it has to do with the his-tag or C9-tag modifying the location of the protein within the cell.

The pCLucf plasmid was able to show expression of GFP and luciferase in multiple cell types using each antibody. The preferences of the CMV and SV40 promoter driving expression of luciferase and GFP were slightly different in each cell type. Hepatocyte staining was relatively light for both GFP and luciferase, and concentration either around portal triads or the central vein. The multiple cell types observed harboring expression included bile ducts, endothelial cells, neurons, and light staining in fibrous tissue.

### Conclusion:

The three secreted reporter genes tested all failed to show any activity or visible detection in pig serum and plasma. Together, this suggests that fundamentally all proteins are not produced at high enough levels to reach a detectable threshold in the serum or plasma. The tentative hypothesis is that the hydrodynamic delivery of plasmid DNA does not result in the expression of secreted reporter proteins by mechanisms that are unclear.

The PAH gene was detectable on immunohistochemistry. It is uncertain if the amount produced would be sufficient to reverse PKU. Further studies in PKU animal models will be necessary to assess this. The results with GFP and luciferase offer promise for targeting other cell types such as bile duct cells and endothelial cells, which are clinically relevant.

### Example 25. Validation of the Delivery of Human ATP7B into Pig Liver

The initial study investigated the delivery of human factor IX into pigs as a potential model for hemophilia B treatment. The next goal was to deliver human ATP7B into pig liver through biliary hydrodynamic injection. ATP7B is the gene, which harbors the causative mutation for Wilson's Disease. Wilson's Disease is a potential candidate disease as a first indication for biliary hydrodynamic injection. The challenge of ATP7B is that it is significant larger than human Factor IX, being ~4.4 kb compared to ~1.3 kb for human Factor IX. Thus, the total plasmid DNA size increases from 5.5 kb to 8.6 kb that would need to be delivered by biliary hydrodynamic injection.

It's uncertain how the additional size of the plasmid would affect the gene delivery efficiency observed in the liver.

ATP7B is naturally located in the Golgi apparatus of cells and can present with a cytoplasmic stain. Upon immunofluorescent staining, a speckled pattern across the cytoplasm is appreciated. Because the human ATP7B is similar to the pig ATP7B and available antibody reagents against ATP7B are not characterized for their species specificity, an approach was chosen of appending a detection tag to human ATP7B, which would be specific to immunostaining within pig liver. For this purpose, the C9-tag was chosen, which is a common c-terminal tag for transmembrane proteins, which is useful in detection and purification. The C9-tag is readily detected by the 1D4 antibody, and the only cross-reactive proteins are found in the retina, since the protein sequence comes from the protein rhodopsin.

### Methods:

With the goal of validating the non-viral gene delivery of ATP7B in a large animal model for the first-time, the plasmid, pT-LP1-ATP7B,C9 was constructed. The plasmid is based off of the previous vector, pT-LP1-hFIX, which was published previously (Kruse, G I E 2018). The human FIX gene was removed and the human ATP7B sequence was put in its place. The sequence of the human ATP7B gene is the same as its native cDNA. The nine amino acids of the C9-tag are located after the final amino acid of ATP7B, and were added by PCR extension.

The plasmid, pT-LP1-ATP7B,C9, was prepared by gigaprep for injection into pigs, and diluted into normal saline solution. Two pigs were selected for pilot studies. Their total weight, liver weight and injection parameters are listed below.

TABLE 13
Plasmid ATP7B prepared by gigaprep

| Animal ID# | Animal | Liver | Injection |
|---|---|---|---|
| (DNA Payload) | Weight | Weight | Parameters |
| Pig #1: | 49.3 kg | 1085.6 g | 40 ml @ 4 ml/sec |
| (ATP7b, dose 10.9 mg) | | | |
| Pig #2: | 41.3 kg | 1071.3 g | 33 ml @ 2 ml/sec |
| (ATP7b, Dose 9.8 mg) | | | |

Pigs were harvested at day 1 to 3 after injection, all 4 of the major lobes were sampled, including the right and left lateral and medial lobes. Pig tissue was fixed in formalin and embedded in paraffin wax before staining.

### Results:

Both pigs exhibited immunostaining for ATP7B. The IHC stain was primarily around the central vein of the hepatic lobule in zone 3. Several lobules interestingly almost had 80-90% expression within the hepatic lobule. All hepatic lobules exhibited immune staining. The efficiency appeared to be on average 30-50% of total hepatocytes. The transfected area data are used as a comparison point for other studies regarding dosage, redosing, and persistence of expression.

### Conclusion:

Human ATP7B can successfully be delivered into pig liver. The pattern of gene delivery matches the previous experience with human FIX. The overall transfected stained area also appears to be comparable to previous studies, which was reported to be 30-50% of hepatocytes in the hFIX publication (Kruse, G I E 2021).

### Example 26-Feasibility of the Procedure in a Non-Human Primate Model

### Rationale:

Previous studies on biliary hydrodynamic injection were all conducted exclusively in pig models. While the pig liver is very similar in size to human liver, there could be differences between the species. This could be macroscopic differences, or alternatively differences in the microscopic histology that cause differences between species. On a larger scale, animals of similar size, such as a 30 kg pig versus a 30 kg baboon may still exhibit significant different sizes and torsos. Moreover, the size of the duodenum and proximity of the biliary orifice with the stomach sphincter are also different across species. To understand if ERCP could be executed in a new animal model then, basic feasibility must be assessed.

### Methods:

Male baboons weighing approximately 30 kg were obtained. A duodenoscope from Fujifilm was advanced through the mouth of the baboon, into the esophagus, stomach, and into the duodenum. The biliary orifice could be visualized on the duodenal wall. In the baboon, the pancreatic duct also opens into the same orifice, increasing the potential risk of inadvertently advancing a guidewire into the pancreas and potentially leading to pancreatitis. With further maneuvering, a guidewire can be advanced into the common bile duct (CBD) and subsequently the common hepatic duct (CHD), mirroring the process observed in pigs. A prominent difference between pigs and baboons is the noticeably wider diameter of the CBD in pigs compared to baboons. Both CBD in each species is relatively long compared to human patients, with the baboon CBD length being especially long.

The CHD length in pigs is 4-5 cm, as noted in our other work. The CHD in baboons, however, is surprisingly short, measuring only ~1.5 cm in length. This is despite similar size animals being used across different experiments. The technical challenge with the CHD length is that the balloon on the catheter is itself about 1 cm in length, approaching the total CHD size. Thus, the balloon sits in close proximity to the cystic duct.

Utilizing fluoroscopy to capture a cholangiogram of the entire biliary system, the biliary tree also appears to be very different in the baboon model, versus prior experiments in pigs and humans. The baboon has an early right hepatic duct, servicing the right lobe of the liver. The left branch later splits into a middle hepatic duct servicing the middle lobe, while the left hepatic duct branches into the left lobe. These main branches subsequently split into many different minor branches which service individual lobes

After positioning the catheter into the CHD, the catheter (Olympus Multi-3V) could be connected to a power injector (Mark V Provis, MedRad). Upon hydrodynamic injection at a given setting (greater than or equal to 2 mL/sec or 30 mL in volume), fluid can be observed to enter into the biliary system. This is observed either by flushing existing contrast solution out of the ducts present, or with the hydrodynamic injection of contrast solution. The latter can be appreciated to acinarize (contrast entry) into the entire liver parenchyma.

Post-injection, repeat fluoroscopic imaging of the biliary anatomy (cholangiogram) was obtained. The imaging in all animals revealed that the biliary system was intact. Thus, primate liver can handle similar pressure and stress to pig liver.

### Results:

Successful canulation of the biliary orifice and guiding of the guidewire into the biliary system could be achieved in all animals (FIG. 39). Sphincterotomy was not required for cannulation in the baboon model. Fluoroscopy illustrates the anatomy of the biliary tree in baboons, showing an early right branch and two main branches on the left side. A small common hepatic duct is appreciated in baboons, which is entirely extrahepatic from the liver. As shown by fluoroscopic images pre- and post-injection, primate liver, represented by baboons, can tolerate high-pressure injection into the biliary system without rupture. Additional safety testing was pursued, trending values from pre-injection through day 7 post-injection (FIG. 40). Liver enzyme elevations resolved within 7 days, while there no were hematological differences. Pancreatic enzymes tested revealed no signs of post-ERCP pancreatitis.

### Conclusion:

Biliary hydrodynamic injection is feasible in primates as it is in porcine liver, with similar execution and steps for gene delivery. However, there are several important differences and necessary procedure modifications, which are outlined in subsequent examples.

### Example 27-Bile Duct Walls in Non-Human Primates Exhibit Increased Elasticity

### Rationale:

A major requirement for successful hydrodynamic injection into the biliary system of baboons is successful generation of pressure. Pressure generation during hydrodynamic injection is thought of as an equilibrium between the exit of the saline solution between tight junctions of hepatocytes from biliary canaliculi into the hepatic sinusoids and out to the hepatic vein, versus the pressure from the input force of high flowing fluid into these very narrow channels and network. The fluid pressure upon leaving the catheter is thus very high, since immediate resistance from the tissue is observed. Given the high fluid pressure, the injection solution will immediately exert pressure on all surfaces as it finds the lowest resistance to escape.

Key to this establishment of pressure is that the fluid can both flow in a forward direction into the liver, but also flow in a backward direction toward the gall bladder, common bile duct and small intestine. The latter tissues, in particular, are surrounding by soft fibrous tissue that allows flexibility in the movement of organs, or in the case of the gallbladder, free movement within the peritoneal cavity. In order to stop the egress of fluid in all directions, a balloon is used to stop any antegrade flow of fluid, such that there is only retrograde flow into the liver. The balloon on these biliary stone extraction catheters is typically soft and filled with air to expand in size. Depending on the surrounding tissue, they can inflate to their defined and intended shape.

### Methods:

Male baboons weighing approximately 30 kg were obtained. Biliary hydrodynamic injection procedures were conducted as described in Example 26. To monitor efficient seal of the balloon, contrast solution was filled above and below the balloon prior to injection. Alternatively, the hydrodynamic injection proceeded with the injection of contrast solution. Live fluoroscopic imaging during the injection commenced in order to monitor the integrity of the balloon during injection. The key observations were if any saline solution of contrast solution moved around the balloon and into the antegrade direction. To confirm findings, some experiments used blue dye mixed in with the saline or contrast solution.

### Results:

After proper positioning, the bile ducts were approximately 4-5 mm in size in the baboon. In all injections, a balloon size of 8.5 mm was utilized, which could be seen on fluoroscopy to expand the width of the duct. Given that the diameter of the duct was physically sealed, the assumption of efficient pressure seal was assumed.

Injection proceeded at injection parameters of flow rate, 3-4 mL/sec, and 30-40 mL in volume across several animals and replicate experiments. Surprisingly and consistently, there was an observation that fluid could escape around the balloon and proceed in the antegrade direction. Fluid could both be seen entering the gallbladder as well as the common bile duct as assessed by live fluoroscopic imaging (FIG. 41). When blue dye was used, blue dye could be appreciated exiting the biliary orifice in the small intestine, as observed with the endoscopic camera.

To understand the problem, review of the fluoroscopic video during the liver injection was reviewed. This demonstrated that the cause was the widening of the bile duct walls, which were already hugging around the balloon's surface. This widening of the duct walls proceeded around the entire balloon during injection.

Another observation is that upon review of the cholangiogram for the entire liver, all bile ducts appeared to increase approximately 2-fold in diameter post-injection, both extrahepatic and intrahepatic. This suggests a more compliant, elastic tissue in primate liver.

### Conclusion:

An unexpected challenge in biliary injection in primates was appreciated. The bile ducts appear to have increased elasticity compared to pigs. Thus, where a specific balloon size in pigs could be used successfully for injection, that same size may not be sufficient for pressure seal in primates.

The result of this problem is that pressure could be rapidly lost during injection, not to mention the loss of solution containing DNA into these different compartments. In order to solve this problem, modifications are necessary in the procedure for primates, as outlined in the subsequent examples.

### Example 28-Increases in Balloon Size and Intrahepatic Positioning Decrease the Rate of Balloon Leak

### Rationale:

The previous experiment demonstrated the unexpected finding that primate bile ducts have increased elasticity compared to porcine bile ducts. This has major implications for the efficiency of biliary hydrodynamic injection, considering that significant leak results in loss of pressure, along with loss of DNA solution. Thus, a new procedure protocol must be established that would solve this issue and result in efficient injections.

Considering the possibilities, further consideration of the biliary system demonstrates that a component of the bile ducts exists in the extrahepatic biliary system, which comprises the common bile duct, cystic duct, gallbladder, and part of the common hepatic duct. The rest of the biliary system is considered as the intrahepatic biliary system, which consists of the remaining common hepatic duct before it bifurcates into left and right branches inside the liver, with the resultant progressively smaller biliary branches. The extrahepatic biliary system is surrounded the loose fibrous tissue, while the intrahepatic biliary system is surrounded by the liver parenchyma.

### Methods:

Male baboons ranging in size around 30 kg were obtained. Biliary hydrodynamic injection procedures were conducted as described in Example 26 with the following modifications. To attempt to counteract the increased elasticity of the primate biliary system, the increasing balloon sizes were tested. Balloon sizes of 11.5 mm and 15 mm were tested for this purpose as exemplary sizes.

In addition, intrahepatic positioning of the balloon versus extrahepatic positioning of the balloon was compared. Specifically, the extrahepatic biliary system, corresponding to the CHD, as well as the intrahepatic biliary system, corresponding to the left biliary branch were evaluated. This would test if the bile ducts were more elastic in the extrahepatic system, which is the traditional location for biliary injection, and/or if the bile ducts in the intrahepatic system were less elastic. Of note, it was uncertain and/or how the balloon would react to inflation within the intrahepatic biliary system.

### Results:

Increasing the balloon size to 11.5 mm in the extrahepatic CHD was tolerated in both animals. However, efficient seal was only intermittently obtained in this location, with leakage still noted at some points. This was particularly true if the balloon itself appeared to be in close proximity or overlap with the cystic duct of the animal.

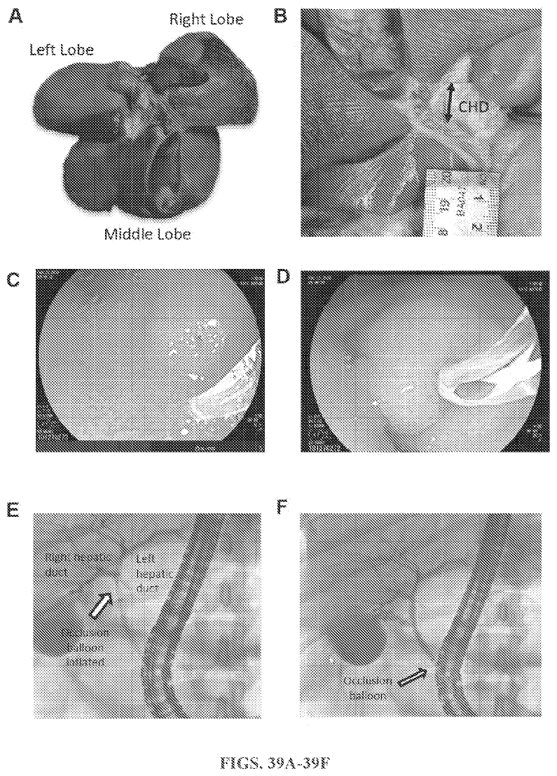

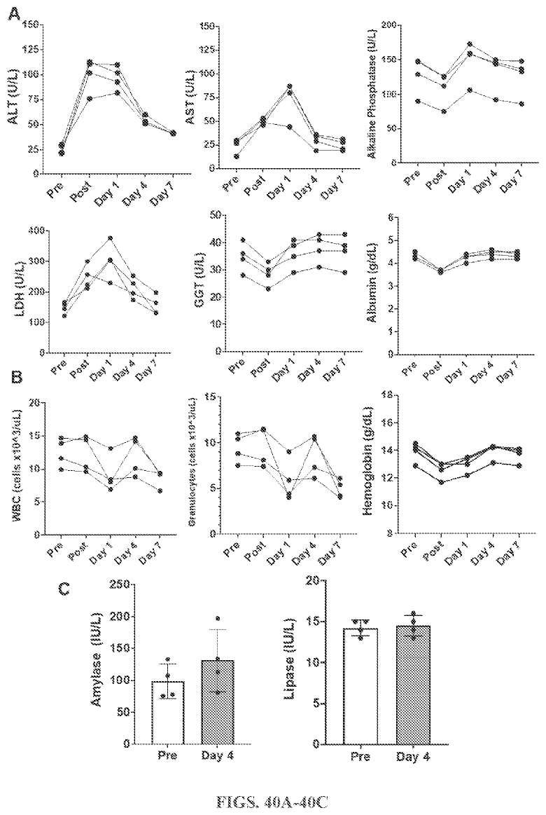

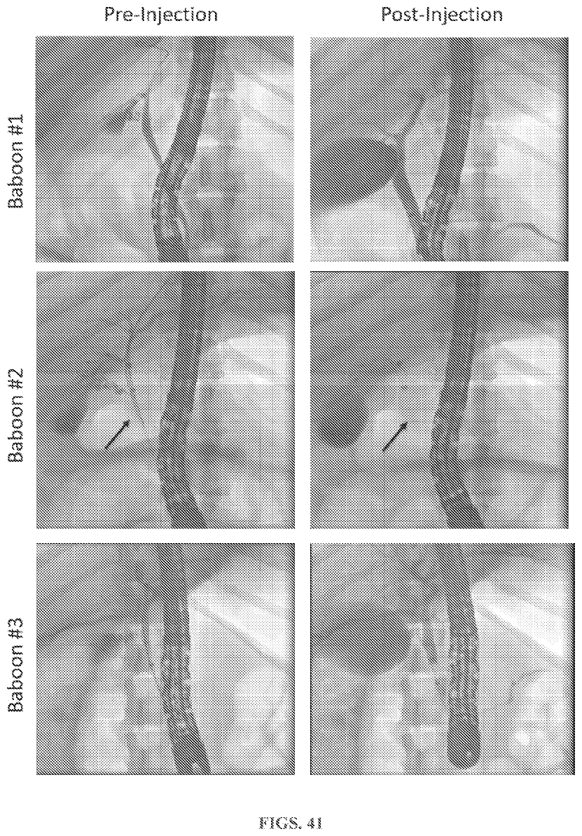

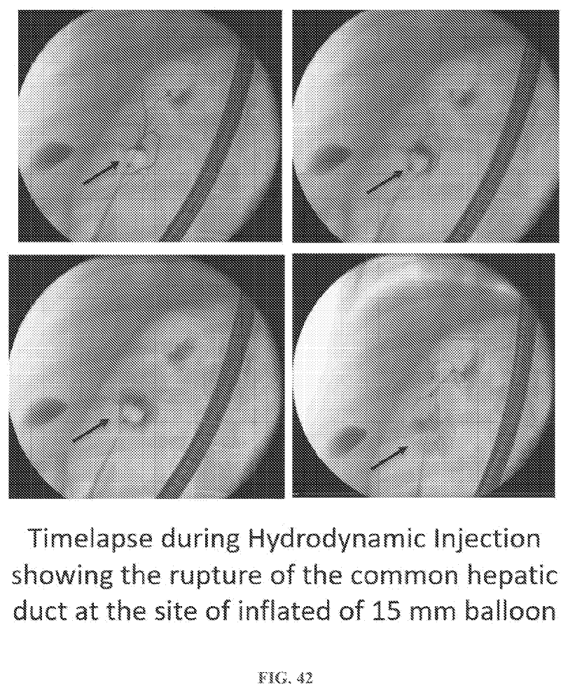

Further increasing of the balloon size to 15 mm led to rupture of the extrahepatic CHD, demonstrating that there is a function limit to elasticity of the extrahepatic bile ducts between 11.5 to 15 mm of the balloon (FIG. 42). A potential concern with this approach is that the balloon kept on getting wider in this location, almost matching inflation when it is done outside the balloon. In an ex vivo experiment, the shape of the extrahepatic CBD could be appreciated to stretch around the entire surface of the balloon. This both indicates that the extrahepatic bile ducts are extremely elastic, but they may also not tolerate additional pressure. Concerning the 15 mm balloon size, it's possible the stretch by the balloon caused the tear, or the stretch plus the pressure from the injection caused the tear. Regardless, the bile duct tear was at the site of the balloon, supporting that fluid pressure upstream is not a major factor to biliary tears.

In further experiments, the balloon sizes were compared in an intrahepatic location within the baboon. In these experiments, it was observed that 8.5 mm, 11.5 mm, and even 15 mm balloons could all be inflated within the intrahepatic ducts. However, while the air amount corresponding to those balloon sizes could be injected into the catheter, the actual size inside the intrahepatic system was less than the stated diameter. This observation would support that the hepatic parenchyma provides significantly more resistance to balloon inflation. Given the amount of injected air is equivalent, the actual air pressure inside the balloon must be larger within intrahepatic locations.

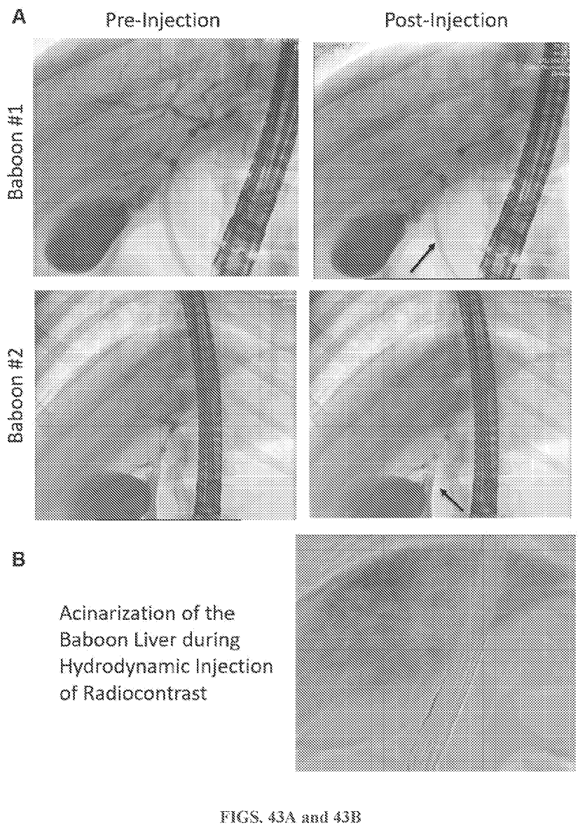

During hydrodynamic injections at intrahepatic locations, it was noted that even an 8.5 mm balloon in a very small duct, 2-3 mm, could potentially still have some leak around it, although this observation was not consistent across experiments. A larger balloon at this intrahepatic location of 11.5 mm appeared to consistently hold seal, with no appreciable leakage of saline or contrast noted during live fluoroscopy during hydrodynamic injection (FIG. 43). The same findings were true with a 15 mm balloon size.

### Conclusion:

In primate liver, extrahepatic injection locations carry a higher risk of fluid leak around the balloon during injection. This can partially be overcome by increasing the balloon size to at least 11.5 mm, but still may have leak appreciated in certain animals. Larger balloon sizes of 15 mm or greater risk the potential of overstretching and tear of bile ducts, however.

By contrast, intrahepatic injection appears to be safer, with a wider range of balloon sizes tolerated. Moreover, the pressure seal was achieved more consistently in animals at sizes greater than 8.5 mm. Thus, injections within the intrahepatic CHD or in individual biliary branches may be more preferable in primate liver, including human patients.

### Example 29-Stable Pressures Can Be Achieved During Biliary Injection When Proper Balloon Seal is Obtained

### Rationale:

The observations of increased elasticity of primate bile ducts have important implications for the ability to conduct biliary hydrodynamic injection in human patients. The prior studies in other examples evaluated the potential for fluid leak around the balloon when assessed by monitoring contrast flow or saline flow into static contrast in the bile ducts. A related measurement toward this efficacy would be the monitoring of pressure during injection. Pressure during hydrodynamic injection should reach an equilibrium point, represented a stable plateau pressure. This equilibrium is a balance between the force of fluid entering the liver, the resistance imposed by the liver, and the exit of fluid from the liver into blood circulation. This observation of plateau pressure was consistently observed in multiple different pig experiments of hydrodynamic injection. Any additional factors or loss of pressure should disturb the pressure curve and be interpreted as a potential balloon leak or other interference in this system. The current experiment will study how pressure curves are modulated during biliary injection in primate liver, according to the location and balloon size.

### Methods:

Male baboons ranging in size around 30 kg were obtained. Biliary hydrodynamic injection procedures were conducted as described in Example 26 with the following modifications and clarifications. In these experiments, the injection of the saline DNA solution proceeds through the guidewire port of the catheter, which has the largest diameter. The injection port, normally used for contrast injection, was connected to a pressure transducer. The pressure transducer is a small, portable, disposable device that measures pressure with the use of a fluid-filled water column. The water column extends from the proximal end of the catheter at the user handle and goes all the way to the side-facing distal tip of the catheter. The water column is in direct communication then with the fluid in the bile ducts. When there is increased pressure in the biliary system, that water pressure would extend both into the liver, as well as into the lumen of the catheter and eventually to the pressure transducer.

As executed before, the hydrodynamic injection was performed at a range of different volumes and pressures, all above 2 mL/sec and 30 mL of volume, respectively. Pressures were recorded during the injections and graphed subsequently to demonstrate time versus pressure.

### Results:

Initial injections were employed in the extrahepatic CHD, wherein the balloon size of 8.5 mm. During these injections at greater than 3 mL/sec, it was noted that an initial peak pressure greater than 80 mmHg could be obtained, but that this would rapidly decrease over time in a linear to exponential manner. A final plateau pressure between 40-60 mmHg would be obtained, or occasionally no stable pressure would be obtained, and the injection would end with pressure still gradually declining.

Slower injections at approximately 1-1.5 mL/sec did not exhibit this phenomenon, with a rapid rise, stable plateau, and then rapid decline in pressure when the injection stopped. However, in this instance, only a stable pressure of 40-50 mmHg was obtained, which is generally much lower than the stable levels achieved during hydrodynamic injection in pigs.

A multitude of attempts were pursued to obtain stable pressure. The first strategy consistent of injecting at faster speed and larger volumes. The rationale of the former is that flow rate generally correlates with higher pressure in pigs. The rationale for the latter is that while the biliary system is not truly closed given the vascular escape that occurs, the small size of the biliary system could potentially be overwhelmed with a massive, large influx of volume into the liver. This could generate more pressure.

The different strategies and results in FIGS. 44A-44B are summarized as follows. A first animal was injected at 6 mL/sec and 60 mL at the extrahepatic CHD with a balloon size of 8.5 mm. The curve, however, looked almost identical to an injection at 3 mL/sec and 30 mL. Another animal injected at 8 mL/sec and 80 mL at the extrahepatic CHD with a balloon size of 8.5 mm also replicated the curve with a loss of pressure and no plateau reached.

The different strategy of multiple escalating flow rates was also attempted in FIG. 44C, wherein flow rates would be increased in the middle of the injection. This strategy also failed to generate significant increases in pressure, although more stable plateau pressure could be generated around 80 mmHg when the second high flow rate occurred. This demonstrates for the first time in primates that flow rate is the key determinant of pressure observed.

Upon review of fluoroscopy in all these experiments, the loss of plateau pressure always seemed to correlate with visually apparent leakage of fluid around the catheter, as demonstrated in FIG. 41. Thus, the two appear to be connected mechanistically as a route cause of failure to replicate the approach from pigs into primates.

Because of these initial failures, more strategies were attempted. The findings from a series of studies conducted to optimize balloon size and placements outlined in the other examples were used to test the hypothesis if better balloon seal would lead to stable pressure generation. In this instance, a series of hydrodynamic injections were tested across three male baboons, the data for two of whom are down. The tip of the catheter was placed in the left hepatic branch of the intrahepatic biliary system of each animal. The balloon was inflated similarly in the location at a size of 11.5 mm. Injection parameters proceeded at 4 mL/sec of flow rate and 40 mL of volume. All three animals were able to achieve stable pressure plateaus ranging from 90-125 mmHg (FIG. 44D). Pressure slowly declined after cessation of injection back to baseline pressure. Thus, similar pressure magnitudes and pressure curve shapes can be produced in primate liver compared to pig liver, when appropriate balloon seal occurs.

### Conclusion:

Balloon seal at the site of the injection is the primary determinant of pressure achieved during biliary hydrodynamic injection. Pig bile ducts are apparently easier to generate balloon seal than in primate bile ducts by comparison, which is why pig experiments readily yield stable pressure plateaus in most circumstances. However, optimization of the approach in primate bile ducts can achieve similar pressure plateaus to pigs, at magnitudes that are sufficient for gene expression. It is noteworthy, however, that a larger flow rate (4 mL/sec) in baboons still generated lower stable pressure (90-120 mmHg in primate vs >150 mmHg in pigs) than similar experiments (3 mL/sec) in pigs, suggesting that intrinsic tissue elasticity beyond the balloon site may modulate the pressure achieved. The larger implication is that efficacious flow rates need to be detailed for primate liver, and that pig findings may not necessarily cross over.

### Example 30-Escalating Flow Rates and Step-Wise Functions are Feasible in a NHP Model

### Rationale:

All the studies described so far in biliary hydrodynamic injection have employed a single flow rate and programmed volume for the entire injection. The feasibility of executing multiple different flow rates and volumes within a single continuous injection have not been explored, nor how that would affect the flow rate.

### Methods:

Biliary hydrodynamic injection was conducted as previously described with the catheter placed into the CHD as a male baboon, weighing around 30 kg in size. In one example, the initial flow rate was set at 3 mL/sec and 30 mL of volume, with a subsequent flow rate set at 8 mL/sec and 61 mL of volume (FIG. 44C). In another experiment, four successive flow rates were designated, 4 mL/sec, 6 mL/sec, 8 mL/sec, and 10 mL/sec each for 3 second intervals. The main outcome was the pressure curves achieved with the escalating flow rates to indicate impact of change, as well as post-injection cholangiogram to show that there was no biliary rupture.

### Results:

Multiple different flow rates could be executed during hydrodynamic injection in an NHP system, with pressure curves reflecting changes from the transition to a new flow rate in one of the animals, while the other animal served to maintain a steady rate of pressure (FIG. 44C). Post-injection fluoroscopy showed no rupture of the biliary system to indicate additional stress or danger from an abrupt change in flow rate.

### Conclusion:

Step-wise functions during hydrodynamic injection are feasible with no anatomical disturbances or risk to the bile ducts of primate liver during injection. Moreover, step-wise injections can have meaningful effects on intraductal pressures that can be of potential utility when designing different injection schemes.

### Example 31-Primates Tolerated Repeated Procedures with the Similar Peak Expression Noted Each Time

### Rationale:

An important feature of non-viral gene delivery is the potential for redosing. Redosing of viral vectors carries significant immunological risk because of potent B-cell and T-cell responses that develop against the viral vector. This is prominently seen with adeno-associated viruses (AAV), wherein high-titer antibody responses develop after a single administration of AAV gene therapy. Furthermore, T cell responses can develop months after administration, targeted remaining capsid protein and eliminating gene-expressing cells.

A major advantage of biliary hydrodynamic gene delivery would be the potential for redosing of the genetic material, as executed through repeat procedures in the animals. There are several potential challenges to this, however, including whether repeat injections would cause any anatomic distortion to the biliary system, as well as if the injection of naked DNA would lead to inflammation, toxicity, or transcriptional imprinting that would prevent additional doses of DNA substance from yielding expression at comparable levels to the original injection.

Another experiment in pigs outlined in a different example in the patent disclosure established the proof-of-principle and feasibility of gene delivery into that species. The experiment demonstrated that a repeat procedure could yield detectable gene expression on the tissue level. Moreover, the efficiency of tissue expression (number of cells transfected by DNA and expressing a reporter gene) was similar from the first gene injected to the second gene injection a month later.

However, a quantified amount of gene expression was not assessed in this experiment, leaving it as an unknown if repeat biliary hydrodynamic procedures could reproduce similar levels of protein. Moreover, given that the immune systems of animals, particularly the innate, differ markedly across species, it's an unknown if the repeated exposure to naked DNA would cause significant changes to the baboon that would make it resistant to infection. Moreover, the potential for repeated procedures to induce adaptive immunity in a primate model is unknown. Finally, it's unknown if more than 2 procedures would yield equivalent expression and be tolerated. The goal of the experiment was to address these questions in a primate liver, which would assure translatability into human patients.

### Methods:

A cohort of male baboons ranging in size around 30 kg were obtained. Biliary hydrodynamic injection procedures were conducted as described in Example 26. Similar injection location, balloon size, DNA doses, and injection parameters were used across experiments. A reporter plasmid DNA of the human FIX (hFIX) gene was injected under the control of a liver-specific promoter, LP1. ERCP procedures were repeated in the same animals across approximately 1-month intervals (ranging from 4-6 weeks). An ELISA kit that distinguishes baboon FIX from hFIX was utilized (LSBio).

### Results:

A series of 3-4 hydrodynamic injections was conducted several months. A single animal received a total of 4 hydrodynamic injections. The peak expression obtained was similar each time, reaching ~8 ng/mL hFIX at each time point (FIG. 45B). There were no differences in liver chemistries between each injection to suggest additional toxicity with subsequent procedures. Cholangiograms within the same animal across different procedures also looked identical, suggesting that there are no differences or disturbances to the biliary anatomy with repeat procedures.

### Conclusion:

Repeat ERCP-mediated procedures delivery DNA into the liver are well tolerated in primates, and identical or greater expression can be obtained from subsequent procedures. Together, this suggests each gene delivery procedure acts independently and each can anticipate similar expression as long as the other parameters of the procedure are the same. These results are novel for the field of biliary hydrodynamic gene delivery.

### Example 32-Tissue Pattern of Gene Delivery in Primates Matches Pigs

### Rationale:

A key feature of biliary hydrodynamic gene delivery is the particular feature of high efficiency transfection into hepatocytes. The network of bile ducts and bile canaliculi effectively deliver DNA throughout the hepatic lobule, but especially as the terminus of the canalicular system around the central vein of the hepatic lobule. As a result, a previous publication by our group demonstrated that delivery efficiency can reach 50% of hepatocytes.

A central question remains if this pattern and efficiency can be replicated in primate liver versus pig liver. Pig liver has distinguishing features from primate liver, which makes it uncertain if the mechanism would be the same. Pig lobules have fibrous tissue surrounding the lobule, which separates them from every single over lobule in the liver. As such, it makes it easy to see, even macroscopically with the human eye. What is uncertain, however, is if this fibrous tissue makes a meaningful difference on the outcome of gene delivery efficiency. For example, it's possible that the fibrous tissue makes each individual lobule stiffer, and thus more pressure is microscopically created in canaliculi and around lobular borders for gene deliver. Thus, it is an essential experiment to do biliary gene delivery in primate liver, which lack this fibrous tissue around hepatic lobules.

### Methods:

A male baboon was obtained weighing approximately 30 kg for a short-term study. A reporter plasmid was obtained encoding green fluorescent protein (GFP) under a SV40 promoter and firefly luciferase under a CMV promoter. 50 mg of plasmid DNA was injected into the baboon at parameters of 10 mL/sec and 100 mL. The baboon was euthanized 24 hours later and different liver sections harvested across all three liver lobes. Tissue sections were fixed in formalin for 24 hours, followed by subsequent embedding in paraffin. Slides were prepared and tissue stained by immunohistochemistry with primary antibodies against GFP and firefly luciferase and rat secondary antibodies conjugated to horseradish peroxidase. In addition, tissue sections were analyzed for any morphological differences using standard H&E staining.

### Results:

Reporter gene expression was detected in baboon liver by immunohistochemical analysis. GFP staining was concentrated around the central vein of baboon lobules, similar to the pattern seen in pigs (FIG. 45D). Efficiency was also similar ranging from approximately 30-50% of hepatocytes. There were no histological differences detected from normal baboon liver, including areas of necrosis.

### Conclusion:

The transfection pattern and efficiency in primate liver matches that from pig liver. Thus, the main determination of transfection remains the DNA solution terminating at the ends of the bile canaliculus near the central vein. The fibrous tissue surrounding pig lobules does not appear to play a significant role in the observed transfection efficiency, by deduction. Similar to the experiment presented in a different example, it does not appear that higher flow rates led to increased transfection across species.

### Example 33-Primates Exhibit a Dose Dependent Relationship with Gene Delivery to a Quantitative Biomarker

### Rationale:

An important element of any therapeutic intervention is understanding the dose response relationships of the drug substance to the intended therapeutic effect. The same is true for gene therapy, where most trials study a series of escalating viral vector dosing. The same will also be true for biliary hydrodynamic gene delivery, wherein a series of different DNA doses will be investigated.

In pig studies, the relationship between DNA dose and transfection area was studied, demonstrating that escalating DNA doses can be used to improve transfection area up until a certain point of saturation. This experiment is covered in another one of the examples in the patent disclosure.

In the current experiment, the nature of DNA dose response in primate liver was studied. Primates have a different innate immune system that could be more sensitive to naked DNA administration, as recognized by TLR4 and STING/cGAS pathways amongst others. Thus, the nature of DNA dose respond may not be transferrable across species. More importantly, for many applications the quantitative protein amount is the most important barometer for the therapeutic outcome. Thus, understanding how DNA dose relates to protein expression is critical. It is theoretically possible, for example, that the DNA entry into the nucleus is saturable, such that there would be a limit to expression observed.

### Methods:

For this experiment, starting DNA doses were chosen that are in the range of the saturable DNA doses in pigs, and indeed exceed them when the dose is adjusted on a per baboon liver weight basis. Two male baboons weighing approximately 30 kg were obtained and were subjected to repeat biliary hydrodynamic procedures spaced one month apart. This strategy was chosen to remove the variables of different liver architecture and potential pressure sealing from affecting the delivery efficiency observed. Hydrodynamic injection parameters were kept similar for all injections of the experiment. The first set of experiments were conducted at the 20 mg DNA dose, while the second experiment was conducted at the 60 mg DNA dose. A reporter plasmid DNA of the human FIX (hFIX) gene was injected under the control of a liver-specific promoter, LP1. An ELISA kit that distinguishes baboon FIX from hFIX was utilized (LSBio). Plasma samples were drawn from the baboons on day 1 post-injection and assessed for peak hFIX levels.

### Results:

All biliary hydrodynamic injection procedures were successful in mediating gene expression and detectable hFIX. As summarized in FIG. 45A, with the 20 mg DNA doses, the first animal achieved an expression of 8 mg/mL hFIX, while the second animal achieved an expression of 3 ng/mL. With the 60 mg DNA doses, the first animal achieved an expression of 25 ng/ml, while the second animal achieved an expression of 10 ng/ml. The relative increase in hFIX levels in the plasma in both animals was approximately 3-fold, which matches the 3-fold increase in DNA dose. Of note, there were no differences in the observed toxicity of the injection, as assessed by liver chemistries for AST and ALT, that would otherwise suggest more toxicity from DNA recognition by adaptive immune responses.

### Conclusion:

During biliary hydrodynamic injection, DNA dose can be increased to increase expression of a reporter gene in a quantitative manner. The mechanism of DNA entry is not saturable, thus more DNA injected will lead to more expression. This is an important and unexpected finding since other areas of gene therapy typically do not have standard dose responses and/or can illustrate unexpected toxicities.

### Example 34-Use of Miniaturized DNA Allows Decreased Dose to be Used Versus Regular Plasmid

### Rationale:

A key factor in the efficacy of hydrodynamic injection is the ability to maximize the expression, which can be achieved through escalating DNA doses, as discussed in a prior example in the patent disclosure. However, DNA dose could be potentially restricted by innate immune recognition and other toxicity in the primate liver, as well as inherent limitations in viscosity of DNA solution, wherein increased DNA concentration leads to a more viscous solution, which could have difficult being project through the catheter and biliary system at high speeds without significant sheering.

In order to address this, one strategy would be to leverage DNA molecules with minimal extraneous sequences outside of the expression cassette of interest, miniaturized DNA molecules. A prominent example of this are minicircles, which are circular DNA molecules without any additional DNA sequences beyond a small recombination sequence. Nanoplasmids are another alternative, wherein the bacterial portion of the plasmid has been reduced to less than 500 bp's in size, allowing for significant reduction of the total plasmid DNA size. Further alternatives include ministring DNA and doggybone DNA, which are linear and only have some recognition sites of <50 bp's at either end. In all these cases, the DNA for the expression cassette only as much as possible would be injected by hydrodynamic injection, which should afford more potency per mass of DNA injected since there are more net molecules of DNA delivered.

### Methods:

In order to test this hypothesis, an exemplar DNA molecule with reduced extraneous sequence, nanoplasmid, was injected alongside a regular plasmid. The regular plasmid in this case has an extraneous DNA size of 2.7 kb, while the nanoplasmid extraneous sequence is approximately 0.45 kb. For total size comparison, the NP-LP1-hFIX plasmid is 2.77 kb in size, while the regular pT-LP1-hFIX plasmid is 5.55 kb in size.

For the experiment, equimolar amounts of DNA molecules were injected. When accounting for the relative size between the two molecules, this meant injected 20 mg of nanoplasmid to 50 mg of regular plasmid. For the biliary hydrodynamic injection, all other aspects were kept consistent, including catheter position in an intrahepatic branch, along with the injection parameters.

To test for the relative expression achieved, a reporter plasmid DNA of the human FIX (hFIX) gene was injected under the control of a liver-specific promoter, LP1. An ELISA kit that distinguishes baboon FIX from hFIX was utilized (LSBio). Plasma samples were drawn from the baboons on day 1 post-injection and assessed for peak hFIX levels. The experiment was repeated across two different animals for comparison.

### Results:

As summarized in FIG. 45C, test results taken at day 1 post-injection demonstrated that the nanoplasmid group yielded an expression of around 4 ng/mL on average. This almost equaled the average of the regular plasmid group, which was also around 5 ng/mL on average. When adjusted for the DNA mass dose, these values are not statistically different.

### Conclusion:

Together, these results demonstrate the utility of minimizing the extraneous DNA sequences in the vector DNA backbone during biliary hydrodynamic delivery to reduce the necessary DNA dose, or alternatively increase the potency of a fixed DNA dose. These results were not necessarily predictable since there could have been some saturation effect on the physical transfer of DNA through pores or within the cell, that would otherwise have influenced the net effect of the process. These unexpected results are useful for the further application of biliary hydrodynamic injection in primate liver.

### Example 35-Relationship of Parameters to Gene Delivery in Non-Human Primates

### Rationale:

A central feature of hydrodynamic injection is defining what injection parameters are optimal for gene delivery. The optimal injection parameters vary widely by the route of administration, and which organ is being targeted, such that they are very particular for a given approach, with the best approaches not predictable. For example, most studies in pig liver use upward of 1 liter of injection volume, which when scaled to a human patient, would not be tolerated.

Our previous published studies largely focused on a single set of injection parameters of around 30 mL of volume and 2 mL/sec into pigs. The scaling of these parameters into different size pigs and potentially humans was undefined. Given the differences between pig and primate liver, the importance of studying injection parameter optimization within primates was appreciated. Crucially, previous studies have only evaluated the difference between injection parameters in the context of influence on transfection efficiency. There have been no studies on the importance of injection parameters on quantitative gene expression from a reporter gene. Thus, the best injection parameters that could be used through the biliary system remain elusive. The experiment herein addressing that gap toward defining unexpected relationships between the variables of hydrodynamic injection through the biliary system. The experiment also helps elucidate the tolerability and toxicity of different flow rates within the context of the primate liver.

### Methods:

A set of four male baboons was obtained, weighing approximately 30 kg in size. The biliary hydrodynamic injection procedure was executed as in Example 26, with the catheters placed within the intrahepatic biliary system in order to maximize balloon seal. The balloon size was 11.5 mm to 15 mm, depending on the animal.

A set of four injection parameters were designed for each animal, that would test low (4 mL/sec) versus high flow rates (10 mL/sec), and low (50 mL) versus high volumes (120 mL). Each animal would thus occupy a unique corner of this square. The DNA doses were all kept the same. Concerning the identity of the DNA, a reporter plasmid DNA of the human FIX (hFIX) gene was injected under the control of a liver-specific promoter, LP1. An ELISA kit that distinguishes baboon FIX from hFIX was utilized (LSBio). Plasma samples were drawn from the baboons on day 1 post-injection and assessed for peak hFIX levels.

### Results:

As summarized in Table 14, expression results demonstrated that Baboon #1 (low flow, low volume) had similar expression to Baboon #2 (low flow, high volume) with both animals measuring approximately 10 ng/ml hFIX. Thus, volume does not appear to be a key parameter for determining expression. Baboon #1 (low flow, low volume) versus Baboon #3 (high flow, low volume) also had similar expression achieved, 10 ng/mL vs 12 ng/ml. It remained possible that the high flow, high volume cohort achieves the highest levels of expression. Baboon #4 (high flow, high volume) in this condition, however, achieved similar hFIX expression to Baboon #1-3 of around 14 ng/mL hFIX. The small differences between all of these groups were note considered to be statistically significant or biologically meaningful.

TABLE 14
Relationship of Parameters to hFIX expression
after Biliary Hydrodynamic Injection

| | |
|---|---|
| #1 Low Flow Rate, Low Volume | #2 Low Flow Rate, High Volume |
| (4 mL/sec, 50 mL) | (4 mL/sec, 120 mL) |
| hFIX level: 10 ng/mL | hFIX level: 25 ng/mL |
| #3 High Flow Rate, Low Volume | #4 High Flow Rate, High Volume |
| (8 mL/sec, 50 mL) | (10 mL/sec, 120 mL) |
| hFIX level: 12 ng/mL | hFIX level: 14 ng/mL |

Of note, the relative liver chemistry toxicities were similar across all conditions, suggesting a similar impact on the baboon liver despite disparate injection parameters. In all experiments, post-injection cholangiograms did not show any biliary rupture, demonstrating the ability of the bile ducts to tolerate significant volume and high flow rates rupture. This represents a new finding for primate liver.

### Conclusion:

The experiment demonstrates many crucial features of biliary hydrodynamic injection. The results suggest that lower flow rates and volumes already surpass the minimal threshold for pore formation and DNA entry. Thus, facilities faster injection is not additive. It's possible that faster injection leads to faster egress of DNA solution into the vasculature, nullifying any improvement in pore formation. Volume is a critical driving factor for vascular hydrodynamic injection but does not appear to be important for biliary injection. This is beneficial in that it allows for minimal volumes to be used in human procedures, making the procedure more compatible with existing clinical machines and less intrusive on human physiology. The results also suggest that the key findings of injection parameters in pigs are in a similar range for translation into primate liver, with only slight increases necessary for exact pressure plateaus to translate across species.

### Example 36-Multiple Injections within a Single Procedure can Yield Additive Gene Expression

### Rationale:

Hydrodynamic injection, regardless of animal model and route of delivery, is typically thought of as a one-time injection. The injection proceeds, pressure is increased, pores form, and DNA slips inside target cells. There is essentially no published literature to understand if you did a repeat hydrodynamic injection directly after a first injection, if the first injection would cause some alterations to the target organ that somehow prevent gene delivery. This includes the potential for more toxicity to the target organ leading to reduced gene expression, the previously diluted cytoplasm making the cells refractory, the cell membrane still being disrupted, amongst other possibilities.

On the other hand, the possibility of a re-injection directly after the procedure would be enticing. This would allow for re-injection of the patient should any mistakes occur during an individual procedure, or alternative the potential for gene addition through a series of multiple injections. We've previously published that multiple hydrodynamic saline injections are well tolerated in pigs, but there is no data to inform how gene expression would be modulated during repeat injections. Herein, the unpredictable effects of this novel strategy have been interrogated.

### Methods:

Two different male baboons were obtained, weighing approximately 30 kg in size. For baboon #1, the first injection was at 6 mL/sec and 31 mL, followed 10 minutes later by a second injection at 4 mL/sec and 22 mL. The approximate pDNA dose was 35 mg for the first dose, and 25 mg for the second dose. The total dose was ~60 mg. A comparator baboon #2 received only a single injection with a similar total dose of 60 mg and at parameters of 6 mL/sec and 50 mL.

Concerning the identity of the DNA, a reporter plasmid DNA of the human FIX (hFIX) gene was injected under the control of a liver-specific promoter, LP1. An ELISA kit that distinguishes baboon FIX from hFIX was utilized (LSBio). Plasma samples were drawn from the baboons on day 1 post-injection and assessed for peak hFIX levels.

### Results:

The baboon #1 injected with two different successive injections had similar hFIX levels (10 ng/ml) on day 1, as the baboon #2 injected with one single hydrodynamic injection on day 1 (12 ng/mL). Liver chemistries were not significantly different between the two animals, indicated there was not significantly more toxicity because of the repeat injections with DNA in a single procedure.

### Conclusion:

Repeat hydrodynamic injections through the biliary system can be conducted within a single procedure with additive expression. This discovery unlocks the ability for schemes to boost DNA dosing within a single procedure should limits of DNA concentration in solution be met, or alternatively limits within the volume of the solution in the power injector be met.

### Example 37-Alteration of Gene Delivery Method Allows the Detection of Fluid Leaks, and Decreases the Rate of Instrumentation Rupture

### Rationale:

A critical aspect of biliary hydrodynamic injection is the ability to access the biliary system of the liver efficiently. The injection procedure itself has potential risks of bile duct rupture, given the additional stress on the ductal walls of the system. Practically, the fluid injection itself, as outlined in other examples in the patent disclosure, does not seem to pose any risk to the bile ducts for potential rupture. However, there are still noticeable potential risks that should be minimized to make the biliary hydrodynamic injection procedure as safe as possible, while increasing its efficacy. These risks are the direct injury of the biliary system through physical trauma from the instrumentation itself. This includes the guidewire inserted into the bile ducts during cannulation. While designed to be very flexible, guidewires can end in particular points, that when advanced with enough force, could cause trauma and be forced through a bile duct wall.

### Methods:

A review of ERCP procedures for hydrodynamic gene delivery in pigs and primate liver was conducted. Example fluoroscopies were obtained which showed the appeared of a contrast leak reflective a bile duct leak. Images were recorded

### Results:

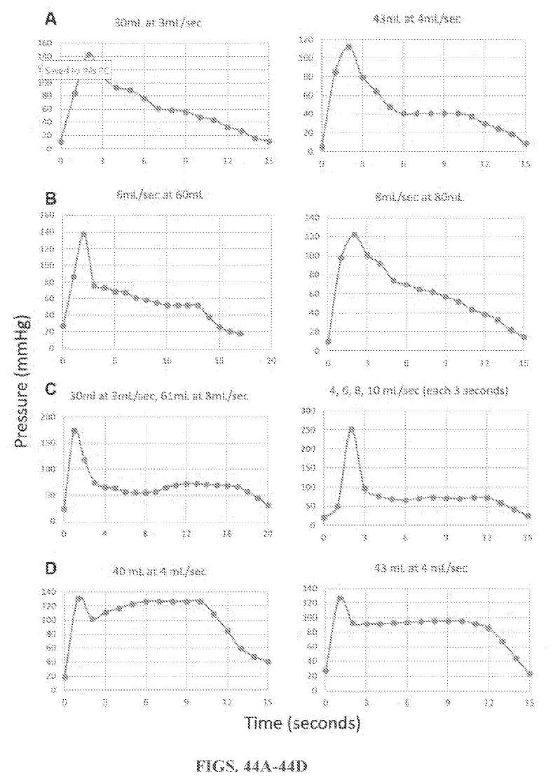

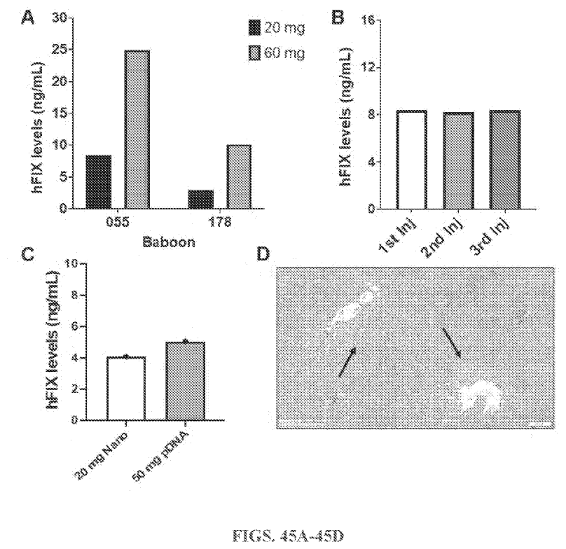

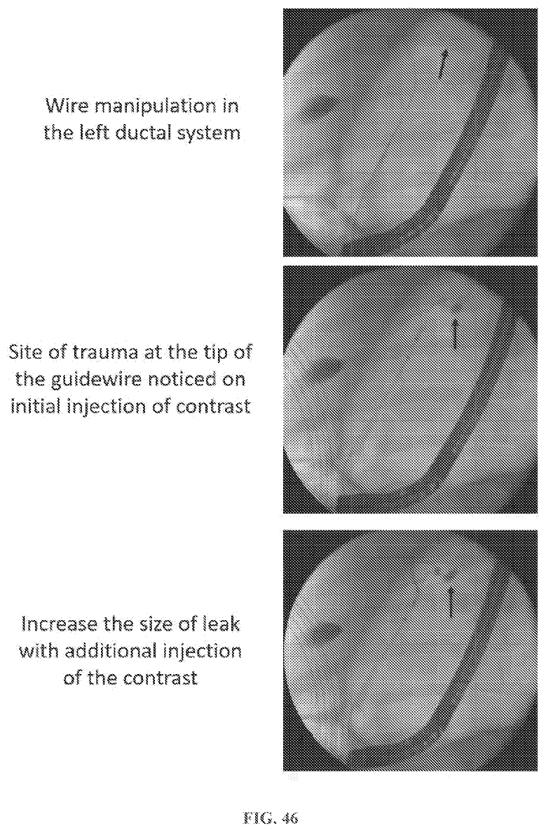

Bile leaks can rarely be detected before any hydrodynamic injection occurs during procedures. This was almost always tied to the advancement of the guidewire (FIG. 46). At least two times, the guidewire could be observed to cause an intrahepatic bile duct leak within the liver. Upon subsequent hydrodynamic injection, the size of the leak expanded, suggesting that the leak was a low-pressure source drawing the injection fluid during the injection. This was confirmed with pressure readings during a hydrodynamic injection with an intrahepatic leak not being as robust as injections where no intrahepatic leak occurred.

### Conclusion:

From a clinical perspective, intrahepatic bile duct leaks are not necessarily important, since they are self-closing and resolving. However, while these small intrahepatic lesions could be created during routine ERCP, it is discovered herein for the first time that they can have deleterious impacts on the efficiency and safety of biliary hydrodynamic injection. For this reason, strategies to avoid guidewire induced injury, such as the minimal advanced of the guidewire into the CBD and at the highest-level CHD can be utilized to eliminate the rate of intrahepatic lesions, thereby avoiding this complicating factor for hydrodynamic gene delivery.

### Example 38-Stent-Mediated Biliary Injection

### Rationale:

Biliary hydrodynamic injection traditionally takes place in a branch of the biliary system upstream of the cystic duct. The purpose of this is to avoid injection of fluid into the gallbladder, which would otherwise result in a significant pressure drop.

Despite those advantages, the use of a balloon catheter for the procedure does pose several challenges. The first challenge is that the balloon can supply tension on the bile duct walls, which can rarely lead to rupture. The second is that in some patients, the common hepatic duct itself may be quite small, and thus it's hard to localize the catheter in the proper location. To address those limitations, an alternative procedure was pursued, wherein a stent would be deployed within bile duct to obstruct the cystic duct and create a facile method of injection into the biliary system.

### Methods:

A biliary stent employment device was obtained (Micro-Tech Endoscopy). The 10×100 mm fully covered stent was utilized, in conjunction with the catheter to deploy the stent. After canulation of the bile duct of a 25 kg pig, the catheter with stent was advanced into the bile duct, with the stent area covering the opening of the cystic duct such that no fluid would enter into the gallbladder. The stent was partially deployed, meaning that the distal end of the stent expanded to cover the walls of the bile duct, while the proximal end of the stent remained beneath the sheath of the catheter, such that the proximal end mimics the shape of a cone. In the center of the stent is an olive through which the stent is deployed. Within the center of the olive is a guidewire channel, which is previously used to help facilitate cannulation of the bile duct in a 25 kg pig. For modeling hydrodynamic injection, the guidewire was removed to allow for this channel to be used for fluid injection.

### Results:

The stent could successfully be partially deployed successfully, thereby creating a sealed bile duct wall on the lateral aspects, while also having a sealed end at the proximal aspect of the catheter at the point of the stent entering the sheath (FIG. 47). Contrast solution could be injected at pressure through the guidewire channel and enter into the biliary tree. No contrast was noted to slip around the walls of the stent. In this application, the olive itself provided resistance to any antegrade flow within the stent, although that was furthermore prevented by the sheath itself. Thus, a stent could be utilized to generate a seal of the bile duct walls and at the proximal end.

### Conclusion:

The use of a stent-based catheter to mediate hydrodynamic delivery of the gene solution represents an alternative to balloon catheter mediated approaches. Both methods have utility then toward the treatment of patients. The stent approach is useful in almost eliminating the potential for bile duct wall rupture, which can occur from overstretching of the bile duct wall, along with force from the guidewire.

### Example 39-Tumor Delivery in Pigs

### Rationale:

Cancer of the liver and pancreas represent devastating diagnosis with poor outcomes. Liver cancer has 36% survival at 5-years after diagnosis, while pancreatic cancer has 12% survival at 5-years diagnosis. Primary liver cancer in adults is typified hepatocellular carcinoma, although primary tumor types can exist. The most common form of pancreatic cancer is pancreatic ductal adenocarcinoma.

Give in the poor pregnancies of these cancers, strategies to better model the tumors are key. While mouse models are useful, they are inherently limited in testing many of the most common strategies used to treat these tumors. This includes surgical approaches for physical removal of the tumor, as well as testing specific delivery routes into tumors, such as those achieved through interventional radiology and endoscopy approaches. As such, mouse models of pancreatic and liver cancer remain most useful for testing of small molecule approaches, as well as modeling any systematically administered agent, whether biologics or cell therapies.

To address these limitations, previous investigators have created the OncoPig model for cancer. Pigs are an ideal model, since they have similar internal organ sizes compared to humans. Thus, the same clinical equipment can be utilized in pigs in order to model potential treatments in patients. The Oncopig model is characterized by mutations in p53 and KRAS, which can be activated by administration of Cre recombinase. Administration of Cre recombinase into soft tissues of the pig can rapidly generate sarcomas quickly. Concerning gastrointestinal organs, cancer cell lines can be generated from hepatocytes isolated from Oncopigs and when engrafted into mouse models appear to have similar histology and phenotype to hepatocellular carcinoma (HCC).

Investigators have established a method of liver and pancreatic tumor generation uncopied models, wherein tissue is extracted via biopsy from the animal, incubated with virus and gel foam and then reintroduced into the animal at the desired site. These tumors form rapidly over the course of one week, reaching one to two centimeters in size. both approaches utilized a percutaneous or CT guide approach for placing the needle into the tissues. alternative approaches for sampling tissue from these organs, including endoscopic ultrasound guided biopsy, have not been described. Ultrasound (US) guidance via endoscopy notably can sample larger tissue sizes compared to the needles employed and percutaneous or CT guided procedures. Beyond the establishment of tumors in the liver and pancreas, only limited studies have been performed to investigate there are therapeutic applications. A previous study looked at the potential to model transarterial chemo-embolization against tumors in the OncoPig, finding similar responses to the human procedure.

Treatment strategy: All investigations of biliary hydrodynamic delivery and pancreatic ductal hydrodynamic gene delivery have focused on the use in delivering to normal liver and pancreatic tissue, respectively. This leverages the natural ductal channels in these organs that connect to almost all hepatocytes and acinar cells, respectively. Thus, these ductal systems form a highway for DNA delivery into normal cell types.

It is uncertain, however, if these same ductal routes could be leveraged for the delivery into non-native tissue, such as tumors that form within the liver and pancreas organs. In these circumstances, the ducal connections are disrupted, such that there are uncertain connections between the tumor to the surrounding tissue. Many pathologists consider the tumors to be disconnected from the ductal system, and thus tumors would not be directly reachable. Beyond transfecting the tumor directly, it's also unknown if the presence of the tumor itself impacts all surrounding tissue in the tumor microenvironment, making it refractory to the delivery of DNA. An example would be tumors impinging on larger bile ducts, thus sealing of an entire area of normal tissue space.

In spite of these challenges, an exciting potential modality to treat liver and pancreatic tumors is gene therapy, wherein nucleic acid encoding antitumor proteins can be delivered into the tumor. Intratumoral delivery of plasmid DNA has been attempted in clinical trials of skin cancer and pancreatic cancer by direct injection into the tumor, although the therapeutic outcomes were suboptimal suggesting alternative delivery strategies are needed. Hydrodynamic delivery uses fluid pressure to mediate DNA delivery inside cells. The technique is efficient in the DNA delivery into mouse liver and has shown promising efficacy in pig liver when delivered through the biliary system. Whether hydrodynamic injection can promote intratumoral gene delivery is unclear.

Herein, it has been sought to explore the establishment of liver and pancreatic tumors in an Oncopig model via endoscopic routes. For the first time, simultaneous tumors in both organs have been established for the first time and the instant disclosure has verified the ability of EUS to help establish liver and pancreatic tumors. The ability of hydrodynamic injection to mediate intratumoral and peritumor DNA delivery and gene expression has also been piloted for the first time, opening future therapeutic possibilities.

### Methods:

Animals: Four Oncopigs were acquired from the National Swine Research Center in the University of Missouri. All pigs were male and castrated prior to the experiments. Oncopigs were shipped to Institute Research Hills at the Mayo Clinic in Rochester, Minnesota for experiments.

Plasmid DNA: The vector, pCLucf, obtained from Addgene, was used as a reporter plasmid for all DNA injections. It was chosen based on the dual reporter expression cassettes, with CMV driving firefly luciferase expression, while SV40 promoter drives GFP expression. Both CMV and SV40 promoters are relatively ubiquitous across cell types. pCLucf plasmid was prepared on mass scale at low endotoxin levels by Gene Universal.

Tumor generation: Tissue samples were taken on biopsy from the Oncopig model by either percutaneous liver biopsy or endoscopic ultrasound guided biopsy. Tissue was pulverized into a cell suspension used a stop cock, mixing with gelfoam to provide a matrix for cells to reside in. Cre recombinase virus was also mixed in with the pulverized cells at this time point. AAV8-ELA1-iCre and Ad-CMV-Cre were used to start pancreatic tumors, while Ad-ALBp-iCre and Ad-CMV-Cre were used to start liver tumors. The suspension was then re-injected into the pig in either the liver or pancreas sites. Within 1-2 weeks, tumors formed at all sites, with multiple tumors within each organ. At this time, biliary hydrodynamic injection was performed in the liver, while ductal hydrodynamic injection was performed in the pancreas. Concerning dose, all pigs were injected with 20 mg of pCLucf into the liver, and 10 mg of pCLucf into the pancreas during hydrodynamic injection. Approximately 24 hours post-injection, the animals were harvested, and organs analyzed for their tumors and surrounding liver tissue

### Results:

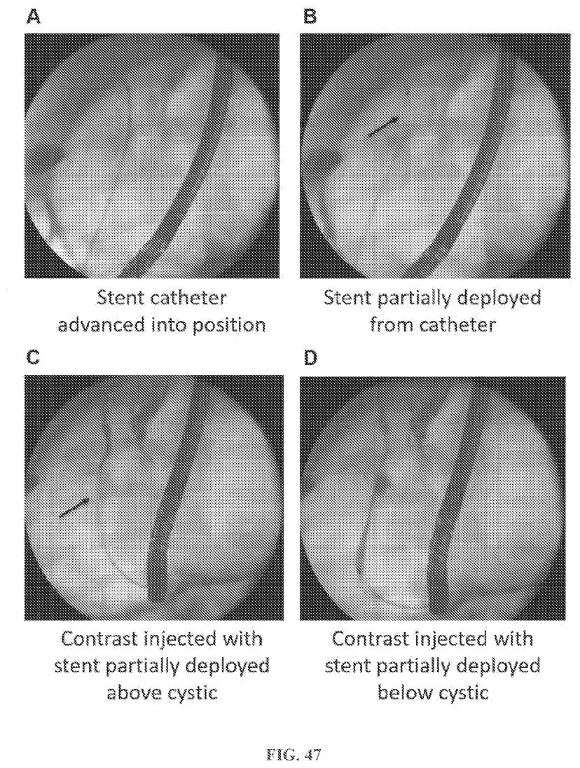

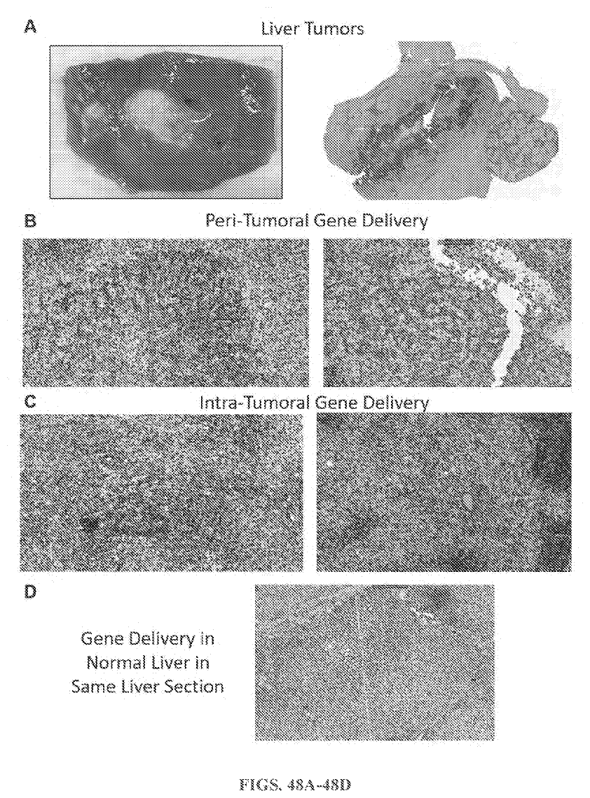

Liver and pancreas tumors amounting to approximately 1-2 centimeters in size were observed in the multiple lobes of the liver (FIG. 48A), along with the pancreas head and tail, respectively. The tumors were sectioned for further analysis. Immunohistochemical staining for GFP and firefly luciferase was pursued. Results demonstrated the presence of positive staining amongst hepatocytes that were on the border of tumors at the zone of infiltration (peri-tumoral space, FIG. 48B). Positive staining for tumor cells themselves could also be appreciated (FIG. 48C). A similar pattern was observed amongst tumors taken from different liver lobes. Importantly, delivery into normal liver lobules and hepatocytes was concurrent (FIG. 48D). Thus, injection of naked DNA at a single site of the common hepatic duct is able to reach the entire liver, including inside and around liver tumor samples. This feature should be useful for the treatment of metastatic liver disease with multiple lesions.

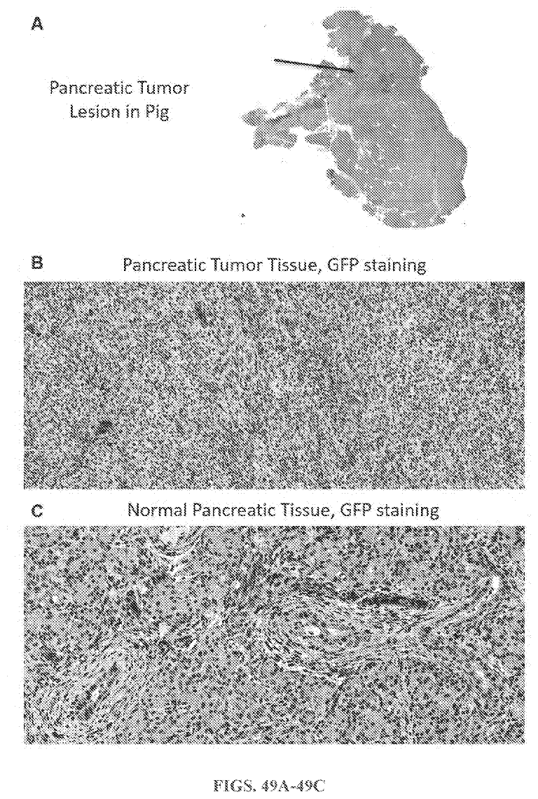

The pancreas tumor delivery findings were also similar. Large pancreatic tumors were observed (FIG. 49A). Scattered GFP positive cells could be detected within the area identified as the pancreatic tumor (FIG. 49B). GFP and Luciferase positive cells staining for ducts, islets and acinar cells could be observed in proximity to the tumor (FIG. 49C).

### Conclusion:

Hydrodynamic injection through the ductal systems of the liver and pancreas are able to target tumor tissue as well as normal tissue, in spite of disrupted ductal networks connecting to the tumors. Thus, high-pressure fluid flow is able to escape these networks and further penetrate into tissues, such as tumors, offering an unexpected avenue for more therapeutic interventions.

### REFERENCES

1. Griffin M A, Restrepo M S, Abu-El-Haija M, et al. A novel gene delivery method transduces porcine pancreatic duct epithelial cells. Gene Ther 2014; 21:123-130.
2. Wang Y, Dorrell C, Naugler W E, et al. Long-Term Correction of Diabetes in Mice by In Vivo Reprogramming of Pancreatic Ducts. Mol Ther 2018; 26:1327-1342.
3. Ogawa K, Kamimura K, Kobayashi Y, et al. Efficacy and Safety of Pancreas-Targeted Hydrodynamic Gene Delivery in Rats. Mol Ther Nucleic Acids 2017; 9:80-88.
4. Yamada Y, Tabata M, Abe J, et al. In Vivo Transgene Expression in the Pancreas by the Intraductal Injection of Naked Plasmid DNA. J Pharm Sci 2018; 107:647-653.

Kumbhari V, Li L, Piontek K, et al. Successful liver-directed gene delivery by ERCP-guided hydrodynamic injection (with videos). Gastrointest Endosc 2018; 88:755-763.e5.
6. Johnson K M, Kines R C, Roberts J N, et al. Role of heparan sulfate in attachment to and infection of the murine female genital tract by human papillomavirus. J Virol 2009; 83:2067-2074.
1. Hum Gene Ther. 1997 Oct. 10; 8(15): 1763-72
2. GIE 2005, T1249 Abstract

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EQUIVALENTS

It is understood that the detailed examples and embodiments described herein are given by way of example for illustrative purposes only and are in no way considered to be limiting to the disclosure. Various modifications or changes in light thereof will be suggested to persons skilled in the art and are included within the spirit and purview of this application and are considered within the scope of the appended claims. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

1.-233. (canceled)

234. A method of hydrodynamic gene delivery through the biliary system of a liver of a subject, comprising:
(a) insertion of catheter into the common hepatic duct, right hepatic duct, or left hepatic duct
(b) inflation of balloon to seal the duct and increase pressure during injection
(c) injection at a flow rate at minimum of 2 mL/sec, or alternatively at a minimum pressure of 50 mmHg
(d) delivery of DNA encoding hepatocyte-specific promoter to drive transgene expression
(e) a DNA vector with substantially reduced or absent non-mammalian sequence elements
(f) DNA dose injected is at minimum 10 mg of DNA per kilogram of liver weight
wherein greater than 50% of hepatocytes expressing the gene of interest; or
A method of hydrodynamic gene delivery through the biliary system of a liver of a subject, comprising:
(a) insertion of catheter into the common hepatic duct
(b) inflation of balloon to seal the duct and increase pressure during injection
(c) injection at a flow rate at minimum of 2 mL/sec, or alternatively at a minimum pressure of 50 mmHg
(d) delivery of DNA encoding hepatocyte-specific promoter to drive transgene expression
(e) a DNA vector with substantially reduced or absent non-mammalian sequence elements
(f) DNA dose injected is at minimum 10 mg of DNA per kilogram of liver weight
wherein greater than 50% of hepatocytes expressing the gene of interest; or
A method of hydrodynamic gene delivery through the biliary system of a liver of a subject, comprising:
(a) insertion of catheter into the bile duct:
(b) inflation of balloon to seal within the bile duct and prevent antegrade flow of solution
i. The inflated size of the balloon is at least 2×, at least 3×, or at least 4× the diameter of the bile duct to overcome elasticity of primate ducts;
ii. wherein a method of verifying balloon seal during injection consists of the placement of radiocontrast solution above and below the balloon to detect antegrade motion of fluid by fluoroscopy
(c) injection at a flow rate at minimum of 2 mL/sec, or alternatively injection at a minimum pressure of 50 mmHg
i. more preferably, a minimum of 4 mL/sec, which can yield minimum of 80 mmHg into primate liver in plateau pressure; wherein the flow rate can be further minimized to less than 12 mL/sec, less than 10 mL/sec, or less than 8 mL/sec without loss in total gene expression;
ii. volume injected is preferably less than 250 mL/kg liver tissue, less than 150 mL/kg liver tissue, or less than 50 mL/kg liver tissue;
iii. wherein a plateau pressure during hydrodynamic injection varying less than 10% of mmHg over the course of the injection is obtained and signals sufficient seal; and
iv. wherein optionally multiple flow rates can be employed within a single injection to alter and vary the pressure obtained; and
(d) delivery a miniaturized DNA vector with substantially reduced or absent non-mammalian sequence elements
i. wherein the miniaturized DNA vector affords greater persistence of expression lasting at least 4 months;
ii. wherein the miniaturized DNA vector affords high potency at a given DNA dose in primates, allowing for smaller doses to be used compared to regular plasmid DNA;
iii. wherein the DNA vector optimally contains a hepatocyte-specific promoter for enhanced expression in the liver.

235. The method of claim 234, wherein a more efficient balloon seal is obtained by advancing the catheter and inflating the balloon into intrahepatic ducts; or
wherein placement of the balloon in extrahepatic ducts results in leakage of fluid around the balloon; or
wherein the balloon size is at least 2 times, at least 3 times, or at least 4 times the duct diameter; or
wherein the balloon can be inflated to maximal size 3 times the duct diameter, when the balloon is placed in an extrahepatic bile duct; or
wherein the balloon can be inflated to a minimal size of 4 times the duct diameter, when the balloon is placed in an intrahepatic bile duct; or
wherein the balloon size does not inflate fully within an intrahepatic duct, but rather additional pressure is generated within the balloon; or
wherein leakage around the balloon occurs with sizes 8.5 mm or less, whether intrahepatic or extrahepatic, such that those sizes should be avoided; or
wherein the balloon is inflated less than 15 mm in size in the common hepatic duct in order to avoid rupture; or
wherein if the balloon is placed in the right or left hepatic duct and the injection occurs subsequently in that location, then the injection is repeated again in the opposing duct to ensure both lobes of the liver are injected equally; or
wherein the balloon seal can be monitored through measuring intraluminal biliary pressure; or
wherein loss of a plateau wave form, defined by greater than 20 mmHg decrease from the start to the end of the plateau, signifies leakage around the balloon; or
wherein the balloon seal can be verified by filling the bile ducts above and below the balloon with radiocontrast solution prior to injection; or
wherein a loss of fluid seal during injection is demonstrated by the clearing of contrast below the balloon, either into the cystic duct and gallbladder, or alternatively into the common bile duct; or
wherein contrast above the balloon is cleared into the liver to indicate successful injection; or
wherein the hydrodynamic injection can be repeated multiple times within a single procedure in order to augment DNA delivery; or
wherein the use of two or more injections is additive toward achieving the final gene expression amount; or
wherein this strategy allows one to overcome inherent limitations of the angiographic or power injection volume through the use of multiple injections; or
wherein primate liver tissue is more elastic than porcine tissue, such that different duct properties necessitate changes in balloon sizes and injection parameters to mediate gene delivery in primates; or
wherein the flow rate must be increased to achieve a given pressure versus injection in porcine models; or
wherein a flow rate of at least 4 mL/sec is required to achieve a pressure plateau of at least 80 mmHg; or
wherein primates can tolerate pDNA doses of at least 80 mg without any significant physiological side effects; or
wherein an injection volume of at least 120 mL volume per 400 gram of liver can be injected into a primate without any significant perturbation in vital signs; or
wherein an injection speed of up to 12 mL/sec can be tolerated by the primate liver without tissue injury, vital sign changes, or bile duct rupture; or
wherein flow rates greater than 4 mL/sec, but less than 8 mL/sec, should be used given lack of improved gene delivery at higher flow rates; or
wherein 30 mL per 400 g of volume can be used, or alternatively up to 150 mL per 400 g of volume can be used; or
wherein the volume utilized does not impact the efficiency of gene delivery at a given DNA dosage; or
wherein the vector composition should be dosed by the copies of the transgene expression cassette, such that differing pDNA doses are required if additional extraneous DNA is included in the DNA vector; or
wherein the use of DNA molecule with reduced backbone allows for a comparatively smaller DNA dose; or
wherein increasing the pDNA dose and/or the vector expression cassette dose per animal leads to quantitatively equivalent increase in expression of a protein therapeutic of interest; or
wherein liver sinusoidal endothelial cells (LSECs) can be targeted for cell-specific expression by biliary hydrodynamic injection with CD36 promoter or FVIII promoter in the vector.

236. The method of claim 234, wherein alternatively, a DNA vector lacks specific modifications, but optimally is injected at a minimum of 20 mg of DNA per kilogram of liver weight to achieve greater than 50% of hepatocytes expressing the gene of interest; or
wherein an optional use of transposon can be utilized to facilitate integration into host chromosomes; or
wherein a substantially reduced means total amount of bacterial or phage DNA sequences less than 1000 bp; or
wherein the DNA is a plasmid DNA vector with vector bacterial backbone or sequence less than 1 kb in size, or more preferably less than 500 bp in size; or
wherein the plasmid DNA is a nanoplasmid, pFAR, or pCOR vector; or
wherein the DNA is a circular and is a minicircle DNA; or
wherein DNA is a linear DNA from closed-ended DNA, ministring DNA, or doggbone DNA; or
wherein the switch from plasmid backbone including bacterial sequences greater than 1 kb to a DNA vector of claims 5 and 6 increases the observed total transfected area of hepatocytes more than 20%; or
wherein the gene expression among at least 40% of hepatocytes for at least 3 months can be achieved with the use of a transposon system for integration into host genome; or
wherein the duration of expression of non-integrating DNA of claims 5 and 6 is at least 4 months after injection; or
wherein the delivery method expresses for at least 4 months and is able to yield immune tolerance to foreign transgenes in the liver; or
wherein DNA vectors sizes greater than 12 kb, 15 kb, or 20 kb in size can be delivered into multiple cell types through the liver, including hepatocytes, endothelial cells, and bile duct cells yielding protein expression; or
wherein transfection efficiency is maintained with larger plasmid DNA sizes at least 12 kb in size; or
wherein DNA dose can be adjusted according to DNA dose (mg) per liver weight (kg) per kilobases of DNA (kb) in order to adjust for the DNA size in order to maintain equivalent transfection efficiency; or
wherein the formula, 1 mg/kg/kb, can be utilized to project the DNA dose to achieve about 50% of hepatocyte transfection inside the liver; or
wherein the formula 2.5 to 5 mg/kg/kb can be utilized to project the DNA dose to achieve about 70% of hepatocyte transfection inside the liver; or
wherein the procedure can be repeated on a second date with a different DNA expressing the same or a different gene, such that the expression of the first gene is not abolished and expression of both genes is now achieved; or
wherein the second injection achieves similar transfection efficiency to the first injection and can target the same cells; or
wherein the same cells can be observed to express genes after injection; or
wherein the promoter can be altered to achieve expression in a different cell type with the second injection, and that the second injection does not alter expression of the first gene; or
wherein the procedure can be repeated within the same injection procedure with a different DNA expressing the same or a different gene, such that expression of both DNAs is now achieved, and the expression of the first DNA injection is not abolished; or
wherein the second injection achieves similar transfection efficiency to the first and can target the same cells; or
wherein the promoter can be altered to achieve expression in a different cell type with the second injection, and that the second injection does alter expression of the first gene; or
wherein two different DNA molecules can be mixed and delivered during a single injection, such that both DNA molecules enter into the same liver cells; or
wherein DNA doses from 20 mg per kg liver weight to 40 mg per kg liver weight achieve similar transfected area; or
wherein DNA doses up to 40 mg per kg liver weight can be injected without causing significant liver toxicity or physiological distress; or
wherein the flow rate below 1 mL/sec yields no gene expression; or
wherein a flow rate between 1 mL/sec and 2 mL/sec exhibits decreased gene expression compared to greater than 2 mL/sec; or
wherein flow rates above 4 mL/sec yield progressively less efficient hepatocyte delivery; or
wherein flow rates greater than or equal to 7 mL/sec achieve efficient gene delivery into cholangiocytes; or
wherein the preferred injection volumes are between 30 mL/kg to 60 mL/kg per liver tissue; or
wherein injection volumes greater than or equal 70 mL/kg liver tissue are associated with decreased efficiency of gene delivery; or
wherein the gene injection procedure is well-tolerated by subjects of 25 kg or 15 kg or 5 kg in size and yields similar gene delivery efficient to larger mammals; or
wherein the transfected hepatocyte area from biliary hydrodynamic delivery can be further increased by at least 10% of total hepatocytes when incorporating two or more flow rates during the injection; or
wherein the flow rate of 2 mL/sec is used first for 50% to 66% of the total injected volume, followed by 4 mL/sec second for the remaining volume; or
wherein the flow rate is 2 mL/sec is used first for 33% of volume injection, 3 mL/sec is used second 33% of the volume, followed by 4 mL/sec for the remaining injection volume; or
wherein the catheter is inserted into the common hepatic duct through ERCP, EUS, or imaging-guided percutaneous routes; or
wherein the DNA concentration of the injected solution is at minimum 0.30 mg/mL, and more preferably greater than 0.40 mg/mL, 0.50 mg/mL, or 0.60 mg/mL in concentration.

237. The method of claim 234, wherein a substantially reduced means total amount of bacterial or phage DNA sequences less than 1000 bp; or
wherein the DNA is a plasmid DNA vector with vector bacterial backbone or sequence less than 1 kb in size, or more preferably less than 500 bp in size; or
wherein the plasmid DNA is a nanoplasmid, GenCircle, pFAR, or pCOR vector; or
wherein the DNA is a circular and is a minicircle DNA or a minivector DNA; or
wherein DNA is a linear DNA from closed-ended DNA, ministring DNA, or doggbone DNA; or
wherein the linear DNA only has small foreign sequences at either end, each less than 100 bp's in size, and the mammalian expression sequence of interest is the rest of the vector; or
wherein a biliary sphincterotomy is performed during the first procedure in a subject to reduce or eliminate the risk of post-ERCP pancreatitis during subsequent ERCP procedures for redosing the genetic medicine; or
wherein the injection procedure can be repeated twice within one session to increase protein expression; or
wherein the repeated injection can use the same DNA to boost individual protein expression, or can use two different DNA solutions to yield two different proteins being expressed; or
wherein the sum of the DNA dose from the repeated injections is similar to or equivalent to the expression obtained from a single DNA injection; or
wherein the injection procedure can repeated again after a single administration, such that the transfection efficiency is the same, and the peak protein expression is equivalent between injections with lack of immunogenicity observed; or
wherein the injection procedure that is repeated can occur at least one month, at least 3 months, at least 6 months, or at least one year apart; or
wherein the pressure is monitored using a pressure transducer detecting a fluid-filled column in the pressure catheter, or alternatively is monitored with a pressure sensor that is threaded into the catheter lumen; or
wherein the pressure sensor readings are recorded in real-time, and the pressure curves interpreted post-injection to ascertain if successful seal and peak expression were achieved; or
wherein optimal hydrodynamic injections achieve at least a 2× rise in liver enzymes such as ALT and AST levels compared to pre-treatment values by day 1 post-injection; or
wherein the liver enzymes return to normal limits within 7 days of injection; or
wherein gene delivery occurs intratumorally and peri-tumorally, in both malignant cells and normal cells, interspersed and surrounding the tumor.

238. A method of gene delivery into the liver of a primate, comprising the steps of:
inserting a catheter into a common hepatic duct of a primate;
inflating a balloon in the common hepatic duct to prevent an antegrade flow; and
injecting a DNA solution at a hydrodynamic pressure in a primate liver
wherein the injection achieves >30% of hepatocytes expressing a gene of interest in the primate liver; or
A method of hydrodynamic retrograde ureter delivery of nucleic acids or viral vectors, compromising steps of:
(i) insertion of a cystoscope through the urethra and into the bladder
(ii) insertion of a balloon catheter through the cystoscope and into the bladder
(iii) canulation of the ureteral orifice with the balloon catheter
(iv) inflation of the balloon catheter at the distal ureter near the ureter orifice entrance
(v) use of power injector to mediate hydrodynamic injection into the kidney
wherein the method does not require the use of fluoroscopy for catheter placement; or
A method of hydrodynamic retrograde ureter delivery of nucleic acids or viral vectors, compromising steps of:
(i) insertion of a cystoscope through the urethra and into the bladder
(ii) insertion of a guidewire through the cystoscope and into the bladder
(iii) canulation of the ureteral orifice with the guidewire
(iv) advancement of the guidewire toward the kidney
(v) removal of cystoscope and exchange of balloon catheter over the guidewire.
(vi) inflation of balloon catheter within ureter at proximal location near kidney
(vii) use of power injector to mediate hydrodynamic injection into the kidney
wherein the method can utilize balloon catheters that do not fit the working channel of cystoscopes; or
A method of hydrodynamic injection into the liver through the biliary system, using a partially deployed fully covered metallic or plastic stent wherein:
a) the deployment of the stent is distal to proximal (with respect to the stent deployment catheter)
b) the location of the released or open component of the stent is in the common hepatic duct and the tip of the stent remains in delivery catheter housing the stent, such that is located in either the common hepatic duct, common bile duct, or in the periampullary duodenum
c) wherein the cystic duct orifice is occluded and/or bypassed by the covered portion of the stent, such that no injected fluid solution is able to enter the cystic duct or gall bladder
d) removing the guidewire from when the stent delivery system
e) injecting a DNA solution at high pressure target and/or flow rate into the biliary system through the guidewire lumen
f) wherein antegrade flow in the biliary system is prevented by the stent forming a closed system with the catheter due to the partial deployment and continued connection,
wherein gene expression can be observed in hepatocytes within the liver on immunostaining; or
A method of hydrodynamic injection through the biliary system, wherein the injection occurs in the common bile duct with the aid of a biliary stent; or
A method of hydrodynamic gene delivery into the pancreas through the ductal system, comprising:
(a) insertion of catheter through endoscopic retrograde cholangio-pancreatography into the pancreatic duct
(b) inflation of balloon to seal and increase pressure during injection
(c) injection at flow rate equal to or between 1 to 2 mL/sec
(d) injection at volume equal to or less than 0.20 mL per gram of pancreas weight
(e) DNA dose injected is at least 10 micrograms per gram of pancreas weight
wherein the method is sufficient to mediate gene expression in all lobes with decreased pancreatic enzyme elevation and tissue necrosis.

239. The method of claim 238, wherein the common hepatic duct is accessed by an endoscopic retrograde cholangio-pancreatography (ERCP); or
wherein the ampulla of Vater may be cut to increase the size of the opening for the ease of canulation of the common hepatic duct during ERCP; or
wherein a radiocontrast injection is used to localize the catheter that is localized in the common hepatic duct past a cystic duct to avoid injection to a gallbladder; or
wherein the radiocontrast injection verifies that the balloon seals the common hepatic duct during the injection, and that a right and a left hepatic duct is visualized; or
wherein the DNA solution is a normal saline solution with pure recombinant DNA dissolved in the solution; or
wherein a DNA in the DNA solution may be plasmid DNA, minicircle DNA, or linear closed-ended DNA; or
wherein the volume injected is at least 30 milliliters per kilogram of the liver weight, or at least 40/mL/kg or greater; or
wherein the flow rate is at least 1 mL/sec, at least 2 mL/sec, or at 3 mL/sec or greater; or
wherein the pressure parameter is at least 50 mmHg, at least 80 mmHg, or greater than 120 mmHg during a pressure-guided injection; or
wherein the DNA dose will be at least 10, 20, 30, 40, or 50 milligrams per kilogram of a liver weight in certain embodiments; or
wherein the DNA solution is a DNA vector composition encoding a hepatocyte-specific promoter; or
wherein the hepatocyte-specific promoter also contains one or more of a hepatocyte-specific enhancer to drive higher levels of transcription; or
wherein the gene of interest is a codon optimized with codons selected for abundance in the hepatocytes will be selected; or
wherein the DNA vector composition and protocol are provided for the treatment of hemophilia B in primates; or
wherein the DNA vector composition encodes the human factor IX (hFIX) gene; or
wherein the DNA vector composition is a nanoplasmid with a bacterial backbone under 500 basepairs; or
wherein the total DNA vector composition size is less than 3 kb for hFIX; or
wherein a DNA vector composition dose of 20 mg per kg primate liver is sufficient to yield detectable hFIX in the plasma of primates; or
wherein the DNA vector composition can be redosed in order to secure further gains in expression; or
wherein the procedure may be repeated a second time in the primate, wherein the expression of two different genes is achieved.

240. The method of claim 238, wherein rupture or tears of the kidney are avoided by using injection parameters equal to or below 20 mL in total volume, and 2 ml/sec; or
wherein flow rates equal to or between 0.5 mL/sec1 to 2 mL/sec are optimal for achieving gene delivery without rupture expression; or
wherein optimal volumes for injection are between 10 mL to 20 mL to mediate effective gene delivery without rupture; or
wherein the balloon is positioned in the muscular wall of bladder in the ureter and can be visualized with by the cystoscope camera without the need of fluoroscopy; or
wherein the cystoscope camera can be monitored during the injection for any outflow from the ureter with the complete absence indicating effective for seal during injection; or
wherein the nucleic acids are minimum dosed at 1 mg, 2 mg, 3 mg, 4 mg, or higher in mass for an individual kidney in a subject 30 kg or greater; or
wherein the hydrodynamic parameters are sufficient to achieve protein expression within cells of the kidney after delivery of DNA; or
wherein cell expression is achieved within cells of the glomerulus, tubule, or endothelium inside the kidney; or
wherein differences in cell expression among the glomerulus, tubule, or endothelium correlate to the uses of different promoters in the plasmid DNA.

241. The method of claim 238, wherein rupture or tears of the kidney are avoided by using injection parameters equal to or below 12 mL and 2 ml/sec; or
wherein flow rates equal to or between 0.5 mL/sec to 2 mL/sec are optimal for achieving gene delivery without rupture; or
wherein optimal volumes for injection are between 7 mL to 12 mL mediate effective gene delivery without rupture; or
wherein the balloon is positioned at least 1 cm, 2 cm, or 3 cm away from the renal pelvis into the ureter in order to assure proper seal; or
wherein radiocontrast injection is utilized to confirm positioning of the catheter and seal of the balloon prior to injection; or
wherein the nucleic acids are minimum dosed at 1 mg, 2 mg, 3 mg, 4 mg, or higher in mass for an individual kidney in a subject 30 kg or greater; or
wherein the hydrodynamic parameters are sufficient to achieve protein expression within cells of the kidney after delivery of DNA; or
wherein cell expression is achieved within cells of the glomerulus, tubule, or endothelium inside the kidney; or
wherein differences in cell expression among the glomerulus, tubule, or endothelium correlate to the uses of different promoters in the plasmid DNA; or
wherein the nucleic acids consist of plasmid DNA, minicircle DNA, mRNA, siRNA, or antisense oligonucleotides; or
wherein the viral vector is selected among adenovirus, adeno-associated virus, lentivirus, baculovirus, anellovirus, or Sindbis virus; or
wherein the hydrodynamic injection is used to facilitate better viral vector penetration into tissue, binding to cells, and entry into cells versus viral vector infused at a non-hydrodynamic flow rate; or
wherein the transduction efficiency of kidney cells through retrograde ureter injection is higher with hydrodynamic injection than with a non-hydrodynamic injection (<0.15 mL/sec flow rate); or
wherein the hydrodynamic injection can be further monitored with a pressure sensor to make sure that pressures at a minimum of 50 mmHg, 60 mmHg, 70 mmHg, or 80 mmHg are reached; or
wherein a distal injection into the ureter is sufficient to yield pressure of at least 80 mmHg at flow rates equal to or greater than 0.5 mL/sec; or
wherein a proximal injection into the ureter is sufficient to yield pressure of at least 100 mmHg at flow rates equal to or greater than 1 mL/sec.

242. The method of claim 238, wherein the high-pressure target is greater than 50 mmHg, greater than 80 mmHg, or greater than 120 mmHg; or
wherein the flow rate is greater than 2 mL/sec, greater than 5 mL/sec, or greater than 10 mL/sec; or
wherein the volume injected is greater than 50 mL per kg of liver weight, or greater than 75 mL per kg of liver weight, or greater than 100 mL per kg of liver weight; or
wherein prior to injection, bile is removed from the biliary system, and normal saline solution is used to flush and wash the biliary system; or
wherein the preferred DNA dose is at least 20 mg per kg liver weight, or larger; or
wherein the preferred DNA concentration of the injection solution is at least 0.5 mg/ml DNA or more; or
wherein the biliary stent is at minimum the diameter of the bile duct, in order to provide sufficient seal with the duct walls and the stent during the injection; or
wherein the biliary stent diameter is at least 150% of the bile duct diameter, or at least 200% of the bile duct diameter; or
wherein the biliary stent is of variable length and can reach from the outside the ampulla to the upstream of the cystic duct; or
wherein the injection occurs from the tip of the “olive” aspect of the stent, where the “olive” may be located between the liver end of the stent and the hepatic hilum at the time of the injection; or
wherein the injection occurs at an opening on the catheter at the proximal location where the stent feeds into the catheter, such that the fluid fills the stent as it proceeds retrograde and that the cone of the stent into the catheter prevents any antegrade flow; or
wherein prior to injection, the contrast is injected through the stent to confirm that the cystic duct and gallbladder are not opacified, and that the biliary tree becomes opacified; or
wherein the stent is made out of solid material, such that fluid cannot go through the walls of the stent; or
wherein the liver end of the stent is opened in the left or right main hepatic duct and not in the common hepatic duct; or
wherein once hydrodynamic injection is complete from either the left or right main hepatic duct, the alternative duct is injected using the same technique; or
wherein the stent is partially deployed, meaning only at most 95% of its length, 75% of its length, 50% of its length, or 25% of its length in some embodiments is deployed outside the catheter, with the rest of the length of the stent remaining within or attached to the catheter; or
wherein the partially deployed stent forms a funnel or cone shape at its proximal end where it is attached to the catheter, thereby forming a closed system.

243. The method of claim 238, wherein the biliary stent is placed prior to hydrodynamic injection; or
wherein the biliary stent is placed over the cystic duct in order to fluid from entering the cystic duct; or
wherein the biliary stent is at minimum the diameter of the bile duct, in order to provide sufficient seal with the duct walls and the stent during the injection; or
wherein the biliary stent is of variable length and can reach from the ampulla to the past the cystic duct; or
wherein the balloon catheter is inserted through the stent after placement; or
wherein the balloon catheter can be located anywhere in the stent, including the common bile duct or the common hepatic duct; or
wherein prior to injection, the contrast is injected through the stent to confirm that the cystic duct and gallbladder are not opacified, and that the biliary tree becomes opacified; or
wherein DNA solution is injected through at set parameters in order to mediate gene delivery into different cells within the liver; or
wherein a preferred flow rate during the injection is at minimum 1 mL/sec, or at least 2 mL/sec; or
wherein the preferred injection pressure is at least 50 mmHg, or at least 80 mmHg; or
wherein the preferred injection volume is at least 30, 40, 50, or 60 mL per kilogram of liver weight; or
wherein the preferred DNA dose is at least 20 mg per kg liver weight, or larger; or
wherein the preferred DNA concentration of the injection solution is at least 0.5 mg/ml DNA or more; or
wherein the stent is made out of solid material, such that fluid cannot go through the walls of the stent.

244. The method of claim 238, wherein the injection parameters achieve gene expression in ductal cells, islet cells, acinar cells, endothelial cells, and neurons; or
wherein alternatively the total volume injected does not exceed 15 mL for pancreas weighing more than 60 grams; or
wherein if a flow rate of 2 mL/sec is utilized, then a maximum volume of 0.15 mL per gram of pancreas weight is used; or
wherein the DNA dose is preferably greater than 20, or 30 micrograms per gram of pancreas weight; or
wherein amylase or lipase levels are maximally increased 4-fold at day 1 post-injection; or
wherein the catheter can be placed:
(i) through the major duodenal papilla into the main pancreatic duct, distal to the portion of where the pancreatic duct the fuses with the common bile duct, or
(ii) through the minor duodenal papilla into the accessory or dorsal pancreatic duct, with option to advance farther into the main pancreatic duct; or
wherein inflation of a balloon in the catheter near the entrance to the pancreatic duct past the common bile duct to prevent retrograde flow of fluid; or
wherein the maximal balloon size used for sealing the pancreatic duct is 9 mm to avoid injury; or
wherein alternatively two or more flow rate are used during hydrodynamic injection in order to further minimize pancreatic tissue injury while preserving gene delivery; or
wherein the flow rate is initially 1 mL/sec for the first 50% of the injection volume and then increased to 2 mL/sec for the remaining injected volume; or
wherein the flow rate is initially 0.5 mL/sec for the first 50% of the injection volume and then increased to 1.5 mL/sec for the remaining injected volume; or
wherein a side-wall injection catheter is not used to avoid ductal wall injury and prevent pancreatitis.

245. A method of delivery of a non-viral DNA vector into a tumor(s) of the liver, compromising the steps of:
placing a catheter into a biliary system, preferably in a common hepatic duct;
inflating a balloon in the common hepatic duct to prevent antegrade flow; and
injecting a DNA solution at hydrodynamic pressure into the biliary system
wherein an injection achieves expression of the non-viral DNA vector within the tumor cells, regardless of the tumor location within the liver; or
A method of delivery of a non-viral DNA vector into the tumor(s) of the pancreas, compromising the steps of:
a) placing a catheter into a pancreatic ductal system, upstream of the tumor;
b) inflating a balloon in the pancreatic duct to prevent antegrade flow; and
c) injecting DNA solution at a hydrodynamic pressure into the pancreatic ductal system;
wherein the injection achieves expression of the non-viral DNA vector within the tumor cells, regardless of the tumor location within the pancreas; or
A method of determining a flow rate for a hydrodynamic injection into an organ, comprising the steps of:
performing a test injection;
measuring a pressure during the test injection; and
evaluating a difference in stiffness and resistance empirically; or
A method of hydrodynamic injection into the gallbladder or liver, compromising:
a) placing a catheter in the common bile duct;
b) inflating a balloon in the common bile duct to prevent antegrade flow;
c) injecting a DNA solution at high pressure target and/or flow rate into the biliary system,
wherein gene expression can be observed in hepatocytes within the liver on immunostaining and cells within the gallbladder.

246. The method of claim 245, wherein the tumor is in proximity to the biliary system in order to achieve efficient delivery; or
wherein a pressure of at least 50 mmHg, 70 mmHg, or at least 120 mmHg is targeted for an efficient tumor gene delivery; or
wherein a flow rate of at least 2 mL/sec, 4 mL/sec, 7 mL/sec, or at least 10 ml/sec is utilized to achieve the efficient tumor gene delivery; or
wherein a volume of at least 30 mL per kg of liver weight is utilized for the injection; or
wherein a non-viral DNA dose of at least 10 mg per kg liver weight, or at least 20 mg per kg liver weight it used for the injection; or
wherein gene expression in tumor cells is highest along the rim of the tumor.

247. The method of claim 245, wherein the tumor is in proximity to the ductal system in order to achieve efficient delivery; or
wherein the pressure of at least 50 mmHg, 70 mmHg, or at least 120 mmHg is controlled for efficient tumor gene delivery; or
wherein the flow rate of at least 1 mL/sec is controlled to achieve efficient tumor delivery; or
wherein the volume of at least 8 mL is injected into the pancreas of an adult human; or
wherein the non-viral DNA dose of at least 1 mg is injected in the pancreas of an adult human; or
wherein the gene expression in tumor cells is highest along the rim of the pancreatic tumor; or
wherein the tumor delivery is most efficient for a pancreatic ductal adenocarcinoma.

248. The method of claim 245, wherein the organ is selected from a liver, pancreas, or kidney; or
wherein the liver is injected through a biliary system; or
wherein the pancreas is injected through a ductal system; or
wherein the kidney is injected through a ureter system; or
wherein a pressure sensor will be inserted into a catheter through a dedicated lumen; or
wherein the pressure sensor is already present in the catheter; or
wherein the method further comprising the steps of:
reading a baseline of the pressure in the biliary system conducted without the balloon inflated;
performing a pressure measurement in the biliary system with balloon inflated;
injecting a test solution or a nucleic acid into a duct or a vessel of interest that does not contain the plasmid DNA with the balloon inflated and monitoring the pressure during the test solution injection; or
wherein the test solution has the same osmolarity, osmolality, and viscosity as the DNA injection solution; or
wherein the test solution further does not contain any other active drug substances included in the therapeutic DNA solution; or
wherein the pressure achieved during hydrodynamic injection is at least 50 mmHg, at least 80 mmHg, or at least 120 mmHg; or
wherein the test flow rate is initially at least 2 mL/sec, or at least 3 mL/sec into the liver; or
wherein the total test volume is at most 40 mL, at most 30 mL or at most 20 mL, at most 10 mL, or at most 5 mL of volume; or
wherein the total test volume is sufficient to measure the column of total fluid resistance in the entire circuit and estimating whether sufficient pressure is achieved; or
wherein the flow rate is increased if the proper pressure is not achieved, repeating the test; or
wherein the flow rate is decreased if the pressure above 250 mmg is achieved, repeating the test; or
wherein the flow rate is increased or decreased by 1 mL/sec or increased or decreased by 0.5 mL/sec for the second test; or
wherein the DNA solution injection proceeds at a programmed flow rate established that will yield the proper pressure; or
wherein a flow rate series is conducted during one single test injection, wherein multiple flow rates are tested and pressure measured throughout the single test injection; or
wherein at least two or more test flow rates are tested within a single test injection; or
wherein the pressure can be correlated to which flow rate yielded the increase; or
wherein the total injection volume is increased to at most 40 mL, 35 mL or 20 mL in total volume, testing all of the injection parameters; or
wherein the minimum pressure for efficient hydrodynamic gene delivery is greater than 50 mmHg, or greater than 80 mmHg, or greater than 100 mmHg; or
wherein the maximum pressure for efficient hydrodynamic gene delivery is less than 200 mmHg or less than 250 mmHg.

249. The method of claim 245, wherein the high pressure target is greater than 50 mmHg, greater than 80 mmHg, or greater than 120 mmHg; or
wherein the flow rate is greater than 2 mL/sec, greater than 5 mL/sec, or greater than 10 mL/sec; or
wherein the volume injected is greater than 50 mL per kg of liver weigh, or greater then 75 mL per kg of liver weight, or greater than 100 mL per kg of liver weight; or
wherein prior to injection, bile is removed from the biliary system, and normal saline solution is used to flush and wash the biliary system; or
wherein saline solution can be optionally used to fill the gallbladder prior to injection to reduce the pressure differential during injection; or
wherein the preferred DNA dose is at least 20 mg per kg liver weight, or larger; or
wherein the preferred DNA concentration of the injection solution is at least 0.5 mg/mL DNA or more.
